(19) United States
(12) Patent Application Publication
(10) Pub. No.: US 2004/0014038 A1
(43) Pub. Date: Jan. 22, 2004

(54) Novel GPCR-like proteins and nucleic acids encoding same

(76) Inventors: Stacie J. Casman, North Haven, CT (US); Shlomit R. Edinger, New Haven, CT (US); Karen Ellerman, Branford, CT (US); Glennda Smithson, Guilford, CT (US); Ramesh Kekuda, Branford, CT (US); Muralidhara Padigaru, Bronx, NY (US)

Correspondence Address: Ivor R. Elrifi, Ph.D., MINTZ, LEVIN, COHN, FERRIS,, GLOVSKY and POPEO, P.C., One Financial Center, Boston, MA, 02111, US

(21) Appl. No.: 10/041,615

(22) Filed: Jan. 3, 2002

Related U.S. Application Data
(60) Provisional application No. 60/259,552, filed on Jan. 3, 2001.
(60) Provisional application No. 60/277,405, filed on Mar. 20, 2001.
(60) Provisional application No. 60/260,544, filed on Jan. 9, 2001.

(51) Int. Cl. C12Q 1/68; G01N 33/574; C07H 21/04; C12P 21/02; C12N 5/06; C07K 14/705; C07K 16/30; A61K 39/395

(52) U.S. Cl. 435/6; 435/7.23; 435/69.1; 435/320.1; 435/325; 530/350; 536/23.5; 530/388.22; 424/143.1

(57) ABSTRACT

Disclosed herein are nucleic acid sequences that encode G-coupled protein-receptor related polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from U.S. provisional patent application serial No. 60/259,552 filed Jan. 3, 2001 (attorney docket CURA-533); Ser. No. 60/277,405 filed Mar. 20, 2001 (attorney docket CURA-533a); and Ser. No. 60/260,544 filed Jan. 09, 2001 (attorney docket CURA-538) each of which is incorporated herein by reference in its entirety.

### FIELD OF THE INVENTION

The invention generally relates to novel GPCR1, GPCR2, GPCR3, GPCR4a, GPCR4b, GPCR5, GPCR6, GPCR7, GPCR8, GPCR9, GPCR10, GPCR11, GPCR12, GPCR13, GPCR14, GPCR15, and GPCR16 nucleic acids and polypeptides encoded therefrom. More specifically, the invention relates to nucleic acids encoding novel polypeptides, as well as vectors, host cells, antibodies, and recombinant methods for producing these nucleic acids and polypeptides.

### BACKGROUND OF THE INVENTION

The invention generally relates to nucleic acids and polypeptides. More particularly, the invention relates to nucleic acids encoding novel G-protein coupled receptor (GPCR) polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as GPCR1, GPCR2, GPCR3, GPCR4a, GPCR4b, GPCR5, GPCR6, GPCR7, GPCR8, GPCR9, GPCR10, GPCR11, GPCR12, GPCR13, GPCR14, GPCR15, and GPCR16 nucleic acids and polypeptides. These nucleic acids and polypeptides, as well as derivatives, homologs, analogs and fragments thereof, will hereinafter be collectively designated as “GPCRX” nucleic acid or polypeptide sequences.

In one aspect, the invention provides an isolated GPCRX nucleic acid molecule encoding a GPCRX polypeptide that includes a nucleic acid sequence that has identity to the nucleic acids disclosed in SEQ ID NOS: 1, 3, 5, 7, 9, 11, 13, 15, 17, 19, 21, 23, 25, 27, 29, 31,and 33. In some embodiments, the GPCRX nucleic acid molecule will hybridize under stringent conditions to a nucleic acid sequence complementary to a nucleic acid molecule that includes a protein-coding sequence of a GPCRX nucleic acid sequence. The invention also includes an isolated nucleic acid that encodes a GPCRX polypeptide, or a fragment, homolog, analog or derivative thereof. For example, the nucleic acid can encode a polypeptide at least 80% identical to a polypeptide comprising the amino acid sequences encoded by SEQ ID NOS: 1, 3, 5, 7, 9, 11, 13, 15, 17, 19, 21, 23, 25, 27, 29, 31, and 33. The nucleic acid can be, for example, a genomic DNA fragment or a cDNA molecule that includes the nucleic acid sequence of any of SEQ ID NOS: 1, 3, 5, 7, 9, 11, 13, 15, 17, 19, 21, 23, 25, 27, 29, 31, and 33.

Also included in the invention is an oligonucleotide, e.g., an oligonucleotide which includes at least 6 contiguous nucleotides of a GPCRX nucleic acid (e.g., SEQ ID NOS: 1, 3, 5, 7, 9, 11, 13, 15, 17, 19, 21, 23, 25, 27, 29, 31, and 33) or a complement of said oligonucleotide.

Also included in the invention are substantially purified GPCRX polypeptides (SEQ ID NOS: 2, 4, 6, 8, 10, 12, 14, 16, 18, 20, 22, 24, 26, 28, 30, 32, and 34). In certain embodiments, the GPCRX polypeptides include an amino acid sequence that is substantially identical to the amino acid sequence of a human GPCRX polypeptide.

The invention also features antibodies that immunoselectively bind to GPCRX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a GPCRX nucleic acid, a GPCRX polypeptide, or an antibody specific for a GPCRX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a GPCRX nucleic acid, under conditions allowing for expression of the GPCRX polypeptide encoded by the DNA. If desired, the GPCRX polypeptide can then be recovered.

In another aspect, the invention includes a method of detecting the presence of a GPCRX polypeptide in a sample. In the method, a sample is contacted with a compound that selectively binds to the polypeptide under conditions allowing for formation of a complex between the polypeptide and the compound. The complex is detected, if present, thereby identifying the GPCRX polypeptide within the sample.

The invention also includes methods to identify specific cell or tissue types based on their expression of a GPCRX.

Also included in the invention is a method of detecting the presence of a GPCRX nucleic acid molecule in a sample by contacting the sample with a GPCRX nucleic acid probe or primer, and detecting whether the nucleic acid probe or primer bound to a GPCRX nucleic acid molecule in the sample.

In a further aspect, the invention provides a method for modulating the activity of a GPCRX polypeptide by contacting a cell sample that includes the GPCRX polypeptide with a compound that binds to the GPCRX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., developmental diseases; MHCII and III diseases (immune diseases); taste and scent detectability disorders; Burkitt's lymphoma; corticoneurogenic disease; signal transduction pathway disorders; metabolic pathway disorders; retinal diseases including those involving photoreception; cell growth rate disorders; cell shape disorders; metabolic disorders; feeding disorders; control of feeding; the metabolic syndrome X; wasting disorders associated with chronic diseases; obesity; potential obesity due to over-eating or metabolic disturbances; potential disorders due to starvation (lack of appetite); diabetes; noninsulin-dependent diabetes mellitus (NIDDM1); infectious disease; bacterial, fungal, protozoal and viral infections (particularly infections caused by HIV-1 or HIV-2); pain; cancer (including but not limited to neoplasm; adenocarcinoma; lymphoma; prostate cancer; uterus cancer); cancer-associated cachexia; anorexia; bulimia; asthma; Parkinson's disease; acute heart failure; hypotension; hypertension; urinary retention; osteoporosis; Crohn's disease; multiple sclerosis; Albright hereditary ostoeodystrophy; angina pectoris; myocardial infarction; ulcers; allergies; benign prostatic hypertrophy; and psychotic and neurological disorders; including anxiety; schizophrenia; manic depression; delirium; dementia; neurodegenerative disorders; Alzheimer's disease; severe mental retardation; dentatorubro-pallidoluysian atrophy (DRPLA); hypophosphatemic rickets; autosomal dominant (2) acrocallosal syndrome and dyskinesias, such as Huntington's disease or Gilles de la Tourette syndrome; immune disorders; adrenoleukodystrophy; congenital adrenal hyperplasia; hemophilia; hypercoagulation; idiopathic thrombocytopenic purpura; autoimmume disease; immunodeficiencies; transplantation; Von Hippel-Lindau (VHL) syndrome; stroke; tuberous sclerosis; hypercalceimia; cerebral palsy; epilepsy; Lesch-Nyhan syndrome; ataxia-telangiectasia; leukodystrophies; behavioral disorders; addiction; neuroprotection; cirrhosis; transplantation; systemic lupus erythematosus; emphysema; scleroderma; ARDS; renal artery stenosis; interstitial nephritis; glomerulonephritis; polycystic kidney disease; renal tubular acidosis; IgA nephropathy; cardiomyopathy; atherosclerosis; congenital heart defects; aortic stenosis; atrial septal defect (ASD); atrioventricular (A-V) canal defect; ductus arteriosus; pulmonary stenosis subaortic stenosis; ventricular septal defect (VSD); valve diseases; scleroderma; fertility; pancreatitis; endocrine dysfunctions; growth and reproductive disorders; inflammatory bowel disease; diverticular disease; graft vesus host disease; hyperthyroidism; endometriosis; hematopoietic disorders and/or other pathologies and disorders of the like. The therapeutic can be, e.g., a GPCRX nucleic acid, a GPCRX polypeptide, or a GPCRX-specific antibody, or biologically-active derivatives or fragments thereof.

For example, the compositions of the present invention will have efficacy for treatment of patients suffering from the diseases and disorders listed above and/or other pathologies and disorders.

The polypeptides can be used as immunogens to produce antibodies specific for the invention, and as vaccines. They can also be used to screen for potential agonist and antagonist compounds. For example, a cDNA encoding GPCRX may be useful in gene therapy, and GPCR-X may be useful when administered to a subject in need thereof. By way of nonlimiting example, the compositions of the present invention will have efficacy for treatment of patients suffering the diseases and disorders listed above and/or other pathologies and disorders.

The invention further includes a method for screening for a modulator of disorders or syndromes including, e.g., diseases and disorders listed above and/or other pathologies and disorders and those disorders related to cell signal processing and metabolic pathway modulation. The method includes contacting a test compound with a GPCRX polypeptide and determining if the test compound binds to said GPCRX polypeptide. Binding of the test compound to the GPCRX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including the diseases and disorders listed above and/or other pathologies and disorders or other disorders related to cell signal processing and metabolic pathway modulation by administering a test compound to a test animal at increased risk for the aforementioned disorders or syndromes. The test animal expresses a recombinant polypeptide encoded by a GPCRX nucleic acid. Expression or activity of GPCRX polypeptide is then measured in the test animal, as is expression or activity of the protein in a control animal which recombinantly-expresses GPCRX polypeptide and is not at increased risk for the disorder or syndrome. Next, the expression of GPCRX polypeptide in both the test animal and the control animal is compared. A change in the activity of GPCRX polypeptide in the test animal relative to the control animal indicates the test compound is a modulator of latency of the disorder or syndrome.

In yet another aspect, the invention includes a method for determining the presence of or predisposition to a disease associated with altered levels of a GPCRX polypeptide, a GPCRX nucleic acid, or both, in a subject (e.g., a human subject). The method includes measuring the amount of the GPCRX polypeptide in a test sample from the subject and comparing the amount of the polypeptide in the test sample to the amount of the GPCRX polypeptide present in a control sample. An alteration in the level of the GPCRX polypeptide in the test sample as compared to the control sample indicates the presence of or predisposition to a disease in the subject. Preferably, the predisposition includes diseases and disorders listed above and/or other pathologies and disorders. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a GPCRX polypeptide, a GPCRX nucleic acid, or a GPCRX-specific antibody to a subject (e.g., a human subject), in an amount sufficient to alleviate or prevent the pathological condition. In preferred embodiments, the disorder, includes the diseases and disorders listed above and/or other pathologies and disorders.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The invention is based, in part, upon the discovery of novel nucleic acid sequences that encode novel polypeptides. The novel nucleic acids and their encoded polypeptides are referred to individually as GPCR1, GPCR2, GPCR3, GPCR4a, GPCR4b, GPCR5, GPCR6, GPCR7, GPCR8, GPCR9, GPCR10, GPCR11, GPCR12, GPCR13, GPCR14, GPCR15 and GPCR16. The nucleic acids, and their encoded polypeptides, are collectively designated herein as “GPCRX”.

The novel GPCRX nucleic acids of the invention include the GPCR1, GPCR2, GPCR3, GPCR4a, GPCR4b, GPCR5, GPCR6, GPCR7, GPCR8, GPCR9, GPCR10, GPCR11, GPCR12, GPCR13, GPCR14, GPCR15, and GPCR16 nucleic acids, or a fragment, derivative, analog or homolog thereof. The novel GPCRX proteins of the invention include the GPCR1, GPCR2, GPCR3, GPCR4a, GPCR4b, GPCR5, GPCR6, GPCR7, GPCR8, GPCR9, GPCR10, GPCR11, GPCR12, GPCR13, GPCR14, GPCR15, and GPCR16 proteins, or a derivative, analog or homolog thereof. The individual GPCRX nucleic acids and proteins are described below. Within the scope of this invention is a method of using these nucleic acids and peptides in the treatment or prevention of a disorder related to cell signaling or metabolic pathway modulation.

The GPCRX proteins of the invention have a high homology to the 7tm_{—}1 domain (PFam Acc. No. pfam00001). The 7tm_{—}1 domain is from the 7 transmembrane receptor family, which includes a number of different proteins, including, for example, serotonin receptors, dopamine receptors, histamine receptors, andrenergic receptors, cannabinoid receptors, angiotensin II receptors, chemokine receptors, opioid receptors, G-protein coupled receptor (GPCR) proteins, olfactory receptors (OR), and the like. Some proteins and the Protein Data Base Ids/gene indexes include, for example: 5-hydroxytryptamine receptors (See, e.g., PMIM 112821, 8488960, 112805, 231454, 1168221, 398971, 112806); rhodopsin (129209); G protein-coupled receptors (119130, 543823, 1730143, 132206, 137159, 6136153, 416926, 1169881, 136882, 134079); gustatory receptors (544463, 462208); c-x-c chemokine receptors (416718, 128999, 416802, 548703, 1352335); opsins (129193, 129197, 129203); and olfactory receptor-like proteins (129091, 1171893, 400672, 548417).

Because of the close homology among the members of the GPCRX family, proteins that are homologous to any one member of the family are also largely homologous to the other members, except where the sequences are different as shown below.

The similarity information for the GPCRX proteins and nucleic acids disclosed herein suggest that GPCR1-GPCR16 may have important structural and/or physiological functions characteristic of the Olfactory Receptor family and the GPCR family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

G-Protein Coupled Receptor proteins (“GPCRs”) have been identified as a large family of G protein-coupled receptors in a number of species. These receptors share a seven transmembrane domain structure with many neurotransmitter and hormone receptors, and underlie the recognition and G-protein-mediated transduction of various signals. Human GPCR's generally do not contain introns and belong to four different gene subfamilies, displaying great sequence variability. These genes are dominantly expressed in olfactory epithelium. See, e.g., Ben-Arie et al., Hum. Mol. Genet. 1994 3:229-235; and, Online Mendelian Inheritance in Man (“OMIM”) entry #164342 (http://www.ncbi.nlm.nih.gov/entrez/dispomim.cgi?), incorporated by reference.

The olfactory receptor (“OR”) gene family constitutes one of the largest GPCR multigene families and is distributed among many chromosomal sites in the human genome. See Rouquier et al., Hum. Mol. Genet. 7(9):1337-45 (1998); Malnic et al., Cell 96:713-23 (1999), incorporated by reference. Olfactory receptors constitute the largest family among G protein-coupled receptors, with up to 1000 members expected. See Vanderhaeghen et al., Genomics 39(3):239-46 (1997); Xie et al., Mamm. Genome 11(12):1070-78 (2000 ); Issel-Tarver et al., Proc. Natl. Acad. Sci. USA 93(20):10897-902 (1996), incorporated by reference. The recognition of odorants by olfactory receptors is the first stage in odor discrimination. See Krautwurst et al., Cell 95(7):917-26 (1998); Buck et al., Cell 65(1):175-87 (1991), incorporated by reference. Many ORs share some characteristic sequence motifs and have a central variable region corresponding to a putative ligand binding site. See Issel-Tarver et al., Proc. Natl. Acad. Sci. USA 93:10897-902 (1996), incorporated by reference.

Other examples of seven membrane spanning proteins that are related to GPCR's are chemoreceptors. See Thomas et al., Gene 178(1-2):1-5 (1996), incorporated by reference. Chemoreceptors have been identified in taste, olfactory, and male reproductive tissues. See id.;

Walensky et al., J. Biol. Chem. 273(16):9378-87 (1998); Parmentier et al., Nature 355(6359):453-55 (1992); Asai et al., Biochem. Biophys. Res. Commun. 221(2):240-47 (1996), incorporated by reference.

The GPCRX nucleic acids of the invention encoding GPCR-like proteins include the nucleic acids whose sequences are provided herein, or fragments thereof. The invention also includes mutant or variant nucleic acids any of whose bases may be changed from the corresponding base shown herein while still encoding a protein that maintains its GPCR-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

The GPCRX proteins of the invention include the GPCR-like proteins whose sequences are provided herein. The invention also includes mutant or variant proteins any of whose residues may be changed from the corresponding residue shown herein while still encoding a protein that maintains its GPCR-like activities and physiological functions, or a functional fragment thereof. The invention further encompasses antibodies and antibody fragments, such as F_{ab} or (F_{ab})₂ that bind immunospecifically to any of the proteins of the invention.

The GPCRX nucleic acids and proteins are useful in potential therapeutic applications implicated in various GPCR-related pathological disorders and/or OR-related pathological disorders, described further below. For example, a cDNA encoding the GPCR (or olfactory-receptor) like protein may be useful in gene therapy, and the receptor-like protein may be useful when administered to a subject in need thereof. The nucleic acids and proteins of the invention are also useful in potential therapeutic applications used in the treatment of developmental diseases; MHC II and III diseases (immune diseases); taste and scent detectability disorders; Burkitt's lymphoma; corticoneurogenic disease; signal transduction pathway disorders; metabolic pathway disorders; retinal diseases including those involving photoreception; cell growth rate disorders; cell shape disorders; metabolic disorders; feeding disorders; control of feeding; the metabolic syndrome X; wasting disorders associated with chronic diseases; obesity; potential obesity due to over-eating or metabolic disturbances; potential disorders due to starvation (lack of appetite); diabetes; noninsulin-dependent diabetes mellitus (NIDDM 1); infectious disease; bacterial, fungal, protozoal and viral infections (particularly infections caused by HIV-1 or HIV-2); pain; cancer (including but not limited to neoplasm; adenocarcinoma; lymphoma; prostate cancer; uterus cancer); cancer-associated cachexia; anorexia; bulimia; asthma; Parkinson's disease; acute heart failure; hypotension; hypertension; urinary retention; osteoporosis; Crohn's disease; multiple sclerosis; Albright Hereditary Ostoeodystrophy; angina pectoris; myocardial infarction; ulcers; allergies; benign prostatic hypertrophy; and psychotic and neurological disorders; including anxiety; schizophrenia; manic depression; delirium; dementia; neurodegenerative disorders; Alzheimer's disease; severe mental retardation; Dentatorubro-pallidoluysian atrophy (DRPLA); Hypophosphatemic rickets; autosomal dominant (2) Acrocallosal syndrome and dyskinesias, such as Huntington's disease or Gilles de la Tourette syndrome; immune disorders; Adrenoleukodystrophy; Congenital Adrenal Hyperplasia; Hemophilia; Hypercoagulation; Idiopathic thrombocytopenic purpura; autoimmume disease; immunodeficiencies; transplantation; Von Hippel-Lindau (VHL) syndrome; Stroke; Tuberous sclerosis; hypercalceimia; Cerebral palsy; Epilepsy; Lesch-Nyhan syndrome; Ataxia-telangiectasia; Leukodystrophies; Behavioral disorders; Addiction; Neuroprotection; Cirrhosis; Transplantation; Systemic lupus erythematosus; Emphysema; Scleroderma; ARDS; Renal artery stenosis; Interstitial nephritis; Glomerulonephritis; Polycystic kidney disease; Systemic lupus erythematosus; Renal tubular acidosis; IgA nephropathy; Cardiomyopathy; Atherosclerosis; Congenital heart defects; Aortic stenosis ; Atrial septal defect (ASD); Atrioventricular (A-V) canal defect; Ductus arteriosus; Pulmonary stenosis; Subaortic stenosis; Ventricular septal defect (VSD); valve diseases; Scleroderma; fertility; Pancreatitis; Endocrine dysfunctions; Growth and reproductive disorders; Inflammatory bowel disease; Diverticular disease; Leukodystrophies; Graft vesus host; Hyperthyroidism; Endometriosis; hematopoietic disorders and/or other pathologies and disorders. Other GPCR-related diseases and disorders are contemplated.

The polypeptides can be used as immunogens to produce antibodies specific for the invention, and as vaccines. They can also be used to screen for potential agonist and antagonist compounds. For example, a cDNA encoding the GPCR-like protein may be useful in gene therapy, and the GPCR-like protein may be useful when administered to a subject in need thereof. By way of nonlimiting example, the anti-GPCRX antibody compositions of the present invention will have efficacy for treatment of patients suffering from the diseases and disorders listed above, as well as other related or associated pathologies. The novel nucleic acid encoding GPCR-like protein, and the GPCR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

GPCR1

A GPCR-like protein of the invention, referred to herein as GPCR1, is an Olfactory Receptor (“OR”)-like protein. Some members of the Olfactory Receptor-Like Protein Family localize to the cell surface, where they exhibit activity. Therefore it is likely that these novel GPCR1 proteins are available at the appropriate sub-cellular localization and hence accessible for the therapeutic uses described in this application.

In one embodiment, the disclosed GPCR1 variant is the novel GPCR1a (alternatively referred to as CG55956-01), which includes the 947 base nucleotide sequence (SEQ ID NO: 1) shown in Table 1A. The disclosed GPCR1 open reading frame (“ORF”) begins at an ATG initiation codon at nucleotides 17-19 and terminates at a TGA codon at nucleotides 932-934.

Putative untranslated regions upstream from the initiation codon and downstream from the termination codon are underlined in Table 1A, and the start and stop codons are in bold letters.

TABLE 1A
GPCR1 polynucleotide sequence.

(SEQ ID NO:1)

TGAAACGAATAACTCTATGGTGACTGAATTCATTTTTCTGGGTCTCTCTGATTCTCAGGAACTCCAGACC
TTCCTATTTATGTTGTTTTTTGTATTCTATGGAGGAATCGTGTTTGGAAACCTTCTTATTGTCATAACAG
TGGTATCTGACTCCCACCTTCACTCTCCCATGTACTTCCTGCTAGCCAACCTCTCACTCATTGATCTGTC
TCTGTCTTCAGTCACAGCCCCCAAGATGATTACTGACTTTTTCAGCCAGCGCAAAGTCATCTCTTTCAAG
GGCTGCCTTGTTCAGATATTTCTCCTTCACTTCTTTGGTGGGAGTGAGATGGTGATCCTCATAGCCATGG
GCTTTGACAGATATATAGCAATATGCAAGCCCCTACACTACACTACAATTATGTGTGGCAACGCATGTGT
CGGCATTATGGCTGTCACATGGGGAATTGGCTTTCTCCATTCGGTGAGCCAGTTGGCGTTTGCCGTGCAC
TTACTCTTCTGTGGTCCCAATGAGGTCGATAGTTTTTATTGTGACCTTCCTAGGGTAATCAAACTTGCCT
GTACAGATACCTACAGGCTAGATATTATGGTCATTGCTAACAGTGGTGTGCTCACTGTGTGTTCTTTTGT
TCTTCTAATCATCTCATACACTATCATCCTAATGACCATCCAGCATCGCCCTTTAGATAAGTCGTCCAAA
GCTCTGTCCACTTTGACTGCTCACATTACAGTAGTTCTTTTGTTCTTTGGACCATGTGTCTTTATTTATG
CCTGGCCATTCCCCATCAAGTCATTAGATAAATTCCTTGCTGTATTTTATTCTGTGATCACCCCTCTCTT
GAACCCAATTATATACACACTGAGGAACAAAGACATGAAGACGGCAATAAGACAGCTGAGAAAATGGGAT
GCACATTCTAGTGTAAAGTTTTAGATCTTATATAACT

The disclosed sequence of GPCR1 was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The disclosed GPCR1 of this invention maps to chromosome 1. Chromosome localization information was assigned using OMIM, the electronic northern bioinformatic tool implemented by CuraGen Corporation, public ESTs, public literature references and/or genomic clone homologies. This was executed to derive the chromosomal mapping of the SeqCalling assemblies, Genomic clones, literature references and/or EST sequences that were included in the invention.

The disclosed GPCR1 polypeptide (SEQ ID NO: 2) encoded by SEQ ID NO: 1 has 305 amino acid residues, and is presented in Table 1B using the one-letter amino acid codes. The Psort profile for GPCR1 predicts that this sequence has a signal peptide and is likely to be a Type IIIb membrane protein, localized at the plasma membrane with a certainty of 0.6000. In alternative embodiments, a GPCR1 polypeptide is located to the Golgi body with a certainty of 0.4000, to the endoplasmic reticulum (membrane) with a certainty of 0.3000, or to the peroxisomal microbody with a certainty of 0.3000. The Signal P predicts a likely cleavage site for a GPCR1 peptide is between positions 13 and 14, i.e., at the dash in the sequence SDS-QE.

TABLE 1B
Encoded CPCR1 protein sequence.

(SEQ ID NO:2)

MVTEFIFLGLSDSQELQTFLFMLFFVFYGGIVFGNLLIVITVVSDSHLHSPMYFLLANLSLIDLSLSSVT
APKMITDFFSQRKVISFKGCLVQIFLLHFFGGSEMVILIAMGFDRYIAICKPLHYTTIMCGNACVGIMAV
TWGIGFLHSVSQLAFAVHLLFCGPNEVDSFYCDLPRVIKLACTDTYRLDIMVIANSGVLTVCSFVLLIIS
YTIILMTIQHRPLDKSSKALSTLTAHITVVLLFFGPCVFIYAWPFPIKSLDKFLAVFYSVITPLLNPIIY
TLRNKDMKTAIRQLRKWDAHSSVKF

Public and proprietary sequence databases were searched for protein sequences with homology to GPCR1 using BLASTP software. In all BLAST alignments herein, the “E-value” or “Expect” value is a numeric indication of the probability that the aligned sequences could have achieved their similarity to the BLAST query sequence by chance alone, within the database that was searched. The Expect value (E) is a parameter that describes the number of hits one can “expect” to see just by chance when searching a database of a particular size. It decreases exponentially with the Score (S) that is assigned to a match between two sequences of a database of comparable complexity. Essentially, the E value describes the random background noise that exists for matches between sequences.

The E value is used as a convenient way to create a significance threshold for reporting results. The default value used for blasting is typically set to 0.0001. In BLAST 2.0, the E value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNN”) or the letter “X” in protein sequences (e.g., “XXX”). Low-complexity regions can result in high scores that reflect compositional bias rather than significant position-by-position alignment (Wootton and Federhen, Methods Enzymol 266:554-571, 1996).

A BLAST analysis of GPCR1 was run against the proprietary PatP GENESEQ Protein Patent database. The amino acid sequence of GPCR1 has high homology to other proteins as shown in Table 1C.

TABLE 1C
BLASTX results from PatP database for GPCR1

| | | Smallest Sum |
|---|---|---|
| Sequences producing | High | Probability |
| High-scoring Segment Pairs: | Score | P(N) |
| patp: AAG71593 Human olfactory | 1573 | 2.5e−161 |
| receptor polypeptide | | |
| patp: AAG72243 Human olfactory | 1557 | 1.3e−159 |
| receptor polypeptide | | |
| patp: AAG72250 Human olfactory | 1554 | 2.6e−159 |
| receptor polypeptide | | |
| patp: AAG72356 Human OR-like | 1554 | 2.6e−159 |
| polypeptide query sequence | | |
| patp: AAG72237 Human olfactory | 1549 | 8.8e−159 |
| receptor polypeptide | | |

In a search of public sequence databases, it was found, for example, that the amino acid sequence of the protein of the invention was found to have 177 of 299 amino acid residues (59%) identical to the 312 amino acid ptnr:SPTREMBL-ACC:095015 protein from Homo sapiens (DJ0855D21.1 protein, E=1.3e-93).

GPCR1 also has homology to the proteins shown in the BLASTP data in Table 1D.

TABLE 1D
GPCR1 BLASTP results

| Gene Index/ | | Length | Identity | Positives | |
|---|---|---|---|---|---|
| Identifier | Protein/Organism | (aa) | (%) | (%) | Expect |
| gi\|15293733\|gb\| | olfactory receptor | 214 | 211/214 | 211/214 | e−100 |
| AAK95059.1\|(AF399574) | [Homo sapiens] | | (98%) | (98%) | |
| gi\|17476676\|ref\| | similar to olfactory receptor | 311 | 180/301 | 234/301 | 2e−87 |
| XP_063304.1(XM_063304) | [Homo sapiens] | | (59%) | (76%) | |
| gi\|17476706\|ref\| | similar to olfactory receptor | 311 | 180/301 | 234/301 | 2e−87 |
| XP_063318.1(XM_063318) | [Homo sapiens] | | (59%) | (76%) | |
| gi\|17464879\|ref\| | similar to OLFACTORY | 312 | 175/296 | 220/296 | 4e−84 |
| XP_069595.1(XM_069595) | RECEPTOR 4F3 | | (59%), | (74%) | |
| | [Homo sapiens] | | | | |
| gi\|17476680\|ref\| | similar to gene for odorant | 324 | 175/294 | 232/294 | 1e−82 |
| XP_063306.1(XM_063306) | receptor MOR83 | | (59%) | (78%) | |
| | [Homo sapiens] | | | | |

A sequence alignment is given in Table 1E, with the GPCR1 protein being shown on line 1 in a ClustalW analysis comparing the protein of the invention with the related protein sequences shown in Table 1D.

[image]

[image]

Domain Analysis

The results indicate that the GPCR1 protein contains the following protein domain (as defined by Interpro): domain name 7tm_{—}1 7 transmembrane receptor (rhodopsin family). DOMAIN results for GPCR1 were collected from the Conserved Domain Database (CDD) with Reverse Position Specific BLAST. This BLAST samples domains found in the Smart and Pfam collections.

As discussed below, all GPCRX proteins of the invention contain significant homology to the 7tm_{—}1 domain. This indicates that the GPCRX sequence has properties similar to those of other proteins known to contain this 7tm_{—}1 domain and similar to the properties of these domains. The 254 amino acid domain termed 7tm_{—}1 (SEQ ID NO: 40) (Pfam acc. no. 00001), a seven transmembrane receptor (rhodopsin family), is shown in Table 1F

TABLE 1F
7tm_1, 7 transmembrane receptor domain

(SEQ ID NO:40)

GNLLVILVILRTKKLRTPTNIFLLNLAVADLLFLLTLPPWALYYLVGGDWVFGDALCKLVGALFVVNGYASILLLTAISIDR
YLAIVHPLRYRRIRTPRRAKVLILLVWVLALLLSLPPLLFSWLRTVEEGNTTVCLIDFPEESVKRSYVLLSTLVGFVLPLLV
ILVCYTRILRTLRKKARSQRSLKRRSSSERKAAKMLLVVVVVFVLCWLPYHIVLLLDSLCLLSIWRVLPTALLITLWLAYVN
SCLNPIIY

The encoded GPCR1 polypeptide was identified as a member of the G protein receptor family due to the presence of a signature consensus sequence (SEQ ID NO: 41) shown in Table 1G below.

TABLE 1G
G-protein coupled receptors signature domain (SEQ ID NO: 41)

| | |
|---|---|
| Entry Name | G_PROTEIN_RECEPTOR |
| Entry Type | PATTERN |
| Primary | PS00237 |
| Accession | |
| Number | |
| Created/Last | 1 Apr. 1990/1 Jul. 1998 |
| Updated | |
| Description | G-protein coupled |
| | receptors signature. |
| Pattern | [GSTALIVMFYWC] - [GSTANCPDE] - {EDPKRH} - |
| | x(2) - [LIVMNQGA] - x(2) - [LIVMFT] - [GSTANC] - |
| | [LIVMFYWSTAC] - [DENH] -R- [FYWCSH] - |
| | x(2)- [LIVM] . |

Table 1 H lists the domain description from DOMAIN analysis results against GPCR1. This indicates that the GPCR1 sequence has properties similar to those of other proteins known to contain this domain as well as to the 254 amino acid 7tm domain (SEQ ID NO: 40).

The DOMAIN results are listed in Table 1H with the statistics and domain description. An alignment of GPCR1 (SEQ ID NO: 2) with the full 7tm_{—}1 domain, residues 1-254 (SEQ ID NO: 40), are shown in Table 1H.

TABLE 1H
Domain Analysis of GPCR1

| | | |
|---|---|---|
| | Score | E |
| PSSMs producing significant alignments: | (bits) | value |

| | | | |
|---|---|---|---|
| 7tm_1 (InterPro) | 7 transmembrane receptor (rhodopsin family) | 82.8 | 3e−17 |

| | | | |
|---|---|---|---|
| GPCR1: | 34 | GNLLIVITVVSDSHLHSPMYFLLANLSLIDLSLSSVTAPKMITDFFSQRKVISFKGCLVQ | 93 |
| | | \|\|\|\|+++ ++ \| +\| \| \|\|++ \|\| \| + \| \| + | |
| Sbjct: | 1 | GNLLVILVILRTKKLRTPTNIFLLNLAVADLLFLLTLPPWALYYLVGGDWVFGDALCKLV | 60 |
| GPCR1: | 94 | IFLLHFFGGSEMVILIAMGFDRYIAICKPLHYTTIMCGNACVGIMAVTWGIGFLHSVSQL | 153 |
| | | \| \| + +++\| \|+ \|\|\|+\|\| \|\| \| \| ++ + \| + \| \|+ \| | |
| Sbjct: | 61 | GALFVVNGYASILLLTAISIDRYLAIVHPLRYRRIRTPRRAKVLILLVWVLALLLSLPPL | 120 |
| GPCR1: | 154 | AFAVHLLFCGPNEVDSFYCDLPRVIKLACTDTYRLDIMVIANSGVLTVCSFVLLIISYTI | 213 |
| | | \|+ \| +\| + \| \|+ +\| + +\| + | |
| Sbjct: | 121 | LFSWLRTVEEGNTTVCLIDFPEESVKRSYVLLSTLVGFVLPLLVILVCYTRILRTLRKRA | 180 |
| GPCR1: | 214 | ILMTIQHRPLDKSSKALSTLTAHITVVLLFFGPC-VFIYAWPF------PIKSLDKFLAV | 266 |
| | | \| \|\| \| + \| +\| + \| + + + + + | |
| Sbjct: | 181 | RSQRSLKRRSSSERKAAKMLLVVVVVFVLCWLPYHIVLLLDSLCLLSIWRVLPTALLITL | 240 |
| GPCR1: | 267 | FYSVITPLLNPIIY 280 | |
| | | + + + \|\|\|\|\|\| | |
| Sbjct: | 241 | WLAYVNSCLNPIIY 254 | |

The rhodopsin-like GPCRs themselves represent a widespread protein family that includes hormone, neurotransmitter and light receptors, all of which transduce extracellular signals through interaction with guanine nucleotide-binding (G) proteins. Although their activating ligands vary widely in structure and character, the amino acid sequences of the receptors are very similar and are believed to adopt a common structural framework comprising 7 transmembrane (TM) helices. G-protein-coupled receptors (GPCRs) constitute a vast protein family that encompasses a wide range of functions (including various autocrine, paracrine and endocrine processes). They show considerable diversity at the sequence level, on the basis of which they can be separated into distinct groups. The term clan is use to describe the GPCRs, as they embrace a group of families for which there are indications of evolutionary relationship, but between which there is no statistically significant similarity in sequence. The currently known clan members include the rhodopsin-like GPCRs, the secretin-like GPCRs, the cAMP receptors, the fungal mating pheromone receptors, and the metabotropic glutamate receptor family.

The homologies shown in the tables above indicates that the GPCR1 sequences of the invention have properties similar to those of other proteins known to contain this/these domain(s) as well as properties similar to the properties of these domains.

The Olfactory Receptor-like GPCR1 disclosed in this invention is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources. Further expression data for GPCR1 is provided in Example 2.

The nucleic acids and proteins of GPCR1 are useful in potential therapeutic applications implicated in various GPCR-related pathological disorders and/or OR-related pathological disorders, described above and further herein. The novel GPCR1 nucleic acid encoding the GPCR-like protein of the invention, or fragments thereof, may further be useful in diagnostic applications, wherein the presence or amount of the nucleic acid or the protein are to be assessed.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-GPCRX Antibodies” section below. The disclosed GPCR1 protein has multiple hydrophilic regions, each of which can be used as an immunogen. In one embodiment, for example, a contemplated GPCR1 epitope comprises from about amino acids 65 to about 80. In another embodiment, for example, a GPCR1 epitope comprises from about amino acids 160 to about 180. In further embodiments, for example, a GPCR1 epitope comprises from about 215 to about 230, and from about 275 to about 305.

GPCR2

The disclosed novel GPCR2 (alternatively referred to herein as CG55952-01) includes the 948 nucleotide sequence (SEQ ID NO: 3) shown in Table 2A. A GPCR2 ORF begins with a Kozak consensus ATG initiation codon at nucleotides 4-6 and ends with a TAG codon at nucleotides 946-948. A putative untranslated region upstream from the initiation codon is underlined in Table 2A, and the start and stop codons are in bold letters.

TABLE 2A
GPCR2 Nucleotide Sequence

(SEQ ID NO:3)

TAAATGAGACCTAATAACAGCATTACAGAATTTGTCCTCCTGGGATTCTCTCAGGATCCTGGTATGCAAA
AAGAATTATTTGTCATGTTTTTATTCACATACGTTGTGACTGTGTTGGGGAACCAGCTCATTGTGGTGAC
TATCATTGCCAGCCCTTCCTTGGGCTCCCCAATGTACTTCTTCCTTGCCTGCCTGTCATTTATAGATGCT
GCATATTTCACTGTCATTTCTCCCAAATTGATTGTGGACTTACTCTGTGATAAAAAGACTATTTCCTTCC
AAACGTTCATGGGCCAACTATTTATAGACCACTTCTTTGGTGGTGCAGAGGCCTTCCTTCTGGTGGTGAT
GGCCTATGATCGCTATGTTGCCATCTGTAAGACATTGCACTATTTGACCATCATGACTCGACAGGTTTGT
ATCCTTGCATTGCTGGTGGCTGCGACAGGCGGTTTTGTGCATTCTGTGTTTCAAATTGTTGTTGTGTACA
GTCTCCCTTTCTGTGGCGCCAATGTCATTGATCATTTCAGTTGTGACATGTATCCATTATTGGAACTGGC
ATGTACTGACACCTACTTTATAGGCCTCACTGTTGTTTTCAGTGGTGGAGCACTCTGTATGGTCATCTTC
ACCCTTCTAATAATTTCCTATAGGGTCATCCTAAACTCCCTTAAAACTTACACTCAGGAAGGGAGGCATA
AAGCCCTGTCTACCTGCAGCTCCCACATCACTGTGATTGTTCTCTTTTTATTCCCTGTATTTTCATATGT
GAGACCTGTTTCAAACTTTTCTATTGACACATTCATGACTGTCTTTTATACAGTTATCACACCCAAGTTG
AATCCTTTAATATACACTTTCAGAAATTCAGAGATGAGAAATGTTATAGAAAAACTCTTGGTGAAAAAGG
TAACTATATTTAGAATAACAGGGTCCATCCTCATGTAG

A GPCR-like protein of the invention, referred to herein as GPCR2, is an Olfactory Receptor (“OR”)-like protein. Some members of the Olfactory Receptor-Like Protein Family end up localized at the cell surface, where they exhibit activity. Therefore it is likely that these novel GPCR2 proteins are available at the appropriate sub-cellular localization and hence accessible for the therapeutic uses described in this application. The GPCR2 polypeptide (SEQ ID NO: 4) encoded by SEQ ID NO: 3 is 314 amino acids in length and is presented using the one-letter amino acid code in Table 2B. The Psort profile for GPCR2 predicts that this sequence has a signal peptide and is likely to be a Type IIIa membrane protein, localized at the plasma membrane with a certainty of 0.6000. In alternative embodiments, a GPCR2 polypeptide is located to the Golgi body with a certainty of 0.4000, to the endoplasmic reticulum (membrane) with a certainty of 0.3000, or to the mitochondrial inner membrane with a certainty of 0.0300. The Signal P predicts a likely cleavage site for a GPCR2 peptide is between positions 39 and 40, i.e., at the dash in the sequence VLG-NQ.

TABLE 2B
GPCR2 protein sequence

(SEQ ID NO:4)

MRPNNSITEFVLLGFSQDPGMQKELFVMFLFTYVVTVLGNQLIVVTIIASPSLGSPMYFFLACLSFIDAA
YFTVISPKLIVDLLCDKKTISFQTFMGQLFIDHFFGGAEAFLLVVMAYDRYVAICKTLHYLTIMTRQVCI
LALLVAATGGFVHSVFQIVVVYSLPFCGANVIDHFSCDMYPLLELACTDTYFIGLTVVFSGGALCMVIFT
LLIISYRVILNSLKTYTQEGRHKALSTCSSHITVIVLFLFPVFSYVRPVSNFSIDTFMTVFYTVITPKLN
PLIYTFRNSEMRNVIEKLLVKKVTIFRITGSILM

A BLAST analysis of GPCR2 was run against the proprietary PatP GENESEQ Protein Patent database. It was found, for example, that the amino acid sequence of GPCR2 had high homology to other proteins as shown in Table 2C.

TABLE 2C

BLASTX results from PatP database for GPCR2

| | | |
|---|---|---|
| | | Smallest |
| | | Sum |
| | High | Probability |
| Sequences producing High-scoring Segment Pairs: | Score | P(N) |

| | | |
|---|---|---|
| patp:AAG72043 Human olfactory receptor polypeptide | 1560 | 6.0e−160 |
| patp:AAG72047 Human olfactory receptor polypeptide | 1255 | 1.3e−127 |
| patp:AAG72026 Human olfactory receptor polypeptide | 1249 | 5.4e−127 |
| patp:AAG72045 Human olfactory receptor polypeptide | 1218 | 1.1e−123 |
| patp:AAU24537 Human olfactory receptor AOLFR22 | 1203 | 4.1e−122 |

In a search of public sequence databases, it was found, for example, that the amino acid sequence of the GPCR2 protein of the present invention was found to have 204 of 302 amino acid residues (67%) identical to the 307 amino acid residue ptnr:SPTREMBL-ACC:Q9QW37 protein from Rattus sp (rat) (OR18 odorant receptor, E=4e-92). GPCR2 also has homology to the proteins shown in the BLASTP data in Table 2D.

TABLE 2D
GPCR2 BLASTP results

| Gene Index/ | | Length | Identity | Positive | |
|---|---|---|---|---|---|
| Identifier | Protein/Organism | (aa) | (%) | (%) | Expect |
| gi\|423702\|pir\|\| | olfactory receptor OR18 | 307 | 204/302 | 240/302 | 4e−92 |
| S29710 | [Rattus norvegicus] | | (67%) | (78%) | |
| gi\|17460099\|ref\| | similar to | 722 | 184/303 | 231/303 | 7e−88 |
| XP_062161.1(XM_062161) | odorant receptor 16 | | (60%) | (75%) | |
| | [Homo sapiens] | | | | |
| gi\|17459946\|ref\| | similar to | 316 | 171/300 | 225/300 | 2e−79 |
| XP_062088.1(XM_062088) | odorant receptor 16 | | (57%) | (75%) | |
| | [Homo sapiens] | | | | |
| gi\|11496249\|ref\| | odorant receptor 16 | 308 | 168/298 | 219/298 | 5e−79 |
| NP_067343.1(NM_021368) | [Mus musculus] | | (56%) | (73%) | |
| gi\|17459952\|ref\| | similar to | 277 | 169/269 | 206/269 | 6e−77 |
| XP_062090.1(XM_062090) | odorant receptor 16 | | (62%) | (75%) | |
| | [Homo sapiens] | | | | |

This BLASTP data is displayed graphically in the ClustalW in Table 2E. A multiple sequence alignment is given in Table 2E, with the GPCR2 protein being shown on line 1, in a ClustalW analysis comparing the protein of the invention with the related protein sequences shown in Table 2D.

[image]

[image]

[image]

Table 2F lists the domain description from DOMAIN analysis results against GPCR2. This indicates that the GPCR2 sequence has properties similar to those of other proteins known to contain this domain as well as to the 254 amino acid 7tm domain (SEQ ID NO: 40) itself.

TABLE 2F
Domain Analysis of GPCR2

| | | |
|---|---|---|
| | Score | E |
| PSSMs producing significant alignments: | (bits) | value |

| | | | |
|---|---|---|---|
| 7tm_1 (InterPro) | 7 transmembrane receptor (rhodopsin family) | 73.9 | 1e−14 |

| | | | | |
|---|---|---|---|---|
| GPCR2: | 39 | GNQLIVVTIIASPSLGSPMYFFLACLSFIDAAYFTVISPKLIVDLLCDKKTISFQTFMGQ | 98 | |
| | | \|\| \|+++ \|+ + \| +\| \|\| \|+ \| + + \| + \|+ | | |
| Sbjct: | 1 | GNLLVILVILRTKKLRTPTNIFLLNLAVADLLFLLTLPPWALYYLVGGDWVFGDALCKLV | 60 | |
| | | o o o o o o o o o o o | | |
| GPCR2: | 99 | LFIDHFFGGAEAFLLVVMAYDRYVAICKTLHYLTIMTRQVCILALLVAATGGFVHSVFQI | | 158 |
| | | + \| \| \|\| ++ \|\|\|+\|\| \| \| \| \| + + +\|+ + \|+ + | | |
| Sbjct: | 61 | GALFVVNGYASILLLTAISIDRYLAIVHPLRYRRIRTPRRAKVLILLVWVLALLLSLPPL | | 120 |
| | | o o o o o o o o | | |
| GPCR2: | 159 | VVVYSLPFCGANVIDHFSCDMYPLLELACTDTYFIGLTVVFSGGALCMVIFTLLII---- | | 214 |
| | | + + + \| + \| + \| \| \| +++ \|+ | | |
| Sbjct: | 121 | LFSWLRTV---EEGNTTVCLIDFPEESVKRSYVLLSTLVGFVLPLLVILVCYTRILRTLR | | 177 |
| GPCR2: | 215 | SYRVILNSLKTYTQEGRHKALSTCSSHITVIVLFL-FPVFSYVRPVSNFS------IDTF | | 267 |
| | | \|\|\| + \| \| + ++ +\| + + + + \| | | |
| Sbjct: | 178 | KRARSQRSLKRRSSSERKAAKMLLVVVVVFVLCWLPYHIVLLLDSLCLLSIWRVLPTALL | | 237 |
| GPCR2: | 268 | MTVFYTVITPKLNPLIY 284 | | |
| | | +\|++ + \|\|\|+\|\| | | |
| Sbjct: | 238 | ITLWLAYVNSCLNPIIY 254 | | |

The Olfactory Receptor-like GPCR2 disclosed in this invention is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalanic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources. Further expression data for GPCR2 is provided in Example 2.

The nucleic acids and proteins of GPCR2 are useful in potential therapeutic applications implicated in various GPCR-related pathological disorders and/or OR-related pathological disorders, described further herein. The novel GPCR2 nucleic acid encoding the GPCR-like protein of the invention, or fragments thereof, may further be useful in diagnostic applications, wherein the presence or amount of the nucleic acid or the protein are to be assessed.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-GPCRX Antibodies” section below. The disclosed GPCR2 protein has multiple hydrophilic regions, each of which can be used as an immunogen. In one embodiment, for example, a contemplated GPCR2 epitope comprises from about amino acids 10 to about 20. In another embodiment, for example, a GPCR2 epitope comprises from about amino acids 90 to about 95. In further embodiments, for example, a GPCR2 epitope comprises from about 220 to about 240, and from about 260 to about 300.

GPCR3

The disclosed novel GPCR3 (alternatively referred to herein as CG55950-01) includes the 959 nucleotide sequence (SEQ ID NO: 5) shown in Table 3A. A GPCR3 ORF begins with a Kozak consensus ATG initiation codon at nucleotides 4-6 and ends with a TAG codon at nucleotides 957-959. Putative untranslated regions upstream from the initiation codon and downstream from the termination codon are underlined in Table 3A, and the start and stop codons are in bold letters.

TABLE 3A
GPCR3 Nucleotide Sequence

(SEQ ID NO:5)

TAAATGAGACCTAATAACAGTATTACAGAATTTGTCCTCCTGGGCTTTTCACAGGATCCTGATATGCAAA
ACACATTATTTGTCATGTTTTTACTCACATACATTGTGACAGTGGTGGGGAACCTACTCGTTGCGGTGAC
TATTATTGTCAGCCCTTCCTTGAGCTCCCCAATGTATTTCTTCCTTGCTTGCCTGTCATTAATAGATGCT
GTATTATCCACCACCATTTCTCCCATATTGATTGTAGACCTACTCTGTGACAAAAAGACTATTTCCTTCC
CAGCTTGCATGGGCCAGCTATTTACAGACCACTTGTTTGGTGGAACTGAGATCTTCCTTCTGGTGGTGAT
GGCCTATGATCGCTACGTGGCCATCTGTAAGCCACTGCACTATTTAACCATCATGAATCGACAGGTTTCC
ATCCTTCTGTTGGTGGTGGCCATGACTGGAGGTTTCCTTCATTCTGTGTTTCAAATTGCTGTTCTGTACA
GTCTCCCTTTCTGTGGCCCCAATGTCATTGACCACTTTTTCTGTGACATGTACCCATTATTGGAACTGGC
GTGCACTGACACCTACTCTATAGGCCTCACTGTAGTTTTCAGTGGTGGAGCAATGTGTATGGTCATCTTC
GCCCTTCTACTAATCTCCTATGGAGTCAGCCTAAACTCCCTTAAAACTTATAGTCAGGAAGGGAGGCGTA
AAGCCCTGTCTACCTGCAGCTCGCACATCACCGTGGTTGTCCTCTTTTTTGTTCCCTGTATTTTCATGTA
TGTTAGACCTGTCTCAAACTTCCCTATTGATAAATTCGTTACTGTGTTTTATACATTTATCACACCCATG
CTGAATCCTTTTTTATACACGTTGAGAAATTCAGAGATGATAAATGCTATAAAACACCTGTTGTGTAAGA
AGCTAACTATAGTTAGAATAAGAGTGTCCCTCCTCATGTAGATAAGGAT

The GPCR3 polypeptide (SEQ ID NO: 6) encoded by SEQ ID NO: 5 is 315 amino acids in length and is presented using the one-letter amino acid code in Table 3B. The Psort profile for GPCR3 predicts that this sequence has a signal peptide and is likely to be a Type IIIb membrane protein, localized at the plasma membrane with a certainty of 0.6000. In alternative embodiments, a GPCR3 polypeptide is located to the Golgi body with a certainty of 0.4000, to the endoplasmic reticulum (membrane) with a certainty of 0.3000, or to the mitochondrial inner membrane with a certainty of 0.0300. The Signal P predicts a likely cleavage site for a GPCR3 peptide is between positions 39 and 40, i.e., at the dash in the sequence VVG-NL.

TABLE 3B
GPCR3 protein sequence

(SEQ ID NO:6)

MRPNNSITEFVLLGFSQDPDMQNTLFVMFLLTYIVTVVGNLLVAVTIIVSPSLSSPMYFFLACLSLIDAV
LSTTISPILIVDLLCDKKTISFPACMGQLFTDHLFGGTEIFLLVVMAYDRYVAICKPLHYLTIMNRQVSI
LLLVVAMTGGFLHSVFQIAVLYSLPFCGPNVIDHFFCDMYPLLELACTDTYSIGLTVVFSGGAMCMVIFA
LLLISYGVSLNSLKTYSQEGRRKALSTCSSHITVVVLFFVPCIFMYVRPVSNFPIDKFVTVFYTFITPML
NPFLYTLRNSEMINAIKHLLCKKLTIVRIRVSLLM

A BLAST analysis of GPCR3 was run against the proprietary PatP GENESEQ Protein Patent database. The amino acid sequence of GPCR3 had high homology to other proteins as shown in Table 3C.

TABLE 3C

BLASTX results for GPCR3

| | | |
|---|---|---|
| | | Smallest |
| | | Sum |
| | High | Probability |
| Sequences producing High-scoring Segment Pairs: | Score | P(N) |

| | | |
|---|---|---|
| patp:AAG72047 Human olfactory receptor polypeptide | 1595 | 1.2e−163 |
| patp:AAG72045 Human olfactory receptor polypeptide | 1307 | 3.9e−133 |
| patp:AAG72299 Human olfactory receptor polypeptide | 1276 | 7.5e−130 |
| patp:AAG72043 Human olfactory receptor polypeptide | 1270 | 3.2e−129 |
| patp:AAG72031 Human olfactory receptor polypeptide | 1259 | 4.7e−128 |

In a search of public sequence databases, it was found, for example, that the amino acid sequence of the protein of the invention was found to have 204 of 302 amino acid residues (67%) identical to the 307 amino acid residue residue ptnr:SPTREMBL-ACC:Q9QW37 protein from Rattus sp (rat) (OR18 odorant receptor, E=4e-92). GPCR3 also has homology to the proteins shown in the BLASTP data in Table 3D.

TABLE 3D
GPCR3 BLASTP results

| | | | | | |
|---|---|---|---|---|---|
| Gene Index/ | | Length | | Positive | |
| Identifier | Protein/Organism | (aa) | Identity (%) | (%) | Expect |

| | | | | | |
|---|---|---|---|---|---|
| gi\|423702\|pir\|\| | olfactory receptor OR18 | 307 | 204/302 | 240/302 | 4e−92 |
| S29710 | [rattus norvegicus] | | (67%) | (78%) | |
| gi\|17460099\|ref\| | similar to odorant receptor 16 | 722 | 184/303 | 231/303 | 7e−88 |
| XP_062161.1 | (H. sapiens) [Homo sapiens] | | (60%) | (75%) | |
| (XM_062161) | | | | | |
| gi\|17459946\|ref\| | similar to odorant receptor 16 | 316 | 171/300 | 225/300 | 2e−79 |
| XP_062088.1)\|(X | (H. sapiens) [Homo sapiens] | | (57%) | (75%) | |
| M_062088) | | | | | |
| gi\|11496249\|ref\| | odorant receptor 16 [Mus | 308 | 168/298 | 219/298 | 5e−79 |
| NP_067343.1\| | musculus] | | (56%) | (73%) | |
| (NM_021368) | | | | | |
| gi\|17459952\|ref\| | similar to odorant receptor 16 | 277 | 169/269 | 206/269 | 6e−77 |
| XP_062090.1\|(XM_062090) | (H. sapiens) [Homo sapiens] | | (62%) | (75%) | |

A multiple sequence alignment is given in Table 3E, with the GPCR3 protein of the invention being shown on line 2, in a ClustalW analysis comparing GPCR3 with related protein sequences disclosed in Table 3D.

[image]

[image]

[image]

Table 3F lists the domain description from DOMAIN analysis results against GPCR3. This indicates that the GPCR3 sequence has properties similar to those of other proteins known to contain the 254 amino acid 7tm domain (SEQ ID NO: 40).

TABLE 3F
Domain Analysis of GPCR3

| | | |
|---|---|---|
| | Score | E |
| PSSMs producing significant alignments: | (bits) | value |

| | | | |
|---|---|---|---|
| 7tm_1 (InterPro) | 7 transmembrane receptor (rhodopsin family) | 73.9 | 1e−14 |

| | | | |
|---|---|---|---|
| GPCR3: | 39 | GNQLIVVTIIASPSLGSPMYFFLACLSFIDAAYFTVISPKLIVDLLCDKKTISFQTFMGQ | 98 |
| | | \|\| \|+++ \|+ + \| +\| \|\| \|+ \| + + \| + \|+ | |
| Sbjct: | 1 | GNLLVILVILRTKKLRTPTNIFLLNLAVADLLFLLTLPPWALYYLVGGDWVFGDALCKLV | 60 |
| | | o o o o o o o o o o o | |
| GPCR3: | 99 | LFIDHFFGGAEAFLLVVMAYDRYVAICKTLHYLTIMTRQVCILALLVAATGGFVHSVFQI | 158 |
| | | + \| \| \|\| ++ \|\|\|+\|\| \| \| \| \| + + +\|+ + \|+ + | |
| Sbjct: | 61 | GALFVVNGYASILLLTAISIDRYLAIVHPLRYRRIRTPRRAKVLILLVWVLALLLSLPPL | 120 |
| | | o o o o o o o o | |
| GPCR3: | 159 | VVVYSLPFCGANVIDHFSCDMYPLLELACTDTYFIGLTVVFSGGALCMVIFTLLII---- | 214 |
| | | + + + \| + \| + \| \| \| +++ \|+ | |
| Sbjct: | 121 | LFSWLRTV---EEGNTTVCLIDFPEESVKRSYVLLSTLVGFVLPLLVILVCYTRILRTLR | 177 |
| GPCR3: | 215 | SYRVILNSLKTYTQEGRHKALSTCSSHITVIVLFL-FPVFSYVRPVSNFS------IDTF | 267 |
| | | \|\|\| + \| \| + ++ +\| + + + + \| | |
| Sbjct: | 178 | KRARSQRSLKRRSSSERKAAKMLLVVVVVFVLCWLPYHIVLLLDSLCLLSIWRVLPTALL | 237 |
| GPCR3: | 268 | MTVFYTVITPKLNPLIY 284 | |
| | | +\|++ + \|\|\|+\|\| | |
| Sbjct: | 238 | ITLWLAYVNSCLNPIIY 254 | |

A GPCR-like protein of the invention, disclosed herein as GPCR3, is an Olfactory Receptor (“OR”)-like protein. Some members of the Olfactory Receptor-Like Protein Family end up localized at the cell surface, where they exhibit activity. Therefore it is likely that these novel GPCR3 proteins are available at the appropriate sub-cellular localization and hence accessible for the therapeutic uses described in this application.

The GPCR3 disclosed in this invention is expressed in at least some of the following tissues: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus. This information was derived by determining the tissue sources of the sequences that were included in the invention. Further expression data for GPCR3 is provided in Example 2.

The nucleic acids and proteins of GPCR3 are useful in potential therapeutic applications implicated in various GPCR-related pathological disorders and/or OR-related pathological disorders, described further above.

The novel nucleic acid encoding the GPCR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-GPCRX Antibodies” section below. The disclosed GPCR3 protein has multiple hydrophilic regions, each of which can be used as an immunogen. In one embodiment, for example, a contemplated GPCR3 epitope comprises from about amino acids 10 to about 20. In another embodiment, for example, a GPCR3 epitope comprises from about amino acids 90 to about 95. In further embodiments, for example, a GPCR3 epitope comprises from about 130 to about 140, from about 220 to about 240, and from about 280 to about 360.

GPCR4

A fourth GPCR-like protein of the invention, referred to herein as GPCR4, is an Olfactory Receptor (“OR”)-like protein. Two alternative novel GPCR4 nucleic acids and encoded polypeptides are provided, disclosed herein as GPCR4a and GPCR4b. The GPCR4a polypeptide differs from the GPCR4b polypeptide by a single amino acid substitution of V254A.

GPCR4a

In one embodiment, a GPCR4 variant is the novel GPCR4a (alternatively referred to herein as CG55928-01), which includes the 949 base nucleotide sequence (SEQ ID NO: 7) shown in Table 4A. A GPCR4a ORF begins with a Kozak consensus ATG initiation codon at nucleotides 7-9 and ends with a TGA codon at nucleotides 943-945. Putative untranslated regions upstream from the initiation codon and downstream from the termination codon are underlined in Table 4A, and the start and stop codons are in bold letters.

TABLE 4A
GPCR4a Nucleotide Sequence

(SEQ ID NO:34)

CTGGCAATGGGGCTCAATACGTCTGCTTCCACCTTCCAGCTTACTGGCTTCCCAGGCATGGAGAAGGCAC
ATCACTGGATATTCATCCCATTATTGGCAGCCTACATCTCCATACTTCTTGGCAGTGGCACTCTTCTCTT
TCTCATCAGGAATGATCATAACCTCCATGAGCCCATGTACTATTTCTTAGCTATGTTGGCAGCTACAGAC
CTCGGAGTGACATTGACCACAATGCCCACAGTGCTAGGTGTTCTGTGGTTAGATCACAGGGAGACTGGCC
ATGGAGCCTGCTTCTCTCAGGCCTATTTTATCCATACTCTTTCTGTCATGGAGTCAGGTGTCTTGCTTGC
CATGGCTTATGACTGTTTCATTGCCATCCACAACCCCTTAAGATATATCTCTATCCTGACCAACACCCAG
GTAATGAAGATTGGTGTGGGGGTATTGACAAGGGCTGGTCTGTCCATTATGCCAATAGTTGTTCGCCTAC
ACTGGTTTCCCTACTGTCGAGCCCATGTATTCTCCCATGCTTTCTGTCTACACCAAGATGTCATCAAGCT
AGCCTGTGCTGACATCACCCTCAACCGTCTCTATCCAGTTGTGGTTTTATTTGCAATGGTCTTGTTGGAC
TTTCTCATCATCTTTTTCTCCTACATTTTGATTCTCAAGACTGTCATGGGCATTGGTTCTGGAGGAGAAA
GGGCCAAGGCCCTCAACACATGTGTCTCTCATATCTGCTGCATCCTGGTCTTCTATGTCACTGTAGTTTG
TCTGACATTTATTCATAGGTTTGGAAAGCATGTTCCTCATGTCGTTCACATCACAATGAGATACATCCAC
TTCCTTTTCCCACCTTTTATGAACCCATTTATCTATAGCATTAAAACTAAGCAGATTCAGAGTGGCATAC
TTCGCTTATTCTCTCTGCCTCACTCTAGAGCATGACATT

The sequence of GPCR4a was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The cDNA coding for the GPCR4a sequence was cloned by the polymerase chain reaction (PCR). Primers were designed based on in silico predictions of the full length or some portion (one or more exons) of the cDNA/protein sequence of the invention. The DNA sequence and protein sequence for a novel Olfactory Receptor-like gene were obtained by exon linking and are reported here as GPCR4a. These primers and methods used to amplify GPCR4a cDNA are described in the Examples.

The GPCR4a polypeptide (SEQ ID NO: 8) encoded by SEQ ID NO: 7 is 312 amino acids in length and is presented using the one-letter amino acid code in Table 4B. The Psort profile for GPCR4a predicts that this sequence has a signal peptide and is likely to be a Type IIIa membrane protein, localized at the plasma membrane with a certainty of 0.6400. In alternative embodiments, a GPCR4a polypeptide is located to the Golgi body with a certainty of 0.4000, to the endoplasmic reticulum (membrane) with a certainty of 0.3000, or to the mitochondrial inner membrane with a certainty of 0.0300. The Signal P predicts a likely cleavage site for a GPCR4a peptide is between positions 39 and 40, i.e., at the dash in the sequence LLG-SG.

TABLE 4B
GPCR4a polypeptide sequence

(SEQ ID NO:8)

MGLNTSASTFQLTGFPGMEKAHHWIFIPLLAAYISILLGSGTLLFLIRNDHNLHEPMYYFLAMLAATDLG
VTLTTMPTVLGVLWLDHRETGHGACFSQAYFIHTLSVMESGVLLAMAYDCFIAIHNPLRYISILTNTQVM
KIGVGVLTRAGLSIMPIVVRLHWFPYCRAHVFSHAFCLHQDVIKLACADITLNRLYPVVVLFAMVLLDFL
IIFFSYILILKTVMGIGSGGERAKALNTCVSHICCILVFYVTVVCLTFIHRFGKHVPHVVHITMRYIHFL
FPPFMNPFIYSIKTKQIQSGILRLFSLPHSRA

GPCR4b

In an alternative embodiment, a GPCR4 variant is the novel GPCR4b (alternatively referred to herein as CG55928-02), which includes the 928 base nucleotide sequence (SEQ ID NO: 9) shown in Table 4C. The GPCR4b ORF was identified at nucleotides 1-3 with a TCT codon and ends with a TGA codon at nucleotides 922-924. In one embodiment, the GPCR4b nucleic acid and polypeptide sequences extend in the 5′ direction to include a complete ORF that begins with methionine encoded by an ATG. In a specific embodiment, these GPCR4b upstream sequences are identical to those disclosed above for the GPCR4a nucleic acid and amino acid sequences. The putative untranslated region downstream from the termination codon is underlined in Table 4C, and the start and stop codons are in bold letters.

TABLE 4C
GPCR4b Nucleotide Sequence

(SEQ ID NO:9)

TCTGCTTCCACCTTCCAGCTTACTGGCTTCCCAGGCATGGAGAAGGCACATCACTGGATATTCATCCCATTATT
GGCAGCCTACATCTCCATACTTCTTGGCAGTGGCACTCTTCTCTTTCTCATCAGGAATGATCATAACCTCCATG
AGCCCATGTACTATTTCTTAGCTATGTTGGCAGCTACAGACCTCGGAGTGACATTGACCACAATGCCCACAGTG
CTAGGTGTTCTGTGGTTAGATCACAGGGAGACTGGCCATGGAGCCTGCTTCTCTCAGGCCTATTTTATCCATAC
TCTTTCTGTCATGGAGTCAGGTGTCTTGCTTGCCATGGCTTATGACTGTTTCATTGCCATCCACAACCCCTTAA
GATATATCTCTATCCTGACCAACACCCAGGTAATGAAGATTGGTGTGGGGGTATTGACAAGGGCTGGTCTGTCC
ATTATGCCAATAGTTGTTCGCCTACACTGGTTTCCCTACTGTCGAGCCCATGTATTCTCCCATGCTTTCTGTCT
ACACCAAGATGTCATCAAGCTAGCCTGTGCTGACATCACCCTCAACCGTCTCTATCCAGTTGTGGTTTTATTTG
CAATGGTCTTGTTGGACTTTCTCATCATCTTTTTCTCCTACATTTTGATTCTCAAGACTGTCATGGGCATTGGT
TCTGGAGGAGAAAGGGCCAAGGCCCTCAACACATGTGTCTCTCATATCTGCTGCATCCTGGTCTTCTATGTCAC
TGTAGCTTGTCTGACATTTATTCATAGGTTTGGAAAGCATGTTCCTCATGTCGTTCACATCACAATGAGATACA
TCCACTTCCTTTTCCCACCTTTTATGAACCCATTTATCTATAGCATTAAAACTAAGCAGATTCAGAGTGGCATA
CTTCGCTTATTCTCTCTGCCTCACTCTAGAGCATGACATT

The GPCR4b protein (SEQ ID NO: 10) encoded by SEQ ID NO: 9 is 307 amino acids in length and is presented using the one-letter code in Table 4D. The Psort profile for GPCR4b predicts that this sequence has a signal peptide and is likely to be a Type IIIa membrane protein, localized at the plasma membrane with a certainty of 0.6000. In alternative embodiments, a GPCR4b polypeptide is located to the Golgi body with a certainty of 0.4000, to the endoplasmic reticulum (membrane) with a certainty of 0.3000, or to the mitochondrial inner membrane with a certainty of 0.0300. The Signal P predicts a likely cleavage site for a GPCR4b peptide is between positions 34 and 35, i.e., at the dash in the sequence LLG-SG.

TABLE 4D
GPCR4b protein sequence

(SEQ ID NO:37)

SASTFQLTGFPGMEKAHHWIFIPLLAAYISILLGSGTLLFLIRNDHNLHEPMYYFLAMLAATDLGVTLTTMPTVLSV
LWLDHRETGHGACFSQAYFIHTLSVMESGVLLAMAYDCFIAIHNPLRYISILTNTQVMKIGVGVLTRAGLSIMPIVV
RLHWFPYCRAHVFSHAFCLHQDVIKLACADITLNRLYPVVVLFAMVLLDFLIIFFSYILILKTVMGIGSGGERAKAL
NTCVSHICCILVFYVTVACLTFIHRFGKHVPHVVHITMRYIHFLFPPFMNPFIYSIKTKQIQSGILRLFSLPHSRA

GPCR4 Clones

Unless specifically addressed as GPCR4a or GPCR4b, any reference to GPCR4 is assumed to encompass all variants. Residue differences between any GPCRX variant sequences herein are written to show the residue in the “a” variant, the residue position with respect to the “a” variant, and the residue in the “b” variant.

A BLAST analysis of GPCR4 was run against the proprietary PatP GENESEQ Protein Patent database. The amino acid sequence of GPCR4 has high homology to other proteins as shown in Table 4E.

TABLE 4E

BLASTX results for GPCR4

| | | |
|---|---|---|
| | | Smallest |
| | | Sum |
| | High | Probability |
| Sequences producing High-scoring Segment Pairs: | Score | P(N) |

| | | |
|---|---|---|
| patp:AAG72616 Murine OR-like polypeptide query sequence | 1394 | 2.3e−142 |
| patp:AAG72615 Murine OR-like polypeptide query sequence | 1180 | 1.1e−119 |
| patp:AAG71433 Human olfactory receptor polypeptide | 1121 | 2.0e−113 |
| patp:AAG73063 Olfactory receptor-like polypeptide | 1121 | 2.0e−113 |
| patp:AAG72617 Murine OR-like polypeptide query sequence | 1117 | 5.3e−113 |

In a search of public sequence databases, it was found, for example, that the nucleic acid sequence of this invention has 666 of 904 bases (73%) identical to a gb:GENBANK-ID:AF137396|acc:AF137396.2 mRNA from Homo sapiens (Homo sapiens ubiquilin 3, HOR5′Beta14, HOR5′Beta13, HOR5′Beta12, and HOR5′Beta11 genes, complete cds; HOR5Beta10 and HOR5′Beta9 pseudogenes, complete sequence; HOR5′Beta8 and HOR5′Beta7 genes, complete cds; CHR11ORF1 and amphiphysin pseudogenes, complete sequence; HOR5′Beta6 and HOR5′Beta5 genes, complete cds; HOR5′Beta4 pseudogene, complete sequence; HOR5′Beta3 genes, complete cds; HOR5′Beta2 pseudogene, complete sequence; and HOR5′Beta1 gene, complete cds)

The full amino acid sequence of the GPCR4 polypeptide of the invention was found to have 259 of 306 amino acid residues (84%) identical to, and 284 of 306 amino acid residues (92%) similar to, the 315 amino acid residue ptnr:SPTREMBL-ACC:Q9WVN6 protein from Mus musculus (Mouse) (MOR 5′BETA3).

Additional similarities are illustrated by the BLASTP results as shown in Table 4F.

TABLE 4F
GPCR4 BLASTP Results

| Gene Index/ | | Length | Identity | Positives | |
|---|---|---|---|---|---|
| Identifier | Protein/Organism | (aa) | (%) | (%) | Expect |
| gi\|11908217\|gb\| | HOR5′Beta6 | 312 | 312/312 | 312/312 | e−154 |
| AAG41682.1\|(AF137396) | [Homo sapiens] | | (100%) | (100%) | |
| gi\|7305343\|ref\| | olfactory receptor 64 | 315 | 263/311 | 288/311 | e−130 |
| NP_038644.1\|(NM_013616) | [Mus musculus] | | (84%) | (92%) | |
| gi\|7305345\|ref\| | olfactory receptor 65 | 307 | 219/307 | 257/307 | e−108 |
| NP_038645.1\|(NM_013617) | [Mus musculus] | | (71%) | (83%) | |
| gi\|14423840\|sp\| | OLFACTORY RECEPTOR | 312 | 207/303 | 250/303 | e−103 |
| Q9Y5P1\|OXB2_HUMAN | 51B2 (HOR5′BETA3) | | (68%) | (82%) | |
| gi\|11908208\|gb\| | HOR5′Beta3 | 312 | 206/303 | 249/303 | e−102 |
| AAD29425.2\|AF137396_1 | [Homo sapiens] | | (67%) | (81%) | |
| (AF137396) | | | | | |

A multiple sequence alignment is given in Table 4G, with the GPCR4 protein of the invention being shown on line 1 (GPCR4a) and line 2 (GPCR4b), in a ClustalW analysis comparing the GPCR4 polypeptides with related protein sequences of Table 4F. The residue that differs between GPCR4a and GPCR4b (V245A) is marked with the (o) symbol.

[image]

[image]

DOMAIN results for GPCR4 were collected from the Conserved Domain Database (CDD) with Reverse Position Specific BLAST. This BLAST samples domains found in the Smart and Pfam collections. The results are listed in Table 4H with the statistics and domain description. The 7tm_{—}1, a seven transmembrane receptor (rhodopsin family), was shown to have significant homology to GPCR4. An alignment of GPCR4 residues 40-290 (SEQ ID NO: 52) with 7tm_{—}1 residues 1-254 (SEQ ID NO: 40) are shown in Table 4H.

TABLE 4H
DOMAIN results for GPCR4

| | | |
|---|---|---|
| | Score | E |
| PSSMs producing significant alignments: | (bits) | value |

| | | | |
|---|---|---|---|
| gnl\|Pfam\|pfam00001 | 7tm_1, 7 transmembrane receptor (rhodopsin family) | 63.5 | 2e−11 |

| | | | |
|---|---|---|---|
| GPCR4: | 39 | GSGTLLFLIRNDHNLHEPMYYFLAMLAATDLGVTLTTMPTVLGVLWLDHRETGHGACFSQ | 98 |
| | | \|+ ++ +\| \| \| \|\| \|\| \|\| \|\| \| \| \| \| \| | |
| Sbjct: | 1 | GNLLVILVILRTKKLRTPTNIFLLNLAVADLLFLLTLPPWALYYLVGGDWVFGDALCKLV | 60 |
| GPCR4: | 99 | AYFIHTLSVMESGVLLAMAYDCFIAIHNPLRYISILTNTQVMKIGVGVLTRAGLSIMPIV | 158 |
| | | +\| \|++ \| ++\|\| +\|\|\|\| \| \| + + + \| \| \| +\| + | |
| Sbjct: | 61 | GALFVVNGYASILLLTAISIDRYLAIVHPLRYRRIRTPRRAKVLILLVWVLALLLSLPPL | 120 |
| GPCR4: | 159 | VRLHWFPYCRAHVFSHAFCLHQDVIKLACADITLNRLYPVVVLFAMVLLDFLIIFFSYIL | 218 |
| | | + + ++ +\| + ++ + + +\| +\| ++ | |
| Sbjct: | 121 | LFSWLRTVEEGNTTVCLIDFPEESVKRSYVLLSTLVGFVLPLLVILVCYTRILRTLRKRA | 180 |
| GPCR4: | 219 | ILKTVMGIGSGGERAKALNTCVSH---ICCILVFYVTVVCLTFIH-RFGKHVPHVVHITM | 274 |
| | | + + \| \|\| \| \| + \| \| +++ ++ + + +\| + \|\|+ | |
| Sbjct: | 181 | RSQRSLKRRSSSERKAAKMLLVVVVVFVLCWLPYHIVLLLDSLCLLSIWRVLPTALLITL | 240 |
| GPCR4: | 275 | RYIHFLFPPFMNPFIY 290 | |
| | | + +\|\| \|\| | |
| Sbjct: | 241 | WLAY--VNSCLNPIIY 254 | |

The cDNA coding for the GPCR4 sequences was cloned by the polymerase chain reaction (PCR). Primers were designed based on in silico predictions of the full length or some portion (one or more exons) of the cDNA/protein sequence of the invention. The DNA sequence and protein sequence for a novel Olfactory Receptor-like gene were obtained by exon linking and are reported here as GPCR4. These primers and methods used to amplify GPCR4 cDNA are described in the Examples.

The GPCR4 disclosed in this invention is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources. This is by no way limiting in that olfactory receptors are a class of G protein-coupled receptor which are known to be expressed in all tissue types. Further tissue expression analysis is provided in Example 2.

The nucleic acids and proteins of GPCR4 are useful in potential therapeutic applications implicated in various GPCR-related pathological disorders and/or OR-related pathological disorders, described further above.

The novel nucleic acid encoding the GPCR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-GPCRX Antibodies” section below. The disclosed GPCR4 polypeptides have multiple hydrophilic regions, each of which can be used as an immunogen. In one embodiment, a contemplated GPCR4 epitope comprises, for example from about amino acids 10 to about 20. In other specific embodiments, GPCR4 epitopes comprise from about amino acids 45 to about 60, from about 75 to about 100, from about 260 to about 267, and from about 270 to about 310.

GPCR5

The disclosed novel GPCR5 (alternatively referred to herein as CG55926-01) includes the 955 nucleotide sequence (SEQ ID NO: 11) shown in Table 5A. A GPCR5 ORF begins with a Kozak consensus ATG initiation codon at nucleotides 2-4 and ends with a TGA codon at nucleotides 938-940. Putative untranslated regions upstream from the initiation codon and downstream from the termination codon are underlined in Table 5A, and the start and stop codons are in bold letters.

TABLE 5A
GPCR5 Nucleotide Sequence

(SEQ ID NO:11)

GATGTCGTCCAGCAGCAGCTCCCATCCCTTCCTATTGACTGGTTTTCCAGGCTTGGAGGAAGCTCATCACTGGATTTCCGTATTTT
TCTTGTTCATGTATATATCCATCCTTTTTGGCAATGGCACCCTCCTTCTTCTCATTAAGGAAGATCACAATCTTCATGAGCCCATG
TACTTCTTTCTGGCCATGCTGGCTGCCACAGACCTGGGGCTGGCCCTGACCACAATGCCCACGGTGCTGGGAGTCCTCTGGCTGGA
TCACAGGGAGATTGGAAGTGCGGCCTGCTTTTCCCAGGCCTACTTTATACACTCACTTTCCTTTCTCGAGTCTGGCATTCTGCTTG
CCATGGCCTATGACCGTTTTATTGCCATCTGCAACCCTCTTAGATATACCTCTGTACTTACTAATACTCGAGTAGTGAAGATTGGG
CTGGGAGTTCTGATGAGGGGATTTGTATCCGTTGTTCCCCCAATCAGGCCCCTCTATTTTTTTCTGTATTGTCACTCCCATGTTCT
TTCACATGCATTCTGCCTTCACCAGGATGTCATTAAACTCGCCTGTGCTGATACCACCTTCAACCGACTGTACCCAGCTGTGCTTG
TAGTCTTTATATTTGTGCTGGATTATCTGATTATCTTCATCTCCTATGTGTTGATACTCAAGACTGTCCTGAGCATTGCCTCCAGA
GAGGAGAGGGCCAAGGCTCTCATTACCTGTGTCTCCCATATCTGCTGTGTCCTGGTTTTTTATGTCACAGTGATTGGATTGTCTCT
GATTCATCGTTTTGGAAAGCAGGTTCCACATATTGTTCACCTCATTATGAGCTATGCCTATTTTCTGTTCCCTCCACTAATGAATC
CTATAACATATAGTGTCAAGACCAAGCAGATTCAGAATGCCATTCTTCACCTTTTTACTACCCATAGAATTGGAACCTGATCTCCA
ATCATCACA

The GPCR5 polypeptide (SEQ ID NO: 12) encoded by SEQ ID NO: 11 is 312 amino acids in length and is presented using the one-letter amino acid code in Table 5B. The Psort profile for GPCR5 predicts that this sequence has a signal peptide and is likely to be a Type IIIa membrane protein, localized at the plasma membrane with a certainty of 0.6000. In alternative embodiments, a GPCR5 polypeptide is located to the Golgi body with a certainty of 0.4000, to the endoplasmic reticulum (membrane) with a certainty of 0.3000, or to the mitochondrial inner membrane with a certainty of 0.0300. The Signal P predicts a likely cleavage site for a GPCR5 peptide is between positions 21 and 22, ie., at the dash in the EEA-HH.

TABLE 5B
GPCR5 protein sequence

(SEQ ID NO:12)

MSSSSSSHPFLLTGFPGLEEAHHWISVFFLFMYISILFGNGTLLLLIKEDHNLHEPMYFFLAMLAATDLG
LALTTMPTVLGVLWLDHREIGSAACFSQAYFIHSLSFLESGILLAMAYDRFIAICNPLRYTSVLTNTRVV
KIGLGVLMRGFVSVVPPIRPLYFFLYCHSHVLSHAFCLHQDVIKLACADTTFNRLYPAVLVVFIFVLDYL
IIFISYVLILKTVLSIASREERAKALITCVSHICCVLVFYVTVIGLSLIHRFGKQVPHIVHLIMSYAYFL
FPPLMNPITYSVKTKQIQNAILHLFTTHRIGT

A BLAST analysis of GPCR5 was run against the proprietary PatP GENESEQ Protein Patent database. The amino acid sequence of GPCR5 had high homology to other proteins as shown in Table 5C.

TABLE 5C

BLASTX results for GPCR5

| | | |
|---|---|---|
| | | Smallest |
| | | Sum |
| | High | Probability |
| Sequence producing High-scoring Segment Pairs: | Score | P(N) |

| | | |
|---|---|---|
| patp:AAG72615 Murine OR-like polypeptide query sequence | 1211 | 5.8e−123 |
| patp:AAG72616 Murine OR-like polypeptide query sequence | 1208 | 1.2e−122 |
| patp:AAG72617 Murine OR-like polypeptide query sequence | 1149 | 2.2e−116 |
| patp:AAG72959 Human olfactory receptor data exploratorium | 1149 | 2.2e−116 |
| patp:AAG71433 Human olfactory receptor polypeptide | 1124 | 9.6e−114 |

In a search of public sequence databases, it was found, for example, that the amino acid sequence of the protein of the invention was found to have 233 of 307 amino acid residues (72%) identical to the 307 amino acid residue ptnr:SPTREMBL-ACC:Q9WVN5 MOR 5′BETA2-Mus musculus (Mouse, E=3.3e-122). Additional BLASTP results are shown in Table 5D.

TABLE 5D
GPCR5 BLASTP results

| Gene Index/ | | Length | Identity | Positives | |
|---|---|---|---|---|---|
| Identifier | Protein/Organism | (aa) | (%) | (%) | Expect |
| gi\|11908218\|gb\| | HOR5′Beta5 | 312 | 312/312 | 312/312 | e−145 |
| AAG41683.1\|(AF137396) | [Homo sapiens] | | (100%) | (100%) | |
| gi\|7305343\|ref\| | olfactory receptor 64 | 315 | 224/301 | 262/301 | e−110 |
| NP_038644.1\|(NM_013616) | [Mus musculus[ | | (74%) | (86%) | |
| gi\|7305345\|ref\| | olfactory receptor 65 | 307 | 223/306 | 258/306 | e−110 |
| NP_038645.1\|(NM_013617) | [Mus musculus] | | (72%) | (83%) | |
| gi\|11908217\|gb\| | HOR5′Beta6 | 312 | 213/306 | 253/306 | e−104 |
| AAG41682.1\|(AF_137396) | [Homo sapiens] | | (69%) | (82%) | |
| gi\|7305347\|ref\| | olfactory receptor 66 | 311 | 214/308 | 250/308 | e−104 |
| NP_038646.1\|(NM_013618) | [Mus musculus] | | (69%) | (80%) | |

A multiple sequence alignment is given in Table 5E, with the GPCR5 protein of the invention being shown on line 1, in a ClustalW analysis comparing GPCR5 with related protein sequences disclosed in Table 5D.

[image]

[image]

Table 5F lists the domain description from DOMAIN analysis results against GPCR5. This indicates that the GPCR5 sequence has properties similar to those of other proteins known to contain this 254 amino acid 7tm domain (SEQ ID NO: 40) itself.

TALBE 5F
Domain Analysis of GPCR5

| | | |
|---|---|---|
| | Score | E |
| PSSMs producing significant alignments: | (bits) | value |

| | | | |
|---|---|---|---|
| gnl\|Pfam\|pfam00001 | 7tm_1, 7 transmembrane receptor (rhodopsin family) | 62.8 | 3e−11 |

| | | | |
|---|---|---|---|
| GPCR5: | 39 | GNGTLLLLIKEDHNLHEPMYFFLAMLAATDLGLALTTMPTVLGVLWLDHREIGSAACFSQ | 98 |
| | | \|\| ++\|+\| \| \| \|\| \|\| \|\| \|\| \| \| \| \| \| \| | |
| Sbjct: | 1 | GNLLVILVILRTKKLRTPTNIFLLNLAVADLLFLLTLPPWALYYLVGGDWVFGDALCKLV | 60 |
| GPCR5: | 99 | AYFIHSLSFLESGILLAMAYDRFIAICNPLRYTSVLTNTRVVKIGLGVLMRGFVSVVPPI | 158 |
| | | + +\| \|++ \|\|++\|\| +\|\|\|\| + \| \| + \| \| + + +\| | |
| Sbjct: | 61 | GALFVVNGYASILLLTAISIDRYLAIVHPLRYRRIRTPRRAKVLILLVWVLALLLSLP-- | 118 |
| GPCR5: | 159 | RPLYFFLYCHSHVLSHAFCLHQDVIKLACADTTFNRLYPAVLVVFIFVLDYLIIFISYVL | 218 |
| | | \|\| \| + \|\| + + \| \| + + \|\|\| \|+\| + \| | |
| Sbjct: | 119 | -PLLFSWLRTVEEGNTTVCLIDFP------EESVKRSYVLLSTLVGFVLPLLVILVCYTR | 171 |
| GPCR5: | 219 | ILKTVLS---------IASREERAKALITCVSHICCVLVF--YVTVIGLSLIHRFGKQVP | 267 |
| | | \|\|+\|+ \| \|\| \| + \| + \|\| + \| \|+ \| + | |
| Sbjct: | 172 | ILRTLRKRARSQRSLKRRSSSERKAAKMLLVVVVVFVLCWLPYHIVLLLDSLCLLSIWRV | 231 |
| GPCR5: | 268 | HIVHLIMSYAYFLFPPLMNPITY 290 | |
| | | \|+++ +\|\|\| \| | |
| Sbjct: | 232 | LPTALLITLWLAYVNSCLNPIIY 254 | |

A GPCR-like protein of the invention, referred to herein as GPCR5, is an Olfactory Receptor (“OR”)-like protein. Some members of the Olfactory Receptor-Like Protein Family end up localized at the cell surface, where they exhibit activity. Therefore it is likely that these novel GPCR5 proteins are available at the appropriate sub-cellular localization and hence accessible for the therapeutic uses described in this application.

The GPCR disclosed in this invention is expressed in at least in some of the following tissues: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus. This information was derived by determining the tissue sources of the sequences that were included in the invention. In addition, the sequence is predicted to be expressed in the following tissues because of the expression pattern of (GENBANK-ID:gb:GENBANK-ID:MMU133430|acc: AJ133430) a closely related or6 gene homolog in species Mus musculus: olfactory epithelium. Expression data for GPCR5 is provided in Example 2.

The nucleic acids and proteins of GPCR5 are useful in potential therapeutic applications implicated in various GPCR-related pathological disorders and/or OR-related pathological disorders, described further above.

The novel nucleic acid encoding the GPCR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-GPCRX Antibodies” section below. The disclosed GPCR5 protein has multiple hydrophilic regions, each of which can be used as an immunogen. In one embodiment, for example, a contemplated GPCR5 epitope comprises from about amino acids 10 to about 20. In another embodiment, for example, a GPCR5 epitope comprises from about amino acids 45 to about 52. In further embodiments, for example, a GPCR5 epitope comprises from about 90 to about 95, from about 180 to about 185, and from about 275 to 308.

GPCR6

A sixth GPCR-like protein of the invention, referred to herein as GPCR6, is an Olfactory Receptor (“OR”)-like protein. Some members of the Olfactory Receptor-Like Protein Family end up localized at the cell surface, where they exhibit activity. Therefore it is likely that these novel GPCR6 proteins are available at the appropriate sub-cellular localization and hence accessible for the therapeutic uses described in this application.

The disclosed novel GPCR6 (alternatively referred to herein as CG55924-01) includes the 988 base nucleotide sequence (SEQ ID NO: 13) shown in Table 6A. A GPCR6 ORF begins with a Kozak consensus ATG initiation codon at nucleotides 5-7 and ends with a TAA codon at nucleotides 969-971. Putative untranslated regions upstream from the initiation codon and downstream from the termination codon are underlined in Table 6A, and the start and stop codons are in bold letters.

TALBE 6A
GPCR6 Nucleotide Sequence

(SEQ ID NO:13)

ACTAATGAATATGTCCAGCATGGAAACGATTAATTTTGTTAGCTACTTTATCCTCATGGGCTTTCCCTCAAGCCCA
GAAATGCAGCTCCTCTACTTTGGTCTCTTCTCATTAGCCTATACTCTCACCCTGATGGGAAATGCATCCATTGTCT
GTGCTGTGTGGTGGGACCAGCACCTTCACACTCCCATGTACACCCTCTTGGGAAATTTCTCTCTCCTGGAAATATG
TTATGTTATTACAACTGTTCCTAAACTGCTGGCCAACTTCCTCTCCACAAGCAAGTCCATCTCATTCATGAGTTGT
TTTGCACAGTTCTACTTCTTCTTATCTTTGGGGTATGATGAGGGCTTCTTCCTTTGCATCATGGCCTTTGACAGGT
ATCTTGCCATCTGCCGCCCTCTACGTTATCCATGCATTATGAATAAGCAAGTATGCACTGGCCTTATCATCTTTGC
ATGGTCATGTGTCTTTGTAATCTTCCTAATTCTGTTGATTCTCATTTCACAGATATCCTACTGTGGCCCAAATATT
ATCAACCATTTTGTTTGTGATCCTGTACCATTGGTGATGCTGTCCTGTTCTGCAGACATAATCATCACCTATCTCA
TTTACTCCACATTCAATTCTATCTTCATGATTGGCACCTTTCTCTTTATCCTTTGTTCCTATGCTCTGGTGATTCT
GGCTGTAATACAGATGCCTTCAGAGGCTGGCAAACGAAAGGCTTTCTCCACTTGTGCCTCTCATTTGGCAGTTGTC
ACCTTGTTTTATGGCTCTATCATGGTGATGTATGTTAGTCCTGGATCAGCACACCCAGTAAAAATGCAAAAAATCA
TTACCTTGTTCTATTCTGTGATAACACCACTCTGTAATCCTCTAATATATAGTCTCAGGAGCAAAGAGATGAAAGA
TTCTCTGAGGAAAATCTTCAGGACTGGAAAAGATGTTAATAAAATATAAATAAGAGACAATTTTCATTTATCAAAT

The GPCR6 protein (SEQ ID NO: 14 encoded by SEQ ID NO: 13 is 318 aa in length and is presented using the one-letter amino acid codes in Table 6B. The Psort profile for GPCR6 predicts that this sequence has a signal peptide and is likely to be a Type IIIa membrane protein, localized at the plasma membrane with a certainty of 0.6400. In alternative embodiments, a GPCR6 polypeptide is located to the Golgi body with a certainty of 0.4600, to the endoplasmic reticulum (membrane) with a certainty of 0.3700, or to the endoplasmic reticulum (lumen) with a certainty of 0.1000. The Signal P predicts a likely cleavage site for a GPCR6 peptide is between positions 39 and 40, i.e., at the dash in the sequence AYT-LT.

TABLE 6B
Encoded GPCR6 protein sequence

(SEQ ID NO:14)

MNMSSMETINFVSYFILMGFPSSPEMQLLYFGLFSLAYTLTLMGNASIVCAVWWDQHLHTPMYTLLGNFS
LLEICYVITTVPKLLANFLSTSKSISFMSCFAQFYFFLSLGYDEGFFLCIMAFDRYLAICRPLRYPCIMN
KQVCTGLIIFAWSCVFVIFLILLILISQISYCGPNIINHFVCDPVPLVMLSCSADIIITYLIYSTFNSIF
MIGTFLFILCSYALVILAVIQMPSEAGKRKAFSTCASHLAVVTLFYGSIMVMYVSPGSAHPVKMQKIITL
FYSVITPLCNPLIYSLRSKEMKDSLRKIFRTGKDVNKI

A BLAST analysis of GPCR6 was run against the proprietary PatP GENESEQ Protein Patent database. The amino acid sequence of GPCR6 have high homology to other proteins as shown in Table 6C.

TABLE 6C

BLASTX results for GPCR6

| | | |
|---|---|---|
| | | Smallest |
| | | Sum |
| | High | Probability |
| Sequences producing High-scoring Segment Pairs: | Score | P(N) |

| | | |
|---|---|---|
| patp:AAG71660 Human olfactory receptor polypeptide | 1472 | 1.3e−150 |
| patp:AAG72457 Human OR-like polypeptide query sequence | 1472 | 1.3e−150 |
| patp:AAU24676 Human olfactory receptor AOLFR175 | 945 | 8.9e−95 |
| patp:AAG71801 Human olfactory receptor polypeptide | 934 | 1.3e−93 |
| patp:AAG71802 Human olfactory receptor polypeptide | 887 | 1.2e−88 |

In a search of public sequence databases, it was found, for example, that the amino acid sequence of the protein of the invention was found to have 168 of 307 amino acid residues (54%) identical to, and 221 of 307 amino acid residues (71%) similar to, the 307 amino acid residue ptnr:SPTREMBL-ACC:Q9WU86 ODORANT RECEPTOR S1-Mus musculus (Mouse, E=8.3e-85). GPCR6 also has homology to the proteins shown in the BLASTP data in Table 6D.

TABLE 6D
GPCR6 BLASTP results

| Gene Index/ | | Length | Identity | Positives | |
|---|---|---|---|---|---|
| Identifier | Protein/Organism | (aa) | (%) | (%) | Expect |
| gi\|9938010\|ref\| | odorant receptor S1 gene | 324 | 166/297 | 216/297 | 2e−73 |
| NP_064684.1(NM_020288) | [Mus musculus] | | (55%) | (71%) | |
| gi\|17464984\|ref\| | Olfactory receptor | 408 | 126/299 | 204/299 | 1e−64 |
| XP_069619.1(XM_069619) | 51E2/rat | | (42%) | (68%) | |
| gi\|17476501\|ref\| | similar to OLFACTORY | 1056 | 123/301 | 179/301 | 4e−61 |
| XP_063251.1(XM_063251) | RECEPTOR-LIKE | | (40%) | (58%) | |
| | PROTEIN F6[Homo | | | | |
| | sapiens] | | | | |
| gi\|17445367\|ref\| | similar to OLFACTORY | 322 | 137/299 | 195/299 | 1e−58 |
| XP_060564.1(XM_060564) | RECEPTOR 6B1 | | (45%) | (64%) | |
| | (OLFACTORY RECEPTOR | | | | |
| | 7-3) (OR7-3) | | | | |
| | [Homo sapiens] | | | | |
| gi\|17464970\|ref\| | similar to olfactory receptor | 310 | 130/301 | 196/301 | 1e−57 |
| XP_069613.1(XM_069613) | [Homo sapiens] | | (43%) | (64%) | |

A multiple sequence alignment is given in Table 6E, with the GPCR6 protein being shown on line 1 in Table 6E in a ClustalW analysis, and comparing the GPCR6 protein with the related protein sequences shown in Table 6D. This BLASTP data is displayed graphically in the ClustalW in Table 6E.

[image]

[image]

[image]

[image]

Table 6F lists the domain description from DOMAIN analysis results against GPCR6. This indicates that the GPCR6 sequence has properties similar to those of other proteins known to contain this 254 amino acid 7tm domain (SEQ ID NO: 40) itself.

TABLE 6F
Domain Analysis of GPCR6

| | | |
|---|---|---|
| PSSMs producing significant alignments: | Score | E |
| | (bits) | value |

| | | | |
|---|---|---|---|
| gnl\|Pfam\|pfam00001 | 7tm_1, 7 transmembrane receptor (rhodopsin family) | 94.4 | 9e−21 |

| | | | |
|---|---|---|---|
| GPCR6: | 44 | GNASIVCAVWWDQHLHTPMYTLLGNFSLLEICYVITTVPKLLANFLSTSKSISFMSCFAQ | 103 |
| | | \|\| ++ + + \| \|\| \| \| ++ ++ +++\| \| \| + \| | 60 |
| Sbjct: | 1 | GNLLVILVILRTKKLRTPTNIFLLNLAVADLLFLLTLPPWALYYLVGGDWVFGDALCKLV | 60 |
| GPCR6: | 104 | FYFFLSLGYDEGFFLCIMAFDRYLAICRPLRYPCIMNKQVCTGLIIFAWSCVFVIFLILL | 163 |
| | | \|+ \|\| \| ++ \|\|\|\|\|\| \|\|\|\| \| + \|\|+ \| ++ \| \| | |
| Sbjct: | 61 | GALFVVNGYASILLLTAISIDRYLAIVHPLRYRRIRTPRRAKVLILLVWVLALLLSLPPL | 120 |
| GPCR6: | 164 | ILISQISYCGPNIINHFVCDPVPLVMLSCSADIIITYLIYSTFNSIFMIGTFLFILC--S | 221 |
| | | + + \| \|\| + ++++ \|+ + ++ + \|\| | |
| Sbjct: | 121 | LFSWLRTVEEG---NTTVCLIDFPEESVKRSYVLLSTLVGFVLPLLVILVCYTRILRTLR | 177 |
| GPCR6: | 222 | YALVILAVIQMPSEAGKRKAFSTCASHLAVVTLFYGSIMVMYVSP----GSAHPVKMQKI | 277 |
| | | ++ \| + ++ \| + \| + +\|+ + + + | |
| Sbjct: | 178 | KRARSQRSLKRRSSSERKAAKMLLVVVVVFVLCWLPYHIVLLLDSLCLLSTWRVLPTALL | 237 |
| GPCR6: | 278 | ITLFYSVITPLCNPLIY 294 | |
| | | \|\|\|+ + + \|\|+\|\| | |
| Sbjct: | 238 | ITLWLAYVNSCLNPITY 254 | |

The GPCR6 disclosed in this invention is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources. Further expression data for GPCR6 is provided in Example 2.

The nucleic acids and proteins of GPCR6 are useful in potential therapeutic applications implicated in various GPCR-related pathological disorders and/or OR-related pathological disorders, described further above.

The novel nucleic acid encoding the GPCR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-GPCRX Antibodies” section below. The disclosed GPCR6 protein has multiple hydrophilic regions, each of which can be used as an immunogen. In one embodiment, for example, a contemplated GPCR6 epitope comprises from about amino acids 55 to about 65. In another embodiment, for example, a GPCR6 epitope comprises from about amino acids 135 to about 145. In further embodiments, for example, a GPCR6 epitope comprises from about 235 to about 250, and from about 290 to about 312.

GPCR7

A further GPCR-like protein of the invention, referred to herein as GPCR7, is an Olfactory Receptor (“OR”)-like protein. Some members of the Olfactory Receptor-Like Protein Family end up localized at the cell surface, where they exhibit activity. Therefore it is likely that these novel GPCR7 proteins are available at the appropriate sub-cellular localization and hence accessible for the therapeutic uses described in this application.

In one embodiment, a GPCR7 variant is the novel GPCR7 (alternatively referred to herein as CG55922-01), which includes the 989 nucleotide sequence (SEQ ID NO: 15) shown in Table 7A. A GPCR7 ORF begins with a Kozak consensus ATG initiation codon at nucleotides 9-11 and ends with a TGA codon at nucleotides 953-955. Putative untranslated regions upstream from the initiation codon and downstream from the termination codon are underlined in Table 7A, and the start and stop codons are in bold letters.

TABLE 7A
GPCR7 Nucleotide Sequence

| | |
|---|---|
| CTGGCCTAATGAATGTCTCTGAGCCAAATTCCAGCTTTGCTTTTGTAAATGAATTTATACTCCAAGGTTTCTCTTGTGA | (SEQ ID NO:15) |
| GTGGACAATTCAGATCTTCCTCTTCTCACTCTTTACTACAATATATGCACTGACTATAACAGGGAATGGAGCCATTGCT | |
| TTTGTCCTGTGGTGTGACCGGCGACTTCACACTCCCATGTACATGTTCCTGGGAAATTTCTCCTTTTTAGAGATATGGT | |
| ATGTCTCTTCTACAGTTCCCAAGATGTTGGTCAACTTCCTTTCAGAGAAAAAAAACATCTCCTTTGCTGGATGTTTTCT | |
| CCAGTTTTATTTCTTCTTCTCTTTGGGTACATCAGAATGCTTGCTTTTGACTGTGATGGCCTTTGATCAGTACCTTGCT | |
| ATCTGCCGTCCCTTGCTCTATCCTAATATCATGACTGGGCATCTCTATGCCAAACTGGTCATACTGTGCTGGGTTTGTG | |
| GATTTCTGTGGTTCCTGATCCCCATTGTTCTCATCTCTCAGAAGCCCTTCTGTGGCCCAAACATTATTGACCATGTTGT | |
| GTGTGACCCAGGGCCACTATTTGCATTGGATTGTGTTTCTGCCCCAAGAATCCAACTGTTTTGCTACACTCTAAGCTCA | |
| TTAGTTATTTTTGGTAACTTCCTCTTTATTATTGGATCCTATACTCTTGTCCTGAAAGCTGTGTTGGGTATGCCTTCAA | |
| GCACTGGGAGACATAAGGCCTTCTCTACCTGTGGGTCTCATTTGGCTGTGGTATCACTGTGCTATAGCCCTCTTATGGT | |
| CATGTATGTGAGCCCAGGACTCGGACATTCTACAGGGATGCAGAAAATTGAAACTTTGTTCTATGCTATGGTGACCCCA | |
| CTCTTCAATCCCCTTATCTATAGCCTCCAGAATAAGGAGATAAAGGCAGCCCTGAGGAAAGTTCTGGGGAGTTCCAACA | |
| TAATCTAAGGCATATTAGATTATTCCTCCATGATCAGATCA | |

The GPCR7 protein (SEQ ID NO: 16) encoded by SEQ ID NO: 15 has 315 amino acid residues and is presented using the one-letter codes in Table 7B. The Psort profile for GPCR7 predicts that this sequence has a signal peptide and is likely to be a Type IIIb membrane protein, localized at the plasma membrane with a certainty of 0.6000. In alternative embodiments, a GPCR7 polypeptide is located to the Golgi body with a certainty of 0.4000, to the endoplasmic reticulum (membrane) with a certainty of 0.3000, or to the peroxisomal microbody with a certainty of 0.300. The Signal P predicts a likely cleavage site for a GPCR7 peptide is between positions 41 and 42, i.e., at the dash in the sequence IYA-LT.

TABLE 7B
Encoded GPCR7 protein sequence

| | |
|---|---|
| MNVSEPNSSFAFVNEFILQGFSCEWTIQIFLFSLFTTIYALTITGNGAIAFVLWCDRRLHTPMYMFLGNFSFLEIWYVS | (SEQ ID NO:58) |
| STVPKMLVNFLSEKKNISFAGCFLQFYFFFSLGTSECLLLTVMAFDQYLAICRPLLYPNTMTGHLYAKLVILCWVCGFL | |
| WFLIPIVLISQKPFCGPNIIDHVVCDPGPLFALDCVSAPRIQLFCYTLSSLVIFGNFLFIIGSYTLVLKAVLGMPSSTG | |
| RHKAFSTCGSHLAVVSLCYSPLMVMYVSPGLGHSTGMQKIETLFYAMVTPLFNPLIYSLQNKEIKAALRKVLGSSNII | |

A BLAST analysis of GPCR7 was run against the proprietary PatP GENESEQ Protein Patent database. The amino acid sequence of GPCR7 has high homology to other proteins as shown in Table 7C.

TABLE 7C
BLASTX results for GPCR7

| | | Smallest |
|---|---|---|
| | | Sum |
| | High | Probability |
| Sequences producing High-scoring Segment Pairs: | Score | P(N) |
| patp:AAG72215 Human olfactory receptor polypeptide | 1638 | 3.3e−168 |
| patp:AAG71688 Human olfactory receptor polypeptide | 1500 | 1.4e−153 |
| patp:AAG71459 Human olfactory receptor polypeptide | 1415 | 1.4e−144 |
| patp:aau24677 Human olfactory receptor AOLFR176 | 1061 | 4.6e−107 |
| patp:AAG71804 Human olfactory receptor polypeptide | 1057 | 1.2e−106 |

In a search of public sequence databases, it was found, for example, that the amino acid sequence of the GPCR7 was found to have 193 of 314 amino acid residues (61%) identical to, and 223 of 314 amino acid residues (71%) similar to, the 324 amino acid residue ptnr:SPTREMBL-ACC:Q9WU86 Odorant Receptor S1-Mus musculus (Mouse, E=7.7e-98). GPCR7 also has homology to the proteins shown in the BLASTP data in Table 7D.

TABLE 7D
GPCR7 BLASTP results

| Gene Index/ | | Length | Identity | Positives | |
|---|---|---|---|---|---|
| Identifier | Protein/Organism | (aa) | (%) | (%) | Expect |
| gi\|15293807\|gb\| | olfactory receptor | 217 | 213/217 | 214/217 | e−104 |
| AAK95096.1\|(AF399611) | [Homo sapiens] | | (98%) | (98%) | |
| gi\|9938010\|ref\| | odorant receptor S1 | 324 | 193/314 | 223/314 | e−88 |
| NP_064684.1\|(NM_020288) | gene [Mus musculus] | | (61%) | (70%) | |
| gi\|17476501\|ref\| | similar to OLFACTORY | 1056 | 135/293 | 181/293 | 3e−66 |
| XP_063251.1(XM_063251) | RECEPTOR-LIKE | | (46%) | (61%) | |
| | PROTEIN F6 (H. | | | | |
| | sapiens) [Homo sapiens] | | | | |
| gi\|15293805\|gb\| | olfactory receptor | 217 | 141/217 | 162/217 | 4e−64 |
| AAK95095.1\|(AF399610) | [Homo sapiens] | | (64%) | (73%) | |
| gi\|17477848\|ref\| | Olfactory | 248 | 138/158 | 143/158 | 5e−64 |
| XP_063689.1(XM_063689) | receptor/chimpanzee | | (87%) | (90%) | |

A multiple sequence alignment is given in Table 7E, with the GPCR7 protein being shown on line 1 in a ClustalW analysis, and comparing the GPCR7 protein with the related protein sequences shown in Table 7D. Only the C-ter portion of gi|17476501|ref|XP_{—}063251.1 (XM_{—}06325 1) is shown in the ClustalW alignment, because residues 1-550 had no homology to any of the other polypeptide sequences disclosed in Tables 7D and 7E. This BLASTP data is displayed graphically in the ClustalW in Table 7E.

[image]

[image]

[image]

Table 7F lists the domain description from DOMAIN analysis results against GPCR7. This indicates that the GPCR7 sequence has properties similar to those of other proteins known to contain this 254 amino acid 7tm domain (SEQ ID NO: 40) itself.

TABLE 7F

Domain Analysis of GPCR7

| | | |
|---|---|---|
| PSSMS producing significant alignments: | Score | E |
| | (bits) | value |

| | | | |
|---|---|---|---|
| gnl\|Pfam\|pfam00001 | 7tm_1, 7 transmembrane receptor (rhodopsin family) | 109 | 2e−25 |

| | | | |
|---|---|---|---|
| GPCR7: | 45 | GNGAIAFVLWCDRRLHTPMYMFLGNFSFLEIWYVSSTVPKMLVNFLSEKKNISFAGCFLQ | 104 |
| | | \|\| + \|+ ++\| \|\| +\|\| \| + ++ ++ + \| \| + \| \| \| | |
| Sbjct: | 1 | GNLLVILVILTRKKLRTPTNIFLLNLAVADLLFLLTLPPWALYYLVGGDWVFGDALCKLV | 60 |
| GPCR7: | 105 | FYFFFSLGTSECLLLTVMAFDQYLAICRPLLYPNIMTGHLYAKLVILCWVCGFLWFLIPI | 164 |
| | | \| \| + \|\|\|\| ++ \|+\|\|\|\| \|\| \| \| \| \|++\| \|\| \| \| \|+ | |
| Sbjct: | 61 | GALFVVNGYASILLLTAISIDRYLAIVHPLRYRRIRTPRRAKVLILLVWVLALLLSLPPL | 120 |
| GPCR7: | 165 | VLISQKPFCGPNIIDHVVCDPGPLFALDCVSAPRIQLFCYTLSSLVIFGNFLFIIGSYTL | 224 |
| | | + + \| ++ \| \| + \| \| +++ + | |
| Sbjct: | 121 | LFSWLRTVEEGNTTVCLIDFPEESVKRSYVLLSTLVGFVLPLLVILVCYTRILRTLRKRA | 180 |
| GPCR7: | 225 | VLKAVLGMPSSTGRHKAFSTCGSHLAVVSLCYSPLMVMYVSPGLGHSTGMQKIE-----T | 279 |
| | | + \| \|\|+ \| \| + \| \|\|+ \| ++ + \| + + + \| | |
| Sbjct: | 181 | RSQRSLKRRSSSERKAAKMLLVVVVVFV-LCWLPYHIVLLLDSLCLLSIWRVLPTALLIT | 239 |
| GPCR7: | 280 | LFYAMVTPLFNPLIY 294 | |
| | | \|+ \| \| \|\|+\|\| | |
| Sbjct: | 240 | LWLAYVNSCLNPIIY 254 | |

The GPCR7 protein predicted here is similar to the “Olfactory Receptor-Like Protein Family”, some members of which end up localized at the cell surface where they exhibit activity. Therefore, it is likely that this novel GPCR7 protein is available at the appropriate sub-cellular localization and hence accessible for the therapeutic uses described in this application.

The Olfactory Receptor-like GPCR7 proteins disclosed is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, public EST sources, literature sources, and/or RACE sources. Further expression data for GPCR7 is provided in Example 2. This is by no way limiting in that olfactory receptors are a class of G protein-coupled receptor which are known to be expressed in all tissue types.

The nucleic acids and proteins of GPCR7 are useful in potential therapeutic applications implicated in various GPCR-related pathological disorders and/or OR-related pathological disorders, described further herein.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-GPCRX Antibodies” section below. The disclosed GPCR7 protein has multiple hydrophilic regions, each of which can be used as an immunogen. In one embodiment, for example, a contemplated GPCR7 epitope comprises from about amino acids 60 to about 65. In another embodiment, for example, a GPCR7 epitope comprises from about amino acids 80 to about 90. In further embodiments, for example, a GPCR7 epitope comprises from about 175 to about 180, from about 130 to about 240, and from about 295 to 308.

GPCR8

The disclosed GPCR8 (also referred to as CG55728-01) includes the 989 base nucleotide sequence (SEQ ID NO: 17) shown in Table 8A. An open reading frame was identified beginning with an ATG initiation codon at nucleotides 1-3 and ending with a TGA codon at nucleotides 932-934. A putative untranslated region downstream from the termination codon is underlined in Table 8A, and the start and stop codons are in bold letters.

TABLE 8A
GPCR8 Nucleotide Sequence

| | |
|---|---|
| ATGGGAGAGCAAACTAAAAGAGAAAAATCAAATGTGACTACAATAATGGAATTTGTTCTTTTGGGGTTTTCTGATATT | (SEQ ID NO:17) |
| CCCAATCTCCACTGGATGCTTTTTAGTATATTTTTACTTATGTATTTGATGATCCTGATGTGCAATGGCATCATAATA | |
| CTACTAATAAAAATTCACCCCGCTCTCCAGACTCCCATGTATTTTTTTCTTAGCAATTTTTCCCTTTTGGAAATCTG | |
| TTATGTAACAATCATTATCCCAAGAATGCTCATGGACATTTGGACCAGAAAGGAAATATTTCTTTGTTTGCTTGTGC | |
| TACACAAATGTGTTTTTTTCTTATGCTTGGAGGCACGGAGTGTCTCCTTCTGACAGTGATGGCCTATGACCGCTACGT | |
| GGCTATTTGTAAGCCTTTGCAGTATCCTCTAGTGATGAACCACAAAGTCTGCATTCAGCTGATAATAGCTTCCTGGAC | |
| CATCACAATTCCTGTAGTAATTGGGGAAACATGCCAAATTTTCCTTTTGCCCTTTTGCGGAACTAACACAATTAATCA | |
| TTTCTTTTGTGACATCCCGCCAATACTCAAGCTTGCTTGTGGAAACATATTTGTGAATGAGATAACAGTCCATGTAGT | |
| AGCGGTGGTGTTTATCACGGTGCCATTTCTGTTGATTGTTGTCTCTTATGGCAAAATTATCTCCAACATTTTGAAATG | |
| TCATCAGCCAGAGGAAAGGCTAAAGCCTTCTCCACCTGCTCATCTCACCTAATAGTTGTAATCTTATTCTTTGGAGCA | |
| GGTACTATCACTTATTTACAGCCCAAACCACATCAGTTTCAAAGGATGGGGAAACTGATTTCTCTTTTCTACACCATT | |
| CTGATTCCAACTTTGAATCCTATTATATATACCCTGAGGAACAAAGATATCATGGTGGCATTGAGAAAATTACTAGCT | |
| AAGTTATTAACATGAGATGAAGACTTGAAATTACAGAAATAATTTCTTTATAG | |

The GPCR8 protein (SEQ ID NO: 18) encoded by SEQ ID NO: 17 is 316 amino acids in length and is presented using the one-letter amino acid codes in Table 8B. The Psort profile for GPCR8 predicts that this sequence has a signal peptide and is likely to be a Type IIIb membrane protein, localized at the plasma membrane with a certainty of 0.6000. In alternative embodiments, a GPCR8 polypeptide is located to the Golgi body with a certainty of 0.4000, to the endoplasmic reticulum (membrane) with a certainty of 0.3000, or to the peroxisomal microbody with a certainty of 0.3000. The Signal P predicts a likely cleavage site for a GPCR8 peptide is between positions 49 and 50, i.e., at the dash in the sequence CNG-II.

TABLE 8B

Encoded GPCR8 protein sequence

| | |
|---|---|
| MGEQTKREKSNVTTIMEFVLLGFSDIPNLHWMLFSIFLLMYLMILMCNGIIILLIKIHPALQTPMYFFLSNFSLLE | (SEQ ID NO:18) |
| ICYVTIIIPRMLMDIWTQKGNISLFACATQMCFFLMLGGTECLLLTVMAYDRYVAICKPLQYPLVMNHKVCIQLII | |
| ASWTITIPVVIGETCQIFLLPFCGTNTINHFFCDIPPILKLACGNIFVNEITVHVVAVVFITVPFLLIVVSYGKII | |
| SNILKLSSARGKAKAFSTCSSHLIVVILFFGAGTITYLQPKPHQFQRMGKLISLFYTILIPTLNPITYTLRNKDIM | |
| VALRKLLAKLLT | |

A BLAST analysis of GPCR8 was run against the proprietary PatP GENESEQ Protein Patent database. The amino acid sequence of GPCR8 has high homology to other proteins as shown in Table 8C.

TALBE 8C

BLASTX results for GPCR8

| | | Smallest |
|---|---|---|
| | | Sum |
| | High | Probability |
| Sequence producing High-scoring Segment Pairs: | Score | P(N) |
| patp:AAG72261 Human olfactory receptor polypeptide | 783 | 1.3e−77 |
| patp:AAU24605 Human olfactory receptor AOLFR96 | 783 | 1.3e−77 |
| patp:AAG71664 Human olfactory receptor polypeptide | 772 | 1.9e−76 |
| patp:AAG72192 Human olfactory receptor polypeptide | 772 | 1.9e−76 |
| patp:AAG72355 Human OR-like polypeptide query sequence | 772 | 1.9e−76 |

In a search of public database sequences, the full amino acid sequence of the protein of the invention was found to have 147 of 306 amino acid residues (48%) identical to, and 200 of 306 amino acid residues (65%) similar to, the 315 amino acid ptnr:SPTREMBL-ACC:Q9JKA6 Olfactory Receptor P2-Mus musculus (Mouse, E=5.2e-76). GPCR8 also has homology to the proteins shown in the BLASTP data in Table 8D.

TABLE 8D
GPCR8 BLASTP results

| Gene Index/ | | Length | Identity | Positives | |
|---|---|---|---|---|---|
| Identifier | Protein/Organism | (aa) | (%) | (%) | Expect |
| gi\|17474799\|ref\| | similar to olfactory | 316 | 144/303 | 207/303 | 8e−69 |
| XP_062598.1(XM_062598) | receptor (H. sapiens) | | (47%) | (67%) | |
| | [Homo sapiens] | | | | |
| gi\|12231029\|sp\| | OLFACTORY RECEPTOR | 316 | 138/303 | 197/303 | 4e−68 |
| Q15062\|O2H3_HUMAN | 2H3 (OLFACTORY | | (45%) | (64%) | |
| | RECEPTOR-LIKE | | | | |
| | PROTEIN FAT11) | | | | |
| gi\|14596253\|emb\| | dM538M10.8 (novel 7 | 321 | 149/304 | 201/304 | 6e−68 |
| CAC43451.1\|(AL136158) | transmembrane receptor | | (49%) | (66%) | |
| | (rhodopsin family) (olfactory | | | | |
| | receptor like) protein similar | | | | |
| | torat Scr d-2, −7, −8 and −9) | | | | |
| | [Mus musculus] | | | | |
| gi\|3153223\|gb\| | olfactory receptor-like protein | 257 | 150/303 | 200/303 | 1e−57 |
| AAC17222.1\|(AF034898) | [Rattus norvegicus] | | (49%) | (65%) | |
| gi\|7363437\|ref\| | olfactory receptor, family 10, | 306 | 143/299 | 202/299 | 1e−57 |
| NP_039229.1\|(NM_013941) | subfamily C, member 1 | | (47%) | (66%) | |
| | [Homo sapiens] | | | | |

A multiple sequence alignment is given in Table 8E, with the GPCR8 protein being shown on line 1, in a ClustalW analysis comparing GPCR8 with the related protein sequences disclosed in Table 8D.

[image]

[image]

Table 8F lists the domain description from DOMAIN analysis results against GPCR8. The results indicate that GPCR8 contains the protein domain 7tm_{—}1(InterPro)7 transmembrane receptor (rhodopsin family). This indicates that the GPCR8 sequence has properties similar to those of other proteins known to contain this domain as well as to the 254 amino acid 7tm domain (SEQ ID NO: 40) itself.

TABLE 8F Domain Analysis of GPCR8

| | | | |
|---|---|---|---|
| PSSMs producing significant alignments: | Score | E | |
| | (bits) | value | |
| gnl\|Pfam\|pfam00001 | \|7tm_1, 7 transmembrane receptor | 97.8 | 8e−22 |
| (rhodopsin family) | | | |

| | | | |
|---|---|---|---|
| GPCR8: | 48 | NGIIILLIKIHPALQTPMYFFLSNFSLLEICYVTIIIPRMLMDIWTQKGNISLFACATQM | 107 |
| | | \| ++\|\|+\| \|+\|\| \|\| \| ++ ++ ++ + \| \| + \| | |
| Sbjct: | 2 | NLLVILVILRTKKLRTPTNIFLLNLAVADLLFLLTLPPWALYYLVGGDWVFGDALCKLVG | 61 |
| GPCR8: | 108 | CFFLMLGGTECLLLTVMAYDRYVAICKPLQYPLVMNHKVCIQLIIASWTITIPVVIGETC | 167 |
| | | \|++ \| \|\|\|\| ++ \|\|\|+\|\| \|\|+\| + + \|\|+ \| + + + + | |
| Sbjct: | 62 | ALFVVNGYASILLLTAISIDRYLAIVHPLRYRRIRTPRRAKVLILLVWVLALLLSLP--- | 118 |
| GPCR8: | 168 | QIFLLPFCGTNTINHFFCDIPPILKLACGNIFVNETVHVVAVVFITVPFLLIVVSYGKI | 227 |
| | | + \| \| \| \| \| + +\| +\| \|+\|+\| \| +\| | |
| Sbjct: | 119 | PLLFSWLRTVEEGNTTVCLIDFP------EESVKRSYVLLSTLVGFVLPLLVILVCYTRI | 172 |
| GPCR8: | 228 | I---------SNILKLSSARGKAKAFSTCSSHLIVVILFFGAGTITYL----QPKPHQFQ | 274 |
| | | + \|\| \|+ + \| ++ \|+ + + \| + | |
| Sbjct: | 173 | LRTLRKRARSQRSLKRRSSSERKAAKMLLVVVVVFVLCWLPYHIVLLLDSLCLLSIWRVL | 232 |
| GPCR8: | 275 | RMGKLISLFYTILIPTLNPIIY | 296 |
| | | \|\|+\|+ + \|\|\|\|\|\| | |
| Sbjct: | 233 | PTALLITLWLAYVNSCLNPIIY | 254 |

The GPCR8 disclosed in this invention is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, public EST sources, literature sources, and/or RACE sources. Further expression data for GPCR8 is provided in Example 2.

The nucleic acids and proteins of GPCR8 are useful in potential therapeutic applications implicated in various GPCR-related pathological disorders and/or OR-related pathological disorders, described further above.

The novel nucleic acid encoding the GPCR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-GPCRX Antibodies” section below. The disclosed GPCR8 protein has multiple hydrophilic regions, each of which can be used as an immunogen. In one embodiment, for example, a contemplated GPCR8 epitope comprises from about amino acids 10 to about 20. In another embodiment, for example, a GPCR8 epitope comprises from about amino acids 135 to about 145. In further embodiments, for example, a GPCR8 epitope comprises from about 225 to about 245, and from about 260 to 278.

GPCR9

The disclosed GPCR9 (also referred to as CG55726-01) includes the 999 nucleotide sequence (SEQ ID NO: 19) shown in Table 9A. An open reading frame was identified beginning with an ATG at nucleotides 3-5 and ending with a TGA codon at nucleotides 944-946. Putative untranslated regions are found upstream from the initiation codon and downstream from the termination codon and are underlined in Table 9A. The start and stop codons are in bold letters.

TABLE 9A.
GPCR9 Nucleotide Sequence

(SEQ ID NO:19)

TCATGGCTGATGATAATTTTACAGTTGTCACTGAGTTTATTCTTTTGGGATTGACAGATCATGCTGAACTAAAAGCT
GTGCTTTTTGTGGTGTTCCTGGTGATTTACGCCATTACCTTGTTGAGGAATCTGGGCATGATCCTCTTAATCCAAAT
CACCTCCAAACTCCACACACCCATGTACTTTTTACTCAGCTGTCTTTCATTTGTGGATGCCTGCTATTCATCTGCAA
TTGCACCCAAAATGCTGGTGAACCTCCTGGTTGTGAAGGCAACAATTTCTTTCTCTGCTTGCATGGTACAGCATTTG
TGTTTCGGAGTGTTCATCACCACAGAAGGCTTCTTACTGTCAGTGATGGCCTATGACCGCTATGTGGCCATTGTGAG
TCCCTTGCTTTACACTGTAGCCATGTCTGATAGAAAGTGTGTGGAGCTTGTCACAGGATCATGGATAGGTGGAATAG
TTAACACATTAATCCACACAATCAGCTTGAGGAGACTGTCCTTTTGTAGGCTAAATGCTGTCAGCCACTTCTTCTGT
GACATTCCTTCACTGCTAAAGCTGTCATGTTCTGACACCTCCATGAATGAGTTGTTGCTGTTAACCTTCTCCGGAGT
CATTGCCATGGCCACCTTCTTGACTGTGATCATTTCCTACATCTTCATTGCTTTTGCTAGCCTAAGGATCCACTCAG
CATCAGGCAGACAGCAAGCCTTCTCCACCTGTGCCTCTCACCTGACTGCTGTGACCATATTCTATGGTACCTTAATC
TTTAGCTACATTCAGCCAAGCTCCCAGTATTTTGTGGAACAAGAGAAAGTGGTTTCTATGTTCTATACGCTAGGGAT
TCCCATGTTAAACCTGTTGATACACAGTTTGAGAAACAAGGACGTAAAGGAGGCAGTGAAAAGGATGGGCCAACACA
ATCTAACAGTGCTTAACTGAATTCATTCTGATGGAACTCACAAGGCGGCCTGAGCTGCAGATTCCCCTTTTTGGA

The GPCR9 protein (SEQ ID NO: 20) encoded by SEQ ID NO: 19 is 313 amino acids in length and is presented using the one-letter amino acid codes in Table 9B. The Psort profile for GPCR9 predicts that this sequence has a signal peptide and is likely to be a Type IIIb membrane protein, localized at the plasma membrane with a certainty of 0.6000. In alternative embodiments, a GPCR9 polypeptide is located to the Golgi body with a certainty of 0.4000, to the endoplasmic reticulum (membrane) with a certainty of 0.3000, or to the peroxisomal microbody with a certainty of 0.3000. The Signal P predicts a likely cleavage site for a GPCR9 peptide is between positions 36 and 37, i.e., at the dash in the sequence IYA-IT.

TABLE 9B.
Encoded GPCR9 protein sequence

(SEQ ID NO:20)

MADDNFTVVTEFILLGLTDHAELKAVLFVVFLVIYAITLLRNLGMILLIQITSKLHTPMYFLLSCLSFVDACYSSAIAP
KMLVNLLVVKATISFSACMVQHLCFGVFITTEGFLLSVMAYDRYVAIVSPLLYTVAMSDRKCVELVTGSWIGGIVNTLI
HTISLRRLSFCRLNAVSHFFCDIPSLLKLSCSDTSMNELLLLTFSGVIAMATFLTVIISYIFIAFASLRIHSASGRQQA
FSTCASHLTAVTIFYGTLIFSYIQPSSQYFVEQEKVVSMFYTLGIPMLNLLIHSLRNKDVKEAVKRMGQHNLTVLN

A BLAST analysis of GPCR9 was run against the proprietary PatP GENESEQ Protein Patent database. The amino acid sequence of GPCR9 had high homology to other proteins as shown in Table 9C.

TABLE 9C.

BLASTX results for GPCR9

| | | |
|---|---|---|
| | | Smallest |
| | | Sum |
| | High | Probability |
| Sequences producing High-scoring Segment Pairs: | Score | P(N) |

| | | |
|---|---|---|
| patp:AAG71968 Human olfactory receptor polypeptide | 1079 | 5.6e−109 |
| patp:AAG72875 Human olfactory receptor data exploratorium | 904 | 2.0e−90 |
| patp:AAG73039 Olfactory receptor-like polypeptide | 904 | 2.0e−90 |
| patp:AAG71834 Human olfactory receptor polypeptide | 844 | 4.5e−84 |
| patp:AAU24664 Human olfactory receptor AOLFR162 | 844 | 4.5e−84 |

In a search of public database sequences, GPCR9 also has homology to the proteins shown in the BLASTP data in Table 9D.

TABLE 9D.

GPCR9 BLASTP results

| Gene Index/ | | Length | Identity | Positives | |
|---|---|---|---|---|---|
| Identifier | Protein/Organism | (aa) | (%) | (%) | Expect |
| gi\|246534\|emb\|C | | | 174/304 | 234/304 | |
| AA64370.1(X947 | olfactory receptor 4 | 312 | (57%) | (76%) | 7e−81 |
| 44) | [Gallus gallus] | | | | |
| | similar to OLFACTORY | | | | |
| gi\|17460285\|ref\|X | RECEPTOR 5I1 (OLFACTORY | | 166/300 | 221/300 | |
| P_062109.1 (XM_ | RECEPTOR-LIKE PROTEIN | 313 | (55%) | (73%) | 4e−76 |
| 062109) | OLF1) [Homo sapiens] | | | | |
| | similar to OLFACTORY | | | | |
| gi\|17460179\|ref\|X | RECEPTOR 8B8 (OLFACTORY | | 167/304 | 218/304 | |
| P_062182.1 (XM_ | RECEPTOR TPCR85) | 452 | (54%) | (70%) | 1e−75 |
| 062182) | [Homo sapiens] | | | | |
| gi\|3746448\|gb\|AA | | | 162/303 | 221/303 | |
| C63971.1\| | olfactory receptor OR93 Gib | 313 | (53%) | (72%) | 8e−75 |
| (AF045580) | [Hylobates lar] | | | | |
| gi\|3746446\|gb\|AA | | | 164/303 | 220/303 | |
| C63970.1\| | olfactory receptor OR93Oo [Pongo | 313 | (54%) | (72%) | 9e−75 |
| (AF045579) | pygmaeus] | | | | |

A multiple sequence alignment is given in Table 9E, with the GPCR9 protein being shown on line 1, in a ClustalW analysis comparing GPCR9 with the related protein sequences disclosed in Table 9D.

[image]

[image]

Table 9F lists the domain description from DOMAIN analysis results against GPCR9. This indicates that the GPCR9 sequence has properties similar to those of other proteins known to contain this 254 amino acid 7tm domain (SEQ ID NO: 40) itself.

TABLE 9F

Domain Analysis of GPCR9

| | | |
|---|---|---|
| PSSMs producing significant alignments: | Score | E |
| | (bits) | value |
| gnl\|Pfam\|pfam00001 7tm_1, 7 transmembrane receptor | 78.2 | 7e−16 |
| (rhodopsin family) | | |

| | | | |
|---|---|---|---|
| GPCR9: | 42 | NLGMILLIQITSKLHTPMYFLLSCLSFVDACYSSAIAPKMLVNLLVVKATISFSACMVQH | 101 |
| | | \|\| +\|\|+\| \| \|\| \|\| \| \|+ \| + + \| \| \|+ + \| + | |
| Sbjct: | 2 | NLLVILVILRTKKLRTPTNIFLLNLAVADLLFLLTLPPWALYYLVGGDWVFGDALCKLVG | 61 |
| GPCR9: | 102 | LCFGVFITTEGFLLSVMAYDRYVAIVSPLLYTVAMSDRKCVELVTGSWIGGIVNTLIHTI | 161 |
| | | \| \| \|\|+ ++ \|\|\|+\|\|\| \|\| \| + \|+ \|+ \|+ ++ +\| + | |
| Sbjct: | 62 | ALFVVNGYASILLLTAISIDRYLAIVHPLRYRRIRTPRRAKVLILLVWVLALLLSLPPLL | 121 |
| GPCR9: | 162 | SLRRLSFCRLNAVSHFFCDIPSLLKLSCSDTSMNELLLLTFSGVIAMATFLTVIISYIFI | 221 |
| | | + \| +\| \| \| +\| ++ \| + + | |
| Sbjct: | 122 | FSWLRTVEEGNTTVCLIDFPEESVKRSYVLLSTLVGFVLPLLVILVCYTRILRTLRKRAR | 181 |
| GPCR9: | 222 | AFASLRIHSASGRQQAFSTCASHLTAVTIFYGTLIF----SYIQPSSQYFVEQEKVVSMF | 277 |
| | | + \|\|+ \|+\| \|+ \| + \| + \| \| \| + +++++ | |
| Sbjct: | 182 | SQRSLKRRSSSERKAAKMLLVVVVVFVLCWLPYHIVLLLDSLCLLSIWRVLPTALLITLW | 241 |
| GPCR9: | 278 | YTLGIPMLNLLI 289 | |
| | | \|\| +\| | |
| Sbjct: | 242 | LAYVNSCLNPII 253 | |

The GPCR9 disclosed in this invention is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, public EST sources, literature sources, and/or RACE sources. Further expression data for GPCR9 is provided in Example 2.

The nucleic acids and proteins of GPCR9 are useful in potential therapeutic applications implicated in various GPCR-related pathological disorders and/or OR-related pathological disorders, described further above.

The novel nucleic acid encoding the GPCR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-GPCRX Antibodies” section below. The disclosed GPCR9 protein has multiple hydrophilic regions, each of which can be used as an immunogen. In one embodiment, for example, a contemplated GPCR9 epitope comprises from about amino acids 225 to about 245. In another embodiment, for example, a GPCR9 epitope comprises from about amino acids 260 to about 275. In further embodiments, for example, a GPCR9 epitope comprises from about 285 to about 310.

GPCR10

The disclosed GPCR10 nucleic acid (SEQ ID NO: 21) of 974 nucleotides (also referred to as CG50325-01) is shown in Table 10A. An open reading frame was identified beginning with an ATG initiation codon at nucleotides 3-5 and ending with a TAG codon at nucleotides 960-962. Putative untranslated regions found upstream from the first codon and downstream from the termination codon are underlined in Table 10A, and the start and stop codons are in bold letters.

TABLE 10A.
GPCR10 Nucleotide Sequence

(SEQ ID NO:21)

ATATGGATCTATACAAGCTTCAATTAAACAATTTTACTGAAGTCACCATGTTTATATTAATAAGCTTCACAGAAGAATTTGAT
GTGCAAGTCTTCCTATTTTTATTATTTTTAGCAATCTATCTATTCACTCTAATAGGCAATTTAGGGCTGGTTGTACCGATCAT
TGGGGATTTCTGGCTTCACAGCCCAATGTACTATTTTCTTGGTGTTTTATCATTCTTGGATGTCTGCTATTCTACAGTTGTCA
CTCCAAAAATGTTGGTCAATTTCCTGGCAAAAAATAAATCTATTTCATTTCTTGGATGTGCAACACAGATGTTTCTTGCTTGT
ACTTTTGGAACCACAGAATGCTTTCTCTTGGCTGCAATGGCTTATGATCGCTATGTAGCCATCTACAACCCTCTCCTGTATTC
AGTGAGCATGTCACCCAGAGTCTATGTGCCACTCATCACTGCTTCCTATGTTGCTAGCATTTTACATGCTACTATACATACAG
TGGCTACATTTAGCCTGTCCTTCTGTGGATCCAATGAAATTAGGCATGTCTTTTGTAATATGCCTCCTCTGCTTGCTATTTCT
TGTTCTGACACTCACGTAATCCAGCTTCTATTCTTCTACTTTGTGGGCTCTATTGAGATAGTCACTATCCTGATTGTCCTGAT
CTCCTATGGTTTTATTCTGTTGGCCATTCTGAAGATGCAGTCTGCTGAAGGGAGGAGAAAAGTCTTCTCTACATGTGGAGCTC
ACCTAACTGGAGTGACAATTTATCATGGGACAATCCTCTTCATGTATGTGAGACCAAGTTCCAGCTACACTTCGGACAATGAC
ATGATAGTGTCAATATTTTATACCATTGTGATTCCCATGCTGAATCCCATCATCTACAGTTTGCGGAACAAAGATGTAAAGGA
GGCAATCAAAAGATTGCTTGTGAGAAATTGGTTCATAAATAAGTTATAGTTTTAAAATTGA

The disclosed GPCR10 polypeptide (SEQ ID NO: 22) encoded by SEQ ID NO: 21 has 319 amino acid residues and is presented using the one-letter amino acid codes in Table 10B. The Psort profile for GPCR10 predicts that this sequence has a signal peptide and is likely to be a Type IIIa membrane protein, localized at the plasma membrane with a certainty of 0.6400. In alternative embodiments, a GPCR10 polypeptide is located to the Golgi body with a certainty of 0.4600, to the endoplasmic reticulum (membrane) with a certainty of 0.3700, or to the endoplasmic reticulum (lumen) with a certainty of 0.1000. The Signal P predicts a likely cleavage site for a GPCR10 peptide is between positions 43 and 44, i.e., at the dash in the sequence LFT-II.

TABLE 10B.
GPCR10 protein sequence

(SEQ ID NO:22)

MDLYKLQLNNFTEVTMFILISFTEEFDVQVFLFLLFLAIYLFTLIGNLGLVVPIIGDFWLHSPMYYFLGV
LSFLDVCYSTVVTPKMLVNFLAKNKSISFLGCATQMFLACTFGTTECFLLAAMAYDRYVAIYNPLLYSVS
MSPRVYVPLITASYVASILHATIHTVATFSLSFCGSNEIRHVFCNMPPLLAISCSDTHVIQLLFFYFVGS
IEIVTILIVLISYGFILLAILKMQSAEGRRKVFSTCGAHLTGVTIYHGTILFMYVRPSSSYTSDNDMIVS
IFYTIVIPMLNPIIYSLRNKDVKEAIKRLLVRNWFINKL

A BLAST analysis of GPCR10 was run against the proprietary PatP GENESEQ Protein Patent database. The disclosed GPCR10 has homology to the amino acid sequences shown in the BLASTX data listed in Table 10C.

TABLE 10C
BLASTX results for GPCR10

| | | Smallest |
|---|---|---|
| | | Sum |
| Sequences producing High-scoring | High | Probability |
| Segment Pairs: | Score | P(N) |
| patp:AAG71588 Human olfactory | 1613 | 1.5e−165 |
| receptor polypeptide | | |
| patp:AAG72448 Human OR-like | 1613 | 1.5e−165 |
| polypeptide query sequence | | |
| patp:AAU24580 Human olfactory | 1332 | 8.7e−136 |
| receptor AOLFR70 | | |
| patp:AAG72303 Human olfactory | 1291 | 1.9e−131 |
| receptor polypeptide | | |
| patp:AAG71644 Human olfactory | 1260 | 3.7e−128 |
| receptor polypeptide | | |

In a search of public sequence databases, it was shown that GPCR10 amino acid sequence has 167 of 306 amino acid residues (54%/o) identical to, and 221 of 306 amino acid residues (72%) similar to, the 311 amino acid residue ptnr:SWISSPROT-ACC:Q95155 Olfactory Receptor-like Protein OLF2 (Canis familiaris, E=1.7e-86). The GPCR10 polypeptide also has homology to the proteins shown in the BLASTP data in Table 10D.

TABLE 10D
BLASTP results for GPCR10

| Gene Index/ | | Length | Identity | Positives | |
|---|---|---|---|---|---|
| Identifier | Protein/Organism | (aa) | (%) | (%) | Expect |
| gi\|17460135\|ref\| | similar to OLFACTORY | 322 | 258/310 | 281/310 | e−115 |
| XP_062171.1\|(XM_062171) | RECEPTOR 5I1 (OLFACTORY | | (83%) | (90%) | |
| | RECEPTOR-LIKE PROTEIN | | | | |
| | OLF1) [Homo sapiens] | | | | |
| gi\|17460139\|ref\| | similar to OLFACTORY | 318 | 240/312 | 274/312 | e−114 |
| XP_062172.1\|(XM_062172) | RECEPTOR 5I1 (OLFACTORY | | (76%) | (86%) | |
| | RECEPTOR-LIKE PROTEIN | | | | |
| | OLF1) [Homo sapiens] | | | | |
| gi\|17460133\|ref\| | similar to OLFACTORY | 197 | 197/197 | 197/197 | 9e−91 |
| XP_062170.1\|(XM_062170) | RECEPTOR 5I1 (OLFACTORY | | (100%) | (100%) | |
| | RECEPTOR-LIKE PROTEIN | | | | |
| | OLF1) [Homo sapiens] | | | | |
| gi\|17460285\|ref\| | similar to OLFACTORY | 313 | 170/301 | 222/301 | 6e−79 |
| XP_062109.1\|(XM_062109) | RECEPTOR 5I1 (OLFACTORY | | (56%) | (73%) | |
| | RECEPTOR-LIKE PROTEIN | | | | |
| | OLF1) [Homo sapiens] | | | | |
| gi\|2495054\|sp\| | OLFACTORY RECEPTOR- | 311 | 167/306 | 221/306 | 7e−78 |
| Q95155\|OLF2_CANFA | LIKE PROTEIN OLF2 | | (54%) | (71%) | |
| | [Canis familiaris] | | | | |

The homology of the GPCR10 sequence and other proteins is shown graphically in the ClustalW analysis shown in Table 10E.

[image]

[image]

Table 10 F lists the domain description from DOMAIN analysis results against GPCR10. This indicates that the GPCR10 sequence has properties similar to those of other proteins known to contain this 377 amino acid 7tm domain (SEQ ID NO: 83) itself.

TABLE 10F

Domain Analysis of GPCR10

(SEQ ID NO:83)

gnl|Pfam|pfam00001, 7tm_1, 7 transmembrane receptor (rhodopsin family).
Length = 254 residues, 100.0% aligned
Score = 95.1 bits (235), Expect = 5e-21

| | | | |
|---|---|---|---|
| GPCR10: | 46 | GNLGLVVPIIGDFWLHSPMYYFLGVLSFLDVCYSTVVTPKMLVNFLAKNKSISFLGCATQ | 105 |
| | | \|\|\| +++ \|+ \| +\| \|\| \|+ \|+ + + \| \| + + \| | |
| Sbjct: | 1 | GNLLVILVILRTKKLRTPTNIFLLNLAVADLLFLLTLPPWALYYLVGGDWVFGDALCKLV | 60 |
| GPCR10: | 106 | MFLACTFGTTECFLLAAMAYDRYVAIYNPLLYSVSMSPRVYVPLITASYVASILHATIHT | 165 |
| | | \| \| \|\| \|++ \|\|\|+\|\| +\|\| \| +\|\| \|\| +\| ++\| + | |
| Sbjct: | 61 | GALFVVNGYASILLLTAISIDRYLAIVHPLRYRRIRTPRRAKVLILLVWVLALLLSLPP- | 119 |
| GPCR10: | 166 | VATFSLSFCGSNEIRHVFCNMPPLLAISCSDTHVIQLLFFYFVGSIEIV--TILIVLISY | 223 |
| | | \|\| \| \| ++ \| + \|+ \| + +\| \| ++ + | |
| Sbjct: | 120 | -LLFSWLRTVEEGNTTVCLIDFPEESVKRSYVLLSTLVGFVLPLLVILVCYTRILRTLRK | 178 |
| GPCR10: | 224 | GFILLAILKMQSAEGRRKVFSTCGAHLTGVTIYHGTILFMYVRP----SSSYTSDNDMIV | 279 |
| | | \|\| +\|+ \|+ + \| + + + + \| +++ | |
| Sbjct: | 179 | RARSQRSLKRRSSSERKAAKMLLVVVVVFVLCWLPYHIVLLLDSLCLLSIWRVLPTALLI | 238 |
| GPCR10: | 280 | SIFYTIVIPMLNPIIY 295 | |
| | | +++ \| \|\|\|\|\|\| | |
| Sbjct: | 239 | TLWLAYVNSCLNPIIY 254 | |

GPCR10 is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, public EST sources, literature sources, and/or RACE sources. Further expression data for GPCR10 is provided in Example 2.

GPCR10 polypeptides are useful in the generation of antibodies that bind immunospecifically to the GPCR10 polypeptides of the invention. These antibodies may be generated according to methods known in the art, using prediction from hydrophobicity charts, as described in the “Anti-GPCRX Antibodies” section below. The disclosed GPCR10 proteins have multiple hydrophilic regions, each of which can be used as an immunogen. In one embodiment, for example, a contemplated GPCR10 epitope comprises from about amino acids 225 to about 255. In another embodiment, for example, a GPCR10 epitope comprises from about amino acids 260 to about 278. In further embodiments, for example, a GPCR10 epitope comprises from about 290 to about 310.

GPCR11

The disclosed GPCR11 nucleic acid (SEQ ID NO: 23) of 966 nucleotides (also referred to as CG50285-01) is shown in Table 11A. An open reading frame was identified beginning with an ATG initiation codon at nucleotides 14-16 and ending with a TAA codon at nucleotides 956-958. Putative untranslated regions upstream from the initiation codon and downstream from the termination codon are underlined in Table 11A, and the start and stop codons are in bold letters.

TABLE 11A

GPCR11 Nucleotide Sequence

(SEQ ID NO:23)

ATTTGTTGGGAATATGGAAAAAAGAAATCTAACAGTTGTCAGGGAATTCG
TCCTTCTGGGACTTCCTAGCTCAGCAGAGCAGCAGCACCTCCTGTCTGTG
CTCTTTCTCTGTATGTATTTAGCCACCACCTTGGGGAACATGCTCATCAT
TGCGACGATTGGCTTTGACTCTCACCTCCATTCCCCTATGTACTTCTTCC
TTAGTAACTTGGCCTTTGTTGACATCTGCTTTACGTCGACTACAGTCCCC
CAAATGGTAGTGAATATCTTGACTGGCACCAAGACTATCTCTTTTGCAGG
CTGCCTCACCCAGCTCTTCTTCTTCGTTTCTTTTGTGAATATGGACAGCC
TCCTTCTGTGTGTGATGGCGTATGATAGATATGTGGCGATTTGCCACCCC
TTACATTACACCGCCAGAATGAACCTGTGCCTTTGTGTCCAGCTAGTGGC
TGGACTGTGGCTTGTTACTTACCTCCACGCCCTCCTGCATACTGTCCTAA
TAGCACAGCTGTCCTTCTGTGCCTCCAATATCATCCATCATTTCTTCTGT
GATCTCAATCCTCTCCTGCAGCTCTCTTGCTCTGACGTCTCCTTCAATGT
AATGATCATTTTTGCAGTAGGAGGTCTATTGGCTCTCACGCCCCTTGTCT
GTATCCTCGTATCTTATGGACTTATCTTCTCCACTGTTCTGAAGATCACC
TCTACTCAGGGCAAGCAGAGAGCTGTTTCCACCTGCAGCTGCCACCTGTC
AGTGGTGGTGTTGTTTTACGGCACAGCCATCGCCGTCTATTTCAGCCCTT
CATCCCCCCATATGCCTGAGAGCGACACTCTGTCAACCATCATGTATTCA
ATGGTGGCTCCGATGCTGAATCCTTTCATCTATACCCTAAGGAACAGGGA
TATGAAGAGGGGACTTCAGAAAATGCTTCTCAAGTGCACAGTCTTTCAGC
AGCAATAATGACCTCA

The disclosed GPCR11 polypeptide (SEQ ID NO: 24) encoded by SEQ ID NO: 23 has 314 amino acid residues and is presented using the one-letter codes in Table 11B. The Psort profile for GPCR11 predicts that this sequence has a signal peptide and is likely to be a Type IIIb membrane protein, localized at the plasma membrane with a certainty of 0.6000. In alternative embodiments, a GPCR11 polypeptide is located to the Golgi body with a certainty of 0.4000, to the mitochondrial inner membrane with a certainty of 0.3750, or to the mitochondrial intermembrane space with a certainty of 0.3263. The Signal P predicts a likely cleavage site for a GPCR11 peptide is between positions 41 and 42, i.e., at the dash in the sequence TLG-NM.

TABLE 11B

Encoded GPCR11 polypeptide sequence.

(SEQ ID NO:24)

MEKRNLTVVREFVLLGLPSSAEQQHLLSVLFLCMYLATTLGNMLIIATIG
FDSHLHSPMYFFLSNLAFVDICFTSTTVPQMVVNILTGTKTISFAGCLTQ
LFFFVSFVNMDSLLLCVMAYDRYVAICHPLHYTARMNLCLCVQLVAGLWL
VTYLHALLHTVLIAQLSFCASNIIHHFFCDLNPLLQLSCSDVSFNVMIIF
AVGGLLALTPLVCILVSYGLIFSTVLKITSTQGKQRAVSTCSCHLSVVVL
FYGTAIAVYFSPSSPHMPESDTLSTIMYSMVAPMLNPFIYTLRNRDMKRG
LQKMLLKCTVFQQQ

A BLAST analysis of GPCR11 was run against the proprietary PatP GENESEQ Protein Patent database. The disclosed GPCR11 has homology to the amino acid sequences shown in the BLASTX data listed in Table 11C.

TABLE 11C

BLASTX results for GPCR11

| | | |
|---|---|---|
| | | Smallest |
| | | Sum |
| Sequences producing High-scoring | High | Probability |
| Segment Pairs: | Score | P(N) |

| | | |
|---|---|---|
| patp:AAG72455 Human OR-like | 1615 | 9.0e−166 |
| polypeptide query sequence | | |
| patp:AAG72209 Human olfactory | 1602 | 2.1e−164 |
| receptor polypeptide | | |
| patp:AAG71710 Human olfactory | 970 | 2.0e−97 |
| receptor polypeptide | | |
| patp:AAG72812 Human olfactory | 966 | 5.3e−97 |
| receptor data exploratorium | | |
| patp:AAG72978 Olfactory | 966 | 5.3e−97 |
| receptor-like polypeptide | | |

In a search of public sequence databases, the disclosed GPCR11 amino acid sequence was shown to have 174 of 284 amino acid residues (61%) identical to, and 221 of 284 amino acid residues (77%) similar to Homo sapiens protein ptnr:SWISSNEW-ACC:043749 OLFACTORY RECEPTOR 1F1 (OLFACTORY RECEPTOR 16-35) (OR16-35) E=5.8e-93). The GPCR11 also has homology to the proteins shown in the BLASTP data in Table 11D.

TABLE 11D

BLASTP results for GPCR11

| | | Length | Identity | Positives | |
|---|---|---|---|---|---|
| Gene Index/Identifier | Protein/Organism | (aa) | (%) | (%) | Expect |
| gi\|14423793\|sp\|Q15619\|O | OLFACTORY | 313 | 311/313 | 311/313 | e−144 |
| 1C1_HUMAN | RECEPTOR 1C1 | | (99%) | (99%) | |
| | (OLFACTORY | | | | |
| | RECEPTOR TPCR27) | | | | |
| gi\|15293705\|gb\|AAK950 | olfactory receptor [Homo | 216 | 214/216 | 214/216 | 8e−95 |
| 45.1\|(AF399560) | sapiens] | | (99%) | (99%) | |
| gi\|6912554\|ref\|NP_03649 | olfactory receptor, family 1, | 312 | 183/305 | 232/305 | 6e−84 |
| 2.1\|(NM_012360) | subfamily F, member 8 | | (60%) | (76%) | |
| | [Homo sapiens] | | | | |
| gi\|2370145\|emb\|CAA747 | olfactory receptor protein | 312 | 182/305 | 232/305 | 2e−83 |
| 94.1\|(Y14442) | [Homo sapiens] | | (59%) | (75%) | |
| gi\|205816\|gb\|AAA41740. | olfactory protein [Rattus | 313 | 177/305 | 228/305 | 4e−80 |
| 1\|(M64377) | norvegicus] | | (58%) | (74%) | |

The homology of these sequences is shown graphically in the ClustalW analysis shown in Table 11E.

[image]

[image]

Table 11F lists the domain description from DOMAIN analysis results against GPCR11. This indicates that the GPCR11 sequence has properties similar to those of other proteins known to contain this 377 amino acid 7tm domain (SEQ ID NO: 40) itself.

TABLE 11F

Domain Analysis of GPCR11

(SEQ ID NO:40)

gnl|Pfam|pfam00001, 7tm_1, 7 transmembrane receptor (rhodopsin family).
Length = 254 residues, 100.0% aligned
Score = 112 bits (280), Expect = 3e−26

| | | | |
|---|---|---|---|
| GPCR11: | 41 | GNMLIIATIGFDSHLHSPMYFFLSNLAFVDICFTSTTVPQMVVNILTGTKTISFAGCLTQ | 100 |
| | | \|\|+\|+\| \| \| +\| \|\| \|\|\| \|+ \| \| \| + ++ \| \| \| | |
| Sbjct: | 1 | GNLLVILVILRTKKLRTPTNIFLLNLAVADLLFLLTLPPWALYYLVGGDWVFGDALCKLV | 60 |
| GPCR11: | 101 | LFFFVSFVNMDSLLLCVMAYDRYVAICHPLHYTARMNLCLCVQLVAGLWLVTYLHALLHT | 160 |
| | | \|\| \|\|\| ++ \|\|\|+\|\| \|\|\| \| \|+ +\|++ \| +\| | |
| Sbjct: | 61 | GALFVVNGYASILLLTAISIDRYLAIVHPLRYRRIRTPRRAKVLILLVWVLALLLSLPPL | 120 |
| GPCR11: | 161 | VLIAQLSFCASNIIHHFFCDLNPLLQLSCSDVSFNVMIIFAVGGLLALTPLVCILVSYGL | 220 |
| | | + + \| ++ \| +\| \| + + +\| + + | |
| Sbjct: | 121 | LFSWLRTVEEGNTTVCLIDFPEESVKRSYVLLSTLVGFVLPLLVILVCYTRILRTLRKRA | 180 |
| GPCR11: | 221 | IFSTVLKITSTQGKQRAVSTCSCHLSVVVLFYGTAIAVYFSP----SSPHMPESDTLSTI | 276 |
| | | \|\| \|+ ++ \| + \|+ + \| + \| + + \| \|+ | |
| Sbjct: | 181 | RSQRSLKRRSSSERKAAKMLLVVVVVFVLCWLPYHIVLLLDSLCLLSIWRVLPTALLITL | 240 |
| GPCR11: | 277 | MYSMVAPMLNPFIY 290 | |
| | | + \| \|\|\| \|\| | |
| Sbjct: | 241 | WLAYVNSCLNPIIY 254 | |

GPCR11 is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, public EST sources, literature sources, and/or RACE sources. Further expression data for GPCR 11 is provided in Example 2.

GPCR11 polypeptides are useful in the generation of antibodies that bind immunospecifically to the GPCR 11 polypeptides of the invention. The antibodies are for use in therapeutic or diagnostic methods. These antibodies may be generated according to methods known in the art, using prediction from hydrophobicity charts, as described in the “Anti-GPCRX Antibodies” section below. The disclosed GPCR11 protein has multiple hydrophilic regions, each of which can be used as an immunogen. In one embodiment, for example, a contemplated GPCR11 epitope comprises from about amino acids 120 to about 130. In another embodiment, for example, a GPCR11 epitope comprises from about amino acids 225 to about 240. In further embodiments, for example, a GPCR11 epitope comprises from about 275 to about 315.

GPCR12

Yet another GPCR-like protein of the invention, referred to herein as GPCR12 (alternatively referred to as CG55995-01), is an Olfactory Receptor like protein.

The novel GPCR12 nucleic acid (SEQ ID NO: 25) of 828 nucleotides encoding a novel Olfactory Receptor-like protein is shown in Table 12A. An open reading frame for the mature protein was identified beginning with an ATG codon which codes for the amino acid methionine at nucleotides 1-3 and ending with a TGA codon at nucleotides 826-828. Putative untranslated regions downstream from the termination codon are underlined in Table 12A, and the start and stop codons are in bold letters.

TABLE 12A

GPCR12 Nucleotide Sequence

(SEQ ID NO:25)

ATGGGAAATGCCATCATTACAGTCATCATCTCCTTAAACCAGAGCCTCCA
CGTTCCCATGTACCTGTTCCTCCTGAACCTATCTGTGGTGGAGGTGAGTT
TCAGTGCAGTCATTACGCCTGAAATGCTGGTGGTGCTCTCTACTGAGAAA
ACTATGATTTCTTTTGTGGGCTGTTTTGCACAGATGTATTTCATCCTTCT
TTTTGGTGGGACTGAATGTTTTCTCCTGGGAGCGATGGCTTATGACCGAT
TTGCTGCAATTTGCCATCCTCTGAACTACCCAGTGATTATGAACAGAGGG
GTTTTTATGAAATTAGTAATATTCTCATGGATCTCAGGGATCATGGTGGC
TACTGTGCAGACCACTTGGGTATTTAGTTTTCCATTTTGTGGCCCCAATG
AAATTAATCATCTCTTCTGTGAGACTCCCCCGGTACTAGAGCTTGTGTGT
GCAGACACCTTCTTATTTGAAATCTATGCCTTCACAGGCACCATTTTGAT
TGTTATGGTTCCTTTCTTGTTGATCCTCTTGTCTTACATTCGAGTTCTGT
TTGCCATCCTGAAGATGCCATCAACTACTGGGAGACAAAAGGCCTTTTCC
ACCTGTGCCTCTCACCTCACATCTGTGACCCTGTTCTATGGCACAGCCAA
TATGACTTATTTACAACCCAAATCTGGCTACTCACCCGAAACCAAGAAAC
TGATCTCATTGGCTTACACGTTGCTTACCCCTCTGCTCAATCCGCTCATC
TATAGCTTACGAAACAGTGAGATGAAGAGGACTTTGATAAAACTATGGCG
AAGAAAAGTGATTTTACACACATTCTGA

In a search of public sequence databases, it was found, for example the nucleic acid sequence of this invention has 510 of 787 bases (64%) identical to a gb:GENBANK-ID:AF247657|acc:AF247657.1 mRNA from Mus musculus (Mus musculus olfactory receptor P2 (Olfr17) gene, complete cds).

The disclosed GPCR12 polypeptide (SEQ ID NO: 26) encoded by SEQ ID NO: 25 has 275 amino acid residues, and is presented using the one-letter codes in Table 12B. The SignalP, Psort and/or Hydropathy results predict that GPCR12 has a signal peptide and is likely to be localized at the plasma membrane with a certainty of 0.6400. In alternative embodiments, a GPCR12 polypeptide is located to the Golgi body with a certainty of 0.4600, the endoplasmic reticulum (membrane) with a certainty of 0.3700, or the endoplasmic reticulum (lumen) with a certainty of 0.1000. The most likely cleavage site for a GPCR12 peptide is between amino acids 35 and 36, i.e. at the dash in the sequence SFS-AV.

TABLE 12B
GPCR12 protein sequence

(SEQ ID NO:26)

MGNAIITVIISLNQSLHVPMYLFLLNLSVVEVSFSAVITPEMLVVLSTEKTMISFVGCFAQMYFILLFGGTECFLLG
AMAYDRFAAICHPLNYPVIMNRGVFMKLVIFSWISGIMVATVQTTWVFSFPFCGPNEINHLFCETPPVLELVCADTF
LFEIYAFTGTILIVMVPFLLILLSYIRVLFAILKMPSTTGRQKAFSTCASHLTSVTLFYGTANMTYLQPKSGYSPET
KKLISLAYTLLTPLLNPLIYSLRNSEMKRTLIKLWRRKVILHTF

In a search of a proprietary PatP database, the amino acid sequence of GPCR12 was found to have high homology to other OR-like proteins, as shown in Table 12C.

TABLE 12C

BLASTX results for GPCR12

| | | |
|---|---|---|
| | | Smallest |
| | | Sum |
| | High | Probability |
| Sequences producing High-scoring Segment Pairs: | Score | P(N) |

| | | |
|---|---|---|
| patp:AAG72191 Human olfactory receptor polypeptide | 1413 | 2.3e-144 |
| patp:AAU24704 Human olfactory receptor AOLFR203 | 1413 | 2.3e-144 |
| patp:AAG72188 Human olfactory receptor polypeptide | 1229 | 7.2e-125 |
| patp:AAU24695 Human olfactory receptor AOLFR194 | 1229 | 7.2e-125 |
| patp:AAG71671 Human olfactory receptor polypeptide | 862 | 5.6e-86 |

In a search of public sequence databases, it was shown that the disclosed GPCR12 amino acid sequence has 151 of 268 amino acid residues (56%) identical to, and 198 of 268 amino acid residues (73%) similar to, the 315 amino acid residue ptnr:SPTREMBL-ACC:Q9JKA6 protein from Mus musculus (Mouse) (OLFACTORY RECEPTOR P2). Additional BLASTP results are shown in Table 12D.

TABLE 12D
GPCR12 BLASTP results

| Gene Index/ | | Length | Identity | Positive | |
|---|---|---|---|---|---|
| Identifier | Protein/Organism | (aa) | (%) | (%) | Expect |
| gi\|14423787\|sp\| | OLFACTORY RECEPTOR | 314 | 275/275 | 275/275 | e−125 |
| P58181\|OAA3_HUMAN | 10A3 (HTPCRX12) | | (100%) | (100%) | |
| gi\|17472935\|ref\| | similar to OLFACTORY | 314 | 234/274 | 256/274 | e−110 |
| XP_061844.1\|(XM_061844) | RECEPTOR 10A3 | | (85%) | (93%) | |
| | (HTPCRX12) (H. sapiens) | | | | |
| | [Homo sapiens] | | | | |
| gi\|15293853\|gb\| | olfactory receptor | 217 | 217/217 | 217/217 | e−101 |
| AAK95119.1\|(AF399634) | [Homo sapiens] | | (100%) | (100%) | |
| gi\|12007436\|gb\| | hP3 olfactory receptor | 317 | 160/271 | 203/271 | 1e−76 |
| AAG45206.1\|AF321237_3 | [Homo sapiens] | | (59%) | (74%) | |
| (AF321237) | | | | | |
| gi\|7638409\|gb\| | olfactory receptor P2 | 315 | 151/268 | 198/268 | 9e−76 |
| AAF65461.1\|AF247657_1 | [Mus musculus] | | (56%) | (73%) | |
| (AF247657) | | | | | |

A multiple sequence alignment is given in Table 12E, with the GPCR12 protein being shown on line 1 in Table 12E in a ClustalW analysis, and comparing the GPCR12 protein with the related protein sequences shown in Table 12D. This BLASTP data is displayed graphically in the ClustalW in Table 12E.

[image]

[image]

Table 12F lists the domain description from DOMAIN analysis results against GPCR12. This protein contains a domain similar to the 7tm_{—}1, 7 transmembrane receptor. This indicates that the GPCR12 sequence has properties similar to those of other proteins known to contain this domain as well as to the 255 amino acid 7tm domain (SEQ ID NO: 40) itself.

TABLE 12F
Domain Analysis of GPCR12

| | Score | E |
|---|---|---|
| PSSMs producing significant alignments: | (bits) | value |
| gn1\|Pfam\|pfam00001 7tm_1, 7 transmembrane receptor (rhodopsin family) | 90.5 | 1e-19 |

| | | | |
|---|---|---|---|
| GPCR12: | 2 | GNAIITVIISLNQSLHVPMYLFLLNLSVVEVSFSAVITPEMLVVLSTEKTMISFVGCFAQ | 61 |
| | | \|\| ++ ++\| + \| \| +\|\|\|\|\|+\| ++ \| + \| \| \| + \| | |
| Sbjct: | 1 | GNLLVILVILRTKKLRTPTNIFLLNLAVADLLFLLTLPPWALYYLVGGDWVFGDALCKLV | 60 |
| GPCR12: | 62 | MYFILLFGGTECFLLGAMAYDRFAAICHPLNYPVIMNRGVFMKLVIFSWISGIMVATVQT | 121 |
| | | ++ \| \|\| \|++ \|\|+ \|\| \|\|\| \| \| \|++ \|+ ++++ | |
| Sbjct: | 61 | GALFVVNGYASILLLTAISIDRYLAIVHPLRYRRIRTPRRAKVLILLVWVLALLLSLPPL | 120 |
| GPCR12: | 122 | TWVFSFPFCGPNEINHLFCETPPVLELVCADTFLFEIYAFTGTILIVMVPFLLILLSYIR | 181 |
| | | + + \| \| ++ + +++\| +\| \| | |
| Sbjct: | 121 | LFSWLRTVEEGNTTVCLIDFPEESVKRSYVLLSTLVGFVLPLLVILVCYTRILRTLRKRA | 180 |
| GPCR12: | 182 | VLFAILKMPSTTGRQKAFSTCASHLTSVTLFYGTANMTYL----QPKSGYSPETKKLISL | 237 |
| | | \|\| \|++ \|+ \| + \| + + \| \| \|\|+\| | |
| Sbjct: | 181 | RSQRSLKRRSSSERKAAKMLLVVVVVFVLCWLPYHIVLLLDSLCLLSIWRVLPTALLITL | 240 |
| GPCR12: | 238 | AYTLLTPLLNPLIY 251 | |
| | | + \|\|\|+\|\| | |
| Sbjct: | 241 | WLAYVNSCLNPIIY 254 | |

The Olfactory Receptor-like GPCR12 disclosed in this invention is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources. Further expression data for GPCR12 is provided in Example 2.

The nucleic acids and proteins of GPCR12 are useful in potential diagnostic and therapeutic applications implicated in various GPCR-related pathological diseases and/or disorders, and/or in various other pathologies, as described above.

The polypeptides can be used as immunogens to produce antibodies specific for the invention, and as vaccines. They can also be used to screen for potential agonist and antagonist compounds. For example, a cDNA encoding the OR-like protein may be useful in gene therapy, and the OR-like protein may be useful when administered to a subject in need thereof. The novel nucleic acid encoding OR-like protein, and the OR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods and other diseases, disorders and conditions of the like.

These antibodies may be generated according to methods known in the art, using predictions from hydrophobicity charts, as described in the “Anti-GPCRX Antibodies” section below. The disclosed GPCR12 protein has multiple hydrophyllic regions, each of which can be used as an immunogen. In one embodiment, for example, a contemplated GPCR12 epitope comprises from about amino acids 190 to about 210. In another embodiment, for example, a GPCR12 epitope comprises from about amino acids 220 to about 240. In further embodiments, for example, a GPCR12 epitope comprises from about 250 to about 260.

GPCR13

Yet another GPCR-like protein of the invention, referred to herein as GPCR13 (alternatively referred to as CG50375-01), is an Olfactory Receptor-like protein.

The novel GPCR13 nucleic acid (SEQ ID NO: 27) of 955 nucleotides encoding a novel Olfactory Receptor-like protein is shown in Table 13A. An open reading frame was identified beginning with an ATG initiation codon at nucleotides 6-8 and ending with a TAA codon at nucleotides 945-947. Putative untranslated regions upstream from the initiation codon and downstream from the termination codon are underlined in Table 13A, and the start and stop codons are in bold letters.

TABLE 13A

GPCR13 Nucleotide Sequence

(SEQ ID NO:27)

| | |
|---|---|
| TAAGAATGCCTATAGCTAACGACACCCCGTTCCATACTTCTTCATTCCTACTGCTGGGTA | 60 |
| TCCCAGGGCTAGAAGATGTGCACATCTGGATTGGATTCCCTTTTTTCTCTGTGTATCTTG | 120 |
| TTGCACTCCTGGGAAATGCTGCTATCTTGTTTGTGATCCAAACTGAGCAGAGTCTCCATG | 180 |
| AGCCCATGTACTACTTCCTGGCCATGTTGGATTCCATTGACCTGAGCTTGTCTACGGCCA | 240 |
| CCATTCCCAAAATGCTGGGCATCTTCTGGTTCAATATCAAGGAAATACCTTTTGGAGGCT | 300 |
| ACCTTTCTCAGATGTTCTTCATCCATTTCTTCACTGTCATGGAGAGCATCGTGTTGGTGG | 360 |
| CCATGGCCTTTGACCGCTACATTGCCATTTGCAAACCTCTTCGGTACACCATGATCCTCA | 420 |
| CCAGCAAAATCATCAGCCTCATTGCAGGCATTGCTGTCCTGAGGAGCTTGTACATGGTCG | 480 |
| TTCCACTGGTGTTTCTCCTCTTAAGGTTGCCCTTCTGTGGACATCGTATCATCCCTCATA | 540 |
| CTTACTGTGAGCACATGGGCATTGCCCGTCTGGCCTGTGCCAGCATCAAAGTCAACATTA | 600 |
| GGTTTGGTCTTGGCAGTATTTCTCTCTTGTTATTGGATGTGCTCCTTATTATTCTCTCCC | 660 |
| ATATCAGGATCCTCTATGCTGTCTTCTGCCTGCCCTCCTGGGAAGCTCGACTCAAAGCTC | 720 |
| TCAACACCTGTGGCTCTCACATTGGTGTTATCTTAGCCTTTTCTACACCAGCATTTTTCT | 780 |
| CTTTCTTTACACACTGCTTTGGCCATGATATTCCCCAATATATCCACATTTTCTTGGCTA | 840 |
| ATCTATATGTGGTTGTTCCTCCCACCCTCAATCCTGTAATCTATGGGGTCAGAACCAAAC | 900 |
| ATATTAGGGAGACAGTGCTGAGGATTTTCTTCAAGACAGATCACTAAGGAGTTGA | |

In a search of public sequence databases, it was found that the disclosed GPCR13 nucleic acid sequence has 647 of 929 bases (69%) identical to a gb:GENBANK-ID:AF121979|acc:AF121979.1 MRNA from Mus musculus (Mus musculus odorant receptor S46 gene, complete cds).

The disclosed GPCR13 polypeptide (SEQ ID NO: 28) encoded by SEQ ID NO: 27 has 314 amino acid residues and is described using the one-letter codes in Table 13B. The SignalP, Psort and/or Hydropathy results predict that GPCR13 is a Type IIIa membrane protein, has a signal peptide, and is likely to be localized at the endoplasmic reticulum (membrane) with a certainty of 0.6850. In alternative embodiments, a GPCR13 polypeptide is located to the Golgi body with a certainty of 0.4600, the plasma membrane with a certainty of 0.6400, or the endoplasmic reticulum (lumen) with a certainty of 0.1000. The most likely cleavage site for a GPCR13 peptide is between amino acids 53 and 54, i.e. at the dash in the sequence IQT-EQ.

TABLE 13B
GPCR13 protein sequence

| | | |
|---|---|---|
| MPIANDTPFHTSSFLLLGIPGLEDVHIWIGFPFFSVYLVALLGNAAILFVIQTEQSLHEP | 60 | (SEQ ID NO:28) |
| MYYFLAMLDSIDLSLSTATIPKMLGIFWFNIKEIPFGGYLSQMFFIHFFTVMESIVLVAM | 120 | |
| AFDRYIAICKPLRYTMILTSKIISLIAGIAVLRSLYMVVPLVFLLLRLPFCGHRIIPHTY | 180 | |
| CEHMGIARLACASIKVNIRFGLGSISLLLLDVLLIILSHIRILYAVFCLPSWEARLKALN | 240 | |
| TCGSHIGVILAFSTPAFFSFFTHCFGHDIPQYIHIFLANLYVVVPPTLNPVIYGVRTKHI | 300 | |
| RETVLRIFFKTDH | | |

In a search of a proprietary PatP database, the amino acid sequence of GPCR13 was found to have high homology to other OR-like proteins, as shown in Table 13C.

TABLE 13C

BLASTX results for GPCR13

| | | Smallest |
|---|---|---|
| | | Sum |
| | High | Probability |
| Sequences producing High-scoring Segment Pairs: | Score | P (N) |
| patp:AAG71643 Human olfactory receptor polypeptide | 1583 | 2.2e−162 |
| patp:AAU24770 Human olfactory receptor AOLFR324B | 1583 | 2.2e−162 |
| patp:AAG72254 Human olfactory receptor polypeptide | 1459 | 3.0e−149 |
| patp:AAU24575 Human olfactory receptor AOLFR65 | 1226 | 1.5e−124 |
| patp:AAG71630 Human olfactory receptor polypeptide | 1120 | 2.5e−113 |

In a search of public sequence databases, the full GPCR13 amino acid sequence was found to have 199 of 307 amino acid residues (64%) identical to, and 243 of 307 amino acid residues (79%) similar to, the 318 amino acid residue ptnr:SPTREMBL-ACC:Q9WU93 protein from Mus musculus (Mouse) (ODORANT RECEPTOR S46). The GPCR13 polypeptide has substantial homology to other proteins, for example those as shown in Table 13D.

TABLE 13D
GPCR13 BLASTP results

| Gene Index/ | | Length | Identity | Positive | |
|---|---|---|---|---|---|
| Identifier | Protein/Organism | (aa) | (%) | (%) | Expect |
| gi\|17461580\|ref\| | similar to olfactory | 313 | 305/313 | 307/313 | e−187 |
| XP_062275.1\|(XM_062275) | receptor (H. sapiens) | | (97%) | (97%) | |
| | [Homo sapiens] | | | | |
| gi\|17461601\|ref\| | similar to olfactory | 340 | 247/295 | 261/295 | e−111 |
| XP_062280.1\|(XM_062280) | receptor (H. sapiens) | | (83%) | (87%) | |
| | [Homo sapiens] | | | | |
| gi\|17461586\|ref\| | similar to odorant receptor | 327 | 201/303 | 245/303 | 2e−98 |
| XP_062277.1\|(XM_062277) | S46 gene (H. sapiens) | | (66%) | (80%) | |
| | [Homo sapiens] | | | | |
| gi\|1745673l\|ref\| | similar to odorant receptor | 325 | 200/310 | 251/310 | 4e−97 |
| XP_061610.1\|(XM_061610) | S46 gene (H. sapiens) | | (64%) | (80%) | |
| | [Homo sapiens] | | | | |
| gi\|4680268\|gb\| | odorant receptor S46 | 318 | 199/307 | 243/307 | 4e−96 |
| AAD27599.1\|AF121979_1 | [Mus musculus] | | (64%) | (78%) | |
| (AF121979) | | | | | |

A multiple sequence alignment is given in Table 13E, with the GPCR13 protein being shown on line 1 in Table 13E in a ClustalW analysis, and comparing the GPCR13 protein with the related protein sequences shown in Table 13D. This BLASTP data is displayed graphically in the ClustalW in Table 13E.

[image]

[image]

[image]

Table 13F lists the domain description from DOMAIN analysis results against GPCR13. This protein contains a domain 7tm_{—}1, 7 transmembrane receptor. This indicates that the GPCR13 sequence has properties similar to those of other proteins known to contain this domain as well as to the 255 amino acid 7tm domain (SEQ ID NO: 40) itself.

TABLE 13F
Domain Analysis of GPCR13

| | | Score | E |
|---|---|---|---|
| PSSMs producing significant alignments: | (bits) | value | |
| gn1\|Pfam\|pfam00001 | 7tm_1,7 transmembrane receptor (rhodopsin family) | 51.6 | 7e−08 |

| | | | |
|---|---|---|---|
| GPCR13: | 43 | GNAAILFVIQTEQSLHEPMYYFLAMLDSIDLSLSTATIPKMLGIFWFNIKEIPFGGYLSQ | 102 |
| | | \|\| ++ \|\| + \| \| \|\| \| \|\| \| \| | |
| Sbjct: | 1 | GNLLVILVILRTKKLRTPTNIFLLNLAVADLLFLLTLPPWALYYLVGGDWVFGDALCKLV | 60 |
| GPCR13: | 103 | MFFIHFFTVMESIVLVAMAFDRYIAICKPLRYTMILT-SKIISLIAGIAVLRSLYMVVPL | 161 |
| | | ++\| \|++ \|\|\|+\|\| \|\|\|\| \| \| + \|\| + \|\| \| + \|\| | |
| Sbjct: | 61 | GALFVVNGYASILLLTAISIDRYLAIVHPLRYRRIRTPRRAKVLILLVWVLALLLSLPPL | 120 |
| GPCR13: | 162 | VFLLLRLPFCGHRIIPHTYCEHMGIARLACASIKVNIRFGLGSISLLLLDVLLIILSHIR | 221 |
| | | +\| \|\| \|+ + + \| + + \|+ +\| \| | |
| Sbjct: | 121 | LFSWLRTVEEGNTTVCLIDFPEESVKRSYVLLSTLVGFVLPLLVILVCYTRILRTLRKRA | 180 |
| GPCR13: | 222 | ILYAVFCLPSWEARLKALNTCGSHIGVILAFSTPAFFSFFT--HCFGHDIPQYIHIFLAN | 279 |
| | | \| \| \| + +\| + + + | |
| Sbjct: | 181 | RSQRSLKRRSSSERKAAKMLLVVVVVFVLCWLPYHIVLLLDSLCLLSIWRVLPTALLITL | 240 |
| GPCR13: | 280 | LYVVVPPTLNPVIY 293 | |
| | | \| \|\|\|+\|\| | |
| Sbjct: | 241 | WLAYVNSCLNPIIY 254 | |

The Olfactory Receptor-like GPCR13 disclosed in this invention is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources. Further expression data for GPCR13 is provided in Example 2.

The nucleic acids and proteins of GPCR1 3 are useful in potential diagnostic and therapeutic applications implicated in various GPCR-related pathological diseases and/or disorders, and/or in various other pathologies, as described above.

The polypeptides can be used as immunogens to produce antibodies specific for the invention, and as vaccines. They can also be used to screen for potential agonist and antagonist compounds. For example, a cDNA encoding the OR-like protein may be useful in gene therapy, and the OR-like protein may be useful when administered to a subject in need thereof. The novel nucleic acid encoding OR-like protein, and the OR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods and other diseases, disorders and conditions of the like. These antibodies may be generated according to methods known in the art, using predictions from hydrophobicity charts, as described in the “Anti-GPCRX Antibodies” section below. The disclosed GPCR13 protein has multiple hydrophyllic regions, each of which can be used as an immunogen. In one embodiment, for example, a contemplated GPCR13 epitope comprises from about amino acids 55 to about 60. In another embodiment, for example, a GPCR13 epitope comprises from about amino acids 170 to about 185. In further embodiments, for example, a GPCR13 epitope comprises from about 230 to about 240. In yet another embodiment, for example, a GPCR13 epitope comprises from about amino acids 290 to about 325.

GPCR14

Yet another GPCR-like protein of the invention, referred to herein as GPCR14 (alternatively referred to as CG5033 1-01), is an Olfactory Receptor (“OR”)-like protein.

The novel GPCR14 nucleic acid (SEQ ID NO: 29) of 946 nucleotides encoding a novel Olfactory Receptor-like protein is shown in Table 14A. An open reading frame was identified beginning with an ATG initiation codon at nucleotides 3-5 and ending with a TAA codon at nucleotides 936-938. Putative untranslated regions upstream of the initiation codon and downstream from the termination codon are underlined in Table 14A, and the start and stop codons are in bold letters.

TABLE 14A
GPCR14 Nucleotide Sequence

| | | |
|---|---|---|
| ACATGGAGATAAAGAACTACAGCAGCAGCACCTCAGGCTTCATCCTCCTGGGCCTCTCTT | 60 | (SEQ ID NO:29) |
| CCAACCCTAAGCTGCAGAAACCTCTCTTTGCCATCTTCCTCATCATGTACCTGCTCGCTG | 120 | |
| CGGTGGGGAATGTGCTCATCATCCCGGCCATCTACTCTGACCCCAGGCTCCACACCCCTA | 180 | |
| TGTACTTTTTTTTCAGCAACTTGTCTTTCATGGATATCTGCTTCACAACAGTCATAGTGC | 240 | |
| CTAAGATGCTGGTGAATTTTCTATCAGAGACAAAGGTTATCTCCTATGTGGGCTGCCTGG | 300 | |
| CCCAGATGTACTTCTTTATGGCATTTGGGAACACTGACAGCTACCTGCTGGCCTCTATGG | 360 | |
| CCATCGACCGGCTGGTGGCCATCTGCAACCCCTTACACTATGATGTGGTTATGAAACCAC | 420 | |
| GGCATTGCCTGCTCATGCTATTGGGTTCTTACAGCATCTCCCACCTACATTCCCTGTTCC | 480 | |
| GCGTGCTACTTATGTCTCGCTTGTCTTTCTGTGCCTCTCACATCATTAAGCACTTTTTCT | 540 | |
| GTGACACCCAGCCTGTGCTAAAGCTCTCCTGCTCTGACACATCCTCCAGCCAGATGGTGG | 600 | |
| TGATGACTGAGACCTTAGCTGTCATTGTGACCCCCTTCCTGTGTACCATCTTCTCCTACC | 660 | |
| TGCAAATCATCGTCACTGTGCTCAGAATCCCCTCTGCAGCCAGGAAGTGGAAGGCCTTCT | 720 | |
| CTACCTGTGGCTCCCACCTCACTGCAGTAGCCCTTTTCTATGGGAGTATTATTTATGTCT | 780 | |
| ATTTTAGGCCCCTGTCCATGTACTCAGTGGTTAGGGACCGGGTAGCCACAGTTATGTACA | 840 | |
| CAGTAGTGACACCCATGCTGAACCCTTTCATCTACAGCCTGAGGAACAAAGATATGAAGA | 900 | |
| GGGGTTTGAAGAAATTACAGGACAGAATTTACCGGTAAAAGGAACA | | |

In a search of public sequence databases, it was found that the disclosed GPCR12 nucleic acid sequence has 615 of 649 bases (94%/o) identical to a gb:GENBANK-ID:AF127882|acc:AF127882.1 mRNA from Callithrix jacchus (Callithrix jacchus olfactory receptor (CJA80) gene, partial cds).

The disclosed GPCR14 polypeptide (SEQ ID NO: 30) encoded by SEQ ID NO: 29 has 311 amino acid residues using the one-letter code in Table 14B. The SignalP, Psort and/or Hydropathy results predict that GPCR14 is a Type I membrane protein, has a signal peptide, and is likely to be localized at the plasma membrane with a certainty of 0.4600. In alternative embodiments, a GPCR14 polypeptide is located to the peroxisomal microbody with a certainty of 0.2245, the endoplasmic reticulum (membrane) with a certainty of 0.1000, or the endoplasmic reticulum (lumen) with a certainty of 0.1000. The most likely cleavage site for a GPCR14 peptide is between amino acids 52 and 53, i.e. at the dash in the sequence IYS-DP.

TABLE 14B
GPCR14 protein sequence

| | | |
|---|---|---|
| MEIKNYSSSTSGFILLGLSSNPKLQKPLFAIFLIMYLLAAVGNVLIIPAIYSDPRLHTPM | 60 | (SEQ ID NO:30) |
| YFFFSNLSFMDICFTTVIVPKMLVNFLSETKVISYVGCLAQMYFFMAFGNTDSYLLASMA | 120 | |
| IDRLVAICNPLHYDVVMKPHECLLMLLGSYSISHLHSLFRVLLMSRLSFCASHIIKHFFC | 180 | |
| DTQPVLKLSCSDTSSSQMVVMTETLAVIVTPFLCTIFSYLQIIVTVLRIPSAARKWKAFS | 240 | |
| TCGSHLTAVALFYGSIIYVYFRPLSMYSVVRDRVATVMYTVVTPMLNPFIYSLRNKDMKR | 300 | |
| GLKKLQDRIYR | | |

In a search of a proprietary PatP database, the amino acid sequence of GPCR14 was found to have high homology to other OR-like proteins, as shown in Table 14C.

TABLE 14C

BLASTX results for GPCR14

| | | Smallest |
|---|---|---|
| | | Sum |
| | High | Probability |
| Sequences producing High-scoring Segment Pairs: | Score | P(N) |
| patp:AAG72228 Human olfactory receptor polypeptide | 1554 | 2.6e−159 |
| patp:AAG72379 Human OR-like polypeptide query sequence | 1554 | 2.6e−159 |
| patp:AAG72859 Human olfactory receptor data exploratorium | 1554 | 2.6e−159 |
| patp:AAE04545 Human G-protein coupled receptor-1 | 1554 | 2.6e−159 |
| patp:AAU24522 Human olfactory receptor AOLFR7 | 1554 | 2.6e−159 |

In a search of the public sequence databases, the full GPCR14 amino acid sequence was found to have 196 of 216 amino acid residues (90%) identical to, and 203 of 216 amino acid residues (93%) similar to, the 216 amino acid residue ptnr:SPTREMBL-ACC:Q9N206 protein from Callithrixjacchus (Common marmoset) (OLFACTORY RECEPTOR). The disclosed GPCR14 also has homology to the proteins shown in the BLASTP data in Table 14D.

TABLE 14D
GPCR14 BLASTP Results

| Gene Index/ | | Length | Identity | Positive | |
|---|---|---|---|---|---|
| Identifier | Protein/Organism | (aa) | (%) | (%) | Expect |
| gi\|15293711\|gb\| | olfactory receptor [Homo | 216 | 211/216 | 213/216 | 6e−97 |
| AAK95048.1\| | sapiens] | | (97%) | (97%) | |
| (AF399563) | | | | | |
| gi\|15293709\|gb\| | olfactory receptor [Homo | 216 | 206/216 | 210/216 | 6e−95 |
| AAK95047.1\| | sapiens] | | (95%) | (96%) | |
| (AF399562) | | | | | |
| gi\|7211306\|gb\| | olfactory receptor | 216 | 196/216 | 203/216 | 1e−90 |
| AAF40279.1\| | [Callithrix jacchus] | | (90%) | (93%) | |
| (AF127877) | | | | | |
| gi\|7211316\|gb\| | olfactory receptor | 216 | 197/216 | 204/216 | 2e−90 |
| AAF40284.1\| | [Callithrix jacchus] | | (91%) | (94%) | |
| (AF127882) | | | | | |
| gi\|7211279\|gb\| | olfactory receptor | 216 | 195/216 | 202/216 | 5e−89 |
| AAF40268.1\| | [Eulemur rubriventer] | | (90%) | (93%) | |
| (AF127861) | | | | | |

A multiple sequence alignment is given in Table 14E, with the GPCR14 protein being shown on line 1 in a ClustalW analysis, and comparing the GPCR14 protein with the related protein sequences shown in Table 14D. This BLASTP data is displayed graphically in the ClustalW.

[image]

[image]

Table 14F lists the domain description from DOMAIN analysis results against GPCR14. This protein contains domain 7tm_{—}1, 7 transmembrane receptor. This indicates that the GPCR14 sequence has properties similar to those of other proteins known to contain this 255 amino acid 7tm domain (SEQ ID NO: 40) itself.

TABLE 14F
Domain Analysis of GPCR14

| PSSMs producing significant alignments: | Score | E |
|---|---|---|
| | (bits) | value |
| gnl/Pfam/pfam00001 7tm_1, 7 transmembrane receptor (rhodopsin family) | 108 | 6e-25 |

| | | | |
|---|---|---|---|
| GPCR14: | 42 | GNVLIIPAIYSDPRLHTPMYFFFSNLSFMDICFTTVIVPKMLVNFLSETKVISYVGCLAQ | 101 |
| | | \|\|+\|+\| \| +\| \|\| \| \|\|+ \|+ \| + \| \| + \| \| | |
| Sbjct: | 1 | GNLLVILVILRTKKLRTPTNIFLLNLAVADLLFLLTLPPWALYYLVGGDWVFGDALCKLV | 60 |
| GPCR14: | 102 | MYFFMAFGNTDSYLLASMAIDRLVAICNPLHYDVVMKPRHCLLMLLGSYSISHLHSLFRV | 161 |
| | | \|+ \| \|\| +++\|\|\| +\|\| +\|\| \| \| \|\| +++\| + ++ \| \|\| + | |
| Sbjct: | 61 | GALFVVNGYASILLLTAISIDRYLAIVHPLRYRRIRTPRRAKVLILLVWVLALLLSLPPL | 120 |
| GPCR14: | 162 | LLMSRLSFCASHIIKHFFCDTQPVLKLSCSDTSSSQMVVMTETLAVIVTPFLCTIFSYLQ | 221 |
| | | \| + \| + + \| + \|+ \|\| \| \| \| + \| + | |
| Sbjct: | 121 | LFSWL---------RTVEEGNTTVCLIDFPEESVKRSYVLLSTLVGFVLPLLVILVCYTR | 171 |
| GPCR14: | 222 | IIVTV---------LRIPSAARKWKAFSTCGSHLTAVALFYGSIIYVYFRPL----SMYS | 268 |
| | | \|+ \|+ \|+ \|++ + \| + \| + \| + \| | |
| Sbjct: | 172 | ILRTLRKRARSQRSLKRRSSSERKAAKMLLVVVVVFVLCWLPYHIVLLLDSLCLLSIWRV | 231 |
| GPCR14: | 269 | VVRDRVATVMYTVVTPMLNPFIY 291 | |
| | | + + \|+ \| \|\|\| \|\| | |
| Sbjct: | 232 | LPTALLITLWLAYVNSCLNPIIY 254 | |

The Olfactory Receptor-like GPCR14 disclosed in this invention is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, public EST sources, literature sources, and/or RACE sources. Further expression data for GPCR14 is provided in Example 2.

The nucleic acids and proteins of GPCR14 are useful in potential diagnostic and therapeutic applications implicated in various GPCR-related pathological diseases and/or disorders, and/or in various other pathologies, as described above.

The polypeptides can be used as immunogens to produce antibodies specific for the invention, and as vaccines. They can also be used to screen for potential agonist and antagonist compounds. For example, a cDNA encoding the OR-like protein may be useful in gene therapy, and the OR-like protein may be useful when administered to a subject in need thereof. The novel nucleic acid encoding OR-like protein, and the OR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods and other diseases, disorders and conditions of the like. These antibodies may be generated according to methods known in the art, using predictions from hydrophobicity charts, as described in the “Anti-GPCRX Antibodies” section below. The disclosed GPCR14 protein has multiple hydrophyllic regions, each of which can be used as an immunogen. In one embodiment, for example, a contemplated GPCR14 epitope comprises from about amino acids 55 to about 60. In another embodiment, for example, a GPCR14 epitope comprises from about amino acids 180 to about 195. In a further embodiment, for example, a GPCR14 epitope comprises from about 230 to about 240. In yet further specific embodiments, for example, a GPCR14 epitope comprises from about amino acids 290 to about 305.

GPCR15

Yet another GPCR-like protein of the invention, referred to herein as GPCR15 (alternatively referred to as CG50349-01), is an Olfactory Receptor (“OR”)-like protein.

The novel GPCR15 nucleic acid (SEQ ID NO: 3 1) of 2673 nucleotides encoding a novel Olfactory Receptor-like protein is shown in Table 15 A. An open reading frame was identified beginning with an ATG initiation codon at nucleotides 5-7 and ending with a TGA codon at nucleotides 2657-2659. Putative untranslated regions upstream from the initiation codon and downstream from the termination codon are underlined in Table 15A, and the start and stop codons are in bold letters.

TABLE 15A
GPCR15 Nucleotide Sequence

| | |
|---|---|
| AAACATGCATTCTTAATTATACTAATTACCTGCTTTGTGATTATTCTTGCTACTTCACAGCCTTGCCAGACC | (SEQ ID NO:31) |
| CCTGATGACTTTGTGGCTGCCACTTCTCCGGGACATATCATAATTGGAGGTTTGTTTGCTATTCATGAAGGTG | |
| ATAATTCTTTTTTTTCTTTTTCTTGGCAGGTTATTAACAAATTCTTTGAAATATCAGTTTTTCTTCAAACTCT | |
| TGCCATGATACACAGCATTGAGATGATCAACAATTCAACACTCTTACCTGGAGTCAAACTGGGGTATGAAATC | |
| TATGACACTTGTACAGAAGTCACAGTGGCAATGGCAGCCACTCTGAGGTTTCTTTCTAAATTCAACTGCTCCA | |
| GAGAAACTGTGGAGTTTAAGTGTGACTATTCCAGCTACATGCCAAGAGTTAAGGCTGTCATAGGTTCTGGGTA | |
| CTCAGAAATAACTATGGCTGTCTCCAGGATGTTGAATTTACAGCTCATGCCACAGGTGGGTTATGAATCAACT | |
| GCAGAAATCCTGAGTGACAAAATTCGCTTTCCTTCATTTTTACGGACTGTGCCCAGTGACTTCATCAAATTAA | |
| AAGCAATGGCTCACCTGATTCAGAAATCTGGTTGGAACTGGATTGGCATCATAACCACAGATGATGACTATGG | |
| ACGATTGGCTCTTAACACTTTTATAATTCAGGCTGAAGCAAATAACGTGTGCATAGCCTTCAAAGAGGTTCTT | |
| CCAGCCTTTCTTTCAGATAATACCATTGAAGTCAGAATCAATCGGACACTGAAGAAAATCATTTTAGAAGCCC | |
| AGGTTAATGTCATTGTGGTATTTCTGAGGCAATTCCATGTTTTTGATCTCTTCAATAAAGCCATTGAAATGAA | |
| TATAAATAAGATGTGGATTGCTAGTGATAATTGGTCAACTGCCACCAAGATTACCACCATTCCTAATGTTAAA | |
| AAGATTGGCAAAGTTGTAGGGTTTGCCTTTAGAAGAGGGAATATATCCTCTTTCCATTCCTTTCTTCAAAATC | |
| TGTCAGAAGCTAAAAGCAGAAACTCCAAGTTCACATCGGTTTTCTTCAGTTTGCCTGAGATATCAGGCAAAGC | |
| TTCATGTGTTAAAATATGCCTTAAACTCATTCATAGTATTCAGCTTGCAGTGTTTGCCCTTGGTTATGCCATT | |
| CGGGATCTGTGTCAAGCTCGTGACTGTCAGAACCCCAACGCCTTTCAACCATGGGAGTTACTTGGTGTGCTAA | |
| AAAATGTGACATTCACTGATGGATGGAATTCATTTCATTTTGATGCTCACGGGGATTTAAATACTGGATATGA | |
| TGTTGTGCTCTGGAAGGAGATCAATGGACACATGACTGTCACTAAGATGGCAGAATATGACCTAGCAAATTCC | |
| GCTTTCTCATTCACTGCAAGAAATTTTAAAAATATTTCCTACATTCAATCTAAATGCTCCAAGGAATGCAGTC | |
| CTGGGCAAATGAAGAAAACTACAAGAAGTCAACACATCTGTTGCTATGAATGTCAGAACTGTCCTGAAAATCA | |
| TTACACTAATCAGACAGATATGCCTCACTGCCTTTTATGCAACAACAAAACTCACTGGGCCCCTGTTAGGAGC | |
| ACTATGTGCTTTGAAAAGGAAGTGGAATATCTCAACTGGAATGACTCCTTGGCCATCCTACTCCTGATTCTCT | |
| CCCTACTGGGAATCATATTTGTTCTGGTTGTTGGCATAATATTTACAAGAAACCTGAACACACCTGTTGTGAA | |
| ATCATCCGGGGGATTAAGAGTCTGCTATGTGATCCTTCTCTGTCATTTCCTCAATTTTGCCAGCACGAGCTTT | |
| TTCATTGGAGAACCACAAGACTTCACATGTAAAACCAGGCAGACAATGTTTGGAGTGAGCTTTACTCTTTGCA | |
| TCTCCTGCATTTTGACGAAGTCTCTGAAAATTTTGCTAGCCTTCAGCTTTGATCCCAAATTACAGAAATTTCT | |
| GAAGTGCCTCTATAGACCGATCCTTATTATCTTCACTTGCACGGGCATCCAGGTTGTCATTTGCACACTCTGG | |
| CTAATCTTTGCAGCACCTACTGTAGAGGTGAATGTCTCCTTGCCCAGAGTCATCATCCTGGAGTGTGAGGAGG | |
| GATCCATACTTGCATTTGGCACCATGCTGGGCTACATTGCCATCCTGGCCTTCATTTGCTTCATATTTGCTTT | |
| CAAAGGCAAATATGAGAATTACAATGAAGCCAAATTCATTACATTTGGCATGCTCATTTACTTCATAGCTTGG | |
| ATCACATTCATCCCTATCTATGCTACCACATTTGGCAAATATGTACCAGCTGTGGAGATTATTGTCATATTAA | |
| TATCTAACTATGGAATCCTGTATTGCACATTCATCCCCAAATGCTATGTTATTATTTGTAAGCAAGAGATTAA | |
| CACAAAGTCTGCCTTTCTCAAGATGATCTACAGTTATTCTTCCCATAGTGTGAGCAGCATTGCCCTGAGTCCT | |
| GCTTCACTGGACTCCATGAGCGGCAATGTCACAATGACCAATCCCAGCTCTAGTGGCAAGTCTGCAACCTGGC | |
| AGAAAAGCAAAGATCTTCAGGCACAAGCATTTGCACACATATGCAGGGAAAATGCCACAAGTGTATCTAAAAC | |
| TTTGCCTCGAAAAAGAATGTCAAGTATATGAATAAGCCTTAGGAG | |

In a search of public sequence databases, it was found that the disclosed GPCR15 nucleic acid sequence has 843 of 1335 bases (63%) identical to a gb:GENBANK-ID:AF158963|acc:AF158963.1 mRNA from Carassius auratus (Carassius auratus odorant receptor 5.24 mRNA, complete cds).

The disclosed GPCR15 polypeptide (SEQ ID NO: 32) encoded by SEQ ID NO: 31 has 884 amino acid residues and is represented using the one-letter codes in Table 15B. The SignalP, Psort and/or Hydropathy results predict that GPCR15 is a Type IIIa membrane protein, has a signal peptide, and is likely to be localized at the plasma membrane with a certainty of 0.6400. In alternative embodiments, a GPCR15 polypeptide is located to the Golgi body with a certainty of 0.4600, the endoplasmic reticulum (membrane) with a certainty of 0.3700, or the endoplasmic reticulum (lumen) with a certainty of 0.1000. The most likely cleavage site for a GPCR15 peptide is between amino acids 18 and 19, ie. at the dash in the sequence ATS-QP.

TABLE 15B
GPCR15 protein sequence

(SEQ ID NO:97)

MAFLIILITCFVIILATSQPCQTPDDFVAATSPGHIIIGGLFAIHEGDNSFFSFSWQVINKFFEISVFLQTLA
MIHSIEMINNSTLLPGVKLGYEIYDTCTEVTVAMAATLRFLSKFNCSRETVEFKCDYSSYMPRVKAVIGSGYS
EITMAVSRMLNLQLMPQVGYESTAEILSDKIRFPSFLRTVPSDFHQIKAMAHLIQKSGWNWIGIITTDDDYGR
LALNTFIIQAEANNVCIAFKEVLPAFLSDNTIEVRINRTLKKIILEAQVNVIVVFLRQFHVFDLFNKAIEMNI
NKMWIASDNWSTATKITTIPNVKKIGKVVGFAFRRGNISSFHSFLQNLSEAKSRNSKFTSVFFSLPEISGKAS
CVKICLKLIHSIQLAVFALGYAIRDLCQARDCQNPNAFQPWELLGVLKNVTFTDGWNSFHFDAHGDLNTGYDV
VLWKEINGHMTVTKMAEYDLANSAFSFTARNFKNISYIQSKCSKECSPGQMKKTTRSQHICCYECQNCPENHY
TNQTDMPHCLLCNNKTHWAPVRSTMCFEKEVEYLNWNDSLAILLLILSLLGIIFVLVVGIIFTRNLNTPVVKS
SGGLRVCYVILLCHFLNFASTSFFIGEPQDFTCKTRQTMFGVSFTLCISCILTKSLKILLAFSFDPKLQKFLK
CLYRPILIIFTCTGIQVVICTLWLIFAAPTVEVNVSLPRVIILECEEGSILAFGTMLGYIAILAFICFIFAFK
GKYENYNEAKFITFGMLIYFIAWITFIPIYATTFGKYVPAVEIIVILISNYGILYCTFIPKCYVIICKQEINT
KSAFLKMIYSYSSHSVSSIALSPASLDSMSGNVTMTNPSSSGKSATWQKSKDLQAQAFAHICRENATSVS
KTLPRKRMSSI

In a search of a proprietary PatP database, the amino acid sequence of GPCR15 was found to have high homology to other OR-like proteins, as shown in Table 15C.

TABLE 15C

BLASTX results for GPCR15

| | | |
|---|---|---|
| | | Smallest |
| | | Sum |
| | High | Probability |
| Sequences producing High-scoring Segment Pairs: | Score | P(N) |

| | | |
|---|---|---|
| patp:AAY72609 Carassius auratus G protein-coupled OR | 1916 | 1.1e−197 |
| patp:AAY72618 Brachydanio rerio protein | 1748 | 7.2e−18O |
| patp:AAU02195 Cynomolgous monkey calcium-sensing receptor | 789 | 3.6e−139 |
| patp:AAW11889 Parathyroid calcium receptor | 789 | 7.4e−139 |
| patp:AAW54846 Human parathyroid calcium receptor 4.0 protein | 789 | 7.4e−139 |

In a search of public sequence databases, the full GPCR15 amino acid sequence was found to have 398 of 860 amino acid residues (46%) identical to, and 547 of 860 amino acid residues (63%) similar to, the 877 amino acid residue ptnr:SPTREMBL-ACC:Q9PW88 protein from Carassius auratus (Goldfish) (ODORANT RECEPTOR 5.24).

The amino acid sequence of GPCR15 was found to have high homology to other OR-like proteins, as shown in Table 15D.

TABLE 15D

GPCR15 BLASTP results

| | | | | | |
|---|---|---|---|---|---|
| Gene Index/ | | Length | Identity | Positive | |
| Identifier | Protein/Organism | (aa) | (%) | (%) | Expect |

| | | | | | |
|---|---|---|---|---|---|
| gi\|17463595\|ref\|XP₋0 | similar to odorant receptor | | 811/909 | 825/909 | |
| 69224.1\| | 5.24 (H. sapiens) | 910 | (89%) | (90%) | 0.0 |
| (XM₋069224) | [Homo sapiens] | | | | |
| gi\|5809686\|gb\|AAD46 | odorant receptor 5.24 | 392/838 | 535/838 | | |
| 570.2\|(AF158963) | [Carassius auratus] | | (46%) | (63%) | |
| gi\|3130153\|dbj\|BAA2 | calcium2 + sensing receptor | | 285/863 | 463/863 | |
| 6122.1\|(AB008857) | [Takifugu rubripes] | 930 | (33%) | (53%) | e−131 |
| gi\|14349134\|emb\|CAC | extracellular calcium | | 287/859 | 459/859 | |
| 41352.1\|(AJ289717) | sensing receptor precursor | 940 | (33%) | (53%) | e−130 |
| | [Sparus aurata] | | | | |
| gi\|453109\|gb\|AAB291 | Ca(2 + )-sensing receptor | | 1085 | 300/878 | 457/878 |
| 71.1\|(S67307) | [Bos taurus] | 1085 | (34%) | (51%) | e−126 |

A multiple sequence alignment is given in Table 15E, with the GPCR15 protein being shown on line 1 in a ClustalW analysis, and comparing the GPCR15 protein with the related protein sequences shown in Table 15D. This BLASTP data is displayed graphically in the ClustalW in Table 15E.

[image]

[image]

[image]

[image]

[image]

Table 15F lists the domain description from DOMAIN analysis results against GPCR15. This protein contains domain 7tm_{—}1, 7 transmembrane receptor. This indicates that the GPCR15 sequence has properties similar to those of other proteins known to contain this 255 amino acid 7tm domain (SEQ ID NO: 83) itself.

TABLE 15F
Domain Analysis of GPCR15

| | | |
|---|---|---|
| | Score | E |
| PSSMs producing significant alignments: | (bits) | value |
| gnl\|Pfam\|pfam00003 7tm₋3, 7 transmembrane receptor | 108 | 6e−25 |
| (metabotropic E family) | | |

| | | | |
|---|---|---|---|
| GPCR15: | 575 | RNLNTPVVKSSGGLRVCYVILLCHFLNFASTSFFIGEPQDFTCKTRQTMFGVSFTLCISC | 634 |
| | | ++ +\|\|+\|\|+\| + \|++\|+ \| + + \|\|\|+\| + +\| \|+ +\|\|+ \|\|\|\| \| | |
| Sbjct: | 25 | KHRDTPIVKASN-RELSYLLLIGLILCYLCSFLFIGKPSETSCILRRILFGLGFTLCYSA | 83 |
| GPCR15: | 635 | ILTKSLKILLAFSFDPKLQKFLKCL--YRPILIIFTCTGIQVVICTLWLIFAAPTVEVNV | 692 |
| | | +\| \|+ ++\| \| \| + + +\|\|+ \|\|\|+\|\| +\|\|+ \| +++ | |
| Sbjct: | 84 | LLAKTNRVLRIFRAKKPGSGKPKFISPWAQVLIVLILVLIQVIICVIWLVVEPPRPTIDI | 143 |
| GPCR15: | 693 | -SLPRVIILECEEGSILAFGTMLGYIAILAFICFIFAFKGKY--ENYNEAKFITFGMLIY | 749 |
| | | \| \|\|\|\|\| +\|\|++\|\| +\|\|\| +\|\| +\| \|\| + \|\|+\|\|\|\|\|\| \| \|\| + | |
| Sbjct: | 144 | YSEKEKIILECNKGSMVAFVVVLGYDGLLAVLCTFLAFLTRNLPENFNEAKFIGFSMLTF | 203 |
| GPCR15: | 750 | FIAWITFIPIYATTFGKYVPAVEIIVILISNYGILYCTFIPKCYVIICKQEIN 802 | |
| | | \| \|+ \|\|\|\|\| +\| \|\| \|\|\|\| \|\| \|+ +\| \| \|+\|\|\|\|+\|+ + \| \| | |
| Sbjct: | 204 | CIVWVAFIPIYLSTPGKVQVAVEIFSILASSTVLLGCLFVPKCYIILFRPEKN 256 | |

The Olfactory Receptor-like GPCR15 disclosed in this invention is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, those that express MIC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, public EST sources, literature sources, and/or RACE sources. Further expression data for GPCR15 is provided in Example 2.

The nucleic acids and proteins of GPCR15 are useful in potential diagnostic and therapeutic applications implicated in various GPCR-related pathological diseases and/or disorders, and/or in various other pathologies, as described above.

The polypeptides can be used as immunogens to produce antibodies specific for the invention, and as vaccines. They can also be used to screen for potential agonist and antagonist compounds. For example, a cDNA encoding the OR-like protein may be useful in gene therapy, and the OR-like protein may be useful when administered to a subject in need thereof. The novel nucleic acid encoding OR-like protein, and the OR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods and other diseases, disorders and conditions of the like. These antibodies may be generated according to methods known in the art, using predictions from hydrophobicity charts, as described in the “Anti-GPCRX Antibodies” section below. The disclosed GPCR15 protein has multiple hydrophyllic regions, each of which can be used as an immunogen. In one embodiment, for example, a contemplated GPCR15 epitope comprises from about amino acids 100 to about 220. In another embodiment, for example, a GPCR15 epitope comprises from about amino acids 240 to about 250. In further embodiments, for example, a GPCR15 epitope comprises from about 290 to about 350, from about amino acids 395 to about 550, from about amino acids 600 to about 620, from about amino acids 720 to about 740, and from about amino acids 810 to about 860.

GPCR16

Yet another GPCR-like protein of the invention, referred to herein as GPCR16 (alternatively referred to as CG50347-01), is an EBV-induced G protein-coupled receptor 2 (EBI2)-like protein.

The novel GPCR16 nucleic acid (SEQ ID NO: 33) of 1019 nucleotides is shown in Table 16A. An open reading frame was identified beginning with an ATG initiation codon at nucleotides 1-3 and ending with a TAA codon at nucleotides 1081-1083. Putative untranslated regions downstream from the termination codon are underlined in Table 16A, and the start and stop codons are in bold letters.

TABLE 16A.
GPCR16 Nucleotide Sequence

(SEQ ID NO:33)

ATGATAAAACTAGGCCCTTACTTCACACCACCCACAAAAATAAAAACTAAGATTAAAGATTTAAATGTGGAAAGCCAA
ACTGTGAAAATTTTTTTGCGCTCTCTGTTCTACACAGCCCTCCTGGTCTTCAGTGCCCTGGGAAACATCCTTGCCCTT
TGCCTTACCTGTCAAAAGAGCAGGAAGATCAACTGCACAGGCATCTACCTGGTGCACCTGGCTGTGTCTGACCTGCTG
TTCACCGTGGCCTTACCGGGAAGGGTGGTGTGTTATGTGCTGGGCTCCAGCTGGCCTTTCGGCAAGGGGCTCTGCAGG
CTGACGGCGTTTGTGCTCTACACCGACACCTACGGGGGGGTCTACCTCATGGCCTGTGTGAGCGTGGACCATTACCCA
GCTGTGGTCTGTGCCCACTGGGGCCCGCGCCTCCGCACGGCTGGCCGCGCCAGGCTGGTCTGCGTGGCCATCTGGACC
TTGGTGCTGCTGCAGACGATGCCCTTGCTCTTGATGCCCATGACCAAGCCGCTGGTGGGCAAGCTGGCCTGCATGGAG
TACAGCAGCATGGAGTCAGTCCTCGGGCTGCCCCTCATGGTCCTGGTGGCCTTTGCCATTGGCTTCTGTGGGCCAGTG
GGGATCATCCTGTCCTGCTATATGAAGATCACCTGGAAGCTGTGCAGCACAGCTCGGGAGGACCCAGTGACCAGCAGG
AAAGGACGCCACTGGCGAGGCTGCCTGCTTACGCTGCTGATGCTGGTGGCCGTGGTGGTCTGCTTCAGCCCCTACCAC
CTCAACATCAAGCAGTTCATGGCGAGAGGGATGCTCCACCTGCCATCCTGTGCCGAGCGGAGGGCTTTCTTACTGTCC
CTTCAGGCCACCGTGGCCCTCATGAACATGAACTGTGGCATTACCCCAATCATTTACTTCTTTGCATCCACCCATTAC
AGGAAATGGCTCCTGGGCATTTTAAAGCTCAAAGGGTCTTCCTCCTCCTCCTCCTCCTCCTCCTCCACCCCAGGAAAA
GCTTCTTCAGAAACACCAAGTATCACCCAGGCCAGAGGTTCTATGTTCTTAGCGGAGCACGTGGTCTAACGTGTCATT
TGCTTTATGACT

In a search of public sequence databases, it was found that the disclosed GPCR16 nucleic acid sequence has 562 of 996 bases (56%) identical to a gb:GENBANK-ID:HUMGPCRB|acc:L08177.1 mRNA from Homo sapiens (Human EBV induced G-protein coupled receptor (EBI2) mRNA, complete cds).

The disclosed GPCR16 polypeptide (SEQ ID NO: 34) encoded by SEQ ID NO: 33 has 360 amino acid residues and is presented using the one-letter code in Table 16B. The SignalP, Psort and/or Hydropathy results predict that GPCR16 is a Type III a membrane protein, has a signal peptide, and is likely to be localized at the plasma membrane with a certainty of 0.6000. In alternative embodiments, a GPCR16 polypeptide is located to the Golgi body with a certainty of 0.4000, or the endoplasmic reticulum (membrane) with a certainty of 0.3000. The most likely cleavage site for a GPCR16 peptide is between amino acids 51 and 52, i.e. at the dash in the sequence ILA-LC.

TABLE 16B.
GPCR16 protein sequence

(SEQ ID NO:34)

MIKLGPYFTPPTKIKTKIKDLNVESQTVKIFLRSLFYTALLVFSALGNILALCLTCQKSRKINCTGIYLVHLAVSDLLF
TVALPGRVVCYVLGSSWPFGKGLCRLTAFVLYTDTYGGVYLMACVSVDHYPAVVCAHWGPRLRTAGRARLVCVAIWTLV
LLQTMPLLLMPMTKPLVGKLACMEYSSMESVLGLPLMVLVAFAIGFCGPVGIILSCYMKITWKLCSTAREDPVTSRKGR
HWRGCLLTLLMLVAVVVCFSPYHLNIKQFMARGMLHLPSCAERRAFLLSLQATVALMNMNCGITPIIYFFASTHYRKWL
LGILKLKGSSSSSSSSSSTPGKASSETPSITQARGSMFLAEHVV

In a search of a proprietary PatP database, the amino acid sequence of GPCR16 was found to have high homology to other OR-like proteins, as shown in Table 16C.

TABLE 16C.

BLASTX results for GPCR16

| | | Smallest |
|---|---|---|
| | | Sum |
| | High | Probability |
| Sequences producing High-scoring Segment Pairs: | Score | P(N) |
| patp:AAR54080 Epstein Barr virus induced (EBI-2) polypeptide | 635 | 6.3e−62 |
| patp:AAW53623 Epstein Barr virus induced gene 2 (EBI-2) | 635 | 6.3e−62 |
| patp:AAY90G30 Human G protein-coupled receptor EBI2 | 635 | 6.3e−62 |
| patp:AAY90664 Human mutant G protein-coupled receptor EBI-2 | 633 | 1.0e−61 |
| patp:AAU25588 Human G Protein-Coupled Receptor (GPCR) | 401 | 4.0e−37 |

In a search of public sequence databases, the full GPCR16 amino acid sequence was found to have 133 of 343 amino acid residues (38%) identical to, and 203 of 343 amino acid residues (59%) similar to, the 361 amino acid residue ptnr:SWISSPROT-ACC:P32249 protein from Homo sapiens (Human) (EBV-INDUCED G PROTEIN-COUPLED RECEPTOR 2 (EBI2). The amino acid sequence of GPCR16 was found to have high homology to other OR-like proteins, as shown in Table 16D.

TABLE 16D
GPCR16 BLASTP results

| Gene Index/ | | Length | Identity | Positive | |
|---|---|---|---|---|---|
| Identifier | Protein/Organism | (aa) | (%) | (%) | Expect |
| gi\|17475475\|ref\| | similar to EBV-induced G | 229 | 143/168 | 148/168 | 2e−69 |
| XP_062863.1\| | protein-coupled receptor 2; | | (85%) | (87%) | |
| (XM_062863) | lymphocyte-specific G protein- | | | | |
| | coupled receptor; | | | | |
| | Epstein-Barr virus induced gene | | | | |
| | 2 (H. sapiens) [Homo sapiens] | | | | |
| gi\|292057\|gb\| | EBI 2: EBV induced G-protein | 361 | 133/344 | 203/344 | 9e−61 |
| AAA35924.1\| | coupled receptor [Homo | | (38%) | (58%) | |
| (L08177) | sapiens] | | | | |
| gi\|2687819\|emb\| | P2Y-like G-protein coupled | 367 | 90/296 | 146/296 | 4e−31 |
| CAA73144.1\| | receptor [Homo sapiens] | | (30%) | (48%) | |
| (Y12546) | | | | | |
| gi\|992700\|gb\| | putative G-protein-coupled | 339 | 90/296 | 146/296 | 7e−81 |
| AAB16746.1\| | receptor [Homo sapiens] | | (30%) | (48%) | |
| (U33447) | | | | | |
| gi\|2695876\|emb\| | P2Y-like G-protein coupled | 298 | 88/290 | 144/290 | 1e29 |
| CAB08108.1\| | receptor [Homo sapiens] | | (30%) | (49%) | |
| (Z94155) | | | | | |

A multiple sequence alignment is given in Table 16E, with the GPCR16 protein being shown on line 1 in a ClustalW analysis, and comparing the GPCR16 protein with the related protein sequences shown in Table 16D. This BLASTP data is displayed graphically in the ClustalW in Table 16E.

[image]

[image]

Table 16F lists the domain description from DOMAIN analysis results against GPCR16. This protein contains domain 7tm_{—}1, 7 transmembrane receptor. This indicates that the GPCR16 sequence has properties similar to those of other proteins known to contain this 255 amino acid 7tm domain (SEQ ID NO: 83) itself.

TABLE 16F.
Domain Analysis of GPCR16

| | | |
|---|---|---|
| PSSMs producing significant alignments: | Score | E |
| | (bits) | value |
| gnl\|Pfam\|pfam00003 7tm_3, 7 transmembrane receptor | 107 | 2e−24 |
| (metabotropic E family) | | |

| | | | |
|---|---|---|---|
| GPCR16: | 47 | GNILALCLTCQKSRKINCTGIYLVHLAVSDLLFTVALPGRVVCYVLGSSWPFGKGLCRLT | 106 |
| | | \|\|+\| + + + + \| \|+\|++\|\|\|+\|\|\|\| + \|\| + \|++\| \| \|\| \|\|+\| | |
| Sbjct: | 1 | GNLLVILVILRTKKLRTPTNIFLLNLAVADLLFLLTLPPWALYYLVGGDWVFGDALCKLV | 60 |
| GPCR16: | 107 | AFVLYTDTYGGVYLMACVSVDHYPAVVCAHWGPRLRTAGRARLVCVAIWTLVLLQTMPLL | 166 |
| | | + + \| + \|++\| \| \|+\| \|+\|\| \|\|+++ 30 +\| \| \|\| ++\| \| | |
| Sbjct: | 61 | GALFVVNGYASILLLTAISIDRYLAIVHPLRYRRIRTPRRAKVLILLVWVLALLLSLPPL | 120 |
| GPCR16: | 167 | LMPMTKPLVGKLACMEYSSMESVLGLPLMVLVAFAIGFCGPVGIILSCYMKITWKLCSTA | 226 |
| | | \| + + + \|\|++ +\|\| \|+ +\|\| \|\| +\| \| \| | |
| Sbjct: | 121 | LFSWLRTVEEGNTTVCLIDFPEESVKRSYVLLSTLVGFVLPLLVILVCYTRILRTLRKRA | 180 |
| GPCR16: | 227 | R-EDPVTSRKGRHWRGCLLTLLMLVAVVVCFSPYHLNIKQFMARGMLHLPSCAERRAFLL | 285 |
| | | \| + + \| + + \|+++\| \|+\|+ \|\|\| \| + \| | |
| Sbjct: | 181 | RSQRSLKRRSSSERKAAKMLLVVVVVFVLCWLPYH--IVLLLDSLCLLSIWRVLPTAL-- | 236 |
| GPCR16: | 286 | SLQATVALMNMNCGITPIIY 305 | |
| | | \|+ \| +\| + \|\|\|\| | |
| Sbjct: | 237 | --LITLWLAYVNSCLNPIIY 254 | |

Since Epstein-Barr virus (EBV) infection of Burkitt's lymphoma (BL) cells in vitro reproduces many of the activation effects of EBV infection of primary B lymphocytes, mRNAs induced in BL cells have been cloned and identified by subtractive hybridization. Nine genes encode RNAs which are 4-to >100-fold more abundant after EBV infection. Two of these, the genes for CD21 and vimentin, were previously known to be induced by EBV infection. Five others, the genes for cathepsin H, annexin VI (p68), serglycin proteoglycan core protein, CD44, and the myristylated alanine-rich protein kinase C substrate (MARCKS), are genes which were not previously known to be induced by EBV infection. Two novel genes, EBV-induced genes 1 and 2 (EBI 1 and EBI 2, respectively) can be predicted from their cDNA sequences to encode G protein-coupled peptide receptors. EBI 1 is expressed exclusively in B- and T-lymphocyte cell lines and in lymphoid tissues and is highly homologous to the interleukin 8 receptors. EBI 2 is most closely related to the thrombin receptor. EBI 2 is expressed in B-lymphocyte cell lines and in lymphoid tissues but not in T-lymphocyte cell lines or peripheral blood T lymphocytes. EBI 2 is also expressed at lower levels in a promyelocytic and a histiocytic cell line and in pulmonary tissue. These predicted G protein-coupled peptide receptors are more likely to be mediators of EBV effects on B lymphocytes or of normal lymphocyte functions than are genes previously known to be up-regulated by EBV infection.

The rhodopsin-like GPCRs themselves represent a widespread protein family that includes hormone, neurotransmitter and light receptors, all of which transduce extracellular signals through interaction with guanine nucleotide-binding (G) proteins. Although their activating ligands vary widely in structure and character, the amino acid sequences of the receptors are very similar and are believed to adopt a common structural framework comprising 7 transmembrane (TM) helices. G-protein-coupled receptors (GPCRs) constitute a vast protein family that encompasses a wide range of functions (including various autocrine, paracrine and endocrine processes). They show considerable diversity at the sequence level, on the basis of which they can be separated into distinct groups. We use the term clan to describe the GPCRs, as they embrace a group of families for which there are indications of evolutionary relationship, but between which there is no statistically significant similarity in sequence. The currently known clan members include the rhodopsin-like GPCRs, the secretin-like GPCRs, the cAMP receptors, the fungal mating pheromone receptors, and the metabotropic glutamate receptor family.

Regulator of G Protein Signaling (RGS) proteins function as GTPase-activating proteins (GAPs) that stimulate the inactivation of heterotrimeric G proteins and are responsible for the rapid turnoff of G protein-coupled receptor signaling pathways. RGS proteins may be regulated on a posttranslational level but the mechanisms controlling the GAP activity of RGS proteins are still poorly understood. There are indications that specific RGS proteins regulate specific G protein-coupled receptor pathways. The RGS domain is present in a number of different proteins that include: G protein-coupled receptor kinase, G-alpha interacting protein and others.

The GPCR16 disclosed in this invention is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, public EST sources, literature sources, and/or RACE sources. Further expression data for GPCR16 is provided in Example 2.

The nucleic acids and proteins of GPCR16 are useful in potential diagnostic and therapeutic applications implicated in various GPCR-related pathological diseases and/or disorders, and/or in various other pathologies, as described above.

The polypeptides can be used as immunogens to produce antibodies specific for the invention, and as vaccines. They can also be used to screen for potential agonist and antagonist compounds. For example, a cDNA encoding GPCR16 may be useful in gene therapy, and the GPCR16 polynucleotide may be useful when administered to a subject in need thereof. The novel nucleic acid encoding GPCR16, and the polypeptide of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods and other diseases, disorders and conditions of the like. These antibodies may be generated according to methods known in the art, using predictions from hydrophobicity charts, as described in the “Anti-GPCRX Antibodies” section below. The disclosed GPCR16 protein has multiple hydrophyllic regions, each of which can be used as an immunogen. In one embodiment, for example, a contemplated GPCR16 epitope comprises from about amino acids 10 to about 40. In another embodiment, for example, a GPCR16 epitope comprises from about amino acids 130 to about 140. In further embodiments, for example, a GPCR16 epitope comprises from about 220 to about 240, from about amino acids 260 to about 270, and from about amino acids 325 to about 350.

GPCRX Nucleic Acids and Polypeptides

A summary of the GPCRX nucleic acids and proteins of the invention is provided in Table 17.

TABLE 17
Summary Of Nucleic Acids And Proteins Of The Invention

| | | Nucleic | Amino |
|---|---|---|---|
| | | Acid | Acid |
| GPCRX | Clone; Description | SEQ ID | SEQ ID |
| Name | of Homolog | NO | NO |
| GPCR1 | GPCR-like; OR-like | 1 | 2 |
| GPCR2 | GPCR-like; OR-like | 3 | 4 |
| GPCR3 | GPCR-like; OR-like | 5 | 6 |
| GPCR4a | GPCR-like; OR-like | 7 | 8 |
| GPCR4b | GPCR-like; OR-like | 9 | 10 |
| GPCR5 | GPCR-like; OR-like | 11 | 12 |
| GPCR6 | GPCR-like; OR-like | 13 | 14 |
| GPCR7 | GPCR-like; OR-like | 15 | 16 |
| GPCR8 | GPCR-like; OR-like | 17 | 18 |
| GPCR9 | GPCR-like; OR-like | 19 | 20 |
| GPCR10 | GPCR-like; OR-like | 21 | 22 |
| GPCR11 | GPCR-like; OR-like | 23 | 24 |
| GPCR12 | GPCR-like; OR-like | 25 | 26 |
| GPCR13 | GPCR-like; OR-like | 27 | 28 |
| GPCR14 | GPCR-like; OR-like | 29 | 30 |
| GPCR15 | GPCR-like; OR-like | 31 | 32 |
| GPCR16 | GPCR-like; EBI2-like | 33 | 34 |

One aspect of the invention pertains to isolated nucleic acid molecules that encode GPCRX polypeptides or biologically active portions thereof. Also included in the invention are nucleic acid fragments sufficient for use as hybridization probes to identify GPCRX-encoding nucleic acids (e.g., GPCRX mRNAs) and fragments for use as PCR primers for the amplification and/or mutation of GPCRX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An GPCRX nucleic acid can encode a mature GPCRX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′-and 3′-termini of the nucleic acid) in the genomic DNA of the organism from which the nucleic acid is derived. For example, in various embodiments, the isolated GPCRX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a CDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NOS: 1, 3, 5, 7, 9, 11, 13, 15, 17, 19, 21, 23, 25, 27, 29, 31 and 33, or a complement of this aforementioned nucleotide sequence, can be isolated using standard molecular biology techniques and the sequence information provided herein. Using all or a portion of the nucleic acid sequence of SEQ ID NOS: 1, 3, 5, 7, 9, 11, 13, 15, 17, 19, 21, 23, 25, 27, 29, 31 and 33 as a hybridization probe, GPCRX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to GPCRX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOS: 1, 3, 5, 7, 9, 11, 13, 15, 17, 19, 21, 23, 25, 27,29, 31 and 33, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS: 1, 3, 5, 7, 9, 11, 13, 15, 17, 19, 21, 23, 25, 27, 29, 31 and 33, or a portion of this nucleotide sequence (e.g., a fragment that can be used as a probe or primer or a fragment encoding a biologically-active portion of an GPCRX polypeptide). A nucleic acid molecule that is complementary to the nucleotide sequence shown in SEQ ID NOS: 1, 3, 5, 7, 9, 11, 13, 15, 17, 19, 21, 23, 25, 27, 29, 31 and 33 is one that is sufficiently complementary to the nucleotide sequence shown in SEQ ID NOS: 1, 3, 5, 7, 9, 11, 13, 15, 17, 19, 21, 23, 25, 27, 29, 31 and 33 that it can hydrogen bond with little or no mismatches to the nucleotide sequence shown SEQ ID NOS: 1, 3, 5, 7, 9, 11, 13, 15, 17, 19, 21, 23, 25, 27, 29, 31 and 33, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of GPCRX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an GPCRX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human GPCRX protein. Homologous nucleic acid sequences include those nucleic acid sequences that encode conservative amino acid substitutions (see below) in SEQ ID NOS: 2, 4, 6, 8, 10, 12, 14, 16, 18, 20, 22, 24, 26, 28, 30, 32, and 34, as well as a polypeptide possessing GPCRX biological activity. Various biological activities of the GPCRX proteins are described below.

As used herein, “identical” residues correspond to those residues in a comparison between two sequences where the equivalent nucleotide base or amino acid residue in an alignment of two sequences is the same residue. Residues are alternatively described as “similar” or “positive” when the comparisons between two sequences in an alignment show that residues in an equivalent position in a comparison are either the same amino acid or a conserved amino acid as defined below.

An GPCRX polypeptide is encoded by the open reading frame (“ORF”) of an GPCRX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human GPCRX genes allows for the generation of probes and primers designed for use in identifying and/or cloning GPCRX homologues in other cell types, e.g. from other tissues, as well as GPCRX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NOS: 1, 3, 5, 7, 9, 11, 13, 15, 17, 19, 21, 23, 25, 27, 29, 31 and 33; or an anti-sense strand nucleotide sequence of SEQ ID NOS: 1, 3, 5, 7, 9, 11, 13, 15, 17, 19, 21, 23, 25, 27, 29, 31 and 33; or of a naturally occurring mutant of SEQ ID NOS: 1, 3, 5, 7, 9, 11, 13, 15, 17, 19, 21, 23, 25, 27, 29, 31 and 33.

Probes based on the human GPCRX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which Mis-express an GPCRX protein, such as by measuring a level of an GPCRX-encoding nucleic acid in a sample of cells from a subject e.g., detecting GPCRX mRNA levels or determining whether a genomic GPCRX gene has been mutated or deleted. “A polypeptide having a biologically-active portion of an GPCRX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of GPCRX” can be prepared by isolating a portion SEQ ID NOS: 1, 3, 5, 7, 9, 11, 13, 15, 17, 19, 21, 23, 25, 27, 29, 31 and 33 that encodes a polypeptide having an GPCRX biological activity (the biological activities of the GPCRX proteins are described below), expressing the encoded portion of GPCRX protein (e.g., by recombinant expression in vitro) and assessing the activity of the encoded portion of GPCRX.

GPCRX Nucleic Acid and Polypeptide Variants

The invention further encompasses nucleic acid molecules that differ from the nucleotide sequences shown SEQ ID NOS: 1, 3, 5, 7, 9, 11, 13, 15, 17, 19, 21, 23, 25, 27, 29, 31 and 33 due to degeneracy of the genetic code and thus encode the same GPCRX proteins as that encoded by the nucleotide sequences shown in SEQ ID NOS: 1, 3, 5, 7, 9, 11, 13, 15, 17, 19, 21, 23, 25, 27, 29, 31, and 33. In another embodiment, an isolated nucleic acid molecule of the invention has a nucleotide sequence encoding a protein having an amino acid sequence shown in SEQ ID NOS: 2, 4, 6, 8, 10, 12, 14, 16, 18, 20, 22, 24, 26, 28, 30, 32, and 34.

In addition to the human GPCRX nucleotide sequences shown in SEQ ID NOS: 1, 3, 5, 7, 9, 11, 13, 15, 17, 19, 21, 23, 25, 27, 29, 31 and 33, it will be appreciated by those skilled in the art that DNA sequence polymorphisms that lead to changes in the amino acid sequences of the GPCRX polypeptides may exist within a population (e.g., the human population). Such genetic polymorphism in the GPCRX genes may exist among individuals within a population due to natural allelic variation. As used herein, the terms “gene” and “recombinant gene” refer to nucleic acid molecules comprising an open reading frame (ORF) encoding an GPCRX protein, preferably a vertebrate GPCRX protein. Such natural allelic variations can typically result in 1-5% variance in the nucleotide sequence of the GPCRX genes. Any and all such nucleotide variations and resulting amino acid polymorphisms in the GPCRX polypeptides, which are the result of natural allelic variation and that do not alter the functional activity of the GPCRX polypeptides, are intended to be within the scope of the invention.

Moreover, nucleic acid molecules encoding GPCRX proteins from other species, and thus that have a nucleotide sequence that differs from the human sequence SEQ ID NOS: 1, 3, 5, 7, 9, 11, 13, 15, 17, 19, 21, 23, 25, 27, 29, 31 and 33 are intended to be within the scope of the invention. Nucleic acid molecules corresponding to natural allelic variants and homologues of the GPCRX cDNAs of the invention can be isolated based on their homology to the human GPCRX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 5, 7, 9, 11, 13, 15, 17, 19, 21, 23, 25, 27, 29, 31, and 33. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding GPCRX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%/o, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences of SEQ ID NOS: 1, 3, 5, 7, 9, 11, 13, 15, 17, 19, 21, 23, 25, 27, 29, 31 and 33 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS: 1, 3, 5, 7, 9, 11, 13, 15, 17, 19, 21, 23, 25, 27, 29, 31 and 33 or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NOS: 1, 3, 5, 7, 9, 11, 13, 15, 17, 19, 21, 23, 25, 27, 29, 31 and 33 or fragments, analogs or derivatives thereof, under conditions of low stringency, is provided. A non-limiting example of low stringency hybridization conditions are hybridization in 35% formamide, 5×SSC, 50 mM Tris-HCI (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of GPCRX sequences that may exist in the population, the skilled artisan will further appreciate that changes can be introduced by mutation into the nucleotide sequences of SEQ ID NOS: 1, 3, 5, 7, 9, 11, 13, 15, 17, 19, 21, 23, 25, 27, 29, 31 and 33 thereby leading to changes in the amino acid sequences of the encoded GPCRX proteins, without altering the functional ability of said GPCRX proteins. For example, nucleotide substitutions leading to amino acid substitutions at “non-essential” amino acid residues can be made in the sequence of SEQ ID NOS: 2, 4, 6, 8, 10, 12, 14, 16, 18, 20, 22, 24, 26, 28, 30, 32 and 34. A “non-essential” amino acid residue is a residue that can be altered from the wild-type sequences of the GPCRX proteins without altering their biological activity, whereas an “essential” amino acid residue is required for such biological activity. For example, amino acid residues that are conserved among the GPCRX proteins of the invention are predicted to be particularly non-amenable to alteration. Amino acids for which conservative substitutions can be made are well-known within the art.

Another aspect of the invention pertains to nucleic acid molecules encoding GPCRX proteins that contain changes in amino acid residues that are not essential for activity. Such GPCRX proteins differ in amino acid sequence from SEQ ID NOS: 2, 4, 6, 8, 10, 12, 14, 16, 18, 20, 22, 24, 26, 28, 30, 32 and 34 yet retain biological activity. In one embodiment, the isolated nucleic acid molecule comprises a nucleotide sequence encoding a protein, wherein the protein comprises an amino acid sequence at least about 45% homologous to the amino acid sequences of SEQ ID NOS: 2, 4, 6, 8, 10, 12, 14, 16, 18, 20, 22, 24, 26, 28, 30, 32 and 34. Preferably, the protein encoded by the nucleic acid molecule is at least about 60% homologous to SEQ ID NOS: 2, 4, 6, 8, 10, 12, 14, 16, 18, 20, 22, 24, 26, 28, 30, 32 and 34; more preferably at least about 70% homologous to SEQ ID NOS: 2, 4, 6, 8, 10, 12, 14, 16, 18, 20, 22, 24, 26, 28, 30, 32 and 34; still more preferably at least about 80% homologous to SEQ ID NOS: 2, 4, 6, 8, 10, 12, 14, 16, 18, 20, 22, 24, 26, 28, 30, 32 and 34; even more preferably at least about 90% homologous to SEQ ID NOS: 2, 4, 6, 8, 10, 12, 14, 16, 18, 20, 22, 24, 26, 28, 30, 32 and 34; and most preferably at least about 95% homologous to SEQ ID NOS: 2, 4, 6, 8, 10, 12, 14, 16, 18, 20, 22, 24, 26, 28, 30, 32 and 34.

An isolated nucleic acid molecule encoding an GPCRX protein homologous to the polypeptide of SEQ ID NOS: 2, 4, 6, 8, 10, 12, 14, 16, 18, 20, 22, 24, 26, 28, 30, 32 and 34 can be created by introducing one or more nucleotide substitutions, additions or deletions into the nucleotide sequence of SEQ ID NOS: 1, 3, 5, 7, 9, 11, 13, 15, 17, 19, 21, 23, 25, 27, 29, 31 and 33 such that one or more amino acid substitutions, additions or deletions are introduced into the encoded protein.

Mutations can be introduced into SEQ ID NOS: 2, 4, 6, 8, 10, 12, 14, 16, 18, 20, 22, 24, 26, 28, 30, 32 and 34 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the GPCRX protein is replaced with another amino acid residue from the same side chain family. Alternatively, in another embodiment, mutations can be introduced randomly along all or part of an GPCRX coding sequence, such as by saturation mutagenesis, and the resultant mutants can be screened for GPCRX biological activity to identify mutants that retain activity. Following mutagenesis of SEQ ID NOS: 1, 3, 5, 7, 9, 11, 13, 15, 17, 19, 21, 23, 25, 27, 29, 31 and 33, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant GPCRX protein can be assayed for (i) the ability to form protein:protein interactions with other GPCRX proteins, other cell-surface proteins, or biologically-active portions thereof, (ii) complex formation between a mutant GPCRX protein and an GPCRX ligand; or (iii) the ability of a mutant GPCRX protein to bind to an intracellular target protein or biologically-active portion thereof; (e.g. avidin proteins).

In yet another embodiment, a mutant GPCRX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS: 1, 3, 5, 7, 9, 11, 13, 15, 17, 19, 21, 23, 25, 27, 29, 31 and 33, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire GPCRX coding strand, or to only a portion thereof. Nucleic acid molecules encoding fragments, homologs, derivatives and analogs of an GPCRX protein of SEQ ID NOS: 2, 4, 6, 8, 10, 12, 14, 16, 18, 20, 22, 24, 26, 28, 30, 32, and 34, or antisense nucleic acids complementary to an GPCRX nucleic acid sequence of SEQ ID NOS: 1, 3, 5, 7, 9, 11, 13, 15, 17, 19, 21, 23, 25, 27, 29, 31 and 33, are additionally provided.

In one embodiment, an antisense nucleic acid molecule is antisense to a “coding region” of the coding strand of a nucleotide sequence encoding an GPCRX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the GPCRX protein. The term “noncoding region” refers to 5′ and 3′ sequences which flank the coding region that are not translated into amino acids (i.e., also referred to as 5′ and 3′ untranslated regions).

Given the coding strand sequences encoding the GPCRX protein disclosed herein, antisense nucleic acids of the invention can be designed according to the rules of Watson and Crick or Hoogsteen base pairing. The antisense nucleic acid molecule can be complementary to the entire coding region of GPCRX mRNA, but more preferably is an oligonucleotide that is antisense to only a portion of the coding or noncoding region of GPCRX mRNA. For example, the antisense oligonucleotide can be complementary to the region surrounding the translation start site of GPCRX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1 -methylguanine, 1 -methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subdloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an GPCRX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an □-anomeric nucleic acid molecule. An □-anomeric nucleic acid molecule forms specific double-stranded hybrids with complementary RNA in which, contrary to the usual □-units, the strands run parallel to each other. See, e.g., Gaultier, et al., 1987. Nucl. Acids Res. 15: 6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave GPCRX mRNA transcripts to thereby inhibit translation of GPCRX mRNA. A ribozyme having specificity for an GPCRX-encoding nucleic acid can be designed based upon the nucleotide sequence of an GPCRX cDNA disclosed herein (i.e., SEQ ID NOS: 1, 3, 5, 7, 9, 11, 13, 15, 17, 19, 21, 23, 25, 27, 29, 31 and 33). For example, a derivative of a Tetrahymena L-19 IVS RNA can be constructed in which the nucleotide sequence of the active site is complementary to the nucleotide sequence to be cleaved in an GPCRX-encoding MRNA. See, e.g., U.S. Pat. No. 4,987,071 to Cech, et al. and U.S. Pat. No. 5,116,742 to Cech, et al. GPCRX mRNA can also be used to select a catalytic RNA having a specific ribonuclease activity from a pool of RNA molecules. See, e.g., Bartel et al., (1993) Science 261:1411-1418.

Alternatively, GPCRX gene expression can be inhibited by targeting nucleotide sequences complementary to the regulatory region of the GPCRX nucleic acid (e.g., the GPCRX promoter and/or enhancers) to form triple helical structures that prevent transcription of the GPCRX gene in target cells. See, e.g., Helene, 1991. Anticancer Drug Des. 6: 569-84; Helene, et al. 1992. Ann. N.Y. Acad. Sci 660: 27-36; Maher, 1992. Bioassays 14: 807-15.

In various embodiments, the GPCRX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of GPCRX can be used in therapeutic and diagnostic applications. For example, PNAs can be used as antisense or antigene agents for sequence-specific modulation of gene expression by, e.g., inducing transcription or translation arrest or inhibiting replication. PNAs of GPCRX can also be used, for example, in the analysis of single base pair mutations in a gene (e.g., PNA directed PCR clamping; as artificial restriction enzymes when used in combination with other enzymes, e.g., S₁ nucleases (see, Hyrup, et al., 1996.supra); or as probes or primers for DNA sequence and hybridization (see, Hyrup, et al., 1996, supra; Perry-O'Keefe, et al., 1996. supra).

In another embodiment, PNAs of GPCRX can be modified, e.g., to enhance their stability or cellular uptake, by attaching lipophilic or other helper groups to PNA, by the formation of PNA-DNA chimeras, or by the use of liposomes or other techniques of drug delivery known in the art For example, PNA-DNA chimeras of GPCRX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et aL, 1975. Bioorg. Med. Chem. Lett. 5:1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. W088/09810) or the blood-brain barier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5:539-549). To this end, the oligonucleotide may be conjugated to another molecule, e.g., a peptide, a hybridization triggered cross-linking agent, a transport agent, a hybridization-triggered cleavage agent, and the like.

GPCRX Polypeptides

A polypeptide according to the invention includes a polypeptide including the amino acid sequence of GPCRX polypeptides whose sequences are provided in SEQ ID NOS: 2, 4, 6, 8, 10, 12, 14, 16, 18, 20, 22, 24, 26, 28, 30, 32, 34. The invention also includes a mutant or variant protein any of whose residues may be changed from the corresponding residues shown in SEQ ID NOS: 2, 4, 6, 8, 10, 12, 14, 16, 18, 20, 22, 24, 26, 28, 30, 32, 34 while still encoding a protein that maintains its GPCRX activities and physiological functions, or a functional fragment thereof.

In general, an GPCRX variant that preserves GPCRX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated GPCRX proteins, and biologically-active portions thereof, or derivatives, fragments, analogs or homologs thereof. Also provided are polypeptide fragments suitable for use as immunogens to raise anti-GPCRX antibodies. In one embodiment, native GPCRX proteins can be isolated from cells or tissue sources by an appropriate purification scheme using standard protein purification techniques. In another embodiment, GPCRX proteins are produced by recombinant DNA techniques. Alternative to recombinant expression, an GPCRX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the GPCRX protein is derived, or substantially free from chemical precursors or other chemicals when chemically synthesized. The language “substantially free of cellular material” includes preparations of GPCRX proteins in which the protein is separated from cellular components of the cells from which it is isolated or recombinantly-produced. In one embodiment, the language “substantially free of cellular material” includes preparations of GPCRX proteins having less than about 30% (by dry weight) of non-GPCRX proteins (also referred to herein as a “contaminating protein”), more preferably less than about 20% of non-GPCRX proteins, still more preferably less than about 10% of non-GPCRX proteins, and most preferably less than about 5% of non-GPCRX proteins. When the GPCRX protein or biologically-active portion thereof is recombinantly-produced, it is also preferably substantially free of culture medium, i.e., culture medium represents less than about 20%, more preferably less than about 10%, and most preferably less than about 5% of the volume of the GPCRX protein preparation.

The language “substantially free of chemical precursors or other chemicals” includes preparations of GPCRX proteins in which the protein is separated from chemical precursors or other chemicals that are involved in the synthesis of the protein. In one embodiment, the language “substantially free of chemical precursors or other chemicals” includes preparations of GPCRX proteins having less than about 30% (by dry weight) of chemical precursors or non-GPCRX chemicals, more preferably less than about 20% chemical precursors or non-GPCRX chemicals, still more preferably less than about 10% chemical precursors or non-GPCRX chemicals, and most preferably less than about 5% chemical precursors or non-GPCRX chemicals.

Biologically-active portions of GPCRX proteins include peptides comprising amino acid sequences sufficiently homologous to or derived from the amino acid sequences of the GPCRX proteins (e.g., the amino acid sequence shown in SEQ ID NOS: 2, 4, 6, 8, 10, 12, 14, 16, 18, 20, 22, 24, 26, 28, 30, 32, 34) that include fewer amino acids than the full-length GPCRX proteins, and exhibit at least one activity of an GPCRX protein. Typically, biologically-active portions comprise a domain or motif with at least one activity of the GPCRX protein. A biologically-active portion of an GPCRX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native GPCRX protein.

In an embodiment, the GPCRX protein has an amino acid sequence shown in SEQ ID NOS: 2, 4, 6, 8, 10, 12, 14, 16, 18, 20, 22, 24, 26, 28, 30, 32, 34. In other embodiments, the GPCRX protein is substantially homologous to SEQ ID NOS: 2, 4, 6, 8, 10, 12, 14, 16, 18, 20, 22, 24, 26, 28, 30, 32, 34, and retains the functional activity of the protein of SEQ ID NOS: 2, 4, 6, 8, 10, 12, 14, 16, 18, 20, 22, 24, 26, 28, 30, 32, 34, yet differs in amino acid sequence due to natural allelic variation or mutagenesis, as described in detail, below. Accordingly, in another embodiment, the GPCRX protein is a protein that comprises an amino acid sequence at least about 45% homologous to the amino acid sequence SEQ ID NOS: 2, 4, 6, 8, 10, 12, 14, 16, 18, 20, 22, 24, 26, 28, 30, 32, 34, and retains the functional activity of the GPCRX proteins of SEQ ID NOS: 2, 4, 6, 8, 10, 12, 14, 16, 18, 20, 22, 24, 26, 28, 30, 32, 34.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS: 1, 3, 5, 7, 9, 11, 13, 15, 17, 19, 21, 23, 25, 27, 29, 31, and 33.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (ie., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides GPCRX chimeric or fusion proteins. As used herein, an GPCRX “chimeric protein” or “fusion protein” comprises an GPCRX polypeptide operatively-linked to a non-GPCRX polypeptide. An “GPCRX polypeptide” refers to a polypeptide having an amino acid sequence corresponding to an GPCRX protein (SEQ ID NOS: 2, 4, 6, 8, 10, 12, 14, 16, 18, 20, 22, 24, 26, 28, 30, 32, 34), whereas a “non-GPCRX polypeptide” refers to a polypeptide having an amino acid sequence corresponding to a protein that is not substantially homologous to the GPCRX protein, e.g., a protein that is different from the GPCRX protein and that is derived from the same or a different organism. Within an GPCRX fusion protein the GPCRX polypeptide can correspond to all or a portion of an GPCRX protein. In one embodiment, an GPCRX fusion protein comprises at least one biologically-active portion of an GPCRX protein. In another embodiment, an GPCRX fusion protein comprises at least two biologically-active portions of an GPCRX protein. In yet another embodiment, an GPCRX fusion protein comprises at least three biologically-active portions of an GPCRX protein. Within the fusion protein, the term “operatively-linked” is intended to indicate that the GPCRX polypeptide and the non-GPCRX polypeptide are fused in-frame with one another. The non-GPCRX polypeptide can be fused to the N-terminus or C-terminus of the GPCRX polypeptide.

In one embodiment, the fusion protein is a GST-GPCRX fusion protein in which the GPCRX sequences are fused to the C-terminus of the GST (glutathione S-transferase) sequences. Such fusion proteins can facilitate the purification of recombinant GPCRX polypeptides.

In another embodiment, the fusion protein is an GPCRX protein containing a heterologous signal sequence at its N-terminus. In certain host cells (e.g., mammalian host cells), expression and/or secretion of GPCRX can be increased through use of a heterologous signal sequence.

In yet another embodiment, the fusion protein is an GPCRX-immunoglobulin fusion protein in which the GPCRX sequences are fused to sequences derived from a member of the immunoglobulin protein family. The GPCRX-immunoglobulin fusion proteins of the invention can be incorporated into pharmaceutical compositions and administered to a subject to inhibit an interaction between an GPCRX ligand and an GPCRX protein on the surface of a cell, to thereby suppress GPCRX-mediated signal transduction in vivo. The GPCRX-immunoglobulin fusion proteins can be used to affect the bioavailability of an GPCRX cognate ligand. Inhibition of the GPCRX ligand/GPCRX interaction may be useful therapeutically for both the treatment of proliferative and differentiative disorders, as well as modulating (e.g. promoting or inhibiting) cell survival. Moreover, the GPCRX-immunoglobulin fusion proteins of the invention can be used as immunogens to produce anti-GPCRX antibodies in a subject, to purify GPCRX ligands, and in screening assays to identify molecules that inhibit the interaction of GPCRX with an GPCRX ligand.

An GPCRX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An GPCRX-encoding nucleic acid can be cloned into such an expression vector such that the fusion moiety is linked in-frame to the GPCRX protein.

GPCRX Agonists and Antagonists

The invention also pertains to variants of the GPCRX proteins that function as either GPCRX agonists (i.e., mimetics) or as GPCRX antagonists. Variants of the GPCRX protein can be generated by mutagenesis (e.g., discrete point mutation or truncation of the GPCRX protein). An agonist of the GPCRX protein can retain substantially the same, or a subset of, the biological activities of the naturally occurring form of the GPCRX protein. An antagonist of the GPCRX protein can inhibit one or more of the activities of the naturally occurring form of the GPCRX protein by, for example, competitively binding to a downstream or upstream member of a cellular signaling cascade which includes the GPCRX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the GPCRX proteins.

Variants of the GPCRX proteins that function as either GPCRX agonists (i.e., mimetics) or as GPCRX antagonists can be identified by screening combinatorial libraries of mutants (e.g., truncation mutants) of the GPCRX proteins for GPCRX protein agonist or antagonist activity. In one embodiment, a variegated library of GPCRX variants is generated by combinatorial mutagenesis at the nucleic acid level and is encoded by a variegated gene library. A variegated library of GPCRX variants can be produced by, for example, enzymatically ligating a mixture of synthetic oligonucleotides into gene sequences such that a degenerate set of potential GPCRX sequences is expressible as individual polypeptides, or alternatively, as a set of larger fusion proteins (e.g., for phage display) containing the set of GPCRX sequences therein. There are a variety of methods which can be used to produce libraries of potential GPCRX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential GPCRX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, etal., 1983. Nucl. Acids Res. 11:477.

Polypeptide Libraries

In addition, libraries of fragments of the GPCRX protein coding sequences can be used to generate a variegated population of GPCRX fragments for screening and subsequent selection of variants of an GPCRX protein. In one embodiment, a library of coding sequence fragments can be generated by treating a double stranded PCR fragment of an GPCRX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the GPCRX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of GPCRX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify GPCRX variants. See, e.g., Arkin and Yourvan, 1992. Proc. Natl. Acad. Sci. USA 89: 7811-7815; Delgrave, et al., 1993. Protein Engineering 6:327-331.

Anti-GPCRX Antibodies

Also included in the invention are antibodies to GPCRX proteins, or fragments of GPCRX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated GPCRX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of GPCRX-related protein that is located on the surface of the protein, e.g., a hydrophilic region. A hydrophobicity analysis of the human GPCRX-related protein sequence will indicate which regions of a GPCRX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subdloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, 131I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an GPCRX protein is facilitated by generation of hybridomas that bind to the fragment of an GPCRX protein possessing such a domain. Thus, antibodies that are specific for a desired domain within an GPCRX protein, or derivatives, fragments, analogs or homologs thereof, are also provided herein.

Anti-GPCRX antibodies may be used in methods known within the art relating to the localization and/or quantitation of an GPCRX protein (e.g., for use in measuring levels of the GPCRX protein within appropriate physiological samples, for use in diagnostic methods, for use in imaging the protein, and the like). In a given embodiment, antibodies for GPCRX proteins, or derivatives, fragments, analogs or homologs thereof, that contain the antibody derived binding domain, are utilized as pharmacologically-active compounds (hereinafter “Therapeutics”).

An anti-GPCRX antibody (e.g., monoclonal antibody) can be used to isolate an GPCRX polypeptide by standard techniques, such as affinity chromatography or immunoprecipitation. An anti-GPCRX antibody can facilitate the purification of natural GPCRX polypeptide from cells and of recombinantly-produced GPCRX polypeptide expressed in host cells. Moreover, an anti-GPCRX antibody can be used to detect GPCRX protein (e.g., in a cellular lysate or cell supernatant) in order to evaluate the abundance and pattern of expression of the GPCRX protein. Anti-GPCRX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, □-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

GPCRX Recombinant Expression Vectors and Host Cells

Another aspect of the invention pertains to vectors, preferably expression vectors, containing a nucleic acid encoding an GPCRX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., GPCRX proteins, mutant forms of GPCRX proteins, fusion proteins, etc.).

The recombinant expression vectors of the invention can be designed for expression of GPCRX proteins in prokaryotic or eukaryotic cells. For example, GPCRX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the GPCRX expression vector is a yeast expression vector. Examples of vectors for expression in yeast Saccharomyces cerivisae include pYepSec1 (Baldari, et al., 1987. EMBO J 6: 229-234), pMFa (Kujan and Herskowitz, 1982. Cell 30: 933-943), pJRY88 (Schultz et al., 1987. Gene 54: 113-123), pYES2 (Invitrogen Corporation, San Diego, Calif.), and picZ (In Vitrogen Corp, San Diego, Calif.).

Alternatively, GPCRX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the □-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to GPCRX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, GPCRX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding GPCRX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) GPCRX protein. Accordingly, the invention further provides methods for producing GPCRX protein using the host cells of the invention. In one embodiment, the method comprises culturing the host cell of invention (into which a recombinant expression vector encoding GPCRX protein has been introduced) in a suitable medium such that GPCRX protein is produced. In another embodiment, the method further comprises isolating GPCRX protein from the medium or the host cell.

Transgenic GPCRX Animals

The host cells of the invention can also be used to produce non-human transgenic animals. For example, in one embodiment, a host cell of the invention is a fertilized oocyte or an embryonic stem cell into which GPCRX protein-coding sequences have been introduced. Such host cells can then be used to create non-human transgenic animals in which exogenous GPCRX sequences have been introduced into their genome or homologous recombinant animals in which endogenous GPCRX sequences have been altered. Such animals are useful for studying the function and/or activity of GPCRX protein and for identifying and/or evaluating modulators of GPCRX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous GPCRX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing GPCRX-encoding nucleic acid into the male pronuclei of a fertilized oocyte (e.g., by microinjection, retroviral infection) and allowing the oocyte to develop in a pseudopregnant female foster animal. The human GPCRX cDNA sequences ancoding polypeptides of SEQ ID NOS: 2, 4, 6, 8, 10, 12, 14, 16, 18, 20, 22, 24, 26, 28, 30, 32, 34 can be introduced as a transgene into the genome of a non-human animal. Alternatively, a non-human homologue of the human GPCRX gene, such as a mouse GPCRX gene, can be isolated based on hybridization to the human GPCRX cDNA (described further supra) and used as a transgene. Intronic sequences and polyadenylation signals can also be included in the transgene to increase the efficiency of expression of the transgene. A tissue-specific regulatory sequence(s) can be operably-linked to the GPCRX transgene to direct expression of GPCRX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the GPCRX transgene in its genome and/or expression of GPCRX mRNA in tissues or cells of the animals. A transgenic founder animal can then be used to breed additional animals carrying the transgene. Moreover, transgenic animals carrying a transgene-encoding GPCRX protein can further be bred to other transgenic animals carrying other transgenes.

To create a homologous recombinant animal, a vector is prepared which contains at least a portion of an GPCRX gene into which a deletion, addition or substitution has been introduced to thereby alter, e.g., functionally disrupt, the GPCRX gene. The GPCRX gene can be a human gene (e.g., the cDNA of SEQ ID NOS: 1, 3, 5, 7, 9, 11, 13, 15, 17, 19, 21, 23, 25, 27, 29, 31 and 33), but more preferably, is a non-human homologue of a human GPCRX gene. For example, a mouse homologue of human GPCRX gene of SEQ ID NOS: 1, 3, 5, 7, 9, 11, 13, 15, 17, 19, 21, 23, 25, 27, 29, 31 and 33 can be used to construct a homologous recombination vector suitable for altering an endogenous GPCRX gene in the mouse genome. In one embodiment, the vector is designed such that, upon homologous recombination, the endogenous GPCRX gene is functionally disrupted (i.e., no longer encodes a functional protein; also referred to as a “knock out” vector).

Alternatively, the vector can be designed such that, upon homologous recombination, the endogenous GPCRX gene is mutated or otherwise altered but still encodes functional protein (e.g., the upstream regulatory region can be altered to thereby alter the expression of the endogenous GPCRX protein). In the homologous recombination vector, the altered portion of the GPCRX gene is flanked at its 5′- and 3′-termini by additional nucleic acid of the GPCRX gene to allow for homologous recombination to occur between the exogenous GPCRX gene carried by the vector and an endogenous GPCRX gene in an embryonic stem cell. The additional flanking GPCRX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced GPCRX gene has homologously-recombined with the endogenous GPCRX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS ANDEMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The GPCRX nucleic acid molecules, GPCRX proteins, and anti-GPCRX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an GPCRX protein or anti-GPCRX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express GPCRX protein (e.g., via a recombinant expression vector in a host cell in gene therapy applications), to detect GPCRX mRNA (e.g., in a biological sample) or a genetic lesion in an GPCRX gene, and to modulate GPCRX activity, as described further, below. In addition, the GPCRX proteins can be used to screen drugs or compounds that modulate the GPCRX protein activity or expression as well as to treat disorders characterized by insufficient or excessive production of GPCRX protein or production of GPCRX protein forms that have decreased or aberrant activity compared to GPCRX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-GPCRX antibodies of the invention can be used to detect and isolate GPCRX proteins and modulate GPCRX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g. peptides, peptidomimetics, small molecules or other drugs) that bind to GPCRX proteins or have a stimulatory or inhibitory effect on, e.g., GPCRX protein expression or GPCRX protein activity. The invention also includes compounds identified in the screening assays described herein.

In one embodiment, the invention provides assays for screening candidate or test compounds which bind to or modulate the activity of the membrane-bound form of an GPCRX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of GPCRX protein, or a biologically-active portion thereof, on the cell surface is contacted with a test compound and the ability of the test compound to bind to an GPCRX protein determined. The cell, for example, can of mammalian origin or a yeast cell. Determining the ability of the test compound to bind to the GPCRX protein can be accomplished, for example, by coupling the test compound with a radioisotope or enzymatic label such that binding of the test compound to the GPCRX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of GPCRX protein, or a biologically-active portion thereof, on the cell surface with a known compound which binds GPCRX to form an assay mixture, contacting the assay mixture with a test compound, and determining the ability of the test compound to interact with an GPCRX protein, wherein determining the ability of the test compound to interact with an GPCRX protein comprises determining the ability of the test compound to preferentially bind to GPCRX protein or a biologically-active portion thereof as compared to the known compound.

In another embodiment, an assay is a cell-based assay comprising contacting a cell expressing a membrane-bound form of GPCRX protein, or a biologically-active portion thereof, on the cell surface with a test compound and determining the ability of the test compound to modulate (e.g., stimulate or inhibit) the activity of the GPCRX protein or biologically-active portion thereof. Determining the ability of the test compound to modulate the activity of GPCRX or a biologically-active portion thereof can be accomplished, for example, by determining the ability of the GPCRX protein to bind to or interact with an GPCRX target molecule. As used herein, a “target molecule” is a molecule with which an GPCRX protein binds or interacts in nature, for example, a molecule on the surface of a cell which expresses an GPCRX interacting protein, a molecule on the surface of a second cell, a molecule in the extracellular milieu, a molecule associated with the internal surface of a cell membrane or a cytoplasmic molecule. An GPCRX target molecule can be a non-GPCRX molecule or an GPCRX protein or polypeptide of the invention. In one embodiment, an GPCRX target molecule is a component of a signal transduction pathway that facilitates transduction of an extracellular signal (e.g. a signal generated by binding of a compound to a membrane-bound GPCRX molecule) through the cell membrane and into the cell. The target, for example, can be a second intercellular protein that has catalytic activity or a protein that facilitates the association of downstream signaling molecules with GPCRX.

Determining the ability of the GPCRX protein to bind to or interact with an GPCRX target molecule can be accomplished by one of the methods described above for determining direct binding. In one embodiment, determining the ability of the GPCRX protein to bind to or interact with an GPCRX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an GPCRX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an GPCRX protein or biologically-active portion thereof with a test compound and determining the ability of the test compound to bind to the GPCRX protein or biologically-active portion thereof. Binding of the test compound to the GPCRX protein can be determined either directly or indirectly as described above. In one such embodiment, the assay comprises contacting the GPCRX protein or biologically-active portion thereof with a known compound which binds GPCRX to form an assay mixture, contacting the assay mixture with a test compound, and determining the ability of the test compound to interact with an GPCRX protein, wherein determining the ability of the test compound to interact with an GPCRX protein comprises determining the ability of the test compound to preferentially bind to GPCRX or biologically-active portion thereof as compared to the known compound.

In still another embodiment, an assay is a cell-free assay comprising contacting GPCRX protein or biologically-active portion thereof with a test compound and determining the ability of the test compound to modulate (e.g. stimulate or inhibit) the activity of the GPCRX protein or biologically-active portion thereof. Determining the ability of the test compound to modulate the activity of GPCRX can be accomplished, for example, by determining the ability of the GPCRX protein to bind to an GPCRX target molecule by one of the methods described above for determining direct binding. In an alternative embodiment, determining the ability of the test compound to modulate the activity of GPCRX protein can be accomplished by determining the ability of the GPCRX protein further modulate an GPCRX target molecule. For example, the catalytic/enzymatic activity of the target molecule on an appropriate substrate can be determined as described, supra.

In yet another embodiment, the cell-free assay comprises contacting the GPCRX protein or biologically-active portion thereof with a known compound which binds GPCRX protein to form an assay mixture, contacting the assay mixture with a test compound, and determining the ability of the test compound to interact with an GPCRX protein, wherein determining the ability of the test compound to interact with an GPCRX protein comprises determining the ability of the GPCRX protein to preferentially bind to or modulate the activity of an GPCRX target molecule.

The cell-free assays of the invention are amenable to use of both the soluble form or the membrane-bound form of GPCRX protein. In the case of cell-free assays comprising the membrane-bound form of GPCRX protein, it may be desirable to utilize a solubilizing agent such that the membrane-bound form of GPCRX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesite®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either GPCRX protein or its target molecule to facilitate separation of complexed from uncomplexed forms of one or both of the proteins, as well as to accommodate automation of the assay. Binding of a test compound to GPCRX protein, or interaction of GPCRX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-GPCRX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or GPCRX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of GPCRX protein binding or activity determined using standard techniques.

Other techniques for immobilizing proteins on matrices can also be used in the screening assays of the invention. For example, either the GPCRX protein or its target molecule can be immobilized utilizing conjugation of biotin and streptavidin. Biotinylated GPCRX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with GPCRX protein or target molecules, but which do not interfere with binding of the GPCRX protein to its target molecule, can be derivatized to the wells of the plate, and unbound target or GPCRX protein trapped in the wells by antibody conjugation. Methods for detecting such complexes, in addition to those described above for the GST-immobilized complexes, include immunodetection of complexes using antibodies reactive with the GPCRX protein or target molecule, as well as enzyme-linked assays that rely on detecting an enzymatic activity associated with the GPCRX protein or target molecule.

In another embodiment, modulators of GPCRX protein expression are identified in a method wherein a cell is contacted with a candidate compound and the expression of GPCRX mRNA or protein in the cell is determined. The level of expression of GPCRX mRNA or protein in the presence of the candidate compound is compared to the level of expression of GPCRX mRNA or protein in the absence of the candidate compound. The candidate compound can then be identified as a modulator of GPCRX mRNA or protein expression based upon this comparison. For example, when expression of GPCRX mRNA or protein is greater (i.e., statistically significantly greater) in the presence of the candidate compound than in its absence, the candidate compound is identified as a stimulator of GPCRX mRNA or protein expression. Alternatively, when expression of GPCRX mRNA or protein is less (statistically significantly less) in the presence of the candidate compound than in its absence, the candidate compound is identified as an inhibitor of GPCRX mRNA or protein expression. The level of GPCRX mRNA or protein expression in the cells can be determined by methods described herein for detecting GPCRX mRNA or protein.

In yet another aspect of the invention, the GPCRX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with GPCRX (“GPCRX-binding proteins” or “GPCRX-bp”) and modulate GPCRX activity. Such GPCRX-binding proteins are also likely to be involved in the propagation of signals by the GPCRX proteins as, for example, upstream or downstream elements of the GPCRX pathway.

The two-hybrid system is based on the modular nature of most transcription factors, which consist of separable DNA-binding and activation domains. Briefly, the assay utilizes two different DNA constructs. In one construct, the gene that codes for GPCRX is fused to a gene encoding the DNA binding domain of a known transcription factor (e.g., GAL4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an GPCRX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with GPCRX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the GPCRX sequences, SEQ ID NOS: 1, 3, 5, 7, 9, 11, 13, 15, 17, 19, 21, 23, 25, 27, 29, 31 and 33, or fragments or derivatives thereof, can be used to map the location of the GPCRX genes, respectively, on a chromosome. The mapping of the GPCRX sequences to chromosomes is an important first step in correlating these sequences with genes associated with disease.

Briefly, GPCRX genes can be mapped to chromosomes by preparing PCR primers (preferably 15-25 bp in length) from the GPCRX sequences. Computer analysis of the GPCRX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the GPCRX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the GPCRX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the GPCRX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The GPCRX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the GPCRX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The GPCRX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs). Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS: 1, 3, 5, 7, 9, 11, 13, 15, 17, 19, 21, 23, 25, 27, 29, 31 and 33 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining GPCRX protein and/or nucleic acid expression as well as GPCRX activity, in the context of a biological sample (e.g., blood, serum, cells, tissue) to thereby determine whether an individual is afflicted with a disease or disorder, or is at risk of developing a disorder, associated with aberrant GPCRX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with GPCRX protein, nucleic acid expression or activity. For example, mutations in an GPCRX gene can be assayed in a biological sample. Such assays can be used for prognostic or predictive purpose to thereby prophylactically treat an individual prior to the onset of a disorder characterized by or associated with GPCRX protein, nucleic acid expression, or biological activity.

Another aspect of the invention provides methods for determining GPCRX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.) Yet another aspect of the invention pertains to monitoring the influence of agents (e.g., drugs, compounds) on the expression or activity of GPCRX in clinical trials.

These and other agents are described in further detail in the following sections.

Diagnostic Assays

An exemplary method for detecting the presence or absence of GPCRX in a biological sample involves obtaining a biological sample from a test subject and contacting the biological sample with a compound or an agent capable of detecting GPCRX protein or nucleic acid (e.g., mRNA, genomic DNA) that encodes GPCRX protein such that the presence of GPCRX is detected in the biological sample. An agent for detecting GPCRX mRNA or genomic DNA is a labeled nucleic acid probe capable of hybridizing to GPCRX mRNA or genomic DNA. The nucleic acid probe can be, for example, a full-length GPCRX nucleic acid, such as the nucleic acid of SEQ ID NOS: 1, 3, 5, 7, 9, 11, 13, 15, 17, 19, 21, 23, 25, 27, 29, 31 and 33, or a portion thereof, such as an oligonucleotide of at least 15, 30, 50, 100, 250 or 500 nucleotides in length and sufficient to specifically hybridize under stringent conditions to GPCRX mRNA or genomic DNA. Other suitable probes for use in the diagnostic assays of the invention are described herein.

An agent for detecting GPCRX protein is an antibody capable of binding to GPCRX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect GPCRX mRNA, protein, or genomic DNA in a biological sample in vitro as well as in vivo. For example, in vitro techniques for detection of GPCRX mRNA include Northern hybridizations and in situ hybridizations. In vitro techniques for detection of GPCRX protein include enzyme linked immunosorbent assays (ELISAs), Western blots, immunoprecipitations, and immunofluorescence. In vitro techniques for detection of GPCRX genomic DNA include Southern hybridizations. Furthermore, in vivo techniques for detection of GPCRX protein include introducing into a subject a labeled anti-GPCRX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting GPCRX protein, mRNA, or genomic DNA, such that the presence of GPCRX protein, mRNA or genomic DNA is detected in the biological sample, and comparing the presence of GPCRX protein, mRNA or genomic DNA in the control sample with the presence of GPCRX protein, mRNA or genomic DNA in the test sample.

The invention also encompasses kits for detecting the presence of GPCRX in a biological sample. For example, the kit can comprise: a labeled compound or agent capable of detecting GPCRX protein or mRNA in a biological sample; means for determining the amount of GPCRX in the sample; and means for comparing the amount of GPCRX in the sample with a standard. The compound or agent can be packaged in a suitable container. The kit can further comprise instructions for using the kit to detect GPCRX protein or nucleic acid.

Prognostic Assays

The diagnostic methods described herein can furthermore be utilized to identify subjects having or at risk of developing a disease or disorder associated with aberrant GPCRX expression or activity. For example, the assays described herein, such as the preceding diagnostic assays or the following assays, can be utilized to identify a subject having or at risk of developing a disorder associated with GPCRX protein, nucleic acid expression or activity. Alternatively, the prognostic assays can be utilized to identify a subject having or at risk for developing a disease or disorder. Thus, the invention provides a method for identifying a disease or disorder associated with aberrant GPCRX expression or activity in which a test sample is obtained from a subject and GPCRX protein or nucleic acid (e.g., mRNA, genomic DNA) is detected, wherein the presence of GPCRX protein or nucleic acid is diagnostic for a subject having or at risk of developing a disease or disorder associated with aberrant GPCRX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant GPCRX expression or activity. For example, such methods can be used to determine whether a subject can be effectively treated with an agent for a disorder. Thus, the invention provides methods for determining whether a subject can be effectively treated with an agent for a disorder associated with aberrant GPCRX expression or activity in which a test sample is obtained and GPCRX protein or nucleic acid is detected (e.g., wherein the presence of GPCRX protein or nucleic acid is diagnostic for a subject that can be administered the agent to treat a disorder associated with aberrant GPCRX expression or activity).

The methods of the invention can also be used to detect genetic lesions in an GPCRX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an GPCRX-protein, or the misexpression of the GPCRX gene. For example, such genetic lesions can be detected by ascertaining the existence of at least one of: (i) a deletion of one or more nucleotides from an GPCRX gene; (ii) an addition of one or more nucleotides to an GPCRX gene; (iii) a substitution of one or more nucleotides of an GPCRX gene, (iv) a chromosomal rearrangement of an GPCRX gene; (v) an alteration in the level of a messenger RNA transcript of an GPCRX gene, (vi) aberrant modification of an GPCRX gene, such as of the methylation pattern of the genomic DNA, (vii) the presence of a non-wild-type splicing pattern of a messenger RNA transcript of an GPCRX gene, (viii) a non-wild-type level of an GPCRX protein, (ix) allelic loss of an GPCRX gene, and (x) inappropriate post-translational modification of an GPCRX protein. As described herein, there are a large number of assay techniques known in the art which can be used for detecting lesions in an GPCRX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the GPCRX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an GPCRX gene under conditions such that hybridization and amplification of the GPCRX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an GPCRX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in GPCRX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in GPCRX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the GPCRX gene and detect mutations by comparing the sequence of the sample GPCRX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the GPCRX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type GPCRX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in GPCRX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an GPCRX sequence, e.g., a wild-type GPCRX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in GPCRX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control GPCRX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an GPCRX gene.

Furthermore, any cell type or tissue, preferably peripheral blood leukocytes, in which GPCRX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on GPCRX activity (e.g., GPCRX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of GPCRX protein, expression of GPCRX nucleic acid, or mutation content of GPCRX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. PharmacoL Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofuirans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of GPCRX protein, expression of GPCRX nucleic acid, or mutation content of GPCRX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an GPCRX modulator, such as a modulator identified by one of the exemplary screening assays described herein.

Monitoring of Effects During Clinical Trials

Monitoring the influence of agents (e.g., drugs, compounds) on the expression or activity of GPCRX (e.g., the ability to modulate aberrant cell proliferation and/or differentiation) can be applied not only in basic drug screening, but also in clinical trials. For example, the effectiveness of an agent determined by a screening assay as described herein to increase GPCRX gene expression, protein levels, or upregulate GPCRX activity, can be monitored in clinical trails of subjects exhibiting decreased GPCRX gene expression, protein levels, or downregulated GPCRX activity. Alternatively, the effectiveness of an agent determined by a screening assay to decrease GPCRX gene expression, protein levels, or downregulate GPCRX activity, can be monitored in clinical trails of subjects exhibiting increased GPCRX gene expression, protein levels, or upregulated GPCRX activity. In such clinical trials, the expression or activity of GPCRX and, preferably, other genes that have been implicated in, for example, a cellular proliferation or immune disorder can be used as a “read out” or markers of the immune responsiveness of a particular cell.

By way of example, and not of limitation, genes, including GPCRX, that are modulated in cells by treatment with an agent (e.g., compound, drug or small molecule) that modulates GPCRX activity (e.g., identified in a screening assay as described herein) can be identified. Thus, to study the effect of agents on cellular proliferation disorders, for example, in a clinical trial, cells can be isolated and RNA prepared and analyzed for the levels of expression of GPCRX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of GPCRX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an GPCRX protein, mRNA, or genomic DNA in the preadministration sample; (iii) obtaining one or more post-administration samples from the subject; (iv) detecting the level of expression or activity of the GPCRX protein, mRNA, or genomic DNA in the post-administration samples; (v) comparing the level of expression or activity of the GPCRX protein, mRNA, or genomic DNA in the pre-administration sample with the GPCRX protein, mRNA, or genomic DNA in the post administration sample or samples; and (vi) altering the administration of the agent to the subject accordingly. For example, increased administration of the agent may be desirable to increase the expression or activity of GPCRX to higher levels than detected, i.e., to increase the effectiveness of the agent. Alternatively, decreased administration of the agent may be desirable to decrease expression or activity of GPCRX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant GPCRX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endoggenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant GPCRX expression or activity, by administering to the subject an agent that modulates GPCRX expression or at least one GPCRX activity. Subjects at risk for a disease that is caused or contributed to by aberrant GPCRX expression or activity can be identified by, for example, any or a combination of diagnostic or prognostic assays as described herein. Administration of a prophylactic agent can occur prior to the manifestation of symptoms characteristic of the GPCRX aberrancy, such that a disease or disorder is prevented or, alternatively, delayed in its progression. Depending upon the type of GPCRX aberrancy, for example, an GPCRX agonist or GPCRX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating GPCRX expression or activity for therapeutic purposes. The modulatory method of the invention involves contacting a cell with an agent that modulates one or more of the activities of GPCRX protein activity associated with the cell. An agent that modulates GPCRX protein activity can be an agent as described herein, such as a nucleic acid or a protein, a naturally-occurring cognate ligand of an GPCRX protein, a peptide, an GPCRX peptidomimetic, or other small molecule. In one embodiment, the agent stimulates one or more GPCRX protein activity. Examples of such stimulatory agents include active GPCRX protein and a nucleic acid molecule encoding GPCRX that has been introduced into the cell. In another embodiment, the agent inhibits one or more GPCRX protein activity. Examples of such inhibitory agents include antisense GPCRX nucleic acid molecules and anti-GPCRX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an GPCRX protein or nucleic acid molecule. In one embodiment, the method involves administering an agent (e.g., an agent identified by a screening assay described herein), or combination of agents that modulates (e.g., up-regulates or down-regulates) GPCRX expression or activity. In another embodiment, the method involves administering an GPCRX protein or nucleic acid molecule as therapy to compensate for reduced or aberrant GPCRX expression or activity.

Stimulation of GPCRX activity is desirable in situations in which GPCRX is abnormally downregulated and/or in which increased GPCRX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The GPCRX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the GPCRX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the GPCRX protein, and the GPCRX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further usefull in the generation of antibodies which immunospecifically-bind to the novel substances of the invention for use in therapeutic or diagnostic methods.

### EXAMPLES

### Example 1

Identification of GPCRX Clones

All novel GPCRX target sequences identified in the present invention were subjected to the exon linking process to confirm the sequence. PCR primers were designed by starting at the most upstream sequence available, for the forward primer, and at the most downstream sequence available for the reverse primer. Table 17A shows the sequences of the PCR primers used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

TABLE 17A.

PCR Primers for Exon Linking

| | | SEQ | | SEQ |
|---|---|---|---|---|
| | Primer Sequence | ID | Primer Sequence | ID |
| Clone | 5′-3′ | NO | 3′-5′ | NO |
| GPCR4 | GCAATGGGGCTCAATACGTCTG | 114 | AATGTCATGCTCTAGAGTGAGGCAGA | 115 |
| GPCR14 | ACATGGAGATAAAGAACTACAGCAGGA | 116 | TGTTCCTTTTACCGGTAAATTCTGTCC | 117 |

Primers were designed based on in silico predictions of the full length or some portion (one or more exons) of the cDNA/protein sequence of the invention. These primers were used to amplify a cDNA from a pool containing expressed human sequences derived from the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

Multiple clones were sequenced and these fragments were assembled together, sometimes including public human sequences, using bioinformatic programs to produce a consensus sequence for each assembly. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clones: The PCR products derived by exon linking, covering the entire open reading frame, were cloned into the pCR2.1 vector from Invitrogen to provide clones 126160::CG55928-01.698615.M4 (GPCR4), 115521::GMAC006313_B.698322.I10 (GPCR14) and 115521::GMAC006313_B.698322.I14 (GPCR14).

### Example 2

Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune diseases), Panel CNSD.01 (containing central nervous system samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 of 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1× TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqManq® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1×TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_screening_panel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, MD) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, BOL-1, KU-812. EOL cells were further differentiated by culture in 0.1 mM dbcAMP at 5×10⁵cells/ml for 8 days, changing the media every 3 days and adjusting the cell concentration to 5×10 cells/ml. For the culture of these cells, we used DMEM or RPMI (as recommended by the ATGC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCG. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochioropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_Comprehensive Panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-i anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus =From Backus Hospital

OA=Osteoarthritis

(SS)(BA)(MF)=Individual patients

Adj=Adjacent tissue

Match control=adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample.

Patient 2: Diabetic Hispanic, overweight, not on insulin

Patient 7-9: Nondiabetic Caucasian and obese (BMI>30)

Patient 10: Diabetic Hispanic, overweight, on insulin

Patient 11: Nondiabetic African American and overweight

Patient 12: Diabetic Hispanic on insulin

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supemuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4 =Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

A. CG55956-01: GPCR

Expression of gene CG55956-01 was assessed using the primer-probe set Ag2193, described in Table 18AA. Results of the RTQ-PCR runs are shown in Tables 18AB, 18AC and 18AD.

TABLE 18AA.

Probe Name Ag2193

| | | | |
|---|---|---|---|
| Primers | Sequences | Length | Start Position |

| | | | | |
|---|---|---|---|---|
| Forward | 5′-gccctttagataagtcgtccaa-3′ | (SEQ ID NO:118) | 22 | 678 |
| Probe | TET-5′-agctctgtccactttgactgctcaca-3′-TAMRA | (SEQ ID NO:119) | 126 | 700 |
| Reverse | 5′-catggtccaaagaacaaaagaa-3′ | (SEQ ID NO:120) | 22 | 735 |

TABLE 18AB
Panel 1.3D

| | Rel. Exp. (%) | | Rel. Exp. (%) |
|---|---|---|---|
| | Ag2193, Run | | Ag2193, Run |
| Tissue Name | 165750872 | Tissue Name | 165750872 |
| Liver adenocarcinoma | 0.0 | Kidney (fetal) | 0.0 |
| Pancreas | 0.0 | Renal ca. 786-0 | 22.7 |
| Pancreatic ca. CAPAN 2 | 0.0 | Renal ca. A498 | 25.5 |
| Adrenal gland | 0.0 | Renal ca. RXF 393 | 0.0 |
| Thyroid | 0.0 | Renal ca. ACHN | 100.0 |
| Salivary gland | 0.0 | Renal ca. UO-31 | 4.1 |
| Pituitary gland | 0.0 | Renal ca. TK-10 | 21.9 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) HepG2 | 1.9 |
| Brain (cerebellum) | 3.6 | Lung | 0.0 |
| Brain (hippocampus) | 0.0 | Lung (fetal) | 0.0 |
| Brain (substantia nigra) | 0.0 | Lung ca. (small cell) LX-1 | 5.0 |
| Brain (thalamus) | 0.0 | Lung ca. (small cell) NCI-H69 | 0.0 |
| Cerebral Cortex | 0.0 | Lung ca. (s. cell var.) SHP-77 | 0.0 |
| Spinal cord | 0.0 | Lung ca. (large cell)NCI-H460 | 0.0 |
| glio/astro U87-MG | 9.0 | Lung ca. (non-sm. cell) A549 | 0.0 |
| glio/astro U-118-MG | 48.0 | Lung ca. (non-s. cell) NCI-H23 | 5.0 |
| astrocytoma SW1783 | 5.9 | Lung ca. (non-s. cell) HOP-62 | 0.0 |
| neuro*; met SK-N-AS | 0.0 | Lung ca. (non-s. cl) NCI-H522 | 0.0 |
| astrocytoma SF-539 | 0.0 | Lung ca. (squam.) SW 900 | 0.0 |
| astrocytoma SNB-75 | 2.8 | Lung ca. (squam.) NCI-H596 | 0.0 |
| glioma SNB-19 | 14.1 | Mammary gland | 0.0 |
| glioma U251 | 43.2 | Breast ca.* (pl. ef) MCF-7 | 0.0 |
| glioma SF-295 | 3.7 | Breast ca.* (pl. ef) MDA-MB-231 | 0.0 |
| Heart (fetal) | 0.0 | Breast ca.* (pl. ef) T47D | 0.0 |
| Heart | 0.0 | Breast ca. BT-549 | 6.2 |
| Skeletal muscle (fetal) | 0.0 | Breast ca. MDA-N | 31.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 4.8 |
| Spleen | 0.0 | Ovarian ca. OVCAR-5 | 0.0 |
| Lymph node | 0.0 | Ovarian ca. OVCAR-8 | 8.6 |
| Colorectal | 28.1 | Ovarian ca. IGROV-1 | 0.0 |
| Stomach | 0.0 | Ovarian ca.* (ascites) SK-OV-3 | 0.0 |
| Small intestine | 0.0 | Uterus | 0.0 |
| Colon ca. SW480 | 3.3 | Plancenta | 0.0 |
| Colon ca.* SW620(SW480 met) | 0.0 | Prostate | 0.0 |
| Colon ca. HT29 | 0.0 | Prostate ca.* (bone met)PC-3 | 0.0 |
| Colon ca. HCT-116 | 0.0 | Testis | 5.2 |
| Colon ca. CaCo-2 | 0.0 | Melanoma Hs688(A).T | 0.0 |
| Colon ca. tissue(ODO3866) | 0.0 | Melanoma* (met) Hs688(B).T | 0.0 |
| Colon ca. HCC-2998 | 4.9 | Melanoma UACC-62 | 27.2 |
| Gastric ca.* (liver met) NCI-N87 | 0.0 | Melanoma M14 | 32.8 |
| Bladder | 0.0 | Melanoma LOX IMVI | 0.0 |
| Trachea | 0.0 | Melanoma* (met) SK-MEL-5 | 11.4 |
| Kidney | 0.0 | Adipose | 4.5 |

TABLE 18AC
Panel 2D

| | Rel. Exp. (%) | | Rel. Exp. (%) |
|---|---|---|---|
| | Ag2193, Run | | Ag2193, Run |
| Tissue Name | 163584683 | Tissue Name | 163584683 |
| Normal Colon | 4.9 | Kidney Margin 8120608 | 2.4 |
| CC Well to Mod Diff (ODO3866) | 32.5 | Kidney Cancer 8120613 | 2.5 |
| CC Margin (ODO3866) | 13.2 | Kidney Margin 8120614 | 0.0 |
| CC Gr.2 rectosigmoid | 0.0 | Kidney Cancer 9010320 | 0.0 |
| (ODO3868) | | | |
| CC Margin (ODO3868) | 0.0 | Kidney Margin 9010321 | 0.0 |
| CC Mod Diff (ODO3920) | 2.2 | Normal Uterus | 0.0 |
| CC Margin (ODO3920) | 0.0 | Uterus Cancer 064011 | 0.0 |
| CC Gr.2 ascend colon | 0.0 | Normal Thyroid | 0.0 |
| (ODO3921) | | | |
| CC Margin (ODO3921) | 15.8 | Thyroid Cancer 064010 | 0.0 |
| CC from Partial Hepatectomy | 0.0 | Thyroid Cancer A302152 | 0.0 |
| (ODO4309) Mets | | | |
| Liver Margin (ODO4309) | 0.0 | Thyroid Margin A302153 | 0.0 |
| Colon mets to lung (OD04451- | 0.0 | Normal Breast | 0.0 |
| 01) | | | |
| Lung Margin (OD04451-02) | 1.7 | Breast Cancer (OD04566) | 8.9 |
| Normal Prostate 6546-1 | 1.6 | Breast Cancer (OD04590-01) | 0.0 |
| Prostate Cancer (OD04410) | 3.8 | Breast Cancer Mets (OD04590-03) | 0.0 |
| Prostate Margin (OD04410) | 0.0 | Breast Cancer Metastasis | 4.0 |
| | | (OD04655-05) | |
| Prostate Cancer (OD04720-01) | 0.0 | Breast Cancer 064006 | 0.0 |
| Prostate Margin (OD04720-02) | 1.6 | Breast Cancer 1024 | 0.0 |
| Normal Lung 061010 | 4.7 | Breast Cancer 9100266 | 0.0 |
| Lung Met to Muscle (ODO4286) | 34.6 | Breast Margin 9100265 | 0.0 |
| Muscle Margin (ODO4286) | 2.5 | Breast Cancer A209073 | 20.7 |
| Lung Malignant Cancer | 0.0 | Breast Margin A2090734 | 1.6 |
| (OD03126) | | | |
| Lung Margin (OD03126) | 0.6 | Normal Liver | 0.0 |
| Lung Cancer (OD04404) | 0.0 | Liver Cancer 064003 | 2.6 |
| Lung Margin (OD04404) | 3.0 | Liver Cancer 1025 | 0.0 |
| Lung Cancer (OD04565) | 0.0 | Liver Cancer 1026 | 0.0 |
| Lung Margin (OD04565) | 0.0 | Liver Cancer 6004-T | 3.2 |
| Lung Cancer (OD04237-01) | 0.0 | Liver Tissue 6004-N | 15.0 |
| Lung Margin (OD04237-02) | 0.0 | Liver Cancer 6005-T | 0.0 |
| Ocular Mel Met to Liver | 39.0 | Liver Tissue 6005-N | 0.0 |
| (ODO4310) | | | |
| Liver Margin (ODO4310) | 5.9 | Normal Bladder | 0.0 |
| Melanoma Mets to Lung | 11.0 | Bladder Cancer 1023 | 0.0 |
| (OD04321) | | | |
| Lung Margin (OD04321) | 0.0 | Bladder Cancer A302173 | 66.9 |
| Normal Kidney | 4.9 | Bladder Cancer (OD04718-01) | 0.0 |
| Kidney Ca, Nuclear grade 2 | 95.9 | Bladder Normal Adjacent | 0.0 |
| (OD04338) | | (OD04718-03) | |
| Kidney Margin (OD04338) | 23.0 | Normal Ovary | 0.0 |
| Kidney Ca Nuclear grade 1/2 | 100.0 | Ovarian Cancer 064008 | 0.0 |
| (OD04339) | | | |
| Kidney Margin (OD04339) | 37.1 | Ovarian Cancer (OD04768-07) | 1.5 |
| Kidney Ca, Clear cell type | 9.9 | Ovary Margin (OD04768-08) | 0.0 |
| (OD04340) | | | |
| Kidney Margin (OD04340) | 5.8 | Normal Stomach | 0.0 |
| Kidney Ca, Nuclear grade 3 | 0.0 | Gastric Cancer 9060358 | 0.0 |
| (OD04348) | | | |
| Kidney Margin (OD04348) | 15.7 | Stomach Margin 9060359 | 0.0 |
| Kidney Cancer (OD04622-01) | 0.0 | Gastric Cancer 9060395 | 5.4 |
| Kidney Margin (OD04622-03) | 2.1 | Stomach Margin 9060394 | 0.0 |
| Kidney Cancer (OD04450-01) | 66.4 | Gastric Cancer 9060397 | 0.0 |
| Kidney Margin (OD04450-03) | 7.1 | Stomach Margin 9060396 | 0.0 |
| Kidney Cancer 8120607 | 1.6 | Gastric Cancer 064005 | 46.0 |

TABLE 18AD
Panel 4D

| | Rel. Exp. (%) | | Rel. Exp. (%) |
|---|---|---|---|
| | Ag2193, Run | | Ag2193, Run |
| Tissue Name | 163603073 | Tissue Name | 163603073 |
| Secondary Th1 act | 12.1 | HUVEC IL-1beta | 14.2 |
| Secondary Th2 act | 0.0 | HUVEC IFN gamma | 0.0 |
| Secondary Tr1 act | 0.0 | HUVEC TNF alpha + IFN gamma | 52.9 |
| Secondary Th1 rest | 0.0 | HUVEC TNF alpha + IL4 | 9.2 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + | 0.0 |
| | | IL-1beta | |
| Primary Th2 act | 5.3 | Microvascular Dermal EC none | 0.0 |
| Primary Tr1 act | 0.0 | Microsvasular Dermal EC | 0.0 |
| | | TNFalpha + IL-1beta | |
| Primary Th1 rest | 0.0 | Bronchial epithelium TNFalpha + | 0.0 |
| | | IL1beta | |
| Primary Th2 rest | 0.0 | Small airway epithelium none | 0.0 |
| Primary Tr1 rest | 0.0 | Small airway epithelium TNFalpha + | 0.0 |
| | | IL-1beta | |
| CD45RA CD4 lymphocyte act | 0.0 | Coronery artery SMC rest | 0.0 |
| CD45RO CD4 lymphocyte act | 0.0 | Coronery artery SMC TNFalpha + | 0.0 |
| | | IL-1beta | |
| CD8 lymphocyte act | 0.0 | Astrocytes rest | 0.0 |
| Secondary CD8 lymphocyte rest | 0.0 | Astrocytes TNFalpha + IL-1beta | 0.0 |
| Secondary CD8 lymphocyte act | 0.0 | KU-812 (Basophil) rest | 0.0 |
| CD4 lymphocyte none | 0.0 | KU-812 (Basophil) | 6.8 |
| | | PMA/ionomycin | |
| 2ry Th1/Th2/Tr1_anti-CD95 | 7.8 | CCD1106 (Keratinocytes) none | 0.0 |
| CH11 | | | |
| LAK cells rest | 0.0 | CCD1106 (Keratinocytes) | 7.6 |
| | | TNFalpha + IL-1beta | |
| LAK cells IL-2 | 7.2 | Liver cirrhosis | 66.0 |
| LAK cells IL-2 + IL-12 | 0.0 | Lupus kidney | 14.9 |
| LAK cells IL-2 + IFN gamma | 0.0 | NCI-H292 none | 68.8 |
| LAK cells IL-2 + IL-18 | 7.1 | NCI-H292 IL-4 | 70.2 |
| LAK cells PMA/ionomycin | 0.0 | NCI-H292 IL-9 | 100.0 |
| NK Cells IL-2 rest | 0.0 | NCI-H292 IL-13 | 31.0 |
| Two Way MLR 3 day | 0.0 | NCI-H292 IFN gamma | 72.2 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 0.0 | Lung fibroblast TNF alpha + IL-1 | 0.0 |
| | | beta | |
| PBMC PHA-L | 0.0 | Lung fibroblast IL-4 | 7.9 |
| Ramos (B cell) none | 0.0 | Lung fibroblast IL-9 | 22.4 |
| Ramos (B cell) ionomycin | 0.0 | Lung fibroblast IL-13 | 7.6 |
| B lymphocytes PWM | 0.0 | Lung fibroblast IFN gamma | 0.0 |
| B lymphocytes CD40L and IL-4 | 0.0 | Dermal fibroblast CCD1070 rest | 0.0 |
| EOL-1 dbcAMP | 0.0 | Dermal fibroblast CCD1070 TNF | 13.7 |
| | | alpha | |
| EOL-1 dbcAMP PMA/ionomycin | 0.0 | Dermal fibroblast CCD1070 IL-1 | 0.0 |
| | | beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 24.3 |
| Monocytes rest | 0.0 | IBD Crohn's | 6.7 |
| Monocytes LPS | 0.0 | Colon | 34.9 |
| Macrophages rest | 90.1 | Lung | 12.4 |
| Macrophages LPS | 0.0 | Thymus | 13.7 |
| HUVEC none | 36.9 | Kidney | 0.0 |
| HUVEC starved | 55.9 | | |

Panel 1.3D Summary: Ag2193 The expression of the CG55956-01 gene appears to be highest in a sample derived from a renal cancer cell line (ACHN) (CT=33.2). In addition, there is substantial expression associated with brain cancer cell lines, a melanoma and a breast cancer cell line. Thus, the expression of this gene could be used to distinguish samples derived from the ACHN cell line form other samples in the panel. Moreover, therapeutic modulation of this gene, through the use of small molecule drugs, antibodies or protein therapeutics might be of use in the treatment of melanoma, breast cancer, renal cancer of brain cancer.

Panel 2D Summary: Ag2193 The expression of the CG55956-01 gene is highest in a sample derived from a kidney cancer (CT=32.1). In addition, there is substantial expression associated with other kidney cancers. Of note is the difference in expression between kidney cancers and their normal adjacent tissues. Thus, the expression of this gene could be used to distinguish kidney cancer samples from other samples in the panel, and in particular, distinguish kidney cancer from normal kidney. Moreover, therapeutic modulation of this gene, through the use of small molecule drugs, antibodies or protein therapeutics might be of use in the treatment of kidney cancer.

Panel 4D Summary: Ag2193 The CG55956-01 gene is expressed at low levels in resting and IL-4, IL-9, and IFN gamma activated-NCI-H292 mucoepidermoid cells, both starved as well as TNF alpha+IFN gamma treated HUVECs, and resting macrophages. The expression of this gene in lung derived cells, endothelial cells and macrophages suggests that this gene may be involved in normal conditions as well as pathological and inflammatory lung disorders that include chronic obstructive pulmonary disease, asthma, allergy and emphysema. The expression in activated endothelial cells Small molecules or antibodies that modulate the function of Ag2193 may be useful therapeutics for the reduction or elimination of the symptoms in chronic obstructive pulmonary disease, asthma, allergy, and emphysema.

B. CG55952-01: GPCR

Expression of gene CG55952-01 was assessed using the primer-probe set Ag3762, described in Table 18BA.

TABLE 18BA.

Probe Name Ag3762

| | | | |
|---|---|---|---|
| | | | Start |
| Primers | Sequences | Length | Position |

| | | | | |
|---|---|---|---|---|
| Forward | 5′-ccattattggaactggcatgta-3′ | (SEQ ID NO:121) | 22 | 544 |
| Probe | TET-5′-tgacacctactttataggcctcactgtt-3′-TAMRA | (SEQ ID NO:122) | 29 | 567 |
| Reverse | 5′-atacagagtgctccaccactga-3′ | (SEQ ID NO:123) | 22 | 599 |

CNS_neurodegeneration_v1.0 Summary: Ag3762 Expression of the CG55952-01 gene is low/undetectable in all samples on this panel (CTs>35). (Data not shown.)

General_screening panel_v1.4 Summary: Ag3762 Expression of the CG55952-01 gene is low/undetectable in all samples on this panel (CTs>35). (Data not shown.)

Panel 4.1D Summary: Ag3762 Expression of the CG55952-01 gene is low/undetectable in all samples on this panel (CTs>35). (Data not shown.)

C. CG55950-01: GPChR

Expression of gene CG55950-01 was assessed using the primer-probe set Ag3761, described in Table 18CA.

TABLE 18CA.

Probe Name Ag3761

| | | | |
|---|---|---|---|
| | | | Start |
| Primers | Sequences | Length | Position |

| | | | | |
|---|---|---|---|---|
| Forward | 5′-tagttttcagtggtggagcaa-3′ | (SEQ ID NO:124) | 21 | 593 |
| Probe | TET-5′-tgtgtatggtcatcttcgcccttcta-3′-TAMRA | (SEQ ID NO:125) | 126 | 614 |
| Reverse | 5′-gggagtttaggctgactccata-3′ | (SEQ ID NO:126) | 22 | 649 |

CNS_neurodegeneration_v1.0 Summary: Ag3761 Expression of gene CG55950-01 is low/undetectable in all samples on this panel. (CTs>35). (Data not shown. )

General_screening_panel_v1.4 Summary: Ag3761 Expression of gene CG55950-01 is low/undetectable in all samples on this panel. (CTs>35). (Data not shown.)

Panel 4.1D Summary: Ag3761 Expression of gene CG55950-01 is low/undetectable in all samples on this panel. (CTs>35). (Data not shown.)

D. CG55928-01 and CG55928-02: GPCR

Expression of gene CG55928-01 and variant CG55928-02 was assessed using the primer-probe set Ag2855, described in Table 18DA. Results of the RTQ-PCR runs are shown in Tables 18DB and 18DC.

TABLE 18DA.

Probe Name Ag2855

| | | | |
|---|---|---|---|
| | | | Start |
| Primers | Sequences | Length | Position |

| | | | | |
|---|---|---|---|---|
| Forward | 5′-tggcactcttctctttctcatc-3′ | (SEQ ID NO: 127) | 22 | 126 |
| Probe | TET-5′-tgatcataacctccatgagcccatgt-3′-TAMRA | (SEQ ID NO:128) | 26 | 153 |
| Reverse | 5′-gtctgtagctgccaacatagct-3′ | (SEQ ID NO:129) | 22 | 189 |

TABLE 18DB
Panel 1.3D

| | Rel. Exp. (%) | | Rel. Exp. (%) |
|---|---|---|---|
| | Ag2855, Run | | Ag2855, Run |
| Tissue Name | 167643873 | Tissue Name | 167643873 |
| Liver adenocarcinoma | 25.5 | Kidney (fetal) | 1.4 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 1.6 | Liver | 0.0 |
| Brain (whole) | 0.0 | Liver (fetal) | 0.0 |
| Brain (amygdala) | 0.0 | Liver ca. (hepatoblast) HepG2 | 0.5 |
| Brain (cerebellum) | 0.0 | Lung | 0.0 |
| Brain (hippocampus) | 0.0 | Lung (fetal) | 1.1 |
| Brain (substantia nigra) | 0.0 | Lung ca. (small cell) LX-1 | 29.3 |
| Brain (thalamus) | 0.0 | Lung ca. (small cell) NCI-H69 | 0.0 |
| Cerebral Cortex | 0.7 | Lung ca. (s. cell var.) SHP-77 | 7.8 |
| Spinal cord | 0.0 | Lung ca. (large cell)NCI-H460 | 0.0 |
| glio/astro U87-MG | 0.0 | Lung ca. (non-sm. cell) A549 | 1.1 |
| glio/astro U-118-MG | 0.0 | Lung ca. (non-s. cell) NCI-H23 | 0.0 |
| astrocytoma SW1783 | 0.0 | Lung ca. (non-s. cell) HOP-62 | 0.0 |
| neuro*; met SK-N-AS | 0.0 | Lung ca. (non-s. cl) NCI-H522 | 0.0 |
| astrocytoma SF-539 | 1.7 | Lung ca. (squam.) SW 900 | 0.0 |
| astrocytoma SNB-75 | 0.0 | Lung ca. (squam.) NCI-H596 | 0.0 |
| glioma SNB-19 | 0.0 | Mammary gland | 0.0 |
| glioma U251 | 1.3 | Breast ca.* (pl. ef) MCF-7 | 0.0 |
| glioma SF-295 | 0.0 | Breast ca.* (pl. ef) MDA-MB-231 | 0.0 |
| Heart (fetal) | 0.0 | Breast ca.* (pl. ef) T47D | 0.0 |
| Heart | 0.0 | Breast ca. BT-549 | 0.0 |
| Skeletal muscle (fetal) | 0.0 | Breast ca. MDA-N | 29.5 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.7 |
| Colorectal | 0.0 | Ovarian ca. IGROV-1 | 0.0 |
| Stomach | 0.0 | Ovarian ca.* (ascites) SK-OV-3 | 4.4 |
| Small intestine | 0.0 | Uterus | 0.0 |
| Colon ca. SW480 | 0.0 | Plancenta | 0.0 |
| Colon ca.* SW620(SW480 met) | 8.7 | Prostate | 0.8 |
| Colon ca. HT29 | 0.0 | Prostate ca.* (bone met)PC-3 | 0.9 |
| Colon ca. HCT-116 | 100.0 | Testis | 0.0 |
| Colon ca. CaCo-2 | 0.0 | Melanoma Hs688(A).T | 0.0 |
| Colon ca. tissue (ODO3866) | 0.0 | Melanoma* (met) Hs688(B).T | 0.0 |
| Colon ca. HCC-2998 | 0.0 | Melanoma UACC-62 | 1.0 |
| Gastric ca.* (liver met) NCI-N87 | 0.7 | Melanoma M14 | 0.0 |
| Bladder | 0.8 | Melanoma LOX IMVI | 7.1 |
| Trachea | 0.0 | Melanoma* (met) SK-MEL-5 | 47.0 |
| Kidney | 1.0 | Adipose | 0.6 |

TABLE 18DC
Panel 4D

| | Rel. Exp. (%) | | Rel. Exp. (%) |
|---|---|---|---|
| | Ag2855, Run | | Ag2855, Run |
| Tissue Name | 164401528 | Tissue Name | 164401528 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + | 0.0 |
| | | IL-1beta | |
| Primary Th2 act | 0.0 | Microvascular Dermal EC none | 0.0 |
| Primary Tr1 act | 3.2 | Microsvasular Dermal EC TNFalpha + | 0.0 |
| | | IL-1beta | |
| Primary Th1 rest | 0.0 | Bronchial epithelium TNFalpha + | 0.0 |
| | | IL1beta | |
| Primary Th2 rest | 0.0 | Small airway epithelium none | 0.0 |
| Primary Tr1 rest | 0.0 | Small airway epithelium TNFalpha + | 0.0 |
| | | IL-1beta | |
| CD45RA CD4 lymphocyte act | 0.0 | Coronery artery SMC rest | 4.2 |
| CD45RO CD4 lymphocyte act | 0.0 | Coronery artery SMC TNFalpha + IL- | 0.0 |
| | | 1beta | |
| CD8 lymphocyte act | 0.0 | Astrocytes rest | 0.0 |
| Secondary CD8 lymphocyte rest | 0.0 | Astrocytes TNFalpha + IL-1beta | 0.0 |
| Secondary CD8 lymphocyte act | 0.0 | KU-812 (Basophil) rest | 0.0 |
| CD4 lymphocyte none | 0.0 | KU-812 (Basophil) PMA/ionomycin | 31.4 |
| 2ry Th1/Th2/Tr1_anti-CD95 CH11 | 0.0 | CCD1106 (Keratinocytes) none | 0.0 |
| LAK cells rest | 0.0 | CCD1106 (Keratinocytes) TNFalpha + | 0.0 |
| | | IL-1beta | |
| LAK cells IL-2 | 2.4 | Liver cirrhosis | 10.8 |
| LAK cells IL-2 + IL-12 | 3.3 | Lupus kidney | 2.7 |
| LAK cells IL-2 + IFN gamma | 0.0 | NCI-H292 none | 0.0 |
| LAK cells IL-2 + IL-18 | 2.0 | NCI-H292 IL-4 | 0.0 |
| LAK cells PMA/ionomycin | 0.0 | NCI-H292 IL-9 | 0.0 |
| NK Cells IL-2 rest | 0.0 | NCI-H292 IL-13 | 0.0 |
| Two Way MLR 3 day | 8.1 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 2.1 | Lung fibroblast none | 0.0 |
| PBMC PWM | 0.0 | Lung fibroblast TNF alpha + IL-1 beta | 2.7 |
| PBMC PHA-L | 0.0 | Lung fibroblast IL-4 | 3.2 |
| Ramos (B cell) none | 29.7 | Lung fibroblast IL-9 | 0.0 |
| Ramos (B cell) ionomycin | 100.0 | Lung fibroblast IL-13 | 0.0 |
| B lymphocytes PWM | 3.6 | Lung fibroblast IFN gamma | 0.0 |
| B lymphocytes CD40L and IL-4 | 2.9 | Dermal fibroblast CCD1070 rest | 0.0 |
| EOL-1 dbcAMP | 0.0 | Dermal fibroblast CCD1070 TNF | 0.0 |
| | | alpha | |
| EOL-1 dbcAMP PMA/ionomycin | 0.0 | Dermal fibroblast CCD1070 IL-1 beta | 0.0 |
| Dendritic cells none | 0.0 | Dermal fibroblast IFN gamma | 0.0 |
| Dendritic cells LPS | 2.0 | Dermal fibroblast IL-4 | 0.0 |
| Dendritic cells anti-CD40 | 0.0 | IBD Colitis 2 | 0.0 |
| Monocytes rest | 0.0 | IBD Crohn's | 2.0 |
| Monocytes LPS | 0.0 | Colon | 0.0 |
| Macrophages rest | 2.6 | Lung | 3.6 |
| Macrophages LPS | 0.0 | Thymus | 30.1 |
| HUVEC none | 0.0 | Kidney | 19.5 |
| HUVEC starved | 0.0 | | |

Panel 1.3D Summary: Ag2855 Expression of the CG55928-01 gene is highest in a colon cancer cell line (CT=32.4). Overall, the expression in this panel is restricted to samples derived from lung, breast, and liver and melanoma cancer cell lines. Thus, expression of this gene could be used to differentiate between these cancer cell lines and other samples on this panel. Furthermore, this expression profile suggests that expression of this gene could be used as a marker to detect the presence of these cancers.

Panel 2.2 Summary: Ag2855 Expression of the CG55928-01 gene is low/undetectable in all samples on this panel (CTs>35).

Panel 4D Summary: Ag2855 Expression of the CG55928-01 gene is limited to a sample derived from the Ramos B cell line (CT=33.5). B cells represent a principle component of immunity and contribute to the immune response in a number of important functional roles, including antibody production. Production of antibodies against self-antigens is a major component in autoimmune disorders. Since B cells play an important role in inflammatory processes and inflammatory cascades, therapeutic modulation of this gene product may reduce or eliminate the symptoms of patients suffering from asthma, allergies, chronic obstructive pulmonary disease, emphysema, Crohn's disease, ulcerative colitis, rheumatoid arthritis, psoriasis, osteoarthritis, and other autoimmune disorders.

E. CG55926-01: GPCR

Expression of gene CG55926-01 was assessed using the primer-probe set Ag2854, described in Table 18EA. Results of the RTQ-PCR runs are shown in Tables 18EB and 18EC.

TABLE 18EA
Probe Name Ag2854

| Primers | Sequences | | Length | Start Position |
|---|---|---|---|---|
| Forward | 5′-gcttttcccaggcctactttat-3′ | (SEQ ID NO:130) | 22 | 285 |
| Probe | TET-5′-cctttctcgagtctggcattctgctt-3′-TAMRA | (SEQ ID NO:131) | 26 | 318 |
| Reverse | 5′-gatggcaataaaacggtcatag-3′ | (SEQ ID NO:132) | 22 | 352 |

TABLE 18EB
Panel 1.3D

| | Rel. Exp. (%) | | Rel. Exp. (%) |
|---|---|---|---|
| | Ag2854,Run | | Ag2854, Run |
| Tissue Name | 167819100 | Tissue Name | 167819100 |
| Liver adenocarcinoma | 9.1 | Kidney (fetal) | 0.9 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.1 | Renal ca. UO-31 | 0.0 |
| Pituitary gland | 0.0 | Renal ca. TK-10 | 0.6 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) HepG2 | 0.0 |
| Brain (cerebellum) | 0.5 | Lung | 0.0 |
| Brain (hippocampus) | 0.0 | Lung (fetal) | 0.2 |
| Brain (substantia nigra) | 0.0 | Lung ca. (small cell) LX-1 | 29.5 |
| Brain (thalamus) | 0.0 | Lung ca. (small cell) NCI-H69 | 0.0 |
| Cerebral Cortex | 0.4 | Lung ca. (s. cell var.) SHP-77 | 2.7 |
| Spinal cord | 0.3 | Lung ca. (large cell)NCI-H460 | 0.0 |
| glio/astro U87-MG | 0.0 | Lung ca. (non-sm. cell) A549 | 0.0 |
| glio/astro U-118-MG | 1.3 | Lung ca. (non-s. cell) NCI-H23 | 0.0 |
| astrocytoma SW1783 | 0.0 | Lung ca. (non-s. cell) HOP-62 | 0.0 |
| neuro*, met SK-N-AS | 0.0 | Lung ca. (non-s. cl) NCI-H522 | 0.0 |
| astrocytoma SF-539 | 0.0 | Lung ca. (squam.) SW 900 | 0.3 |
| astrocytoma SNB-75 | 0.0 | Lung ca. (squam.) HCI-H596 | 0.0 |
| glioma SNB-19 | 0.0 | Mammary gland | 0.0 |
| glioma U251 | 0.6 | Breast ca.* (pl. ef) MCF-7 | 0.0 |
| glioma SF-295 | 0.0 | Breast ca.* (pl. ef) MDA-MB-231 | 0.2 |
| Heart (fetal) | 0.0 | Breast ca.* (pl. ef) T47D | 0.0 |
| Heart | 0.4 | Breast ca. BT-549 | 0.0 |
| Skeletal muscle (fetal) | 0.0 | Breast ca. MDA-N | 7.5 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.5 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.6 |
| Stomach | 0.0 | Ovarian ca.* (ascites) SK-OV-3 | 4.6 |
| Small intestine | 0.0 | Uterus | 0.0 |
| Colon ca. SW480 | 0.0 | Plancenta | 0.0 |
| Colon ca.* SW620(SW480 met) | 12.2 | Prostate | 0.0 |
| Colon ca. HT29 | 0.0 | Prostate ca.* (bone met)PC-3 | 0.0 |
| Colon ca. HCT-116 | 100.0 | Testis | 0.0 |
| Colon ca. CaCo-2 | 0.3 | Melanoma Hs688(A).T | 0.0 |
| Colon ca. tissue(ODO3866) | 0.0 | Melanoma* (met) Hs688(B).T | 0.0 |
| Colon ca. HCC-2998 | 0.3 | Melanoma UACC-62 | 0.5 |
| Gastric ca.* (liver met) NCI-N87 | 0.0 | Melanoma M14 | 0.5 |
| Bladder | 2.1 | Melanoma LOX IMVI | 7.2 |
| Trachea | 0.0 | Melanoma* (met) SK-MEL-5 | 34.9 |
| Kidney | 0.0 | Adipose | 1.1 |

TABLE 18EC
Panel 4D

| | Rel. Exp. (%) | | Rel. Exp. (%) |
|---|---|---|---|
| | Ag2854, Run | | Ag2854, Run |
| Tissue Name | 164299492 | Tissue Name | 164299492 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.1 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1beta | |
| Primary Th2 act | 0.0 | Microvascular Dermal EC none | 0.9 |
| Primary Tr1 act | 0.0 | Microsvasular Dermal EC | 0.0 |
| | | TNFalpha + IL-1beta | |
| Primary Th1 rest | 1.6 | Bronchial epithelium TNFalpha + | 0.0 |
| | | IL1beta | |
| Primary Th2 rest | 0.4 | Small airway epithelium none | 0.0 |
| Primary Tr1 rest | 0.0 | Small airway epithelium | 0.2 |
| | | TNFalpha + IL-1beta | |
| CD45RA CD4 lymphocyte act | 0.0 | Coronery artery SMC rest | 0.0 |
| CD45RO CD4 lymphocyte act | 0.0 | Coronery artery SMC TNFalpha + | 0.0 |
| | | IL-1beta | |
| CD8 lymphocyte act | 0.9 | Astrocytes rest | 0.0 |
| Secondary CD8 lymphocyte rest | 0.0 | Astrocytes TNFalpha + IL-1beta | 1.5 |
| Secondary CD8 lymphocyte act | 0.0 | KU-812 (Basophil) rest | 0.0 |
| CD4 lymphocyte none | 0.0 | KU-812 (Basophil) | 7.8 |
| | | PMA/ionomycin | |
| 2ry Th1/Th2/Tr1_anti-CD95 CH11 | 0.0 | CCD1106 (Keratinocytes) none | 0.0 |
| LAK cells rest | 0.0 | CCD1106 (Keratinocytes) | 0.0 |
| | | TNFalpha + IL-1beta | |
| LAK cells IL-2 | 0.0 | Liver cirrhosis | 2.5 |
| LAK cells IL-2 + IL-12 | 0.0 | Lupus kidney | 0.0 |
| LAK cells IL-2 + IFN gamma | 1.0 | NCI-H292 none | 0.0 |
| LAK cells IL-2 + IL-18 | 0.0 | NCI-H292 IL-4 | 0.0 |
| LAK cells PMA/ionomycin | 0.0 | NCI-H292 IL-9 | 0.0 |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.8 | HPAEC none | 0.6 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 0.0 | Lung fibroblast TNF alpha + IL-1 | 0.9 |
| | | beta | |
| PBMC PHA-L | 0.0 | Lung fibroblast IL-4 | 0.0 |
| Ramos (B cell) none | 9.8 | Lung fibroblast IL-9 | 0.0 |
| Ramos (B cell) ionomycin | 100.0 | Lung fibroblast IL-13 | 0.0 |
| B lymphocytes PWM | 0.0 | Lung fibroblast IFN gamma | 0.0 |
| B lymphocytes CD40L and IL-4 | 0.0 | Dermal fibroblast CCD1070 rest | 0.0 |
| EOL-1 dbcAMP | 0.0 | Dermal fibroblast CCD1070 TNF | 0.0 |
| | | alpha | |
| EOL-1 dbcAMP PMA/ionomycin | 0.0 | Dermal fibroblast CCD1070 IL-1 | 0.0 |
| | | beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 1.3 | IBD Colitis 2 | 0.5 |
| Monocytes rest | 0.0 | IBD Crohn's | 0.0 |
| Monocytes LPS | 0.0 | Colon | 0.0 |
| Macrophages rest | 0.0 | Lung | 1.4 |
| Macrophages LPS | 0.0 | Thymus | 6.7 |
| HUVEC none | 0.0 | Kidney | 3.1 |
| HUVEC starved | 0.0 | | |

Panel 1.3D Summary: Ag2854 Highest expression of the CG55926-01 gene is seen in a colon cancer cell line (CT=30). There is also significant expression in ovarian, breast, lung, melanoma and liver cancer cell lines. In general, expression of this gene is associated with cancer cell lines. Thus, expression of this gene could be used to differentiate between these cell lines and other samples on this panel. Furthermore, expression of this gene could potentially be used to detect the presence of these cancers.

Panel 2.2 Summary: Ag2854 Data from this experiment with the of CG55926-01 gene is not included. A bad amp plot indicates that there were experimental difficulties with this run.

Panel 4D Summary: Ag2854 Highest expression of the CG55926-01 gene is seen in Ramos B cells stimulated with ionomycin (CT=29.7). Lower but still significant levels of expression are seen in untreated Ramos B cells (CT=34.16). B cells represent a principle component of immunity and contribute to the immune response in a number of important functional roles, including antibody production. Production of antibodies against self-antigens is a major component in autoimmune disorders. Since B cells play an important role in inflammatory processes and inflammatory cascades, therapeutic modulation of this gene product may reduce or eliminate the symptoms of patients suffering from asthma, allergies, chronic obstructive pulmonary disease, emphysema, Crohn's disease, ulcerative colitis, rheumatoid arthritis, psoriasis, osteoarthritis, and other autoimmune disorders.

This gene is also expressed at a low but significant levels in KU-812 basophil cells treated with PMA/ionomycin (CT=33.4). These cells are a reasonable model for the inflammatory cells that take part in various inflammatory lung and bowel diseases, such as asthma, Crohn's disease, and ulcerative colitis. Therefore, therapeutics that modulate the function of this gene product may reduce or eliminate the symptoms of patients suffering from asthma, Crohn's disease, and ulcerative colitis.

This transcript is also expressed in the thymus. The putative GPCR encoded for this gene could therefore play an important role in T cell development. Small molecule therapeutics, or antibody therapeutics designed against the GPCR encoded for by this gene could be utilized to modulate immune function (T cell development) and be important for organ transplant, AIDS treatment or post chemotherapy immune reconstitiution.

F. CG55924-01: GPCR

Expression of gene CG55924-01 was assessed using the primer-probe set Ag2853, described in Table 18FA.

TABLE 18FA
Probe Name Ag2853

| | | | | Start |
|---|---|---|---|---|
| Primers | Sequences | | Length | Position |
| Forward | 5′-ctgtcctgttctgcagacataa-3′ | (SEQ ID NO:133) | 22 | 572 |
| Probe | TET-5′-tcatcacctatctcatttactccacattca-3′-TAMRA | (SEQ ID NO:134) | 30 | 594 |
| Reverse | 5′-aggtgccaatcatgaagataga-3′ | (SEQ ID NO:135) | 22 | 626 |

CNS_neurodegeneration_v1.0 Summary: Ag2853 Expression is low/undetectable in all samples in this panel (CT>35).

Panel 1.3D Summary: Ag2853 Expression is low/undetectable in all samples in this panel (CT>35).

Panel 2.2 Summary: Ag2853 Expression is low/undetectable in all samples in this panel (CT>35).

Panel 4D Summary: Ag2853 Expression is low/undetectable in all samples in this panel (CT>35).

G. CG55922-01: GPCR

Expression of gene CG55922-01 was assessed using the primer-probe set Ag2852, described in Table 18GA. Results of the RTQ-PCR runs are shown in Tables 18GB and 18GC.

TABLE 18GA
Probe Name Ag2852

| | | | | Start |
|---|---|---|---|---|
| Primers | Sequences | | Length | Position |
| Forward | 5′-catgtacatgttcctgggaaat-3′ | (SEQ ID NO:136) | 22 | 194 |
| Probe | TET-5′-tggtatgtctcttctacagttcccaagatg-3′-TAMRA | (SEQ ID NO:137) | 30 | 234 |
| Reverse | 5′-tctctgaaaggaagttgaccaa-3′ | (SEQ ID NO:138) | 22 | 264 |

TABLE 18GB
Panel 1.3D

| | Rel. Exp. (%) | | Rel. Exp. (%) |
|---|---|---|---|
| | Ag2852, Run | | Ag2852, Run |
| Tissue Name | 160657265 | Tissue Name | 160657265 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 7.6 |
| Adrenal gland | 0.0 | Renal ca. RXF 393 | 3.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 2.3 | Liver (fetal) | 0.0 |
| Brain (amygdala) | 0.0 | Liver ca. (hepatoblast) HepG2 | 0.0 |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) LX-1 | 0.0 |
| Brain (thalamus) | 3.4 | Lung ca. (small cell) NCI-H69 | 0.0 |
| Cerebral Cortex | 3.1 | Lung ca. (s. cell var.) SHP-77 | 0.0 |
| Spinal cord | 0.0 | Lung ca. (large cell)NCI-H460 | 0.0 |
| glio/astro U87-MG | 0.0 | Lung ca. (non-sm. cell) A549 | 100.0 |
| glio/astro U-118-MG | 9.0 | Lung ca. (non-s. cell) NCI-H23 | 10.8 |
| astrocytoma SW1783 | 0.0 | Lung ca. (non-s. cell) HOP-62 | 0.0 |
| neuro*; met SK-N-AS | 0.0 | Lung ca. (non-s. cl) NCI-H522 | 0.0 |
| astrocytoma SF-539 | 9.5 | Lung ca. (squam.) SW 900 | 0.0 |
| astrocytoma SNB-75 | 12.0 | Lung ca. (squam.) NCI-H596 | 0.0 |
| glioma SNB-19 | 11.1 | Mammary gland | 2.7 |
| glioma U251 | 3.2 | Breast ca* (pl. ef) MCF-7 | 0.0 |
| glioma SF-295 | 0.0 | Breast ca* (pl. ef) MDA-MB- | 0.0 |
| | | 231 | |
| Heart (fetal) | 0.0 | Breast ca.* (pl. ef) T47D | 0.0 |
| Heart | 0.0 | Breast ca. BT-549 | 18.7 |
| Skeletal muscle (fetal) | 15.0 | Breast ca. MDA-N | 17.3 |
| Skeletal muscle | 0.0 | Ovary | 0.0 |
| Bone marrow | 0.0 | Ovarian ca. OVCAR-3 | 4.7 |
| Thymus | 0.0 | Ovarian ca. OVCAR-4 | 0.0 |
| Spleen | 2.3 | Ovarian ca. OVCAR-5 | 4.5 |
| Lymph node | 0.0 | Ovarian ca. OVCAR-8 | 0.0 |
| Colorectal | 26.2 | Ovarian ca. IGROV-1 | 0.0 |
| Stomach | 2.7 | Ovarian ca.* (ascites) SK-OV-3 | 0.0 |
| Small intestine | 0.0 | Uterus | 0.0 |
| Colon ca. SW480 | 0.0 | Plancenta | 0.0 |
| Colon ca.* SW620(SW480 met) | 0.0 | Prostate | 0.0 |
| Colon ca. HT29 | 0.0 | Prostate ca.* (bone met)PC-3 | 5.7 |
| Colon ca.HCT-1 16 | 0.0 | Testis | 99.3 |
| Colon ca. CaCo-2 | 0.0 | Melanoma Hs688(A).T | 0.0 |
| Colon ca. tissue(ODO3866) | 0.0 | Melanoma* (met) Hs688(B).T | 0.0 |
| Colon ca. HCC-2998 | 8.7 | Melanoma UACC-62 | 0.0 |
| Gastric ca.* (liver met) NCI-N87 | 0.0 | Melanoma M14 | 2.9 |
| Bladder | 2.6 | Melanoma LOX IMVI | 0.0 |
| Trachea | 0.0 | Melanoma* (met) SK-MEL-5 | 0.0 |
| Kidney | 0.0 | Adipose | 0.0 |

TABLE 18GC
Panel 4D

| | Rel. Exp. (%) | | Rel. Exp. (%) |
|---|---|---|---|
| | Ag2852, Run | | Ag2852, Run |
| Tissue Name | 160658369 | Tissue Name | 160658369 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNFalpha + IL-1beta | |
| Primary Th2 act | 2.0 | Microvascular Dermal EC none | 0.0 |
| Primary Tr1 act | 0.0 | Microsvasular Dermal EC | 0.0 |
| | | TNFalpha + IL-1beta | |
| Primary Th1 rest | 0.0 | Bronchial epithelium TNFalpha + | 0.0 |
| | | IL1beta | |
| Primary Th2 rest | 0.0 | Small airway epithelium none | 0.0 |
| Primary Tr1 rest | 0.0 | Small airway epithelium | 0.0 |
| | | TNFalpha + IL-1beta | |
| CD45RA CD4 lymphocyte act | 0.0 | Coronery artery SMC rest | 0.0 |
| CD45RO CD4 lymphocyte act | 0.0 | Coronery artery SMC TNFalpha + | 0.0 |
| | | IL-1beta | |
| CD8 lymphocyte act | 0.0 | Astrocytes rest | 0.0 |
| Secondary CD8 lymphocyte rest | 0.0 | Astrocytes TNFalpha + IL-1beta | 0.0 |
| Secondary CD8 lymphocyte act | 0.0 | KU-812 (Basophil) rest | 0.0 |
| CD4 lymphocyte none | 0.0 | KU-812 (Basophil) | 0.0 |
| | | PMA/ionomycin | |
| 2ry Th1/Th2/Tr1_anti-CD95 CH11 | 0.0 | CCD1106 (Keratinocytes) none | 0.0 |
| LAK cells rest | 0.0 | CCD1106 (Keratinocytes) | 0.0 |
| | | TNFalpha + IL-1beta | |
| LAK cells IL-2 | 0.0 | Liver cirrhosis | 12.7 |
| LAK cells IL-2 + IL-12 | 0.0 | Lupus kidney | 0.0 |
| LAK cells IL-2 + IFN gamma | 0.0 | NCI-H292 none | 79.0 |
| LAK cells IL-2 + IL-18 | 0.0 | NCI-H292 IL-4 | 67.8 |
| LAK cells PMA/ionomycin | 0.0 | NCI-H292 IL-9 | 100.0 |
| NK Cells IL-2 rest | 0.0 | NCI-H292 IL-13 | 27.5 |
| Two Way MLR 3 day | 0.0 | NCI-H292 IFN gamma | 26.4 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 0.0 | Lung fibroblast TNF alpha + IL- | 0.0 |
| | | 1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and IL-4 | 0.0 | Dermal fibroblast CCD1070 rest | 0.0 |
| EOL-1 dbcAMP | 0.0 | Dermal fibroblast CCD1070 | 0.0 |
| | | TNF alpha | |
| EOL-1 dbcAMP PMA/ionomycin | 0.0 | Dermal fibroblast CCD1070 IL- | 0.0 |
| | | 1 beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 7.1 |
| Monocytes rest | 2.7 | IBD Crohn's | 0.0 |
| Monocytes LPS | 0.0 | Colon | 1.0 |
| Macrophages rest | 0.0 | Lung | 1.3 |
| Macrophages LPS | 0.0 | Thymus | 1.2 |
| HUVEC none | 0.0 | Kidney | 0.8 |
| HUVEC starved | 2.6 | | |

Panel 1.3D Summary: Ag2852 Significant expression of of the CG55922-01 gene, a GPCR homolog, is restricted to a lung cancer cell line and the testis. Thus, expression of this gene could be used to differentiate between these samples and other samples on this panel. In addition, expression of this gene could potentially be used as a marker for the presence of lung cancer. The high levels of expression in the testis also suggest that the gene product may be involved in the normal function of this organ. Thus, therapeutic modulation of the expression or function of the protein encoded by this gene may be useful in the treatment of infertility. Please note that a second run, with the same probe and primer set showed low/undetectable levels of expression.

Panel 2D Summary: Ag2852 Expression of the CG55922-01 gene is low/undetected (CTs>34.5) in all samples in this panel. (Data not shown.)

Panel 3D Summary: Ag2852 Expression of the CG55922-01 gene is low/undetected (CTs>34.5) in all samples in this panel. (Data not shown.)

Panel 4D Summary: Ag2852 The CG55922-01 gene is most highly expressed in the mucoepidermoid cell line (NCI-H292) stimulated by IL-9 (CT=32.8). The gene is also expressed in a cluster of treated and untreated NCI-H292 mucoepidermoid cell line samples. This cell line is often used as a model for airway epithelium and this highly specific pattern of expression suggests that this transcript may be important in the proliferation or activation of airway epithelium. Therefore, therapuetics designed with the GPCR encoded for by the transcript could be important in the treatment of diseases which include lung airway inflammation such as asthma and chronic obstructive pulmonary disease.

H. CG55728-01: GPCR

Expression of gene CG55728-01 was assessed using the primer-probe set Ag2586, described in Table 18HA. Results of the RTQ-PCR runs are shown in Table 18HB.

TABLE 18HA
Probe Name Ag2586

| Primers | Sequences | | Length | Start Position |
|---|---|---|---|---|
| Forward | 5′-agaggaaaggctaaagccttct-3′ | (SEQ ID NO:139) | 22 | 712 |
| Probe | TET-5′-ccacctgctcatctcacctaatagttg-3′-TAMRA | (SEQ ID NO:140) | 27 | 734 |
| Reverse | 5′-tggtttgggctgtaaataagtg-3′ | (SEQ ID NO:141) | 22 | 789 |

TABLE 18HB
Panel 5D

| | Rel. Exp. (%) | | Rel. Exp. (%) |
|---|---|---|---|
| | Ag2586, Run | | Ag2586, Run |
| Tissue Name | 169269909 | Tissue Name | 169269909 |
| 97457_Patient-02go_adipose | 0.0 | 94709_Donor 2 AM - A_adipose | 9.9 |
| 97476_Patient-07sk_skeletal muscle | 4.4 | 94710_Donor 2 AM - B_adipose | 0.0 |
| 97477_Patient-07ut_uterus | 0.0 | 94711_Donor 2 AM - C_adipose | 4.7 |
| 97478_Patient-07pl_placenta | 9.7 | 94712_Donor 2 AD - A_adipose | 0.0 |
| 97481_Patient-08sk_skeletal muscle | 0.0 | 94713_Donor 2 AD - B_adipose | 0.0 |
| 97482_Patient-08ut_uterus | 0.0 | 94714_Donor 2 AD - C_adipose | 0.0 |
| 97483_Patient-08pl_placenta | 8.1 | 94742_Donor 3 U- | 0.0 |
| | | A_Mesenchymal Stem Cells | |
| 97486_Patient-09sk_skeletal muscle | 0.0 | 94743_Donor 3 U- | 0.0 |
| | | B_Mesenchymal Stem Cells | |
| 97487_Patient-09ut_uterus | 3.0 | 94730_Donor 3 AM - A_adipose | 0.0 |
| 97488_Patient-09pl_placenta | 0.0 | 94731_Donor 3 AM - B_adipose | 0.0 |
| 97492_Patient-10ut_uterus | 10.1 | 94732_Donor 3 AM - C_adipose | 0.0 |
| 97493_Patient-10pl_placenta | 0.0 | 94733_Donor 3 AD - A_adipose | 0.0 |
| 97495_Patient-11go_adipose | 0.0 | 94734_Donor 3 AD - B_adipose | 0.0 |
| 97496_Patient-11sk_skeletal muscle | 0.0 | 94735_Donor 3 AD - C_adipose | 0.0 |
| 97497_Patient-11ut_uterus | 0.0 | 77138_Liver_HepG2untreated | 0.0 |
| 97498_Patient-11pl_placenta | 0.0 | 73556_Heart_Cardiac stromal | 0.0 |
| | | cells (primary) | |
| 97500_Patient-12go_adipose | 0.0 | 81735_Small Intestine | 0.0 |
| 97501_Patient-12sk_skeletal muscle | 0.0 | 72409_Kidney_Proximal | 0.0 |
| | | Convoluted Tubule | |
| 97502_Patient-12ut_uterus | 4.7 | 82685_Small intestine_Duodenum | 0.0 |
| 97503_Patient-12pl_placenta | 0.0 | 90650_Adrenal_Adrenocortical | 0.0 |
| | | adenoma | |
| 94721_Donor 2 U - A_Mesenchymal | 0.0 | 72410_Kidney_HRCE | 0.0 |
| Stem Cells | | | |
| 94722_Donor 2 U - B_Mesenchymal | 100.0 | 72411_Kidney_HRE | 0.0 |
| Stem Cells | | | |
| 94723_Donor 2 U - C_Mesenchymal | 5.8 | 73139_Uterus_Uterine smooth | 0.0 |
| Stem Cells | | muscle cells | |

CNS_neurodegeneration_v1.0 Summary: Ag2586 Expression of the CG55728-01 gene is low/undetectable in all samples in this panel (CTs>35). (Data not shown.)

Panel 1.3D Summary: Ag2586 Expression of the CG55728-01 gene is low/undetectable in all samples in this panel (CTs>35). (Data not shown.)

Panel 2.2 Summary: Ag2586 Expression of the CG55728-01 gene is low/undetectable in all samples in this panel (CTs>35). (Data not shown.)

Panel 4D Summary: Ag2586 Expression of the CG55728-01 gene is low/undetectable in all samples in this panel (CTs>35). (Data not shown.)

Panel 5D Summary: Ag2586Expression of the CG55728-01 gene is restricted to a sample derived from mesenchymal stem cells (CT=33.9). These cells can be differentiated in vitro to adipocytes, chondrocytes and osteocytes. Thus, this gene product may be a small molecule drug target for the treatment of any disease involving adipose, cartilage and bone.

I. CG55726-01: GPCR

Expression of gene CG55726-01 was assessed using the primer-probe set Ag2585, described in Table 18IA. Results of the RTQ-PCR runs are shown in Table 18IB.

TABLE 18IA
Probe Name Ag2585

| Primers | Sequences | | Length | Start Position |
|---|---|---|---|---|
| Forward | 5′-agcttgaggagactgtcctttt-3′ | (SEQ ID NO:142) | 22 | 486 |
| Probe | TET-5′-aatgctgtcagccacttcttctgtga-3′-TAMRA | (SEQ ID NO:143) | 26 | 516 |
| Reverse | 5′-cagctttagcagtgaaggaatg-3′ | (SEQ ID NO:144) | 22 | 542 |

TABLE 18IB
Panel 2.2

| | Rel. Exp. (%) | | Rel. Exp. (%) |
|---|---|---|---|
| | Ag2585, Run | | Ag2585, Run |
| Tissue Name | 175141934 | Tissue Name | 175141934 |
| Normal Colon | 0.0 | Kidney Margin (OD04348) | 0.0 |
| Colon cancer (OD06064) | 0.0 | Kidney malignant cancer | 0.0 |
| | | (OD06204B) | |
| Colon Margin (OD06064) | 0.0 | Kidney normal adjacent tissue | 0.0 |
| | | (OD06204E) | |
| Colon cancer (OD06159) | 0.0 | Kidney Cancer (OD04450-01) | 0.0 |
| Colon Margin (OD06159) | 0.0 | Kidney Margin (OD04450-03) | 0.0 |
| Colon cancer (OD06297-04) | 0.0 | kidney Cancer 8120613 | 0.0 |
| Colon Margin (OD06297-015) | 0.0 | Kidney Margin 8120614 | 0.0 |
| CC Gr.2 ascend colon (ODO3921) | 0.0 | Kidney Cancer 9010320 | 3.9 |
| CC Margin (ODO3921) | 0.0 | Kidney Margin 9010321 | 0.0 |
| Colon cancer metastasis (OD06104) | 0.0 | Kidney Cancer 8120607 | 0.0 |
| Lung Margin (OD06104) | 0.0 | Kidney Margin 8120608 | 0.0 |
| Colon mets to lung (OD04451-01) | 0.0 | Normal Uterus | 0.0 |
| Lung Margin (OD04451-02) | 0.0 | Uterine Cancer 064011 | 66.9 |
| Normal Prostate | 0.0 | Normal Thyroid | 0.0 |
| Prostate Cancer (OD04410) | 0.0 | Thyroid Cancer 064010 | 0.0 |
| Prostate Margin (OD04410) | 0.0 | Thyroid Cancer A302152 | 0.0 |
| Normal Ovary | 0.0 | Thyroid Margin A302153 | 0.0 |
| Ovarian cancer (OD06283-03) | 0.0 | Normal Breast | 0.0 |
| Ovarian Margin (OD06283-07) | 0.0 | Breast Cancer (OD04566) | 0.0 |
| Ovarian Cancer 064008 | 0.0 | Breast Cancer 1024 | 0.0 |
| Ovarian cancer (OD06145) | 0.0 | Breast Cancer (OD04590-01) | 0.0 |
| Ovarian Margin (OD06145) | 0.0 | Breast Cancer Mets (OD04590-03) | 0.0 |
| Ovarian cancer (OD06455-03) | 0.0 | Breast Cancer Metastasis | 0.0 |
| | | (OD04655-05) | |
| Ovarian Margin (OD06455-07) | 0.0 | Breast Cancer 064006 | 0.0 |
| Normal Lung | 0.0 | Breast Cancer 9100266 | 0.0 |
| Invasive poor diff. lung adeno | 0.0 | Breast Margin 9100265 | 0.0 |
| (ODO4945-01 | | | |
| Lung Margin (ODO4945-03) | 0.0 | Breast Cancer A209073 | 0.0 |
| Lung Malignant Cancer (OD03126) | 0.0 | Breast Margin A2090734 | 0.0 |
| Lung Margin (OD03126) | 0.0 | Breast cancer (OD06083) | 0.0 |
| Lung Cancer (OD05014A) | 0.0 | Breast cancer node metastasis | 0.0 |
| | | (OD06083) | |
| Lung Margin (OD05014B) | 0.0 | Normal Liver | 0.0 |
| Lung cancer (OD06081) | 0.0 | Liver Cancer 1026 | 0.0 |
| Lung Margin (OD06081) | 0.0 | Liver Cancer 1025 | 4.8 |
| Lung Cancer (OD04237-01) | 0.0 | Liver Cancer 6004-T | 0.0 |
| Lung Margin (OD04237-02) | 0.0 | Liver Tissue 6004-N | 0.0 |
| Ocular Melanoma Metastasis | 0.0 | Liver Cancer 6005-T | 0.0 |
| Ocular Melanoma Margin (Liver) | 0.0 | Liver Tissue 6005-N | 0.0 |
| Melanoma Metastasis | 100.0 | Liver Cancer 064003 | 0.0 |
| Melanoma Margin (Lung) | 0.0 | Normal Bladder | 0.0 |
| Normal Kidney | 0.0 | Bladder Cancer 1023 | 0.0 |
| Kidney Ca, Nuclear grade 2 | 0.0 | Bladder Cancer A302173 | 0.0 |
| (OD04338) | | | |
| Kidney Margin (OD04338) | 0.0 | Normal Stomach | 0.0 |
| Kidney Ca Nuclear grade 1/2 | 0.0 | Gastric Cancer 9060397 | 0.0 |
| (OD04339) | | | |
| Kidney Margin (OD04339) | 0.0 | Stomach Margin 9060396 | 0.0 |
| Kidney Ca, Clear cell type | 0.0 | Gastric Cancer 9060395 | 0.0 |
| (OD04340) | | | |
| Kidney Margin (OD04340) | 0.0 | Stomach Margin 9060394 | 0.0 |
| Kidney Ca, Nuclear grade 3 | 0.0 | Gastric Cancer 064005 | 0.0 |
| (OD04348) | | | |

CNS_neurodegeneration_v1.0 Summary: Ag2585 Expression of gene CG55726-01 is low/undetectable in all samples in this panel (CTs>35). (Data not shown.)

Panel 1.3D Summary: Ag2585 Expression of gene CG55726-01 is low/undetectable in all samples in this panel (CTs>35). (Data not shown.)

Panel 2.2 Summary: Ag2585 Significant expression of gene CG55726-01, a GPCR homolog. is restricted to a melanoma metastasis and a uterine cancer (CTs=33). Thus, expression of this gene could be used to differentiate between these samples and other samples in this panel. Furthermore, expression of this gene could potentially be used as a marker to detect the presence of these cancers.

Panel 4D Summary: Ag2585 Expression of gene CG55726-01 is low/undetectable in all samples in this panel (CTs>35). (Data not shown.)

J. CG50325-01: GPCR

Expression of gene CG50325-01 was assessed using the primer-probe set Ag2564, described in Table 18JA. Results of the RTQ-PCR runs are shown in Table 18JB.

TABLE 18JA
Probe Name Ag2564

| Primers | Sequences | | Length | Start Position |
|---|---|---|---|---|
| Forward | 5′-agctcacctaactggagtgaca-3′ | (SEQ ID NO:145) | 22 | 743 |
| Probe | TET-5′-tcatgggacaatcctcttcatgtatg-3′-TAMRA | (SEQ ID NO:146) | 26 | 770 |
| Reverse | 5′-gtgtagctggaacttggtctca-3′ | (SEQ ID NO:147) | 22 | 796 |

TABLE 18JB
Panel 4D

| | Rel. Exp. (%) | | Rel. Exp. (%) |
|---|---|---|---|
| | Ag2564, Run | | Ag2564, Run |
| Tissue Name | 164184243 | Tissue Name | 164184243 |
| Secondary Th1 act | 0.0 | HUVEC IL-1beta | 0.0 |
| Secondary Th2 act | 0.0 | HUVEC IFN gamma | 0.0 |
| Secondary Tr1 act | 4.5 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + | 33.9 |
| | | IL-1beta | |
| Primary Th2 act | 0.0 | Microvascular Dermal EC none | 0.0 |
| Primary Tr1 act | 0.0 | Microsvasular Dermal EC | 17.0 |
| | | TNFalpha + IL-1beta | |
| Primary Th1 rest | 0.0 | Bronchial epithelium TNFalpha + | 0.0 |
| | | IL1beta | |
| Primary Th2 rest | 3.4 | Small airway epithelium none | 0.0 |
| Primary Tr1 rest | 0.0 | Small airway epithelium TNFalpha + | 0.0 |
| | | IL-1beta | |
| CD45RA CD4 lymphocyte act | 0.0 | Coronery artery SMC rest | 0.0 |
| CD45RO CD4 lymphocyte act | 0.0 | Coronery artery SMC TNFalpha + | 7.6 |
| | | IL-1beta | |
| CD8 lymphocyte act | 0.0 | Astrocytes rest | 0.0 |
| Secondary CD8 lymphocyte rest | 24.8 | Astrocytes TNFalpha + IL-1beta | 0.0 |
| Secondary CD8 lymphocyte act | 0.0 | KU-812 (Basophil) rest | 0.0 |
| CD4 lymphocyte none | 0.0 | KU-812 (Basophil) | 0.0 |
| | | PMA/ionomycin | |
| 2ry Th1/Th2/Tr1_anti-CD95 CH11 | 0.0 | CCD1106 (Keratinocytes) none | 0.0 |
| LAK cells rest | 0.0 | CCD1106 (Keratinocytes) | 2.6 |
| | | TNFalpha + IL-1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN gamma | 0.0 | NCI-H292 none | 0.0 |
| LAK cells IL-2 + IL-18 | 0.0 | NCI-H292 IL-4 | 7.9 |
| LAK cells PMA/ionomycin | 0.0 | NCI-H292 IL-9 | 3.1 |
| NK Cells IL-2 rest | 0.0 | NCI-H292 IL-13 | 0.0 |
| Two Way MLR 3 day | 0.0 | NCI-H292 IFN gamma | 3.4 |
| Two Way MLR 5 day | 0.0 | HPAEC none | 0.0 |
| Two Way MLR 7 day | 0.0 | HPAEC TNF alpha + IL-1 beta | 8.8 |
| PBMC rest | 0.0 | Lung fibroblast none | 0.0 |
| PBMC PWM | 0.0 | Lung fibroblast TNF alpha + IL-1 | 0.0 |
| | | beta | |
| PBMC PHA-L | 2.4 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and IL-4 | 0.0 | Dermal fibroblast CCD1070 rest | 0.0 |
| EOL-1 dbcAMP | 0.0 | Dermal fibroblast CCD1070 TNF | 0.0 |
| | | alpha | |
| EOL-1 dbcAMP PMA/ionomycin | 0.0 | Dermal fibroblast CCD1070 IL-1 | 0.0 |
| | | beta | |
| Dendritic cells none | 4.3 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 7.3 |
| Monocytes rest | 0.0 | IBD Crohn's | 0.0 |
| Monocytes LPS | 0.0 | Colon | 0.0 |
| Macrophages rest | 0.0 | Lung | 5.9 |
| Macrophages LPS | 0.0 | Thymus | 0.0 |
| HUVEC none | 21.2 | Kidney | 0.0 |
| HUVEC starved | 0.0 | | |

CNS_neurodegeneration_v1.0 Summary: Ag2564 Expression of gene CG50325-01 is low/undetectable in all samples in this panel (CTs>35). (Data not shown.)

Panel 1.3D Summary: Ag2564 Expression of gene CG50325-01 is low/undetectable in all samples in this panel (CTs>35). (Data not shown.)

Panel 2.2 Summary: Ag2564 Expression of gene CG50325-01 is low/undetectable in all samples in this panel (CTs>35). (Data not shown.)

Panel 4D Summary: Ag2564 The CG50325-01 gene transcript is only detected in liver cirrhosis. Furthermore, this transcript is not detected in normal liver in Panel 1.3D, suggesting that this gene expression is unique to liver cirrhosis. This gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis.

K. CG50285-01: GPCR

Expression of gene CG50285-01 was assessed using the primer-probe set Ag2539, described in Table 18KA. Results of the RTQ-PCR runs are shown in Tables 18KB and 18KC.

TABLE 18KA
Probe Name Ag2539

| Primers | Sequences | | Length | Start Position |
|---|---|---|---|---|
| Forward | 5′-cacctccattcccctatgtact-3′ | (SEQ ID NO:148) | 22 | 173 |
| Probe | TET-5′-tccttagtaacttggcctttgttgaca-3′-TAMRA | (SEQ ID NO:149) | 27 | 198 |
| Reverse | 5′-ggactgtagtcgacgtaaagca-3′ | (SEQ ID NO:150) | 22 | 227 |

TABLE 18KB
Panel 1.3D

| | Rel. Exp. (%) | | Rel. Exp. (%) |
|---|---|---|---|
| | Ag2539, Run | | Ag2539, Run |
| Tissue Name | 166177198 | Tissue Name | 166177198 |
| Liver adenocarcinoma | 0.0 | Kidney (fetal) | 0.0 |
| Pancreas | 13.6 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 11.9 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 7.6 | Liver | 0.0 |
| Brain (whole) | 0.0 | Liver (fetal) | 0.0 |
| Brain (amygdala) | 0.0 | Liver ca. (hepatoblast) HepG2 | 0.0 |
| Brain (cerebellum) | 0.0 | Lung | 0.0 |
| Brain (hippocampus) | 13.7 | Lung (fetal) | 0.0 |
| Brain (substantia nigra) | 0.0 | Lung ca. (small cell) LX-1 | 0.0 |
| Brain (thalamus) | 0.0 | Lung ca. (small cell) NCI-H69 | 0.0 |
| Cerebral Cortex | 0.0 | Lung ca. (s. cell var.) SHP-77 | 0.0 |
| Spinal cord | 0.0 | Lung ca. (large cell)NCI-H460 | 0.0 |
| glio/astro U87-MG | 0.0 | Lung ca. (non-sm. cell) A549 | 0.0 |
| glio/astro U-118-MG | 0.0 | Lung ca. (non-s. cell) NCI-H23 | 7.0 |
| astrocytoma SW1783 | 0.0 | Lung ca. (non-s. cell) HOP-62 | 0.0 |
| neuro*; met SK-N-AS | 0.0 | Lung ca. (non-s. cl) NCI-H522 | 0.0 |
| astrocytoma SF-539 | 0.0 | Lung ca. (squam.) SW 900 | 0.0 |
| astrocytoma SNB-75 | 0.0 | Lung ca. (squam.) NCI-H596 | 0.0 |
| glioma SNB-19 | 0.0 | Mammary gland | 0.0 |
| glioma U251 | 0.0 | Breast ca.* (pl. ef) MCF-7 | 0.0 |
| glioma SF-295 | 0.0 | Breast ca.* (pl. ef) MDA-MB-231 | 0.0 |
| Heart (fetal) | 0.0 | Breast ca.* (pl. ef) T47D | 0.0 |
| Heart | 0.0 | Breast ca. BT-549 | 0.0 |
| Skeletal muscle (fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 11.4 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 28.3 |
| Colorectal | 21.2 | Ovarian ca. IGROV-1 | 0.0 |
| Stomach | 0.0 | Ovarian ca.* (ascites) SK-OV-3 | 7.0 |
| Small intestine | 0.0 | Uterus | 0.0 |
| Colon ca. SW480 | 0.0 | Plancenta | 0.0 |
| Colon ca.* SW620(SW480 met) | 0.0 | Prostate | 0.0 |
| Colon ca. HT29 | 0.0 | Prostate ca.* (bone met)PC-3 | 12.2 |
| Colon ca. HCT-116 | 0.0 | Testis | 100.0 |
| Colon ca. CaCo-2 | 0.0 | Melanoma Hs688(A).T | 0.0 |
| Colon ca. tissue(ODO3866) | 0.0 | Melanoma* (met) Hs688(B).T | 0.0 |
| Colon ca. HCC-2998 | 0.0 | Melanoma UACC-62 | 0.0 |
| Gastric ca.* (liver met) NCI-N87 | 0.0 | Melanoma M14 | 0.0 |
| Bladder | 0.0 | Melanoma LOX IMVI | 0.0 |
| Trachea | 0.0 | Melanoma* (met) SK-MEL-5 | 2.1 |
| Kidney | 11.4 | Adipose | 0.0 |

TABLE 18KC
Panel 4D

| | Rel. Exp. (%) | | Rel. Exp. (%) |
|---|---|---|---|
| | Ag2539, Run | | Ag2539, Run |
| Tissue Name | 164295847 | Tissue Name | 164295847 |
| Secondary Th1 act | 0.0 | HUVEC IL-1beta | 0.0 |
| Secondary Th2 act | 0.0 | HUVEC IFN gamma | 0.0 |
| Secondary Tr1 act | 3.3 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + | 0.0 |
| | | IL-1beta | |
| Primary Th2 act | 0.0 | Microvascular Dermal EC none | 0.0 |
| Primary Tr1 act | 0.0 | Microsvasular Dermal EC TNFalpha + | 0.0 |
| | | IL-1beta | |
| Primary Th1 rest | 0.0 | Bronchial epithelium TNFalpha + | 0.0 |
| | | IL1beta | |
| Primary Th2 rest | 0.0 | Small airway epithelium none | 0.0 |
| Primary Tr1 rest | 0.0 | Small airway epithelium TNFalpha + | 0.0 |
| | | IL-1beta | |
| CD45RA CD4 lymphocyte act | 0.0 | Coronery artery SMC rest | 0.0 |
| CD45RO CD4 lymphocyte act | 0.0 | Coronery artery SMC TNFalpha + IL- | 3.0 |
| | | 1beta | |
| CD8 lymphocyte act | 0.0 | Astrocytes rest | 0.0 |
| Secondary CD8 lymphocyte rest | 0.0 | Astrocytes TNFalpha + IL-1beta | 0.0 |
| Secondary CD8 lymphocyte act | 0.0 | KU-812 (Basophil) rest | 32.5 |
| CD4 lymphocyte none | 0.0 | KU-812 (Basophil) PMA/ionomycin | 100.0 |
| 2ry Th1/Th2/Tr1_anti-CD95 | 0.0 | CCD1106 (Keratinocytes) none | 0.0 |
| CH11 | | | |
| LAK cells rest | 0.0 | CCD1106 (Keratinocytes) TNFalpha + | 0.0 |
| | | IL-1beta | |
| LAK cells IL-2 | 0.0 | Liver cirrhosis | 16.0 |
| LAK cells IL-2 + IL-12 | 2.0 | Lupus kidney | 0.0 |
| LAK cells IL-2 + IFN gamma | 0.0 | NCI-H292 none | 0.0 |
| LAK cells IL-2 + IL-18 | 0.0 | NCI-H292 IL-4 | 0.0 |
| LAK cells PMA/ionomycin | 2.9 | NCI-H292 IL-9 | 0.0 |
| NK Cells IL-2 rest | 0.0 | NCI-H292 IL-13 | 0.0 |
| Two Way MLR 3 day | 4.1 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 0.0 | Lung fibroblast TNF alpha + IL-1 | 0.0 |
| | | beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and IL-4 | 0.0 | Dermal fibroblast CCD1070 rest | 0.0 |
| EOL-1 dbcAMP | 0.0 | Dermal fibroblast CCD1070 TNF | 0.0 |
| | | alpha | |
| EOL-1 dbcAMP PMA/ionomycin | 0.0 | Dermal fibroblast CCD1070 IL-1 beta | 0.0 |
| Dendritic cells none | 0.0 | Dermal fibroblast IFN gamma | 0.0 |
| Dendritic cells LPS | 2.3 | Dermal fibroblast IL-4 | 0.7 |
| Dendritic cells anti-CD40 | 5.1 | IBD Colitis 2 | 15.4 |
| Monocytes rest | 0.0 | IBD Crohn's | 0.0 |
| Monocytes LPS | 0.0 | Colon | 0.0 |
| Macrophages rest | 3.3 | Lung | 2.6 |
| Macrophages LPS | 0.0 | Thymus | 12.2 |
| HUVEC none | 0.0 | Kidney | 0.0 |
| HUVEC starved | 0.0 | | |

CNS_neurodegeneration_v1.0 Summary: Ag2539 Expression of the CG50285-01 gene is low/undetected (CT>34.5) for all the samples in this panel (Data not shown.)

Panel 1.3D Summary: Ag2539 Expression of the CG50285-01 gene is restricted to the testis (CT=34.2). Thus, expression of this gene could be used as a marker for testis tissue. The expression of the gene at significant levels in testis only suggests that the CG50285-01 gene product may be involved in fertility. Therefore, therapeutic modulation of the function or expression of the protein encoded by the CG50285-01 gene may be useful in treating disease states where fertility is compromised.

Panel 2.2 Summary: Ag2539 Expression of the CG50285-01 gene is low/undetected (CT>35) for all the samples in this panel (Data not shown.)

Panel 4D Summary: Ag2539 The CG50285-01 transcript is expressed in the PMA and ionomycin treated basophil cell line KU-812 and to a lesser extent in untreated KU-812 cells. This gene encodes a putative GPCR and it is known that GPCR-type receptors are important in multiple physiological responses mediated by basophils (ref. 1). Therefore, antibody or small molecule therapies designed with the protein encoded for by this gene could block or inhibit inflammation or tissue damage due to basophil activation in response to asthma, allergies, hypersensitivity reactions, psoriasis, and viral infections.

References:

1. Heinemann A., Hartnell A., Stubbs V. E., Murakami K., Soler D., LaRosa G., Askenase P. W., Williams T. J., Sabroe 1. (2000) Basophil responses to chemokines are regulated by both sequential and cooperative receptor signaling. J. Immunol. 165: 7224-7233.

To investigate human basophil responses to chemokines, we have developed a sensitive assay that uses flow cytometry to measure leukocyte shape change as a marker of cell responsiveness. PBMC were isolated from the blood of volunteers. Basophils were identified as a single population of cells that stained positive for IL-3Ralpha (CDw123) and negative for HLA-DR, and their increase in forward scatter (as a result of cell shape change) in response to chemokines was measured. Shape change responses of basophils to chemokines were highly reproducible, with a rank order of potency: monocyte chemoattractant protein (MCP) 4 (peak at /=eotaxin-2=eotaxin-3>/=eotaxin>MCP-1=MCP-3>macrophage-inflammatory protein-1alpha>RANTES=MCP-2=IL-8. The CCR4-selective ligand macrophage-derived chemokine did not elicit a response at concentrations up to 10 nM. Blocking mAbs to CCR2 and CCR3 demonstrated that responses to higher concentrations (>10 nM) of MCP-1 were mediated by CCR3 rather than CCR2, whereas MCP-4 exhibited a biphasic response consistent with sequential activation of CCR3 at lower concentrations and CCR2 at 10 nM MCP-4 and above. In contrast, responses to MCP-3 were blocked only in the presence of both mAbs, but not after pretreatment with either anti-CCR2 or anti-CCR3 mAb alone. These patterns of receptor usage were different from those seen for eosinophils and monocytes. We suggest that cooperation between CCRs might be a mechanism for preferential recruitment of basophils, as occurs in tissue hypersensitivity responses in vivo.

PMID: 11120855

L. CG55995-01: GPCR

Expression of gene CG55995-01 was assessed using the primer-probe set Ag2181, described in Table 18LA. Results of the RTQ-PCR runs are shown in Table LB.

TABLE 18LA
Probe Name Ag2181

| Primers | Sequences | | Length | Start Position |
|---|---|---|---|---|
| Forward | 5′-ctatggcacagccaatatgact-3′ | (SEQ ID NO:151) | 22 | 636 |
| Probe | TET-5′-aacccaaatctggctactcacccgaa-3′-TAMRA | (SEQ ID NO:152) | 26 | 665 |
| Reverse | 5′-ccaatgagatcagtttcttggt-3′ | (SEQ ID NO:153) | 22 | 691 |

TABLE 18LB
Panel 41D

| | Rel. Exp. (%) | | Rel. Exp. (%) |
|---|---|---|---|
| | Ag2181, Run | | Ag2181, Run |
| Tissue Name | 163578426 | Tissue Name | 163578426 |
| Secondary Th1 act | 0.0 | HUVEC IL-1beta | 2.3 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + | 0.0 |
| | | IL-1beta | |
| Primary Th2 act | 0.0 | Microvascular Dermal EC none | 0.0 |
| Primary Tr1 act | 0.0 | Microsvasular Dermal EC TNFalpha + | 0.0 |
| | | IL-1beta | |
| Primary Th1 rest | 0.0 | Bronchial epithelium TNFalpha + | 5.4 |
| | | IL1beta | |
| Primary Th2 rest | 0.0 | Small airway epithelium none | 2.4 |
| Primary Tr1 rest | 0.0 | Small airway epithelium TNFalpha + | 88.9 |
| | | IL-1beta | |
| CD45RA CD4 lymphocyte act | 0.0 | Coronery artery SMC rest | 0.0 |
| CD45RO CD4 lymphocyte act | 0.0 | Coronery artery SMC TNFalpha + IL- | 3.4 |
| | | 1beta | |
| CD8 lymphocyte act | 0.0 | Astrocytes rest | 0.0 |
| Secondary CD8 lymphocyte rest | 0.0 | Astrocytes TNFalpha + IL-1beta | 6.7 |
| Secondary CD8 lymphocyte act | 0.0 | KU-812 (Basophil) rest | 0.0 |
| CD4 lymphocyte none | 0.0 | KU-812 (Basophil) PMA/ionomycin | 0.0 |
| 2ry Th1/Th2/Tr1_anti-CD95 CH11 | 0.0 | CCD1106 (Keratinocytes) none | 5.5 |
| LAK cells rest | 0.0 | CCD1106 (Keratinocytes) TNFalpha + | 0.0 |
| | | IL-1beta | |
| LAK cells IL-2 | 0.0 | Liver cirrhosis | 15.4 |
| LAK cells IL-2 + IL-12 | 0.0 | Lupus kidney | 3.8 |
| LAK cells IL-2 + IFN gamma | 0.0 | NCI-H292 none | 84.7 |
| LAK cells IL-2 + IL-18 | 0.0 | NCI-H292 IL-4 | 100.0 |
| LAK cells PMA/ionomycin | 0.0 | NCI-H292 IL-9 | 75.8 |
| NK Cells IL-2 rest | 0.0 | NCI-H292 IL-13 | 46.3 |
| Two Way MLR 3 day | 0.0 | NCI-H292 IFN gamma | 30.1 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 13.1 |
| PBMC PWM | 0.0 | Lung fibroblast TNF alpha + IL-1 | 3.0 |
| | | beta | |
| PBMC PHA-L | 0.0 | Lung fibroblast IL-4 | 18.0 |
| Ramos (B cell) none | 0.0 | Lung fibroblast IL-9 | 6.5 |
| Ramos (B cell) ionomycin | 0.0 | Lung fibroblast IL-13 | 10.1 |
| B lymphocytes PWM | 0.0 | Lung fibroblast IFN gamma | 11.8 |
| B lymphocytes CD40L and IL-4 | 0.0 | Dermal fibroblast CCD1070 rest | 3.3 |
| EOL-1 dbcAMP | 0.0 | Dermal fibroblast CCD1070 TNF | 0.0 |
| | | alpha | |
| EOL-1 dbcAMP PMA/ionomycin | 0.0 | Dermal fibroblast CCD1070 IL-1 beta | 0.0 |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 6.1 |
| Monocytes rest | 0.0 | IBD Crohn's | 0.0 |
| Monocytes LPS | 0.0 | Colon | 0.0 |
| Macrophages rest | 0.0 | Lung | 0.0 |
| Macrophages LPS | 0.0 | Thymus | 0.0 |
| HUVEC none | 0.0 | Kidney | 3.1 |
| HUVEC starved | 0.0 | | |

CNS_neurodegeneration_v1.0 Summary: Ag2l81 Expression of the CG55995-01 gene is low/undetectable in all samples in this panel (CTs>35). (Data not shown.)

Panel 1.3D Summary: Ag2181 Expression of the CG55995-01 gene is low/undetectable in all samples in this panel (CTs>35). (Data not shown.)

Panel 2D Summary: Ag2181 Expression of the CG55995-01 gene is low/undetectable in all samples in this panel (CTs>35). (Data not shown.)

Panel 4D Summary: Ag2181The CG55995-01 transcript is most highly expressed in NCI-H292 cells stimulated by IL-4 (CT=34.1). The gene is also expressed in a cluster of treated and untreated NCI-H292 mucoepidermoid cell line samples and in small airway epithelium treated with IL-1beta and TNFalpha. In comparison, expression in the normal lung is very low. The expression of the transcript in activated normal epithelium as well as a cell line that is often used as a model for airway epithelium (NCI-H292 cells) suggests that this transcript may be important in the proliferation or activation of airway epithelium. Therefore, therapeutics designed with the GPCR encoded by the transcript could be important in the treatment of diseases that include lung airway inflammation such as asthma and chronic obstructive pulmonary disorder.

M. CG50375-01: Olfactory Receptor

Expression of gene CG50375-01 was assessed using the primer-probe sets Ag2576 and Ag2645, described in Tables 18MA and 18MB. Results of the RTQ-PCR runs are shown in Tables 18MC and 18MD.

TABLE 18MA
Probe Name Ag2576

| Primers | Sequences | | Length | Start Position |
|---|---|---|---|---|
| Forward | 5′-gggcatcttctggttcaatatc-3′ | (SEQ ID NO:154) | 22 | 257 |
| Probe | TET-5′-ccttttggaggctacctttctcagatg-3′-TAMRA | (SEQ ID NO:155) | 27 | 288 |
| Reverse | 5′-gctctccatgacagtgaagaaa-3′ | (SEQ ID NO:156) | 22 | 326 |

TABLE 18MB
Probe Name Ag2645

| Primers | Sequences | | Length | Start Position |
|---|---|---|---|---|
| Forward | 5′-gggcatcttctggttcaatatc-3′ | (SEQ ID NO:157) | 22 | 257 |
| Probe | TET-5′-ccttttggaggctacctttctcagatg-3′-TAMRA | (SEQ ID NO:155) | 27 | 288 |
| Reverse | 5′-gctctccatgacagtgaagaaa-3′ | (SEQ ID NO:159) | 22 | 326 |

TABLE 18MC
Panel 1.3D

| | Rel. Exp. (%) | | Rel. Exp. (%) |
|---|---|---|---|
| | Ag2645, Run | | Ag2645, Run |
| Tissue Name | 167819098 | Tissue Name | 1167819098 |
| Liver adenocarcinoma | 0.0 | Kidney (fetal) | 0.4 |
| Pancreas | 0.0 | Renal ca. 786-0 | 0.0 |
| Pancreatic ca. CAPAN 2 | 0.0 | Renal ca. A498 | 1.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 2.2 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) HepG2 | 0.0 |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) LX-1 | 0.0 |
| Brain (thalamus) | 0.0 | Lung ca. (small cell) NCI-H69 | 0.0 |
| Cerebral Cortex | 0.0 | Lung ca. (s. cell var.) SHP-77 | 0.6 |
| Spinal cord | 0.0 | Lung ca. (large cell)NCI-H460 | 0.0 |
| glio/astro U87-MG | 0.0 | Lung ca. (non-sm. cell) A549 | 0.0 |
| glio/astro U-118-MG | 0.0 | Lung ca. (non-s. cell) NCI-H23 | 0.0 |
| astrocytoma SW1783 | 0.0 | Lung ca. (non-s. cell) HOP-62 | 0.0 |
| neuro*; met SK-N-AS | 0.8 | Lung ca. (non-s. cl) NCI-H522 | 0.0 |
| astrocytoma SF-539 | 0.5 | Lung ca. (squam.) SW 900 | 0.0 |
| astrocytoma SNB-75 | 0.0 | Lung ca. (squam.) NCI-H596 | 0.0 |
| glioma SNB-19 | 0.0 | Mammary gland | 0.0 |
| glioma U251 | 0.8 | Breast ca.* (pl. ef) MCF-7 | 0.0 |
| glioma SF-295 | 0.5 | Breast ca.* (pl. ef) MDA-MB-231 | 0.0 |
| Heart (fetal) | 0.0 | Breast ca.* (pl. ef) T47D | 28.7 |
| Heart | 0.0 | Breast ca. BT-549 | 0.0 |
| Skeletal muscle (fetal) | 0.0 | Breast ca. MDA-N | 0.0 |
| Skeletal muscle | 0.0 | Ovary | 0.5 |
| Bone marrow | 0.0 | Ovarian ca. OVCAR-3 | 8.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.8 | Ovarian ca. IGROV-1 | 1.0 |
| Stomach | 0.0 | Ovarian ca.* (ascites) SK-OV-3 | 100.0 |
| Small intestine | 0.0 | Uterus | 0.0 |
| Colon ca. SW480 | 0.0 | Plancenta | 0.0 |
| Colon ca.* SW620(SW480 met) | 0.0 | Prostate | 0.0 |
| Colon ca. HT29 | 0.0 | Prostate ca.* (bone met)PC-3 | 0.0 |
| Colon ca. HCT-116 | 0.0 | Testis | 0.4 |
| Colon ca. CaCo-2 | 0.8 | Melanoma Hs688(A).T | 0.0 |
| Colon ca. tissue(ODO3866) | 0.0 | Melanoma* (met) Hs688(B).T | 0.0 |
| Colon ca. HCC-2998 | 0.0 | Melanoma UACC-62 | 4.2 |
| Gastric ca.* (liver met) NCI-N87 | 0.0 | Melanoma M14 | 0.0 |
| Bladder | 0.7 | Melanoma LOX IMVI | 0.0 |
| Trachea | 0.0 | Melanoma* (met) SK-MEL-5 | 0.0 |
| Kidney | 0.0 | Adipose | 0.0 |

TABLE 18MD
Panel 2.2

| | Rel. Exp. (%) | | Rel. Exp. (%) |
|---|---|---|---|
| | Ag2645, Run | | Ag2645, Run |
| Tissue Name | 175063797 | Tissue Name | 175063797 |
| Normal Colon | 0.0 | Kidney Margin (OD04348) | 15.7 |
| Colon cancer (OD06064) | 0.0 | Kidney malignant cancer | 0.0 |
| | | (OD06204B) | |
| Colon Margin (OD06064) | 0.0 | Kidney normal adjacent tissue | 0.0 |
| | | (OD06204E) | |
| Colon cancer (OD06159) | 0.0 | Kidney Cancer (OD04450-01) | 0.0 |
| Colon Margin (OD06159) | 0.0 | Kidney Margin (OD04450-03) | 0.0 |
| Colon cancer (OD06297-04) | 0.0 | Kidney Cancer 8120613 | 0.0 |
| Colon Margin (OD06297-015) | 15.4 | Kidney Margin 8120614 | 0.0 |
| CC Gr.2 ascend colon (ODO3921) | 0.0 | Kidney Cancer 9010320 | 0.0 |
| CC Margin (ODO3921) | 0.0 | Kidney Margin 9010321 | 0.0 |
| Colon cancer metastasis (OD06104) | 0.0 | Kidney Cancer 8120607 | 0.0 |
| Lung Margin (OD06104) | 0.0 | Kidney Margin 8120608 | 0.0 |
| Colon mets to lung (OD04451-01) | 0.0 | Normal Uterus | 0.0 |
| Lung Margin (OD04451-02) | 0.0 | Uterine Cancer 06401 | 0.0 |
| Normal Prostate | 0.0 | Normal Thyroid | 0.0 |
| Prostate Cancer (OD04410) | 0.0 | Thyroid Cancer 064010 | 0.0 |
| Prostate Margin (OD04410) | 0.0 | Thyroid Cancer A302152 | 0.0 |
| Normal Ovary | 0.0 | Thyroid Margin A302153 | 0.0 |
| Ovarian cancer (OD06283-03) | 0.0 | Normal Breast | 0.0 |
| Ovarian Margin (OD06283-07) | 0.0 | Breast Cancer (OD04566) | 13.5 |
| Ovarian Cancer 064008 | 20.0 | Breast Cancer 1024 | 0.0 |
| Ovarian cancer (OD06145) | 23.2 | Breast Cancer (OD04590-01) | 0.0 |
| Ovarian Margin (OD06145) | 5.7 | Breast Cancer Mets (OD04590-03) | 0.0 |
| Ovarian cancer (OD06455-03) | 0.0 | Breast Cancer Metastasis | 0.0 |
| | | (OD04655-05) | |
| Ovarian Margin (OD06455-07) | 0.0 | Breast Cancer 064006 | 0.0 |
| Normal Lung | 0.0 | Breast Cancer 9100266 | 0.0 |
| Invasive poor diff. lung adeno | 97.9 | Breast Margin 9100265 | 0.0 |
| (ODO4945-01 | | | |
| Lung Margin (ODO4945-03) | 0.0 | Breast Cancer A209073 | 0.0 |
| Lung Malignant Cancer (OD03126) | 13.5 | Breast Margin A2090734 | 0.0 |
| Lung Margin (OD03126) | 0.0 | Breast cancer (OD06083) | 100.0 |
| Lung Cancer (OD05014A) | 0.0 | Breast cancer node metastasis | 0.0 |
| | | (OD06083) | |
| Lung Margin (OD05014B) | 0.0 | Normal Liver | 0.0 |
| Lung cancer (OD06081) | 0.0 | Liver Cancer 1026 | 0.0 |
| Lung Margin (OD06081) | 0.0 | Liver Cancer 1025 | 0.0 |
| Lung Cancer (OD04237-01) | 0.0 | Liver Cancer 6004-T | 0.0 |
| Lung Margin (OD04237-02) | 0.0 | Liver Tissue 6004-N | 0.0 |
| Ocular Melanoma Metastasis | 0.0 | Liver Cancer 6005-T | 0.0 |
| Ocular Melanoma Margin (Liver) | 0.0 | Liver Tissue 6005-N | 0.0 |
| Melanoma Metastasis | 0.0 | Liver Cancer 064003 | 38.7 |
| Melanoma Margin (Lung) | 0.0 | Normal Bladder | 0.0 |
| Normal Kidney | 0.0 | Bladder Cancer 1023 | 0.0 |
| Kidney Ca, Nuclear grade 2 | 0.0 | Bladder Cancer A302173 | 56.6 |
| (OD04338) | | | |
| Kidney Margin (OD04338) | 0.0 | Normal Stomach | 0.0 |
| Kidney Ca Nuclear grade 1/2 | 0.0 | Gastric Cancer 9060397 | 0.0 |
| (OD04339) | | | |
| Kidney Margin (OD04339) | 0.0 | Stomach Margin 9060396 | 0.0 |
| Kidney Ca, Clear cell type | 0.0 | Gastric Cancer 9060395 | 13.0 |
| (OD04340) | | | |
| Kidney Margin (OD04340) | 0.0 | Stomach Margin 9060394 | 17.8 |
| Kidney Ca, Nuclear grade 3 | 0.0 | Gastric Cancer 064005 | 0.0 |
| (OD04348) | | | |

CNS_neurodegeneration_v1.0 Summary: Ag2576 Expression of gene CG50375-01 is low/undetectable in all samples in this panel (CTs>35). (Data not shown.)

General_screening_panel_v1.4 Summary: Ag2645 Expression of gene CG50375-01 is low/undetectable in all samples in this panel (CTs>35). (Data not shown.)

Panel 1.3D Summary: Ag2645 Expression of gene CG50375-01 is restricted to samples derived from ovarian and breast cancer cell lines. Thus, expression of this gene could be used to differentiate between these samples and other samples on this panel. Furthermore, expression of this gene could be used as a marker to detect the presence of these cancers.

Panel 2.2 Summary: Ag2645 Expression of gene CG50375-01 is restricted to a breast cancer and a lung adenocarcinoma. Thus, expression of this gene could be used to differentiate between these samples and other samples on this panel.

Panel 4D Summary: Ag2576 Expression of gene CG50375-01 is low/undetectable in all samples in this panel (CTs>35). (Data not shown.)

Panel CNS_{—}1 Summary: Ag2645 Expression is low/undetectable in all samples in this panel (CTs>35). (Data not shown.)

N. CG50331-01: Olfactory Receptor

Expression of gene CG50331-01 was assessed using the primer-probe sets Ag2562, Ag1568 and Ag1569, described in Tables 18NA, 18NB and 18NC. Results of the RTQ-PCR runs are shown in Tables 18ND, 18NE, 18NF and 18NG.

TABLE 18NA

Probe Name Ag2562

| | | | |
|---|---|---|---|
| Primers | Sequences | Length | Start Position |

| | | | |
|---|---|---|---|
| Forward | 5′-gtctttctgtgcctctcacatc-3′ (SEQ ID NO:160) | 122 | 503 |
| Probe | TET-5′-ttttctgtgacacccagcctgtg-3′-TAMRA (SEQ ID NO:161) | 23 | 535 |
| Reverse | 5′-tgtcagagcaggagagctttag-3′ (SEQ ID NO:162) | 22 | 558 |

TABLE 18NB

Probe Name Ag1568

| | | | |
|---|---|---|---|
| Primers | Sequences | Length | Start Position |

| | | | |
|---|---|---|---|
| Forward | 5′-gtctttctgtgcctctcacatc-3′ (SEQ ID NO:163) | 22 | 503 |
| Probe | TET-5′-tttttgtgacacccagcctgtg-3′-TAMRA (SEQ ID NO:164) | 23 | 535 |
| Reverse | 5′-tgtcagagcaggagagctttag-3′ (SEQ ID NO:165) | 22 | 558 |

TABLE 18NC

Probe Name Ag1569

| | | | |
|---|---|---|---|
| Primers | Sequences | Length | Start Position |

| | | | |
|---|---|---|---|
| Forward | 5′-gtctttctgtgcctctcacatc-3′ (SEQ ID NO:166) | 22 | 503 |
| Probe | TET-5′-ttttctgtgacacccagcctgtg-3′-TAMRA (SEQ ID NO:167) | 23 | 535 |
| Reverse | 5′-tgtcagagcaggagagctttag-3′ (SEQ ID NO:168) | 22 | 558 |

TABLE 18ND
CNS neurodegeneration v1.0

| | Rel. Exp. (%) Ag2562, | | Rel. Exp. (%) Ag2562, |
|---|---|---|---|
| Tissue Name | Run 208779724 | (Tissue Name | Run 208779724 |
| AD 1 Hippo | 12.8 | Control (Path) 3 Temporal Ctx | 15.9 |
| AD 2 Hippo | 35.1 | Control (Path) 4 Temporal Ctx | 61.1 |
| AD 3 Hippo | 4.7 | AD 1 Occipital Ctx | 13.4 |
| AD 4 Hippo | 36.1 | AD 2 Occipital Ctx (Missing) | 0.0 |
| AD 5 Hippo | 36.1 | AD 3 Occipital Ctx | 1.7 |
| AD 6 Hippo | 37.9 | AD 4 Occipital Ctx | 42.6 |
| Control 2 Hippo | 17.1 | AD 5 Occipital Ctx | 36.1 |
| Control 4 Hippo | 19.5 | AD 6 Occipital Ctx | 23.5 |
| Control (Path) 3 Hippo | 19.5 | Control 1 Occipital Ctx | 4.4 |
| AD 1 Temporal Ctx | 23.0 | Control 2 Occipital Ctx | 20.6 |
| AD 2 Temporal Ctx | 38.7 | Control 3 Occipital Ctx | 37.4 |
| AD 3 Temporal Ctx | 3.3 | Control 4 Occipital Ctx | 25.0 |
| AD 4 Temporal Ctx | 61.6 | Control (Path) 1 Occipital Ctx | 100.0 |
| AD 5 Inf Temporal Ctx | 37.4 | Control (Path) 2 Occipital Ctx | 18.6 |
| AD 5 Sup Temporal Ctx | 34.2 | Control (Path) 3 Occipital Ctx | 4.4 |
| AD 6 Inf Temporal Ctx | 50.0 | Control (Path) 4 Occipital Ctx | 45.4 |
| AD 6 Sup Temporal Ctx | 67.8 | Control 1 Parietal Ctx | 15.1 |
| Control 1 Temporal Ctx | 14.1 | Control 2 Parietal Ctx | 48.3 |
| Control 2 Temporal Ctx | 13.1 | Control 3 Parietal Ctx | 18.4 |
| Control 3 Temporal Ctx | 18.9 | Control (Path) 1 Parietal Ctx | 19.6 |
| Control 3 Temporal Ctx | 34.9 | Control (Path) 2 Parietal Ctx | 35.6 |
| Control (Path) 1 Temporal Ctx | 58.2 | Control (Path) 3 Parietal Ctx | 20.6 |
| Control (Path) 2 Temporal Ctx | 33.2 | Control (Path) 4 Parietal Ctx | 81.8 |

TABLE 18NE
Panel 1.3D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag1568, | Ag1569, | Ag2562, | | Ag1568, | Ag1569, | Ag2562, |
| | Run | Run | Run | Tissue | Run | Run | Run |
| Tissue Name | 165534724 | 165529566 | 165672220 | Name | 165534724 | 165529566 | 165672220 |
| Liver | 0.0 | 8.7 | 7.7 | Kidney | 8.1 | 0.0 | 27.7 |
| adenocarcinoma | | | | (fetal) | | | |
| Pancreas | 12.6 | 0.0 | 0.0 | Renal ca. | 0.0 | 14.5 | 0.0 |
| | | | | 786-0 | | | |
| Pancreatic ca. | 0.0 | 8.2 | 0.0 | Renal ca. | 34.2 | 24.0 | 11.8 |
| CAPAN 2 | | | | A498 | | | |
| Adrenal gland | 7.7 | 0.0 | 21.5 | Renal ca. | 17.3 | 43.8 | 9.1 |
| | | | | RXF 393 | | | |
| Thyroid | 13.1 | 0.0 | 0.0 | Renal ca. | 0.0 | 0.0 | 8.3 |
| | | | | ACHN | | | |
| Salivary gland | 29.7 | 61.1 | 67.8 | Renal ca. | 0.0 | 0.0 | 0.0 |
| | | | | UO-31 | | | |
| Pituitary gland | 9.3 | 20.2 | 8.7 | Renal ca. | 0.0 | 0.0 | 14.7 |
| | | | | TK-10 | | | |
| Brain (fetal) | 7.1 | 0.0 | 14.3 | Liver | 22.1 | 26.4 | 0.0 |
| Brain (whole) | 0.0 | 16.4 | 0.0 | Liver (fetal) | 6.6 | 10.3 | 0.0 |
| Brain (amygdala) | 12.8 | 51.1 | 32.3 | Liver ca. | 10.3 | 0.0 | 0.0 |
| | | | | (hepatoblast) | | | |
| | | | | HepG2 | | | |
| Brain | 27.9 | 7.2 | 19.6 | Lung | 7.3 | 10.6 | 47.3 |
| (cerebellum) | | | | | | | |
| Brain | 24.7 | 16.3 | 45.1 | Lung (fetal) | 25.2 | 17.2 | 13.2 |
| (hippocampus) | | | | | | | |
| Brain (substantia | 34.2 | 23.2 | 31.2 | Lung ca. | 0.0 | 0.0 | 0.0 |
| nigra) | | | | (small cell) | | | |
| | | | | LX-1 | | | |
| Brain (thalamus) | 100.0 | 39.8 | 22.1 | Lung ca. | 0.0 | 0.0 | 0.0 |
| | | | | (small cell) | | | |
| | | | | NCI-H69 | | | |
| Cerebral Cortex | 6.3 | 43.8 | 11.2 | Lung ca. | 0.0 | 0.0 | 0.0 |
| | | | | (s. cell var.) | | | |
| | | | | SHP-77 | | | |
| Spinal cord | 75.3 | 76.8 | 6.3 | Lung ca. | 0.0 | 33.0 | 18.8 |
| | | | | (large | | | |
| | | | | cell) NCI- | | | |
| | | | | H460 | | | |
| glio/astro U87- | 5.8 | 15.5 | 35.4 | Lung ca. | 9.3 | 14.8 | 0.0 |
| MG | | | | (non-sm. | | | |
| | | | | cell) A549 | | | |
| glio/astro U-118- | 0.0 | 0.0 | 21.0 | Lung ca. | 4.5 | 0.0 | 0.0 |
| MG | | | | (non-s. cell) | | | |
| | | | | NCI-H23 | | | |
| astrocytoma | 28.1 | 44.1 | 6.7 | Lung ca. | 0.0 | 9.3 | 4.7 |
| SW1783 | | | | (non-s. cell) | | | |
| | | | | HOP-62 | | | |
| neuro*; met SK-N-AS | 6.0 | 4.0 | 6.5 | Lung ca. | 0.0 | 0.0 | 0.0 |
| | | | | (non-s. cl) | | | |
| | | | | NCI-H522 | | | |
| astrocytoma SF- | 9.3 | 15.0 | 0.0 | Lung ca. | 0.0 | 2.4 | 0.0 |
| 539 | | | | (squam.) SW | | | |
| | | | | 900 | | | |
| astrocytoma | 10.7 | 10.4 | 4.7 | Lung ca. | 0.0 | 0.0 | 7.7 |
| SNB-75 | | | | (squam.) | | | |
| | | | | NCl-H596 | | | |
| glioma SNB-19 | 9.2 | 10.8 | 22.1 | Mammary | 0.0 | 10.0 | 11.1 |
| | | | | gland | | | |
| glioma U251 | 12.2 | 57.8 | 51.1 | Breast ca.* | 0.0 | 8.3 | 0.0 |
| | | | | (pl. ef) MCF- | | | |
| | | | | 7 | | | |
| glioma SF-295 | 36.6 | 7.7 | 26.6 | Breast ca.* | 0.0 | 9.3 | 7.6 |
| | | | | (pl. ef) | | | |
| | | | | MDA-MB- | | | |
| | | | | 231 | | | |
| Heart (fetal) | 0.0 | 0.0 | 0.0 | Breast ca.* | 0.0 | 20.7 | 0.0 |
| | | | | (pl. ef) T47D | | | |
| Heart | 6.1 | 0.0 | 0.0 | Breast ca. | 0.0 | 0.0 | 0.0 |
| | | | | BT-549 | | | |
| Skeletal muscle | 6.0 | 0.0 | 0.0 | Breast ca. | 0.0 | 26.6 | 0.0 |
| (fetal) | | | | MDA-N | | | |
| Skeletal muscle | 0.0 | 0.0 | 15.3 | Ovary | 0.0 | 0.0 | 6.7 |
| Bone marrow | 0.0 | 32.1 | 6.3 | Ovarian ca | 8.8 | 0.0 | 0.0 |
| | | | | OVCAR-3 | | | |
| Thymus | 36.1 | 9.6 | 0.0 | Ovarian ca. | 7.9 | 17.6 | 16.6 |
| | | | | OVCAR-4 | | | |
| Spleen | 18.4 | 29.9 | 0.0 | Ovarian ca. | 0.0 | 0.0 | 6.0 |
| | | | | OVCAR-5 | | | |
| Lymph node | 27.2 | 30.4 | 100.0 | Ovarian ca. | 3.4 | 0.0 | 15.3 |
| | | | | OVCAR-8 | | | |
| Colorectal | 77.9 | 31.2 | 39.0 | Ovarian ca. | 0.0 | 0.0 | 0.0 |
| | | | | IGROV-1 | | | |
| Stomach | 7.4 | 2.8 | 31.9 | Ovarian ca.* | 60.3 | 12.3 | 8.3 |
| | | | | (ascites) SK- | | | |
| | | | | OV-3 | | | |
| Small intestine | 31.6 | 25.0 | 17.4 | Uterus | 8.4 | 100.0 | 45.4 |
| Colon ca. SW480 | 7.6 | 6.7 | 7.5 | Plancenta | 12.2 | 51.4 | 24.7 |
| Colon ca.* | 0.0 | 0.0 | 0.0 | Prostate | 0.0 | 7.6 | 0.0 |
| SW620(SW480 | | | | | | | |
| met) | | | | | | | |
| Colon ca. HT29 | 0.0 | 0.0 | 0.0 | Prostate ca.* | 0.0 | 7.7 | 7.6 |
| | | | | (bone | | | |
| | | | | met)PC-3 | | | |
| Colon ca. HCT- | 8.5 | 0.0 | 0.0 | Testis | 0.0 | 8.5 | 0.0 |
| 116 | | | | | | | |
| Colon ca. CaCo-2 | 15.4 | 0.0 | 0.0 | Melanoma | 0.0 | 0.0 | 7.5 |
| | | | | Hs688(A).T | | | |
| Colon ca. | 0.0 | 0.0 | 0.0 | Melanoma* | 8.2 | 0.0 | 15.6 |
| tissue(ODO3866) | | | | (met) | | | |
| | | | | Hs688(B).T | | | |
| Colon ca. HCC- | 28.9 | 0.0 | 0.0 | Melanoma | 0.0 | 0.0 | 12.4 |
| 2998 | | | | UACC-62 | | | |
| Gastric ca.* (liver | 9.3 | 17.4 | 6.7 | Melanoma | 0.0 | 0.0 | 0.0 |
| met) NCI-N87 | | | | M14 | | | |
| Bladder | 62.4 | 62.0 | 17.8 | Melanoma | 0.0 | 0.0 | 0.0 |
| | | | | LOX IMVI | | | |
| Trachea | 45.1 | 15.8 | 0.0 | Melanoma* | 9.6 | 0.0 | 0.0 |
| | | | | (met) SK- | | | |
| | | | | MEL-5 | | | |
| Kidney | 0.0 | 7.9 | 0.0 | Adipose | 6.1 | 25.7 | 16.0 |

TABLE 18NF
Panel 2.2

| | Rel. | Rel. | | Rel. | Rel. |
|---|---|---|---|---|---|
| | Exp. (%) | Exp. (%) | | Exp. (%) | Exp. (%) |
| | Ag1568, | Ag1569, | | Ag1568, | Ag1569, |
| | Run | Run | | Run | Run |
| Tissue Name | 173968822 | 173850036 | Tissue Name | 173968822 | 173850036 |
| Normal Colon | 36.6 | 4.6 | Kidney Margin | 21.9 | 53.2 |
| | | | (OD04348) | | |
| Colon cancer (OD06064) | 0.0 | 0.0 | Kidney malignant cancer | 0.0 | 8.6 |
| | | | (OD06204B) | | |
| Colon Margin (OD06064) | 2.8 | 0.0 | Kidney normal adjacent | 6.7 | 0.0 |
| | | | tissue (OD06204E) | | |
| Colon cancer (OD06159) | 0.0 | 0.0 | Kidney Cancer | 3.2 | 4.3 |
| | | | (OD04450-01) | | |
| Colon Margin (OD06159) | 35.4 | 12.4 | Kidney Margin | 23.0 | 27.0 |
| | | | (OD04450-03) | | |
| Colon cancer (OD06297- | 0.0 | 0.0 | Kidney Cancer 8120613 | 0.0 | 0.0 |
| 04) | | | | | |
| Colon Margin (OD06297- | 2.8 | 19.1 | Kidney Margin 8120614 | 15.2 | 2.9 |
| 015) | | | | | |
| CC Gr.2 ascend colon | 0.0 | 2.8 | Kidney Cancer 9010320 | 7.2 | 0.0 |
| (ODO3921) | | | | | |
| CC Margin (ODO3921) | 4.9 | 4.2 | Kidney Margin 9010321 | 11.7 | 0.0 |
| Colon cancer metastasis | 14.3 | 0.0 | Kidney Cancer 8120607 | 0.0 | 0.0 |
| (OD06104) | | | | | |
| Lung Margin (OD06104) | 0.0 | 7.3 | Kidney Margin 8120608 | 0.0 | 0.0 |
| Colon mets to lung | 11.2 | 27.4 | Normal Uterus | 51.4 | 46.3 |
| (OD04451-01) | | | | | |
| Lung Margin (OD04451- | 38.4 | 37.6 | Uterine Cancer 064011 | 23.0 | 28.1 |
| 02) | | | | | |
| Normal Prostate | 21.6 | 13.1 | Normal Thyroid | 6.1 | 0.0 |
| Prostate Cancer (OD04410) | 22.7 | 23.7 | Thyroid Cancer 064010 | 0.0 | 0.0 |
| Prostate Margin (OD04410) | 15.7 | 5.0 | Thyroid Cancer A302152 | 27.7 | 26.1 |
| Normal Ovary | 0.0 | 0.0 | Thyroid Margin A302153 | 0.0 | 19.2 |
| Ovarian cancer (OD06283- | 6.5 | 9.7 | Normal Breast | 66.4 | 15.0 |
| 03) | | | | | |
| Ovarian Margin (OD06283- | 25.3 | 30.1 | Breast Cancer | 22.5 | 15.8 |
| 07) | | | (OD04566) | | |
| Ovarian Cancer 064008 | 12.0 | 10.3 | Breast Cancer 1024 | 0.0 | 7.5 |
| Ovarian cancer (OD06145) | 0.0 | 0.0 | Breast Cancer | 41.8 | 18.4 |
| | | | (OD04590-01) | | |
| Ovarian Margin (OD06145) | 21.8 | 7.5 | Breast Cancer Mets | 18.2 | 12.0 |
| | | | (OD04590-03) | | |
| Ovarian cancer (OD06455- | 0.0 | 0.0 | Breast Cancer Metastasis | 37.1 | 17.6 |
| 03) | | | (OD04655-05) | | |
| Ovarian Margin (OD06455- | 4.5 | 0.0 | Breast Cancer 064006 | 31.0 | 17.9 |
| 07) | | | | | |
| Normal Lung | 12.0 | 5.9 | Breast Cancer 9100266 | 19.2 | 3.2 |
| Invasive poor diff. lung | 6.5 | 3.9 | Breast Margin 9100265 | 0.0 | 11.9 |
| adeno (ODO4945-01 | | | | | |
| Lung Margin (ODO4945- | 84.7 | 42.6 | Breast Cancer A209073 | 0.0 | 4.1 |
| 03) | | | | | |
| Lung Malignant Cancer | 18.9 | 4.9 | Breast Margin A2090734 | 44.4 | 29.5 |
| (OD03126) | | | | | |
| Lung Margin (OD03126) | 0.0 | 7.9 | Breast cancer (OD06083) | 42.9 | 37.9 |
| Lung Cancer (OD05014A) | 0.0 | 31.2 | Breast cancer node | 30.6 | 37.4 |
| | | | metastasis (OD06083) | | |
| Lung Margin (OD05014B) | 100.0 | 100.0 | Normal Liver | 20.3 | 13.2 |
| Lung cancer (OD06081) | 23.2 | 9.7 | Liver Cancer 1026 | 0.0 | 4.5 |
| Lung Margin (OD06081) | 45.4 | 7.3 | Liver Cancer 1025 | 0.0 | 7.7 |
| Lung Cancer (OD04237- | 5.7 | 0.0 | Liver Cancer 6004-T | 0.0 | 0.0 |
| 01) | | | | | |
| Lung Margin (OD04237- | 16.7 | 9.3 | Liver Tissue 6004-N | 0.0 | 3.9 |
| 02) | | | | | |
| Ocular Melanoma | 0.0 | 0.0 | Liver Cancer 6005-T | 0.0 | 0.0 |
| Metastasis | | | | | |
| Ocular Melanoma Margin | 0.0 | 4.3 | Liver Tissue 6005-N | 7.4 | 0.0 |
| (Liver) | | | | | |
| Melanoma Metastasis | 0.0 | 0.0 | Liver Cancer 064003 | 10.7 | 0.0 |
| Melanoma Margin (Lung) | 11.1 | 10.7 | Normal Bladder | 12.8 | 16.0 |
| Normal Kidney | 22.1 | 4.0 | Bladder Cancer 1023 | 0.0 | 4.1 |
| Kidney Ca. Nuclear grade 2 | 19.9 | 24.1 | Bladder Cancer A302173 | 12.7 | 10.9 |
| (OD04338) | | | | | |
| Kidney Margin (OD04338) | 0.0 | 15.1 | Normal Stomach | 51.4 | 31.9 |
| Kidney Ca Nuclear grade | 31.9 | 24.3 | Gastric Cancer 9060397 | 0.0 | 0.0 |
| 1/2 (OD04339) | | | | | |
| Kidney Margin (OD04339) | 10.3 | 0.7 | Stomach Margin | 11.3 | 8.7 |
| | | | 9060396 | | |
| Kidney Ca, Clear cell type | 12.3 | 0.0 | Gastric Cancer 9060395 | 19.8 | 0.0 |
| (OD04340) | | | | | |
| Kidney Margin (OD04340) | 5.5 | 14.4 | Stomach Margin | 2.2 | 8.5 |
| | | | 9060394 | | |
| Kidney Ca, Nuclear grade 3 | 0.0 | 3.6 | Gastric Cancer 064005 | 5.2 | 4.3 |
| (OD04348) | | | | | |

TABLE 18NG
Panel 4D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag1568, | Ag1569, | Ag2562, | | Ag1568, | Ag1569, | Ag2562, |
| | Run | Run | Run | | Run | Run | Run |
| Tissue Name | 163479584 | 165301526 | 164393478 | Tissue Name | 163479584 | 165301526 | 164393478 |
| Secondary Th1 act | 13.0 | 9.5 | 31.0 | HUVEC IL- | 4.5 | 14.9 | 2.4 |
| | | | | 1beta | | | |
| Secondary Th2 act | 31.0 | 33.9 | 24.3 | HUVEC IFN | 69.7 | 60.7 | 65.1 |
| | | | | gamma | | | |
| Secondary Tr1 act | 27.5 | 25.5 | 35.4 | HUVEC TNF | 9.6 | 9.2 | 14.7 |
| | | | | alpha + IFN | | | |
| | | | | gamma | | | |
| Secondary Th1 | 39.8 | 12.2 | 22.7 | HUVEC TNF | 3.6 | 3.8 | 16.2 |
| rest | | | | alpha + IL4 | | | |
| Secondary Th2 | 94.6 | 59.9 | 27.2 | HUVEC IL-11 | 57.8 | 46.3 | 35.6 |
| rest | | | | | | | |
| Secondary Tr1 | 48.3 | 41.8 | 28.7 | Lung | 68.3 | 46.3 | 53.6 |
| rest | | | | Microvascular | | | |
| | | | | EC none | | | |
| Primary Th1 act | 6.1 | 0.0 | 5.5 | Lung | 33.0 | 51.8 | 41.8 |
| | | | | Microvascular | | | |
| | | | | EC TNFalpha + | | | |
| | | | | IL-1beta | | | |
| Primary Th2 act | 19.2 | 11.2 | 3.5 | Microvascular | 55.5 | 77.4 | 55.9 |
| | | | | Dermal EC none | | | |
| Primary Tr1 act | 10.8 | 2.3 | 14.2 | Microsvasular | 23.3 | 51.1 | 28.3 |
| | | | | Dermal EC | | | |
| | | | | TNFalpha + IL- | | | |
| | | | | 1beta | | | |
| Primary Th1 rest | 62.9 | 44.4 | 41.8 | Bronchial | 8.9 | 8.5 | 15.8 |
| | | | | epithelium | | | |
| | | | | TNFalpha + | | | |
| | | | | IL1beta | | | |
| Primary Th2 rest | 47.6 | 26.8 | 57.0 | Small airway | 14.8 | 6.4 | 9.7 |
| | | | | epithelium none | | | |
| Primary Tr1 rest | 14.6 | 13.9 | 8.1 | Small airway | 21.6 | 27.2 | 31.6 |
| | | | | epithelium | | | |
| | | | | TNFalpha + IL- | | | |
| | | | | 1beta | | | |
| CD45RA CD4 | 16.3 | 5.8 | 2.7 | Coronery artery | 13.5 | 0.0 | 0.0 |
| lymphocyte act | | | | SMC rest | | | |
| CD45RO CD4 | 19.5 | 18.2 | 25.5 | Coronery artery | 0.0 | 0.0 | 2.6 |
| lymphocyte act | | | | SMC TNFalpha + | | | |
| | | | | IL-1beta | | | |
| CD8 lymphocyte | 15.6 | 9.3 | 21.0 | Astrocytes rest | 13.6 | 3.2 | 7.8 |
| act | | | | | | | |
| Secondary CD8 | 19.8 | 15.4 | 13.9 | Astrocytes | 3.4 | 2.6 | 2.2 |
| lymphocyte rest | | | | TNFalpha + IL- | | | |
| | | | | 1beta | | | |
| Secondary CD8 | 21.0 | 11.8 | 21.0 | KU-812 | 11.9 | 12.5 | 6.9 |
| lymphocyte act | | | | (Basophil) rest | | | |
| CD4 lymphocyte | 0.0 | 9.6 | 18.8 | KU-812 | 29.1 | 24.8 | 25.5 |
| none | | | | (Basophil) | | | |
| | | | | PMA/ionomycin | | | |
| 2ry | 100.0 | 85.9 | 70.7 | CCD1106 | 0.0 | 0.0 | 0.0 |
| Th1/Th2/Tr1_anti- | | | | (Keratinocytes) | | | |
| CD95 CH11 | | | | none | | | |
| LAK cells rest | 28.3 | 23.0 | 10.7 | CCD1106 | 0.0 | 5.1 | 0.0 |
| | | | | (Keratinocytes) | | | |
| | | | | TNFalpha + IL- | | | |
| | | | | 1beta | | | |
| LAK cells IL-2 | 21.9 | 25.5 | 29.1 | Liver cirrhosis | 44.1 | 49.3 | 55.5 |
| LAK cells IL- | 30.8 | 11.3 | 26.6 | Lupus kidney | 0.0 | 0.0 | 9.0 |
| 2 + IL-12 | | | | | | | |
| LAK cells IL- | 0.0 | 50.7 | 34.4 | NCI-H292 none | 26.2 | 33.4 | 14.8 |
| 2 + IFN gamma | | | | | | | |
| LAK cells IL-2 + | 44.8 | 39.8 | 37.6 | NCI-H292 IL-4 | 12.3 | 2.4 | 19.2 |
| IL-18 | | | | | | | |
| LAK cells | 1.8 | 3.0 | 0.0 | NCI-H292 IL-9 | 16.7 | 7.7 | 11.3 |
| PMA/ionomycin | | | | | | | |
| NK Cells IL-2 rest | 14.6 | 33.4 | 26.2 | NCI-H292 IL- | 5.8 | 1.6 | 6.8 |
| | | | | 13 | | | |
| Two Way MLR 3 | 32.5 | 18.9 | 25.0 | NCI-H292 IFN | 4.2 | 0.0 | 4.5 |
| day | | | | gamma | | | |
| Two Way MLR 5 | 14.8 | 6.8 | 9.5 | HPAEC none | 58.2 | 35.4 | 71.2 |
| day | | | | | | | |
| Two Way MLR 7 | 12.2 | 3.4 | 22.2 | HPAEC TNF | 43.5 | 20.7 | 28.3 |
| day | | | | alpha + IL-1 | | | |
| | | | | beta | | | |
| PBMC rest | 13.2 | 6.0 | 7.8 | Lung fibroblast | 10.2 | 7.0 | 22.7 |
| | | | | none | | | |
| PBMC PWM | 33.2 | 48.6 | 39.5 | Lung fibroblast | 9.5 | 18.9 | 10.0 |
| | | | | TNF alpha + IL- | | | |
| | | | | 1beta | | | |
| PBMC PHA-L | 15.4 | 11.7 | 11.9 | Lung fibroblast | 7.9 | 21.3 | 14.5 |
| | | | | IL-4 | | | |
| Ramos (B cell) | 0.0 | 7.2 | 2.9 | Lung fibroblast | 10.5 | 23.0 | 29.7 |
| none | | | | IL-9 | | | |
| Ramos (B cell) | 12.8 | 7.1 | 24.5 | Lung fibroblast | 25.2 | 9.7 | 24.1 |
| ionomycin | | | | IL-13 | | | |
| B lymphocytes | 48.6 | 29.3 | 24.1 | Lung fibroblast | 14.6 | 9.1 | 13.7 |
| PWM | | | | IFN gamma | | | |
| B lymphocytes | 40.6 | 47.3 | 38.7 | Dermal | 15.0 | 9.0 | 5.4 |
| CD40L and IL-4 | | | | fibroblast | | | |
| | | | | CCD1070 rest | | | |
| EOL-1 dbcAMP | 16.6 | 10.6 | 7.0 | Dermal | 57.8 | 43.2 | 48.0 |
| | | | | fibroblast | | | |
| | | | | CCD1070 TNF | | | |
| | | | | alpha | | | |
| EOL-1 dbcAMP | 5.6 | 17.8 | 5.7 | Dermal | 7.1 | 2.3 | 10.7 |
| PMA/ionomycin | | | | fibroblast | | | |
| | | | | CCD1070 IL-1 | | | |
| | | | | beta | | | |
| Dendritic cells | 10.4 | 12.7 | 14.7 | Dermal | 13.9 | 10.9 | 7.0 |
| none | | | | fibroblast IFN | | | |
| | | | | gamma | | | |
| Dendritic cells | 14.4 | 15.1 | 13.6 | Dermal | 28.9 | 5.8 | 24.7 |
| LPS | | | | fibroblast IL-4 | | | |
| Dendritic cells | 18.3 | 12.9 | 26.6 | IBD Colitis 2 | 11.4 | 7.3 | 2.7 |
| anti-CD40 | | | | | | | |
| Monocytes rest | 2.5 | 1.3 | 13.9 | IBD Crohn's | 17.7 | 0.9 | 21.6 |
| Monocytes LPS | 11.0 | 8.5 | 11.5 | Colon | 29.1 | 17.7 | 15.0 |
| Macrophages rest | 9.2 | 14.9 | 12.2 | Lung | 8.1 | 12.2 | 10.6 |
| Macrophages LPS | 11.2 | 13.1 | 7.9 | Thymus | 33.2 | 25.3 | 27.2 |
| HUVEC none | 28.9 | 19.3 | 28.3 | Kidney | 194.0 | 100.0 | 100.0 |
| HUVEC starved | 84.7 | 65.1 | 93.3 | | | | |

CNS_neurodegeneration_v1.0 Summary: Ag2562 No difference is detected in the expression of the gene in the postmortem brains of Alzheimer's diseased patients when compared to controls; however this panel demonstrates the expression of this gene in the brains of an independent group of subjects. Please see panel 1.3 d for a discussion of utility of this gene in the central nervous system.

Panel 1.3D Summary: Ag1568/Ag1569/Ag2562 Three experiments with the same probe and primer set show expression of the CG50331-01 gene in samples from many different tissues. This gene represents a novel G-protein coupled receptor (GPCR) with expression in the brain. The GPCR family of receptors contains a large number of neurotransmitter receptors, including the dopamine, serotonin, a and b-adrenergic, acetylcholine muscarinic, histamine, peptide, and metabotropic glutamate receptors. GPCRs are excellent drug targets in various neurologic and psychiatric diseases. All antipsychotics have been shown to act at the dopamine D2 receptor; similarly novel antipsychotics also act at the serotonergic receptor, and often the muscarinic and adrenergic receptors as well. While the majority of antidepressants can be classified as selective serotonin reuptake inhibitors, blockade of the 5-HT1A and a2 adrenergic receptors increases the effects of these drugs. The GPCRs are also of use as drug targets in the treatment of stroke. Blockade of the glutamate receptors may decrease the neuronal death resulting from excitotoxicity; further more the purinergic receptors have also been implicated as drug targets in the treatment of cerebral ischemia. The b-adrenergic receptors have been implicated in the treatment of ADHD with Ritalin, while the a-adrenergic receptors have been implicated in memory. Therefore this gene may be of use as a small molecule target for the treatment of any of the described diseases.

There is also significant expression in a sample from uterus and a brain cancer cell line. Thus, expression of this gene could be used to differentiate between these and tissues and other samples on this panel.

Significant expression is also seen in the lymph node. Please see Panel 4D for discussion of potential utility in the immune system.

Overall, expression of this gene appears to be associated with normal tissue samples. This observation is in concordance with the results seen in Panel 2.2.

References:

El Yacoubi M, Ledent C, Parmentier M, Bertorelli R, Ongini E, Costentin J, Vaugeois J M. Adenosine A2A receptor antagonists are potential antidepressants: evidence based on pharmacology and A2A receptor knockout mice. Br J Pharmacol 2001 Sep;134(1):68-77

1. Adenosine, an ubiquitous neuromodulator, and its analogues have been shown to produce ‘depressant’ effects in animal models believed to be relevant to depressive disorders, while adenosine receptor antagonists have been found to reverse adenosine-mediated ‘depressant’ effect. 2. We have designed studies to assess whether adenosine A2A receptor antagonists, or genetic inactivation of the receptor would be effective in established screening procedures, such as tail suspension and forced swim tests, which are predictive of clinical antidepressant activity. 3. Adenosine A2A receptor knockout mice were found to be less sensitive to ‘depressant’ challenges than their wildtype littermates. Consistently, the adenosine A2A receptor blockers SCH 58261 (1-10 mg kg(−1), i.p.) and KW 6002 (0.1-10 mg kg(−1), p.o.) reduced the total immobility time in the tail suspension test. 4. The efficacy of adenosine A2A receptor antagonists in reducing immobility time in the tail suspension test was confirmed and extended in two groups of mice. Specifically, SCH 58261 (1-10 mg kg(−1)) and ZM 241385 (15-60 mg kg(−1)) were effective in mice previously screened for having high immobility time, while SCH 58261 at 10 mg kg(−1) reduced immobility of mice that were selectively bred for their spontaneous ‘helplessness’ in this assay. 5. Additional experiments were carried out using the forced swim test. SCH 58261 at 10 mg kg(−1) reduced the immobility time by 61%, while KW 6002 decreased the total immobility time at the doses of 1 and 10 mg kg(−1) by 75 and 79%, respectively. 6. Administration of the dopamine D2 receptor antagonist haloperidol (50-200 microg kg(−1) i.p.) prevented the antidepressant-like effects elicited by SCH 58261 (10 mg kg(−1) i.p.) in forced swim test whereas it left unaltered its stimulant motor effects. 7. In conclusion, these data support the hypothesis that A2A receptor antagonists prolong escape-directed behaviour in two screening tests for antidepressants. Altogether the results support the hypothesis that blockade of the adenosine A2A receptor might be an interesting target for the development of effective antidepressant agents.

Blier P. Pharmacology of rapid-onset antidepressant treatment strategies. Clin Psychiatry 2001;62 Suppl 15:12-7

Although selective serotonin reuptake inhibitors (SSRIs) block serotonin (5-HT) reuptake rapidly, their therapeutic action is delayed. The increase in synaptic 5-HT activates feedback mechanisms mediated by 5-HT1A (cell body) and 5-HT1B (terminal) autoreceptors, which, respectively, reduce the firing in 5-HT neurons and decrease the amount of 5-HT released per action potential resulting in attenuated 5-HT neurotransmission. Long-term treatment desensitizes the inhibitory 5-HT1 autoreceptors, and 5-HT neurotransmission is enhanced. The time course of these events is similar to the delay of clinical action. The addition of pindolol, which blocks 5-HT1A receptors, to SSRI treatment decouples the feedback inhibition of 5-HT neuron firing and accelerates and enhances the antidepressant response. The neuronal circuitry of the 5-HT and norepinephrine (NE) systems and their connections to forebrain areas believed to be involved in depression has been dissected. The firing of 5-HT neurons in the raphe nuclei is driven, at least partly, by alpha1-adrenoceptor-mediated excitatory inputs from NE neurons. Inhibitory alpha2-adrenoceptors on the NE neuroterminals form part of a feedback control mechanism. Mirtazapine, an antagonist at alpha2-adrenoceptors, does not enhance 5-HT neurotransmission directly but disinhibits the NE activation of 5-HT neurons and thereby increases 5-HT neurotransmission by a mechanism that does not require a time-dependent desensitization of receptors. These neurobiological phenomena may underlie the apparently faster onset of action of mirtazapine compared with the SSRIs.

Tranquillini M E, Reggiani A. Glycine-site antagonists and stroke. Expert Opin Investig Drugs 1999 Nov;8(11):1837-1848

The excitatory amino acid, (S)-glutamic acid, plays an important role in controlling many neuronal processes. Its action is mediated by two main groups of receptors: the ionotropic receptors (which include NMDA, AMPA and kainic acid subtypes) and the metabotropic receptors (mGluR(1-8)) mediating G-protein coupled responses. This review focuses on the strychnine insensitive glycine binding site located on the NMDA receptor channel, and on the possible use of selective antagonists for the treatment of stroke. Stroke is a devastating disease caused by a sudden vascular accident. Neurochemically, a massive release of glutamate occurs in neuronal tissue; this overactivates the NMDA receptor, leading to increased intracellular calcium influx, which causes neuronal cell death through necrosis. NMDA receptor activation strongly depends upon the presence of glycine as a co-agonist. Therefore, the administration of a glycine antagonist can block overactivation of NMDA receptors, thus preserving neurones from damage. The glycine antagonists currently identified can be divided into five main categories depending on their chemical structure: indoles, tetrahydroquinolines, benzoazepines, quinoxalinediones and pyrida-zinoquinolines.

Monopoli A, Lozza G, Forlani A, Mattavelli A, Ongini E. Blockade of adenosine A2A receptors by SCH 58261 results in neuroprotective effects in cerebral ischaemia in rats. Neuroreport 1998 Dec 1;9(17):3955-9

Blockade of adenosine receptors can reduce cerebral infarct size in the model of global ischaemia. Using the potent and selective A2A adenosine receptor antagonist, SCH 58261, we assessed whether A2A receptors are involved in the neuronal damage following focal cerebral ischaemia as induced by occluding the left middle cerebral artery. SCH 58261 (0.01 mg/kg either i.p. or i.v.) administered to normotensive rats 10 min after ischaemia markedly reduced cortical infarct volume as measured 24 h later (30% vs controls, p<0.05). Similar effects were observed when SCH 58261 (0.01 mg/kg, i.p.) was administered to hypertensive rats (28% infarct volume reduction vs controls, p<0.05). Neuroprotective properties of SCH 58261 administered after ischaemia indicate that blockade of A2A adenosine receptors is a potentially useful biological target for the reduction of brain injury.

Panel 2.2 Summary: Ag1568/Ag1569 Two experiments with the same probe and primer set show highest expression of the CG50331-01 gene in normal lung tissue adjacent to a tumor (CTs=32.5). Furthermore, there appears to be higher expression in a cluster of lung tissue samples when compared to matched tumor samples. There is also significant expression in normal kidney and uterus samples when compared to matched kidney and uterus cancers. Thus, expression of this gene could be used to differentiate between these samples and other samples in this panel. In addition, expression of this gene could also potentially be used as a marker to test for the presence of lung, kidney or uterine cancers. Therefore, therapeutic modulation of the expression or function of the protein encoded by this gene could potentially be useful in the treatment of lung, kidney and uterine cancers.

Panel 4D Summary: Ag1568/Ag1569/Ag2562 Three experiments with the same probe and primer set all show highest expression of the CG50331-01 transcript in kidney and secondary Th2 rest and secondary Th1/TH2/Tr1 cells treated with anti-CD95 (CTs=32.4). The expression of this transcript is decreased upon activation of these T (Th1 or Th2) cells. This transcript is also found at lower but still significant levels in B cells activated by PWM stimulation or treatment with CD40L and IL-4, where the latter condition promotes B cell survival and differentiation. This transcript encodes for a putative GPCR that may therefore function as regulator of Fas or other cell death pathways in T and B cells.

Expression of this transcript is also found in starved HUVEC, lung and dermal microvasculature. In addition, this expression of this transcript is down regulated in these tissues by TNF-a, a cytokine with cytotoxic activity on these cell types. Therefore, protein therapeutics designed with the putative GPCR encoded for by this protein could reduce or eliminate inflammation and tissue damage observed in lung and skin inflammatory diseases such as asthma, chronic bronchitis, psoriasis, and atopic dermatitis. Thereapeutic modulation of the function or expression of the protein encoded by this gene may also reduce or eliminate inflammation and tissue damage that result from diseases associated with hyperactivated T cells including lupus erythematosus, rheumatoid arthritis, and inflammatory bowel diseases.

O. CG50349-01: GPCR

Expression of gene CG50349-01 was assessed using the primer-probe set Ag2574, described in Table 18OA. Results of the RTQ-PCR runs are shown in Table OD.

TABLE 18OA

Probe Name Ag2574

| | | | |
|---|---|---|---|
| Primers | Sequences | Length | Start Position |

| | | | |
|---|---|---|---|
| Forward | 5′-tccttctctgtcatttcctcaa-3′ (SEQ ID NO:169) | 122 | 1785 |
| Probe | TET-5′-tttcattggagaaccacaagacttca-3′-TAMRA (SEQ ID NO:170) | 26 | 1825 |
| Reverse | 5′-cattgtctgcctggttttacat-3′ (SEQ ID NO:171) | 22 | 1852 |

TABLE 18OB
Panel 4D

| | Rel. Exp. (%) | | Rel. Exp. (%) |
|---|---|---|---|
| | Ag2574, Run | | Ag2574, Run |
| Tissue Name | 164226642 | Tissue Name | 164226642 |
| Secondary Th1 act | 0.0 | HUVEC IL-lbeta | 0.0 |
| Secondary Th2 act | 0.0 | HUVEC IFN gamma | 0.0 |
| Secondary Tr1 act | 12.2 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + | 0.0 |
| | | IL-1beta | |
| Primary Th2 act | 0.0 | Microvascular Dermal EC none | 0.0 |
| Primary Tr1 act | 0.0 | Microsvasular Dermal EC TNFalpha1 + | 0.0 |
| | | IL-1beta | |
| Primary Th1 rest | 0.0 | Bronchial epithelium TNFalpha + | 0.0 |
| | | IL1beta | |
| Primary Th2 rest | 0.0 | Small airway epithelium none | 0.0 |
| Primary Tr1 rest | 0.0 | Small airway epithelium TNFalpha + | 0.0 |
| | | IL-1beta | |
| CD45RA CD4 lymphocyte act | 0.0 | Coronery artery SMC rest | 0.0 |
| CD45RO CD4 lymphocyte act | 0.0 | Coronery artery SMC TNFalpha + IL- | 0.0 |
| | | 1beta | |
| CD8 lymphocyte act | 0.0 | Astrocytes rest | 0.0 |
| Secondary CD8 lymphocyte rest | 0.0 | Astrocytes TNFalpha + IL-1beta | 0.0 |
| Secondary CD8 lymphocyte act | 0.0 | KU-812 (Basophil) rest | 0.0 |
| CD4 lymphocyte none | 0.0 | KU-812 (Basophil) PMA/ionomycin | 45.4 |
| 2ry Th1/Th2/Tr1_anti-CD95 CH11 | 0.0 | CCD1106 (Keratinocytes) none | 9.6 |
| LAK cells rest | 0.0 | CCD1106 (Keratinocytes) TNFalpha + | 0.0 |
| | | IL-1beta | |
| LAK cells IL-2 | 0.0 | Liver cirrhosis | 47.0 |
| LAK cells IL-2 + IL-12 | 0.0 | Lupus kidney | 0.0 |
| LAK cells IL-2 + IFN gamma | 0.0 | NCI-H292 none | 5.0 |
| LAK cells IL-2 + IL-18 | 0.0 | NCI-H292 IL-4 | 0.0 |
| LAK cells PMA/ionomycin | 0.0 | NCI-H292 IL-9 | 0.0 |
| NK Cells IL-2 rest | 11.2 | NCI-H292 IL-13 | 5.7 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 beta | 0.0 |
| PBMC rest | 0.0 | Lung fibroblast none | 0.0 |
| PBMC PWM | 0.0 | Lung fibroblast TNF alpha + IL-1 | 0.0 |
| | | beta | |
| PBMC PHA-L | 12.4 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 0.0 | Lung fibroblast IFN gamma | 0.0 |
| B lymphocytes CD40L and IL-4 | 0.0 | Dermal fibroblast CCD1070 rest | 15.3 |
| EOL-1 dbcAMP | 0.0 | Dermal fibroblast CCD1070 TNF | 0.0 |
| | | alpha | |
| EOL-1 dbcAMP PMA/ionomycin | 0.0 | Dermal fibroblast CCD1070 IL-1 beta | 0.0 |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 0.0 |
| Monocytes rest | 0.0 | IBD Crohn's | 0.0 |
| Monocytes LPS | 0.0 | Colon | 11.1 |
| Macrophages rest | 0.0 | Lung | 0.0 |
| Macrophages LPS | 0.0 | Thymus | 100.0 |
| HUVEC none | 0.0 | Kidney | 0.0 |
| HUVEC starved | 10.0 | | |

CNS_neurodegeneration_v1.0 Summary: Ag2574 Expression of gene CG50349-01 is low/undetectable in all samples on this panel (CTs>34.5). (Data not shown.)

Panel 1.3D Summary: Ag2574 Expression of gene CG50349-01 is low/undetectable in all samples on this panel (CTs>35). (Data not shown.)

Panel 4D Summary: Ag2574 Expression of gene CG50349-01 is restricted to the thymus (CT=34). The putative GPCR encoded for by this gene could therefore play an important role in T cell development. Small molecule therapeutics, or antibody therapeutics designed against the GPCR encoded for by this gene could be utilized to modulate immune function (T cell development) and be important for organ transplant, AIDS treatment or post chemotherapy immune reconstitution.

P. CG50347-01: EBV-INDUCED G PROTEIN-COUPLED RECEPTOR 2 (EB12)-Like Protein

Expression of gene CG50347-01 was assessed using the primer-probe set Ag2572, described in Table 18PA. Results of the RTQ-PCR runs are shown in Tables 18PB and 18PC.

TABLE 18PA

Probe Name Ag2572

| | | | |
|---|---|---|---|
| Primers | Sequences | Length | Start Position |

| | | | |
|---|---|---|---|
| Forward | 5′-cctgtcaaaagagcaggaagat-3′ (SEQ ID NO:172) | 22 | 164 |
| Probe | TET-5′-caactgcacaggcatctacctggtg-3′-TAMRA (SEQ ID NO:173) | 125 | 186 |
| Reverse | 5′-gtagaacagcaggtcagacaca-3′ (SEQ ID NO:174) | 21 | 186 |

TABLE 18PB
Panel 1.3D

| | Rel. Exp. (%) | | RelExp. (%) |
|---|---|---|---|
| | Ag2572, Run | | Ag2572, Run |
| Tissue Name | 166190392 | Tissue Name | 166190392 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 3.6 | Liver (fetal) | 7.1 |
| Brain (amygdala) | 0.0 | Liver ca. (hepatoblast) HepG2 | 0.0 |
| Brain (cerebellum) | 0.0 | Lung | 0.0 |
| Brain (hippocampus) | 0.0 | Lung (fetal) | 0.0 |
| Brain (substantia nigra) | 0.6 | Lung ca. (small cell) LX-1 | 0.0 |
| Brain (thalamus) | 0.0 | Lung ca. (small cell) NCI-H69 | 0.0 |
| Cerebral Cortex | 0.0 | Lung ca. (s. cell var.) SHP-77 | 0.0 |
| Spinal cord | 0.0 | Lung ca (large cell) NCI-H460 | 0.4 |
| glio/astro U87-MG | 0.0 | Lung ca. (non-sm. cell) A549 | 0.5 |
| glio/astro U-118-MG | 1.3 | Lung ca. (non-s. cell) NCI-H23 | 0.3 |
| astrocytoma SW1783 | 0.7 | Lung ca. (non-s. cell) HOP-62 | 0.0 |
| neuro*; met SK-N-AS | 0.0 | Lung ca (non-s. cl) NCI-H522 | 0.0 |
| astrocytoma SF-539 | 0.0 | Lung ca. (squam.) SW 900 | 0.3 |
| astrocytoma SNB-75 | 0.0 | Lung ca. (squam.) NCI-H596 | 0.0 |
| glioma SNB-19 | 0.0 | Mammary gland | 0.0 |
| glioma U251 | 0.0 | Breast ca* (pl. ef) MCF-7 | 0.0 |
| glioma SF-295 | 0.0 | Breast ca.* (pl. ef) MDA-MB-231 | 0.0 |
| Heart (fetal) | 0.5 | Breast ca.* (pl. ef) T47D | 0.0 |
| Heart | 0.4 | Breast ca. BT-549 | 0.0 |
| Skeletal muscle (fetal) | 0.0 | Breast ca. MDA-N | 0.4 |
| Skeletal muscle | 3.6 | Ovary | 0.3 |
| Bone marrow | 0.0 | Ovarian ca OVCAR-3 | 0.0 |
| Thymus | 100.0 | Ovarian ca OVCAR-4 | 0.7 |
| Spleen | 0.0 | Ovarian ca OVCAR-5 | 0.0 |
| Lymph node | 45.4 | Ovarian ca OVCAR-8 | 0.4 |
| Colorectal | 3.5 | Ovarian ca IGROV-1 | 2.8 |
| Stomach | 0.0 | Ovarian ca* (ascites) SK-OV-3 | 0.0 |
| Small intestine | 0.0 | Uterus | 1.1 |
| Colon ca. SW480 | 0.0 | Placenta | 3.4 |
| Colon ca.* SW620 (SW480 met) | 0.0 | Prostate | 0.0 |
| Colon ca. HT29 | 0.0 | Prostate ca.* (bone met) PC-3 | 0.0 |
| Colon ca. HCT-116 | 0.0 | Testis | 0.5 |
| Colon ca. CaCo-2 | 0.0 | Melanoma Hs688(A).T | 0.0 |
| Colon ca. tissue(ODO3866) | 0.0 | Melanoma* (met) Hs688(B).T | 0.0 |
| Colon ca. HCC-2998 | 0.4 | Melanoma UACC-62 | 0.0 |
| Gastric ca.* (liver met) NCI-N87 | 0.7 | Melanoma M14 | 0.0 |
| Bladder | 0.4 | Melanoma LOX IMVI | 0.0 |
| Trachea | 0.0 | Melanoma* (met) SK-MEL-5 | 0.4 |
| Kidney | 0.0 | Adipose | 0.0 |

TABLE 18PC
Panel 4D

| | ReL Exp. (%) | | Rel. Exp. (%) |
|---|---|---|---|
| | Ag2572,Run | | Ag2572, Run |
| Tissue Name | 164331423 | Tissue Name | 164331423 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNFalpha + | 0.1 |
| | | IL-1beta | |
| Primary Th2 act | 0.7 | Microvascular Dermal EC none | 0.0 |
| Primary Tr1 act | 2.1 | Microsvasular Dermal EC TNFalpha + | 0.0 |
| | | IL-1beta | |
| Primary Th1 rest | 3.4 | Bronchial epithelium TNFalpha + | 0.0 |
| | | IL1beta | |
| Primary Th2 rest | 1.7 | Small airway epithelium none | 0.0 |
| Primary Tr1 rest | 3.0 | Small airway epithelium TNFalpha + | 0.0 |
| | | IL-1beta | |
| CD45RA CD4 lymphocyte act | 32.8 | Coronery artery SMC rest | 0.0 |
| CD45RO CD4 lymphocyte act | 16.6 | Coronery artery SMC TNFalpha + | 0.1 |
| | | IL-1beta | |
| CD8 lymphocyte act | 23.8 | Astrocytes rest | 0.0 |
| Secondary CD8 lymphocyte rest | 16.8 | Astrocytes TNFalpha + IL-1beta | 0.0 |
| Secondary CD8 lymphocyte act | 1.8 | KU-812 (Basophil) rest | 0.1 |
| CD4 lymphocyte none | 21.0 | KU-812 (Basophil) PMA/ionomycin | 0.0 |
| 2ry Th1/Th2/Tr1_anti-CD95 CH11 | 10.9 | CCD1106 (Keratinocytes) none | 0.1 |
| LAK cells rest | 5.4 | CCD1106 (Keratinocytes) TNFalpha + | 0.1 |
| | | IL-1beta | |
| LAK cells IL-2 | 12.7 | Liver cirrhosis | 3.3 |
| LAK cells IL-2 + IL-12 | 13.8 | Lupus kidney | 0.0 |
| LAK cells IL-2 + IFN gamma | 17.4 | NCI-H292 none | 0.1 |
| LAK cells IL-2 + IL-18 | 12.2 | NCI-H292 IL-4 | 0.0 |
| LAK cells PMA/ionomycin | 2.6 | NCI-H292 IL-9 | 0.1 |
| NK Cells IL-2 rest | 0.8 | NCI-H292 IL-13 | 0.0 |
| Two Way MLR 3 day | 17.8 | NCI-H292 IFN gamma | 0.0 |
| Two Way MLR 5 day | 7.3 | HPAEC none | 0.0 |
| Two Way MLR 7 day | 9.2 | HPAEC TNF alpha + IL-1 beta | 0.0 |
| PBMC rest | 10.6 | Lung fibroblast none | 0.0 |
| PBMC PWM | 36.6 | Lung fibroblast TNF alpha + IL-1 | 0.0 |
| | | beta | |
| PBMC PHA-L | 92.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 28.5 | Lung fibroblast IFN gamma | 0.0 |
| B lymphocytes CD40L and IL-4 | 4.4 | Dermal fibroblast CCD1070 rest | 0.0 |
| EOL-1 dbcAMP | 0.0 | Dermal fibroblast CCD1070 TNF | 0.0 |
| | | alpha | |
| EOL-1 dbcAMP PMA/ionomycin | 0.1 | Dermal fibroblast CCD1070 IL-1 | 0.0 |
| | | beta | |
| Dendritic cells none | 0.9 | Dermal fibroblast IFN gamma | 0.0 |
| Dendritic cells LPS | 0.2 | Dermal fibroblast IL-4 | 0.0 |
| Dendritic cells anti-CD40 | 1.6 | IBD Colitis 2 | 0.6 |
| Monocytes rest | 1.0 | IBD Crohn's | 0.8 |
| Monocytes LPS | 0.2 | Colon | 1.1 |
| Macrophages rest | 4.4 | Lung | 4.4 |
| Macrophages LPS | 1.1 | Thymus | 0.0 |
| HUVEC none | 0.0 | Kidney | 100.0 |
| HUVEC starved | 0.0 | | |

Panel 1.3D Summary: Ag2572 Expression of the CG50347-01 gene is restricted to the thymus and lymph nodes in this panel (CTs=31-32). The putative GPCR encoded by this gene could therefore play an important role in T cell development. Small molecule therapeutics, or antibody therapeutics designed against the GPCR encoded for by this gene could be utilized to modulate immune function (T cell development) and be important for organ transplant, AIDS treatment or post chemotherapy immune reconstitution.

Panel 2.2 Summary: Ag2572 Expression of the CG50347-01 gene is low/undetectable in all samples in this panel. (CTs>35). (Data not shown.)

Panel 4D Summary: Ag2572: The transcript is expressed in almost exclusively in lymphocytes, which is consistent with the expression profile in panel 1.3 where the transcript is expressed in the thymus and lymph node. The transcript is expressed in resting T cells and T cells that are acutely stimulated but not chronically stimulated. Likewise, stimulated B cells express the transcript but activated tumor cells (RAMOS) do not. Kidney expression is inconsistent with other panels. Therapeutics designed with this sequence or the protein it encodes could be important in regulating T cell activation and be important for immune modulation and in treating T cell mediated diseases such as asthma, allergy, COPD, arthritis, psoriasis and IBD.

### Example 3

SNP Analysis of GPCRX Clones

SeqCalling™ Technology: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing (Pyrosequencing, Westborough, Mass.). Detailed protocols for Pyrosequencing can be found in: Alderborn et al. Determination of Single Nucleotide Polymorphisms by Real-time Pyrophosphate DNA Sequencing. (2000). Genome Research. 10, Issue 8, August. 1249-1265. In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels. The DNA and protein sequences for the novel single nucleotide polymorphic variants are reported. Variants are reported individually but any combination of all or a select subset of variants are also included. In addition, the positions of the variant bases and the variant amino acid residues are underlined.

Equivalents

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, 26, 28, 30, 32 and 34;
(b) a variant of a mature form of an amino acid sequence selected from the group consisting of SEQ ID NOS: 2, 4, 6, 8, 10, 12, 14, 16, 18, 20, 22, 24, 26, 28, 30, 32 and 34, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24, 26, 28, 30, 32 and 34 and
(d) a variant of an amino acid sequence selected from the group consisting of SEQ ID NOS: 2, 4, 6, 8, 10, 12, 14, 16, 18, 20, 22, 24, 26, 28, 30, 32 and 34, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting of SEQ ID NOS: 2, 4, 6, 8, 10, 12, 14, 16, 18, 20, 22, 24, 26, 28, 30, 32 and 34.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS: 1, 3, 5, 7, 9, 11, 13, 15, 17, 19, 21,23, 25, 27, 29, 31, and 33.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, 26, 28, 30, 32 and 34;
(b) a variant of a mature form of an amino acid sequence selected from the group consisting of SEQ ID NOS: 2, 4, 6, 8, 10, 12, 14, 16, 18, 20, 22, 24, 26, 28, 30, 32 and 34, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24, 26, 28, 30, 32 and 34;
(d) a variant of an amino acid sequence selected from the group consisting SEQ ID NOS: 2, 4, 6, 8, 10, 12, 14, 16, 18, 20, 22, 24, 26, 28, 30, 32 and 34, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS: 2, 4, 6, 8, 10, 12, 14, 16, 18, 20, 22, 24, 26, 28, 30, 32 and 34, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS: 1, 3, 5, 7, 9, 11, 13, 15, 17, 19, 21, 23, 25, 27, 29, 31 and 33.

9. The nucleic acid molecule of claim 5, wherein said nucleic acid molecule comprises a nucleotide sequence selected from the group consisting of:
(a) a nucleotide sequence selected from the group consisting of SEQ ID NOS: 1, 3, 5, 7, 9, 11, 13, 15, 17, 19, 21, 23, 25, 27, 29, 31 and 33
(b) a nucleotide sequence differing by one or more nucleotides from a nucleotide sequence selected from the group consisting of SEQ ID NOS: 1, 3, 5, 7, 9, 11, 13, 15, 17, 19, 21, 23, 25, 27, 29, 31 and 33, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NOS: 1, 3, 5, 7, 9, 11, 13, 15, 17, 19, 21, 23, 25, 27, 29, 31 and 33, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide t hat is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule, thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide, whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a GPCRX-associated disorder, said method comprising administering to a subject in which such treatment or prevention is desired the polypeptide of claim 1 in an amount sufficient to treat or prevent said GPCRX-associated disorder in said subject.

27. The method of claim 26 wherein the disorder is selected from the group consisting of cardiomyopathy and atherosclerosis.

28. The method of claim 26 wherein the disorder is related to cell signal processing and metabolic pathway modulation.

29. The method of claim 26, wherein said subject is a human.

30. A method of treating or preventing a GPCRX-associated disorder, said method comprising administering to a subject in which such treatment or prevention is desired the nucleic acid of claim 5 in an amount sufficient to treat or prevent said GPCRX-associated disorder in said subject.

31. The method of claim 30 wherein the disorder is selected from the group consisting of cardiomyopathy and atherosclerosis.

32. The method of claim 30 wherein the disorder is related to cell signal processing and metabolic pathway modulation.

33. The method of claim 30, wherein said subject is a human.

34. A method of treating or preventing a GPCRX-associated disorder, said method comprising administering to a subject in which such treatment or prevention is desired the antibody of claim 15 in an amount sufficient to treat or prevent said GPCRX-associated disorder in said subject.

35. The method of claim 34 wherein the disorder is diabetes.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to cancers.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS: 2, 4, 6, 8, 10, 12, 14, 16, 18, 20, 22, 24, 26, 28, 30, 32 and 34, or a biologically active fragment thereof.

49. A method of treating a pathological state in a mammal, the method comprising administering to the mammal the antibody of claim 15 in an amount sufficient to alleviate the pathological state.

50. A method for the screening of a candidate substance interacting with an olfactory receptor polypeptide selected from the group consisting of SEQ ID NOS: 2, 4, 6, 8, 10, 12, 14, 16, 18, 20, 22, 24, 26, 28, 30, 32 and 34, or fragments or variants thereof, comprises the following steps:
a) providing a polypeptide selected from the group consisting of the sequences of SEQ ID NOS: 2, 4, 6, 8, 10, 12, 14, 16, 18, 20, 22, 24, 26, 28, 30, 32 and 34, or a peptide fragment or a variant thereof;
b) obtaining a candidate substance;
c) bringing into contact said polypeptide with said candidate substance; and
d) detecting the complexes formed between said polypeptide and said candidate substance.

51. A method for the screening of ligand molecules interacting with an olfactory receptor polypeptide selected from the group consisting of SEQ ID NOS: 2, 4, 6, 8, 10, 12, 14, 16, 18, 20, 22, 24, 26, 28, 30, 32 and 34, wherein said method comprises:
a) providing a recombinant eukaryotic host cell containing a nucleic acid encoding a polypeptide selected from the group consisting of the polypeptides comprising the amino acid sequences SEQ ID NOS: 2, 4, 6, 8, 10, 12, 14, 16, 18, 20, 22, 24, 26, 28, 30, 32 and 34;
b) preparing membrane extracts of said recombinant eukaryotic host cell;
c) bringing into contact the membrane extracts prepared at step b) with a selected ligand molecule; and
d) detecting the production level of second messengers metabolites.

52. A method for the screening of ligand molecules interacting with an olfactory receptor polypeptide selected from the group consisting of SEQ ID NOS: 2, 4, 6, 8, 10, 12, 14, 16, 18, 20, 22, 24, 26, 28, 30, 32 and 34, wherein said method comprises:
a) providing an adenovirus containing a nucleic acid encoding a polypeptide selected from the group consisting of polypeptides comprising the amino acid sequences SEQ ID NOS: 2, 4, 6, 8, 10, 12, 14, 16, 18, 20, 22, 24, 26, 28, 30, 32 and 34;
b) infecting an olfactory epithelium with said adenovirus;
c) bringing into contact the olfactory epithelium b) with a selected ligand molecule; and
d) detecting the increase of the response to said ligand molecule.
